SEQUENCE LISTING (2 sequences)

SEQ ID NO: 1 - protein, 483 aa, Homo sapiens
MSGGGPSGGGPGGSGRARTSSFAEPGGGGGGGGGGPGGSASGPGGTGGGKASVGAMGGGVGASSSGGGPGGSGGGGSGGPGAGTSFPPPGVKLGRDSGKVTTVVATLGQGPERSQEVAYTDIKVIGNGSFGVVYQARLAETRELVAIKKVLQDKRFKNRELQIMRKLDHCNIVRLRYFFYSSGEKKDELYLNLVLEYVPETVYRVARHFTKAKLTIPILYVKVYMYQLFRSLAYIHSQGVCHRDIKPQNLLVDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFKSRTPPEAIALCSSLLEYTPSSRLSPLEACAHSFFDELRCLGTQLPNNRPLPPLFNFSAGELSIQPSLNAILIPPHLRSPAGTTTLTPSSQALTETPTSSDWQSTDATPTLTNSS

SEQ ID NO: 2 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

(19) United States
(12) Patent Application Publication
(10) Pub. No.: US 2009/0171641 A1
(43) Pub. Date: Jul. 2, 2009

(54) NOVEL HOMOLOGY MODEL OF THE GLYCOGEN SYNTHASE KINASE 3 ALPHA AND ITS USES THEREOF

(75) Inventors: Samiron Phukan, Bangalore (IN); Balaji Narayana Iyengar Vitukudi, Bangalore (IN); Prasanna Dattatreya Marahanakuli, Bangalore (IN)

Correspondence Address: SUGHRUE MION, PLLC, 2100 PENNSYLVANIA AVENUE, N.W., SUITE 800, WASHINGTON, DC, 20037, US

(73) Assignee: Jubilant Biosys Ltd., Bangalore (IN)

(21) Appl. No.: 12/096,849

(22) PCT Filed: Aug. 14, 2006
(86) PCT No.: PCT/IN2006/000290

§ 371 Date: Jun. 10, 2008

(30) Foreign Application Priority Data
Aug. 17, 2005 (IN) 1145/CHE/2005

(51) Int. Cl. G06G 7/48; C12N 9/12

(52) U.S. Cl. 703/11; 435/194

(57) ABSTRACT

The present invention provides a novel homology three-dimensional structure of Glycogen Synthase Kinase 3-Alpha (GSK3-Alpha) or a variant thereof for screening, designing and/or synthesizing a modulator that modulates the activity of GSK3-Alpha and method of generating said three dimensional structure thereof. The three-dimensional structure of GSK3-Alpha is defined by at least a portion of structural co-ordinates of Table 1. Further, the present invention provides a computer assisted system for generating said three-dimensional structure of GSK3-Alpha or a variant thereof. Further, the present invention relates to a method of screening, designing and/or synthesizing a modulator for modulating the activity of GSK3-? or a variant thereof.

### TECHNICAL FIELD

The present invention relates to a novel three-dimensional homology model (structure) of Glycogen Synthase Kinase3-Alpha (GSK3-Alpha) and uses thereof. More specifically, the invention relates to a method of screening modulators which modulate the activity of GSK3-Alpha using a novel homology three-dimensional model of GSK3-Alpha.

### BACKGROUND OF THE INVENTION

The Glycogen Synthase Kinase3 (GSK3), a serine/threonine protein kinase family, comprises of Alpha and Beta isoforms, with integral roles in many aspects of cellular metabolism, proliferation, differentiation and development by phosphorylating and modulating the activity of a number of regulatory proteins in certain cell signaling pathways. Hence, GSK3 has been implicated in various diseases like diabetes, Alzheimer's disease, CNS disorders such as manic depressive disorder and neurodegenerative diseases, and cardiomyocete hypertrophy [WO 99/65897; WO 00/38675; and Haq et al., J. Cell Biol. (2000) 151, 117]. The regulatory proteins include the glycogen synthase which is the rate limiting enzyme necessary for glycogen synthesis, the microtubule associated protein Tau, the gene transcription factor β-catenin, the translation initiation factor e1F2B, as well as ATP citrate lyase, axin, heat shock factor-1, c-Jun, c-Myc, c-Myb, CREB, and CEPBα. It can thus be seen that these enzymes represent interesting pharmaceutical targets since complications arising from the expression of these enzymes could be treated by kinase inhibitors specific for the GSK3. Small molecules that inhibit GSK3 have recently been reported [WO 1999/65897 and WO 2000/38675].

The approach of homology modeling has been undertaken in cases of other proteins. There are few homology built models reported in the prior art such as U.S. Pat. No. 6,355,678 which discloses the homology model for EGF-R Kinase domain and the docking procedure for the same which is useful to rationally design compounds predicted to bind favorably to EGF-R Kinase & inhibit EGF-R TK activity. United States Patent 2005/4766 discloses the methods for obtaining and sequencing the proteins that are product of the genes exhibiting polymorphisms and determining the 3-D protein structural variant models for the proteins exhibiting the polymorphism and building a database. PCT application 2000/52050 discloses the homology model of a glucocorticoid receptor. PCT application 2005/47526 relates to the homology model of PLK and to the use thereof in assays for the identification of small molecules PLK modulators. PCT application 2005/2147 discloses the homology model for CDK4 and use thereof in assays for the identification of small molecule CDK 4 modulators. PCT application 1998/56812 discloses the Homology model for ER β receptor. A number of compounds have been described in literature that show preferential binding to GSK3-Beta. However, to date there has been no significant progress in determining the structural features required for selectivity towards GSK3-α. A major reason for this lack of insight has been the paucity of structural information for this isoform. In contrast, there is a plethora of structural data for GSK3-Beta as it has been crystallised in many forms including in complex with natural ligands as inhibitor complexes with the monomeric and activated forms. There is therefore tremendous scope for further development of new modulators of GSK3-Alpha family.

### SUMMARY OF THE INVENTION

Systems and methods for building a three-dimensional homology model/structure of Glycogen Synthase Kinase 3-Alpha (GSK-3 Alpha) or its variants are described. The 3-D structure thus constructed is defined by at least a portion of the structure co-ordinates shown in Table 1. Methods to screen modulators for modulating the activity of GSK-3 Alpha using the structure coordinates of the model are also described. To screen a modulator of GSK3-α or its variant, the modulator is identified by using at least a portion of the structure co-ordinates shown in Table 1. The identified modulator is contacted with GSK3-α or its variant and the modulating activity of the identified modulator is determined.

### BRIEF DESCRIPTION OF DRAWINGS

FIG. 1: Flow chart representation of homology modeling process.

FIG. 2: Illustrates the GSK3-Alpha homology model structure.

FIG. 3: Flow chart representation of the docking process.

FIG. 4: Depicts the docking pose of GSK3-Alpha.

### DETAILED DESCRIPTION OF THE INVENTION

The present invention relates to a three-dimensional model/structure of Glycogen Synthase Kinase 3-Alpha (GSK3-Alpha) or a variant thereof for screening a modulator that modulates the activity of GSK3-Alpha.

### DEFINITIONS AND ABBREVIATIONS

The Glycogen Synthase Kinase 3-Alpha is herein after referred to as “GSK3-Alpha”. The GSK3-Alpha described is intended to include any polypeptide, which has the activity of the naturally occurring GSK3-Alpha and includes all vertebrate and mammalian forms. Such terms also include polypeptides that differ from naturally occurring forms of GSK3-Alpha by having amino acid deletions, substitutions, and additions, but which retain the activity of GSK3-Alpha.

The terms “model” and “structure” are used interchangeable hereinafter.

The amino acid sequence of GSK3-Alpha is shown in SEQ ID NO: 1. The amino acid sequence of GSK3-Beta is shown in SEQ ID NO: 2.

A fragment, a homologue, a variant, a mutant, or a derivative of GSK3-Alpha or a naturally occurring polypeptide or nucleotide sequences which differs from a wild-type or a native sequence is herein after referred to as “variant” of GSK3-Alpha. The term variant also encompasses amino acid sequence comprising at least 50%, more preferably at least 65%, more preferably at least 80%, most preferably at least 90% of the wild-type sequence.

The term “fragment” indicates that a polypeptide or nucleotide sequence comprises a fraction of a wild-type or a native sequence. It may comprise one or more large contiguous sections of sequence or a plurality of small sections.

The term “homologue” refers to an entity that shares a certain homology with amino acid sequences or nucleotide sequences of GSK3-Alpha. Here, the term “homology” can be equated with “identity”. In the present context, a homologous sequence is taken to include an amino acid sequence which may be at least 70, 80 or 90% identical, to the subject sequence. Although homology can also be considered in terms of similarity (i.e. amino acid residues having similar chemical properties/functions), it is preferred to express homology in terms of sequence identity. An homologous sequence is taken to include a nucleotide sequence which may be at least 70, 80 or 90% identical, to the subject sequence. Homology comparisons can be conducted by eye, or more usually, with the aid of readily available sequence comparison programs. These commercially available computer programs can calculate % homology between two or more sequences. % homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

The term “derivative” or “derivatised” herein after includes chemical modification of an entity, such as candidate compound or a GSK3-Alpha modulator.

The compounds that are can affect the activity of GSK-3 Alpha by preventing, suppressing, inhibiting, alleviating, restorating, elevating, increasing or otherwise is herein after referred to as modulator(s). The GSK3-Alpha modulator may be an antagonist or an agonist of GSK3-Alpha.

The term “agonist” means any entity, which is capable of interacting (eg. binding) with GSK3-Alpha and which is capable of increasing a proportion of the GSK-3 Alpha that is in an active form, resulting in an increased biological response.

The term “antagonist” means any entity, which is capable of interacting (eg. binding) with GSK3-Alpha and which is capable of decreasing (eg. inhibiting) a proportion of the GSK3-Alpha that is in an active form, resulting in a decreased biological response.

The term “amino acid sequence” as herein after is synonymous with the term “polypeptide” and/or the term “protein”. In some instances, the term “amino acid sequence” is synonymous with the term “peptide”.

An embodiment of the present invention relates to said three-dimensional model structure of GSK3-α or a variant thereof for screening a modulator that modulates the activity of GSK3-Alpha by using at least a portion of structure co-ordinates as shown in Table 1.

Another embodiment of the present invention relates to said three-dimensional model structure of GSK3-Alpha or a variant thereof wherein GSK3-Alpha comprises the amino acid sequence set forth in SEQ ID NO: 1.

Another embodiment of the present invention relates to said three-dimensional model structure, wherein said structure co-ordinate comprises a ligand binding domain defined by amino acid residue selected from VAL 49, ASP 106, GLU 39, GLN 91, ASN 92, LYS 89 and combinations thereof.

Another embodiment of the present invention relates to a computer assisted system for producing a three-dimensional structure of GSK3-Alpha or a variant thereof which comprises: a computer readable data storage medium comprising a data storage material encoded with computer readable data, wherein said data comprises at least a portion of the structure co-ordinates of Table 1; a working memory for storing instructions for processing said computer readable data; a central processing unit coupled to said computer readable data storage medium and said working memory for processing said computer readable data into said three-dimensional structure; and a display coupled to said central processing unit for displaying said three-dimensional structure.

Another embodiment of the present invention relates to a method of generating said three dimensional structure of GSK3-Alpha or a variant thereof by employing said computer assisted system.

Another embodiment of the present invention relates to a method of designing a modulator that modulates the activity of GSK3-Alpha or a variant thereof, wherein said method comprises: providing at least a portion of the structure co-ordinates of Table 1; employing said structure co-ordinates to design a modulator of GSK3-Alpha or a variant thereof; contacting said modulator with GSK3-α or variant to determine the modulating activity of said modulator.

Another embodiment of the present invention relates to said method of designing a modulator, wherein said structure co-ordinate comprises a ligand binding domain defined by amino acid residue selected from VAL 49, ASP 106, GLU 39, GLN 91, ASN 92, LYS 89 and combinations thereof.

Another embodiment of the present invention relates to a method of screening a modulator for modulating the activity of GSK3-Alpha or a variant thereof comprising the following: providing at least a portion of the structure co-ordinates of Table 1; employing said structure co-ordinates to identify a modulator of GSK3-Alpha or variant; contacting said modulator with GSK3-α or variant, and determining the modulating activity of said modulator. GSK3-α comprises the amino acid sequence set forth in SEQ ID NO: 1.

Another embodiment of the present invention relates to a method of screening a modulator for modulating the activity of GSK3-Alpha or a variant thereof, wherein said structure co-ordinate comprises a ligand binding domain defined by amino acid residue selected from VAL 49, ASP 106, GLU 39, GLN 91, ASN 92, LYS 89 and combinations thereof.

Another embodiment of the present invention relates to a method of screening a modulator for modulating the activity of GSK3-Alpha or a variant thereof, wherein the modulator inhibits GSK3-α activity. The steps prior to screening of the modulator that modulates the activity of GSK3-Alpha includes designing, selecting and synthesizing of the modulators

Another embodiment of the present invention relates to a method of screening a modulator for modulating the activity of GSK3-Alpha or a variant thereof, wherein the modulator is associated with preventing and/or treating GSK3-Alpha related diseases selected from HIV/AIDS, angiogenesis, breast cancer, colon cancer, type II diabetes, glioblastoma, pancreatic cancer, xaelanoma, prostrate cancer, and schizophrenia.

Another embodiment of the present invention relates to the use of the three-dimensional structure for designing, selecting and synthesizing a modulator that modulates the activity of GSK3-Alpha.

Another embodiment of the present invention relates to the use of a computer assisted system for producing a three-dimensional structure of GSK3-Alpha or a variant thereof for screening a modulator that modulates the activity of GSK3-Alpha.

Another embodiment of the present invention relates to the use of a computer assisted system for producing a three-dimensional structure of GSK3-Alpha or a variant thereof for designing, selecting and synthesizing a modulator that modulates the activity of GSK3-Alpha.

Another embodiment of the present invention relates to a compound for modulating the activity of GSK3-Alpha or a variant thereof by using the method of screening of modulators of GSK3-Alpha or a variant thereof.

Another embodiment of the present invention relates to the use of the three-dimensional structure of GSK3-Alpha or a variant thereof to determine the structure of the crystalline form of a protein with significant amino acid sequence homology to any functional domain of GSK3 Alpha.

Another embodiment of the present invention relates to the use of the three-dimensional model structure of GSK3-Alpha or a variant thereof, wherein the structure of the crystalline form of a protein with significant amino acid sequence homology to any functional domain of GSK3 Alpha is determined using molecular replacement.

Further, the present invention relates to a three-dimensional model structure that is used in conjunction with automated flexible docking methods to generate a set of binding hypotheses for the ligand-GSK3-Alpha complexes of a number of modulators. Based on analysis of the sequence and residues lining the ATP binding pocket, the specific residues and side chains which differ in the GSK3-Alpha structure were determined and the interactions that could be exploited in the design of selective compounds were identified. This information could be used in two ways, both in the enhancement of GSK3-Alpha selectivity and in the engineering of modulators with greater binding specificity for GSK3-Alpha.

In one particularly preferred embodiment, the GSK3-Alpha modulator or candidate compound of the invention has an ionizable functional group capable of forming associations with the amino acid residues corresponding to GSK3-Alpha amino acid residues V41, D106, E39, Q91, N92, K89 of GSK3-Alpha. More preferably still, the ionizable functional group is a group which is positively charged at physiological pH. Even more preferably, the ionizable functional group is an NH group which is positively charged at physiological pH.

In one preferred embodiment, the GSK3-Alpha modulator or candidate compound of the invention comprises a functional group capable of forming a hydrogen bond with the amino acid residue corresponding to V41, D106, E39, Q91, N92, K89.

In one preferred embodiment, the GSK3-Alpha modulator or candidate compound of the invention comprises a group which is capable of steric interaction with the amino acid residues corresponding to V41, D106, E39, Q91, N92, K89 of the ATP binding site of GSK3-Beta.

In a preferred embodiment, the GSK3-Alpha modulator or candidate compound of the invention is an irreversible antagonist.

By way of example, the structure co-ordinates of Table 1 may be used to design compounds that bind to GSK3-Alpha and may alter the physical properties of the compounds (eg. solubility) or GSK3-Alpha itself. This invention may be used to design compounds that act as modulators, such as competitive inhibitors-of GSK3-Alpha by binding to all or a portion of the active site of GSK3-Alpha. Compounds may also be designed that act as non-competitive inhibitors of GSK3-Alpha. These non-competitive inhibitors may bind to all or a portion of GSK3-Alpha already bound to its substrate and may be more potent and specific than known GSK3-Alpha inhibitors that compete only for the GSK3-Alpha active site. Similarly, non-competitive inhibitors that bind to and inhibit GSK3-Alpha whether or not it is bound to another chemical entity may be designed using the structure co-ordinates of GSK3-Alpha described herein.

The present invention also allows the development of compounds or its isomers that can act as reaction intermediates in a chemical reaction of a substrate that binds to GSK3-Alpha. Thus, the time-dependent analysis of structural changes in GSK-3 Alpha during its interaction with other molecules may be performed. The reaction intermediates of GSK3-Alpha may also be deduced from the reaction product in co-complex with GSK3-Alpha. Such information is especially useful to design improved analogues of known GSK3-Alpha modulators or to design new GSK3-Alpha modulators based on the reaction intermediates of the GSK3-Alpha enzyme and GSK3-Alpha-modulator complex. This provides a new route for designing GSK3-Alpha modulators with high specificity and stability. Preferably, this provides a new route for designing GSK3-Alpha modulators with high specificity, high stability and low toxicity.

Small molecule databases for modulators or candidate compounds may be screened for chemical entities that can bind in whole, or; in part, to GSK3-Alpha. Thus, in a preferred embodiment, the GSK3-Alpha modulators form a library of compounds or a database. In this screening, the quality of fit of such entities or compounds to the binding site may be judged by various methods-such as shape complementarity or estimated interaction energy (Meng, E. C. et al., J. Comp. Chem., 13, pp. 505-524 (1992)).

The structure co-ordinates of Table 1, or portions thereof, may also be useful in solving the structure of crystal forms of GSK3-Alpha. They may also be used to solve the structure of variants of GSK3-Alpha or GSK3-Alpha complexes.

Preferably, the structure co-ordinates of Table 1 are used to solve the structure of the crystalline form of proteins having significant amino acid sequence homology to any functional domain of GSK3-Alpha.

The structure co-ordinates of the homology model of the present invention may also facilitate the identification of related proteins or enzymes analogous to GSK3-Alpha in function, structure or both, thereby further leading to novel therapeutic modes for treating or preventing GSK3-Alpha related diseases.

The design of compounds that bind to or modulate GSK3-Alpha according to the present invention involves consideration of two factors. Firstly, the compound must be capable of physically and structurally associating with GSK3-Alpha. Non-covalent molecular interactions important in the association of GSK3-Alpha with its substrate may include hydrogen bonding, van der Waals and hydrophobic interactions. Secondly, the compound must be able to assume a conformation that allows it to associate with GSK-3Alpha. Although certain portions of the compound may not directly participate in the association with GSK3-Alpha, those portions may still influence the overall conformation of the molecule. This will have a significant impact on the activity. Such conformational requirements include the overall three-dimensional structure and orientation of the chemical entity or compound in relation to all or a portion of a binding site of GSK-3Alpha, or the spacing between functional groups of a compound comprising several chemical entities that directly interact with GSK3-Alpha.

The potential modulating or binding effect of a chemical compound on GSK3-Alpha may be analyzed prior to its actual synthesis and testing by the use of computer modeling techniques. If the theoretical structure of the given compound suggests insufficient interaction and association with GSK3-Alpha, then synthesis and testing of the compound may be obviated. However, if computer modeling indicates a strong interaction, the molecule may be synthesized and tested for its ability to bind to GSK3-Alpha and modulate the activity of GSK3-Alpha (e.g. inhibit) using the fluorescent substrate assay of Thornberry et al. (2000) Methods.

The modulator of GSK3-Alpha may be an organic compound or a biochemical compound or any other chemical compound. The modulator of GSK3-Alpha may be a compound, which is obtainable from or produced by any suitable source, whether natural or artificial. The modulator of GSK3-Alpha may be an amino acid molecule, a polypeptide, or a chemical derivative thereof, or a combination thereof. The modulator of GSK3-Alpha may even be a polynucleotide molecule, which can be a sense or an anti-sense molecule. The modulator of GSK3-Alpha may even be an antibody.

The modulator of GSK3-Alpha may be designed or obtained from a library of compounds, which may comprise peptides, as well as other compounds, such as small organic molecules.

By way of example, the modulator of GSK3-Alpha may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesized synthetically (such as, by way of example, either using a peptide synthesizer or by recombinant techniques or combinations thereof, a recombinant agent, an antibody, a natural or a non-natural agent, a fusion protein or equivalent thereof and mutants, derivatives or combinations thereof).

In case the modulator of GSK3-Alpha is an organic compound, the organic compound will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C, then those carbons need not necessarily be linked to each other. For example, at least two of the carbons may be linked via a suitable element or group. Thus, the hydrocarbyl group may contain hetero atoms. Some of the examples of suitable hetero atoms are sulphur, nitrogen and oxygen. For some applications, preferably the modulator of GSK3-Alpha comprises at least one cyclic group. The cyclic group may be a polycyclic group, such as a non-fused polycyclic group. For some applications, the modulator of GSK3-Alpha comprises at least one of said cyclic groups linked to another hydrocarbyl group.

The modulator of GSK3-Alpha which modulates the activity of GSK3-Alpha can be agonist or antagonist in nature.

The term “agonist” means any entity, which is capable of interacting (eg. binding) with GSK3-Alpha and which is capable of increasing a proportion of the GSK-3 Alpha that is in an active form, resulting in an increased biological response.

The term “antagonist” means any entity, which is capable of interacting (eg. binding) with GSK3-Alpha and which is capable of decreasing (eg. inhibiting) a proportion of the GSK3-Alpha that is in an active form, resulting in a decreased biological response.

The modulator of GSK3-Alpha may contain halo groups, for example, fluoro, chloro, bromo or iodo groups, or one or more of alkyl, alkoxy, alkenyl, alkylene and alkenylene groups, each of which may be branched or unbranched.

The modulator of GSK3-Alpha is a structurally novel modulator of GSK3-Alpha, or an analogue of a known modulator of GSK3-Alpha.

The modulator of GSK3-Alpha may be a mimetic, or may be chemically modified. The modulator of GSK3-Alpha may be capable of displaying various therapeutic properties.

The modulator of GSK3-Alpha may be used in combination with one or more other pharmaceutically active agents. If combinations of active agents are administered, then they may be administered simultaneously, separately or sequentially.

### Percentage Homology Calculation and Substituents

The % homology calculation of a protein is a very simple and consistent method, but it fails to take into consideration factors such as in an otherwise identical pair, of sequences, one insertion or deletion that will cause the following amino acid residues to be put out of alignment, thus potentially resulting in a large reduction in % homology when a global alignment is performed.

Consequently, most sequence comparison methods are designed to produce optimal alignments that take into consideration possible insertions and deletions without penalizing unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible-reflecting higher relatedness between the two compared sequences-will achieve a higher score than one with many gaps; “Affine gap costs” are typically used when relatively high cost is charged for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the Clustal W package the default gap penalty for amino acid sequences is 10 for a gap and 0.2 for each extension and End Gap as −1 with GONNET matrix.

Calculation of maximum % homology therefore firstly requires the production of an optimal alignment, taking into consideration gap penalties. A suitable computer program for carrying out such an alignment is the Clustal W (Ref: www.ebi.ac.uk/clustalw) package. Examples of other software than can perform sequence comparisons include, but are not limited to, the BLAST package (see Ausubel et al., 1999 ibid-Chapter18), FASTA (Altschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). A new tool, called BLAST 2 Sequences is also available for comparing protein and nucleotide sequence (see FEMS Microbiol Lett 1999 174 (2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pair wise comparison based on chemical similarity or evolutionary distance. An example of such a matrix commonly used is the GONNET matrix—the default matrix for the Clustal W suite of programs. Clustal W programs generally use either the public default values or a custom symbol comparison table if supplied (see user manual for further details). For some applications, it is preferred to use the public default values for the Clustal W, or in the case of other software, the default matrix, such as GONNET.

Once the software has produced an optimal alignment, it is possible to calculate % homology, preferably % sequence identity. The software typically does this as part of the sequence comparison and generates a numerical result.

The amino acid sequence of GSK3-Alpha may be isolated from a suitable source, or it may be made synthetically or it may be prepared by use of recombinant DNA techniques. The amino acid sequence of GSK3-Alpha is shown in SEQ ID NO: 1. The three dimensional structure of GSK3-Alpha or variant thereof is constructed using at least a portion of the structure co-ordinates provided in Table 1. The variant of GSK3-Alpha encompasses of a fragment, a homologue, a variant, a mutant, or a derivative of GSK3-Alpha or a naturally occurring polypeptide or nucleotide sequences which differ from a wild-type or a native sequence of GSK3-Alpha as shown in SEQ ID NO: 1. The term variant also encompasses amino acid sequence comprising at least 50%, more preferably at least 65%, more preferably at least 80%, most preferably at least 90% of the wild-type sequence.

The term “fragment” indicates that a polypeptide or nucleotide sequence comprises a fraction of a wild-type or a native sequence. It may comprise one or more large contiguous sections of sequence or a plurality of small sections.

The term “homologue” refers to an entity that shares a certain homology with amino acid sequences or nucleotide sequences of GSK3-Alpha. Here, the term “homology” can be equated with “identity”. In the present context, a homologous sequence is taken to include an amino acid sequence which may be at least 70, 80 or 90% identical, to the subject sequence. Although homology can also be considered in terms of similarity (i.e. amino acid residues having similar chemical properties/functions), it is preferred to express homology in terms of sequence identity.

An homologous sequence is taken to include a nucleotide sequence which may be at least 70, 80 or 90% identical, to the subject sequence.

The term “derivative” or “derivatised” herein after includes chemical modification of an entity, such as candidate compound or a GSK3-Alpha modulator.

The amino acid sequences may also have deletions, insertions or substitutions of amino acid residues, which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table 2 given below. Amino acids in the same block in the second column and preferably in the same line in the third column may be substituted for each other.

Homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural, amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, 3-alanine*, L-α-amino butyric acid*, L-γ-aminobutyric acid*, L-α-amino isobutyric acid*, L-s-amino caproic acid #, 7-amino heptanoic acid*, L-methionine sulfone#, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid* and L-Phe (4-benzyl)*. The notation * has been used for the purpose of the discussion above (relating to homologous or non-homologous substitution), to indicate the hydrophobic nature of the derivative. The notation # has been used to indicate the hydrophilic nature of the derivative and #* indicates amphipathic characteristics.

Illustrative of chemical modifications/derivatives would be replacement of hydrogen by a halo group, an alkyl group, an acyl group or an amino group. Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form.

The term “peptoid form” is used to refer to variant amino acid residues wherein the a-carbon substituent group is on the residue's nitrogen atom rather than the a-carbon. Processes for preparing peptides in the peptoid form are known in the art, for example Simon R J et al., PNAS (1992)89 (20), 9367-9371 and Horwell D C, Trends Biotechnol. (1995) 13 (4), 132-134.

### Homology Model Construction

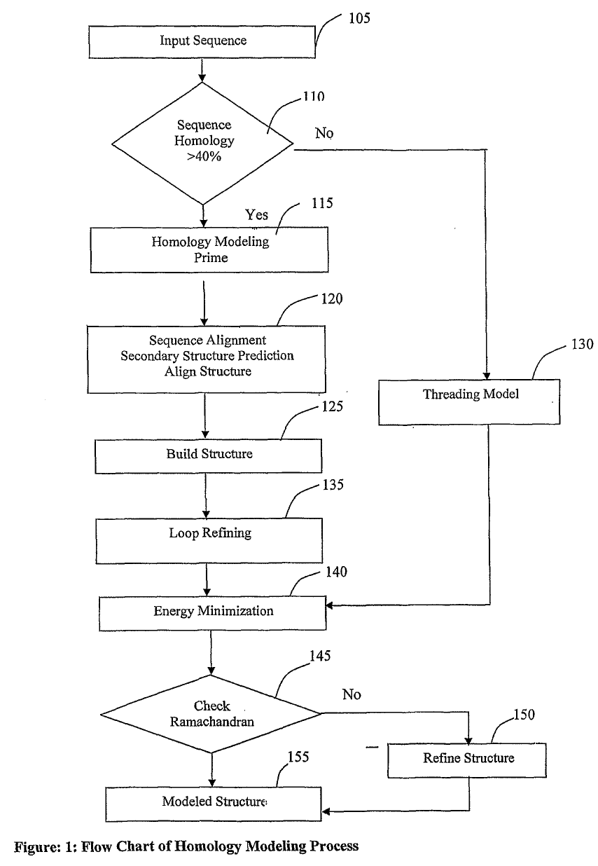

The homology model for GSK3-Alpha was generated using the program module Prime within the Schrodinger Discovery Suite (Schrodinger Inc, NY, USA). The flow chart for homology modeling approach is shown in FIG. 1.

At block 105, the GSK3-Alpha sequence (SEQ ID No. 1) was given as the input sequence. At block 110, a sequence homology search was conducted to identify templates having sequence homology greater than 40%. A template having greatest sequence homology amongst the identified templates was selected for model construction. For the present model construction, the GSK-3 Beta structure was used as template. At block 115, the GSK3-Alpha was subjected to modeling based on the selected template (GSK-3 Beta) using the program module Prime. At block 120, sequence alignment of GSK3-Alpha with the selected template was performed using the PAM 120 multiple scoring matrix with a dimension block of 0.6 and a significance p value of 0.0001. The strategy involved using the selected template to define the structurally conserved regions (SCR's) from which the coordinates were subsequently transferred to build the structure at block 125. This was then followed by loop construction at block 135, where the non-SCR's were generated from the selected template and additionally by de-novo building and subsequent evaluation of the most realistic coordinates (in terms of energetic of the loop itself and the exclusion of loops leading to overlapping atoms). After loop building was completed for missing coordinates, the raw coordinates were refined using successive rounds of end repair and energy minimization (500 steps of OPLS 2005) at block 140. The model was then completed through using a further minimization. 1 ps molecular dynamics was used to fully explore the conformational space of the loop regions. At block 145, the final model structure was checked against databases of protein structures for bond length and dihedral angle violations using Ramachandran plot. If the Ramachandran plot was not found to be within acceptable ranges, then further refinement was carried out at block 150. Finally, at block 155, a model was built using the acceptable structure. The output of the final modeled structure was obtained in PDB format.

In case any sequence of homology greater than 40% is not identified from the sequence homology search at block 110, an alternate method of modeling by threading methodology is used at block 130 to build a model. Here the different parts of the proteins are taken from the fold libraries and the model is built. This model is also further refined by energy minimization and using Ramachandran plot as before.

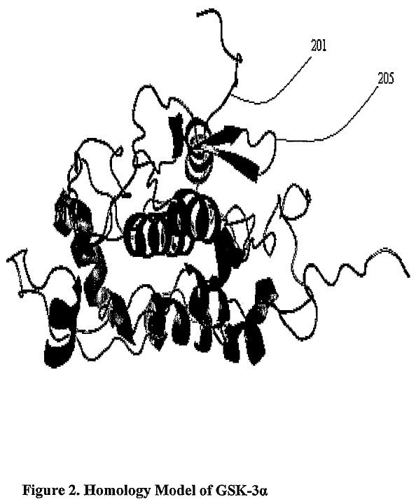

For the final model structure for GSK3-Alpha built based on the selected template, the results indicated that bond length and dihedral angle violations as a whole were within acceptable limits with greater than 80% of residues having phi-psi plots with the allowed region in Ramachandran space. The final model structure that was built had the structure coordinates given in table 1. The final structure is shown in FIG. 2. The binding region or active site 201 is just above the activation loop site 205.

### Molecular Modeling of GSK-3Alpha Modulator

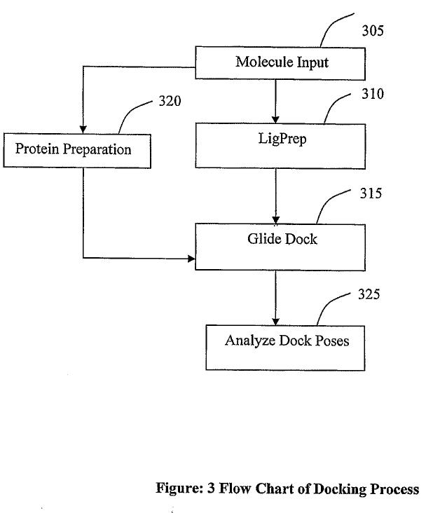

The structure coordinates of the ternary complex of GSK3-Beta were obtained from the RCSB (accession code 1J1B). GSK-3Alpha inhibitors were then docked into the ATP site of the GSK-3 Beta or the GSK-3 Alpha model structure using the Glide program (Schrodinger Inc, NY, USA). The flow chart of the docking protocol is described in FIG. 3.

The GSK3 Alpha was given as an input molecule at 305 and the binding site was defined as an 8A radius from the centre of a ligand binding mode. The calculation was performed using the OPLS force field in a two-step process using an implicitly derived solvation model and geometric H-bond restraints. The refinement phase involved conformational sampling using molecular dynamics calculated over 5 ps in 100 fs stages, where the temperature was at 300 K. The Ligand was prepared at 310 by the Ligprep module of the Schrodinger discovery suite where around five conformations were generated. The modeled protein is subjected to protein preparation under protein refinement method at 320. These conformations were subjected to XP docking protocol of Glide without any constraints at 315. The docked pose was then analyzed at 325.

This molecular docking routine incorporates a full molecular mechanics approach and allows for flexibility both in the ligand and in the side chains and backbone of the receptor (the positions of the alpha carbon atoms were fixed during the simulation). The specificity analysis can be evident in the docking studies that were carried out using Glide module of Schrodinger Discovery suite and are discussed below.

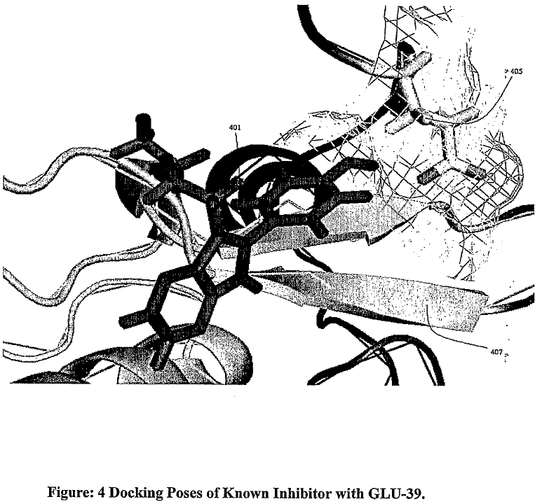

For example, the highly active compound 4-(7-Butyl-5H-pyrrolo[2,3-b]pyrazin-6-yl)-phenol was docked to analyze the interaction. It was identified that L35, V36.L37 residues form a hydrophobic surface in the structure of GSK3Alpha which is depicted in FIG. 4. Cyclopentyl moiety of the modulator interacts with this hydrophobic core of the ligand binding site at 401. D106 is in the solvent polar region amino functionality of the fused heterocyclic ring interacts with D106 at 405. The molecule was docked near the activation loop at 407. This figure exhibits the binding of the known active ligand for GSK3-Alpha in its active site. This validates the utility of the built in model.

### Structural Specificity of GSK-3 Alpha Modulator

The coordinate of the model given in PDB format enables the visualization of the structure in relation to its 3D space. The various pockets/cavities were analyzed and the active site was identified by superimposition of the GSK3-Alpha specific ligand to the structure. The interacting residues were identified and the neighboring residues were analyzed for specificity details. The model built for GSK3-Alpha was superimposed onto the GSK-3 Beta structure and the differences of the pockets were analyzed.

It was found that the alpha isoform active site consists of the residues Val 41, Asp 106, Glu 39, Gln 91, Asn 92 and Lys 89. For validating the results the GSK3-Alpha active molecule was docked to the Alpha structure. Glide docking protocol was used to dock the active molecules as shown in FIG. 3 and discussed earlier. To validate the model for specificity against GSK3-Beta isoform, some highly active GSK3-Beta compounds we docked to the structure. The docking of the beta active inhibitor and alpha active inhibitor exhibited a glide score of −8.97 and −6.78 respectively as given in Table 3 and Table 4.

The model in its 3D structure exhibits many pockets which can be identified visual inspection and the active site was identified as described earlier. The active site residues are identified and analyzed. Using this information various method can be employed to design modulators to inhibit or modulate the activity of GSK3-Alpha. For example, when the residues comprising the ATP cleft of both GSK3-Beta and GSK3-Alpha are compared, a key difference is found in the residue, E39. However, as this residue provides contacts through the backbone, they would not be expected to impact inhibitor binding significantly. E39 has a strong hydrogen bonding interaction with the phenolic hydroxyl group of the inhibitor in the predicted binding mode. E39 is a specific conserved residue in GSK3-Alpha and a strong hydrogen bonding with this residue would potentially help in enhancing the specificity towards GSK3-Alpha.

Further, the residue GLU39 in place of ASP in the same place in the Beta isoform is the key residue for specificity. Moreover, the distance differences between the residues can be used to design pharmacophore based hypothesis. Various known Alpha isoform specific modulators can be docked and analyzed and from the gathered information new scaffolds can be designed as modulators for GSK3-Alpha. Based on the designed scaffolds various focused combinatorial libraries can be designed and generated for synthesis and analyzed. In the process new hit compounds can be identified as potential modulators for GSK3-Alpha.

The GSK-3 Alpha model in its 3D structure exhibits many pockets which can be identified by visual inspection and the active site can be identified as described earlier. The active site residues are thus identified and analyzed. Using this information various method can be employed to design modulators to inhibit GSK3-Alpha. The residue GLU39 in place of ASP in the same place in its beta isoform is the key residue for specificity. Moreover, the distance differences between the residues can be used to design pharmacophore based hypothesis. Various known alpha isoform specific inhibitor can be docked and analyzed and from the gathered information new scaffolds can be designed as modulators for GSK3-Alpha. Based on the designed scaffolds various focused combinatorial libraries can be designed and generated for synthesis and analyzed. In the process new hit compounds can be identified as potential modulators for GSK3-Alpha.

TABLE 1
Structure co-ordinates of GSK-3Alpha

| | | | | | | | | | | | |
|---|---|---|---|---|---|---|---|---|---|---|---|
| ATOM | 619 | N | LEU | A | 36 | 10.626 | 21.403 | −9.076 | 1.00 | 0.00 | N |
| ATOM | 620 | CA | LEU | A | 36 | 10.985 | 20.939 | −7.738 | 1.00 | 0.00 | C |
| ATOM | 621 | C | LEU | A | 36 | 12.116 | 21.798 | −7.181 | 1.00 | 0.00 | C |
| ATOM | 622 | O | LEU | A | 36 | 13.222 | 21.808 | −7.733 | 1.00 | 0.00 | O |
| ATOM | 623 | CB | LEU | A | 36 | 11.431 | 19.475 | −7.783 | 1.00 | 0.00 | C |
| ATOM | 624 | CG | LEU | A | 36 | 11.813 | 18.826 | −6.425 | 1.00 | 0.00 | C |
| ATOM | 625 | CD1 | LEU | A | 36 | 10.687 | 18.904 | −5.378 | 1.00 | 0.00 | C |
| ATOM | 626 | CD2 | LEU | A | 36 | 12.243 | 17.365 | −6.632 | 1.00 | 0.00 | C |
| ATOM | 627 | H | LEU | A | 36 | 11.198 | 21.103 | −9.851 | 1.00 | 0.00 | H |
| ATOM | 628 | HA | LEU | A | 36 | 10.133 | 21.003 | −7.057 | 1.00 | 0.00 | H |
| ATOM | 629 | 1HB | LEU | A | 36 | 10.626 | 18.889 | −8.228 | 1.00 | 0.00 | H |
| ATOM | 630 | 2HB | LEU | A | 36 | 12.275 | 19.376 | −8.469 | 1.00 | 0.00 | H |
| ATOM | 631 | HG | LEU | A | 36 | 12.678 | 19.351 | −6.017 | 1.00 | 0.00 | H |
| ATOM | 632 | 1HD1 | LEU | A | 36 | 10.977 | 18.400 | −4.456 | 1.00 | 0.00 | H |
| ATOM | 633 | 2HD1 | LEU | A | 36 | 10.454 | 19.932 | −5.102 | 1.00 | 0.00 | H |
| ATOM | 634 | 3HD1 | LEU | A | 36 | 9.771 | 18.436 | −5.739 | 1.00 | 0.00 | H |
| ATOM | 635 | 1HD2 | LEU | A | 36 | 12.560 | 16.907 | −5.695 | 1.00 | 0.00 | H |
| ATOM | 636 | 2HD2 | LEU | A | 36 | 11.428 | 16.762 | −7.035 | 1.00 | 0.00 | H |
| ATOM | 637 | 3HD2 | LEU | A | 36 | 13.082 | 17.295 | −7.326 | 1.00 | 0.00 | H |
| ATOM | 638 | N | VAL | A | 37 | 11.840 | 22.528 | −6.102 | 1.00 | 0.00 | N |
| ATOM | 639 | CA | VAL | A | 37 | 12.854 | 23.380 | −5.487 | 1.00 | 0.00 | C |
| ATOM | 640 | C | VAL | A | 37 | 13.508 | 22.597 | −4.357 | 1.00 | 0.00 | C |
| ATOM | 641 | O | VAL | A | 37 | 12.891 | 22.332 | −3.322 | 1.00 | 0.00 | O |
| ATOM | 642 | CB | VAL | A | 37 | 12.254 | 24.686 | −4.931 | 1.00 | 0.00 | C |
| ATOM | 643 | CG1 | VAL | A | 37 | 13.326 | 25.661 | −4.400 | 1.00 | 0.00 | C |
| ATOM | 644 | CG2 | VAL | A | 37 | 11.389 | 25.395 | −5.996 | 1.00 | 0.00 | C |
| ATOM | 645 | H | VAL | A | 37 | 10.946 | 22.441 | −5.631 | 1.00 | 0.00 | H |
| ATOM | 646 | HA | VAL | A | 37 | 13.617 | 23.663 | −6.216 | 1.00 | 0.00 | H |
| ATOM | 647 | HB | VAL | A | 37 | 11.608 | 24.415 | −4.113 | 1.00 | 0.00 | H |
| ATOM | 648 | 1HG1 | VAL | A | 37 | 12.880 | 26.589 | −4.043 | 1.00 | 0.00 | H |
| ATOM | 649 | 2HG1 | VAL | A | 37 | 13.886 | 25.239 | −3.565 | 1.00 | 0.00 | H |
| ATOM | 650 | 3HG1 | VAL | A | 37 | 14.043 | 25.924 | −5.179 | 1.00 | 0.00 | H |
| ATOM | 651 | 1HG2 | VAL | A | 37 | 10.984 | 26.336 | −5.632 | 1.00 | 0.00 | H |
| ATOM | 652 | 2HG2 | VAL | A | 37 | 11.974 | 25.615 | −6.889 | 1.00 | 0.00 | H |
| ATOM | 653 | 3HG2 | VAL | A | 37 | 10.537 | 24.786 | −6.301 | 1.00 | 0.00 | H |
| ATOM | 654 | N | LEU | A | 38 | 14.769 | 22.232 | −4.569 | 1.00 | 0.00 | N |
| ATOM | 655 | CA | LEU | A | 38 | 15.513 | 21.441 | −3.604 | 1.00 | 0.00 | C |
| ATOM | 656 | C | LEU | A | 38 | 16.692 | 22.176 | −3.005 | 1.00 | 0.00 | C |
| ATOM | 657 | O | LEU | A | 38 | 17.122 | 23.209 | −3.511 | 1.00 | 0.00 | O |
| ATOM | 658 | CB | LEU | A | 38 | 16.032 | 20.163 | −4.272 | 1.00 | 0.00 | C |
| ATOM | 659 | CG | LEU | A | 38 | 15.978 | 18.888 | −3.405 | 1.00 | 0.00 | C |
| ATOM | 660 | CD1 | LEU | A | 38 | 14.540 | 18.361 | −3.247 | 1.00 | 0.00 | C |
| ATOM | 661 | CD2 | LEU | A | 38 | 16.878 | 17.792 | −3.989 | 1.00 | 0.00 | C |
| ATOM | 662 | H | LEU | A | 38 | 15.243 | 22.519 | −5.412 | 1.00 | 0.00 | H |
| ATOM | 663 | HA | LEU | A | 38 | 14.876 | 21.129 | −2.774 | 1.00 | 0.00 | H |
| ATOM | 664 | 1HB | LEU | A | 38 | 15.462 | 19.963 | −5.181 | 1.00 | 0.00 | H |
| ATOM | 665 | 2HB | LEU | A | 38 | 17.053 | 20.316 | −4.631 | 1.00 | 0.00 | H |
| ATOM | 666 | HG | LEU | A | 38 | 16.381 | 19.121 | −2.423 | 1.00 | 0.00 | H |
| ATOM | 667 | 1HD1 | LEU | A | 38 | 14.522 | 17.432 | −2.677 | 1.00 | 0.00 | H |
| ATOM | 668 | 2HD1 | LEU | A | 38 | 13.895 | 19.072 | −2.732 | 1.00 | 0.00 | H |
| ATOM | 669 | 3HD1 | LEU | A | 38 | 14.088 | 18.151 | −4.217 | 1.00 | 0.00 | H |
| ATOM | 670 | 1HD2 | LEU | A | 38 | 16.871 | 16.898 | −3.364 | 1.00 | 0.00 | H |
| ATOM | 671 | 2HD2 | LEU | A | 38 | 16.552 | 17.502 | −4.988 | 1.00 | 0.00 | H |
| ATOM | 672 | 3HD2 | LEU | A | 38 | 17.914 | 18.126 | −4.060 | 1.00 | 0.00 | H |
| ATOM | 673 | N | GLU | A | 39 | 17.211 | 21.631 | −1.912 | 1.00 | 0.00 | N |
| ATOM | 674 | CA | GLU | A | 39 | 18.395 | 22.202 | −1.280 | 1.00 | 0.00 | C |
| ATOM | 675 | C | GLU | A | 39 | 19.505 | 22.015 | −2.307 | 1.00 | 0.00 | C |
| ATOM | 676 | O | GLU | A | 39 | 19.567 | 20.976 | −2.977 | 1.00 | 0.00 | O |
| ATOM | 677 | CB | GLU | A | 39 | 18.764 | 21.417 | −0.027 | 1.00 | 0.00 | C |
| ATOM | 678 | CG | GLU | A | 39 | 20.113 | 21.752 | 0.666 | 1.00 | 0.00 | C |
| ATOM | 679 | CD | GLU | A | 39 | 20.481 | 20.711 | 1.711 | 1.00 | 0.00 | C |
| ATOM | 680 | OE1 | GLU | A | 39 | 20.109 | 19.535 | 1.537 | 1.00 | 0.00 | O |
| ATOM | 681 | OE2 | GLU | A | 39 | 21.053 | 21.053 | 2.763 | 1.00 | 0.00 | O |
| ATOM | 682 | H | GLU | A | 39 | 16.846 | 20.761 | −1.563 | 1.00 | 0.00 | H |
| ATOM | 683 | HA | GLU | A | 39 | 18.221 | 23.258 | −1.063 | 1.00 | 0.00 | H |
| ATOM | 684 | 1HB | GLU | A | 39 | 17.965 | 21.530 | 0.701 | 1.00 | 0.00 | H |
| ATOM | 685 | 2HB | GLU | A | 39 | 18.758 | 20.359 | −0.298 | 1.00 | 0.00 | H |
| ATOM | 686 | 1HG | GLU | A | 39 | 20.944 | 21.762 | −0.036 | 1.00 | 0.00 | H |
| ATOM | 687 | 2HG | GLU | A | 39 | 20.073 | 22.738 | 1.128 | 1.00 | 0.00 | H |
| ATOM | 688 | N | TYR | A | 40 | 20.369 | 23.012 | −2.444 | 1.00 | 0.00 | N |
| ATOM | 689 | CA | TYR | A | 40 | 21.470 | 22.895 | −3.383 | 1.00 | 0.00 | C |
| ATOM | 690 | C | TYR | A | 40 | 22.657 | 22.233 | −2.675 | 1.00 | 0.00 | C |
| ATOM | 691 | O | TYR | A | 40 | 23.008 | 22.599 | −1.548 | 1.00 | 0.00 | O |
| ATOM | 692 | CB | TYR | A | 40 | 21.865 | 24.274 | −3.914 | 1.00 | 0.00 | C |
| ATOM | 693 | CG | TYR | A | 40 | 23.094 | 24.282 | −4.815 | 1.00 | 0.00 | C |
| ATOM | 694 | CD1 | TYR | A | 40 | 23.007 | 23.816 | −6.143 | 1.00 | 0.00 | C |
| ATOM | 695 | CD2 | TYR | A | 40 | 24.342 | 24.704 | −4.305 | 1.00 | 0.00 | C |
| ATOM | 696 | CE1 | TYR | A | 40 | 24.160 | 23.763 | −6.950 | 1.00 | 0.00 | C |
| ATOM | 697 | CE2 | TYR | A | 40 | 25.494 | 24.650 | −5.112 | 1.00 | 0.00 | C |
| ATOM | 698 | CZ | TYR | A | 40 | 25.403 | 24.178 | −6.436 | 1.00 | 0.00 | C |
| ATOM | 699 | OH | TYR | A | 40 | 26.515 | 24.118 | −7.222 | 1.00 | 0.00 | O |
| ATOM | 700 | H | TYR | A | 40 | 20.395 | 23.718 | −1.707 | 1.00 | 0.00 | H |
| ATOM | 701 | HA | TYR | A | 40 | 21.187 | 22.292 | −4.249 | 1.00 | 0.00 | H |
| ATOM | 702 | 1HB | TYR | A | 40 | 21.030 | 24.701 | −4.471 | 1.00 | 0.00 | H |
| ATOM | 703 | 2HB | TYR | A | 40 | 22.042 | 24.954 | −3.079 | 1.00 | 0.00 | H |
| ATOM | 704 | HD1 | TYR | A | 40 | 22.060 | 23.483 | −6.545 | 1.00 | 0.00 | H |
| ATOM | 705 | HD2 | TYR | A | 40 | 24.426 | 25.052 | −3.285 | 1.00 | 0.00 | H |
| ATOM | 706 | HE1 | TYR | A | 40 | 24.088 | 23.398 | −7.965 | 1.00 | 0.00 | H |
| ATOM | 707 | HE2 | TYR | A | 40 | 26.443 | 24.967 | −4.704 | 1.00 | 0.00 | H |
| ATOM | 708 | HH | TYR | A | 40 | 27.295 | 24.447 | −6.792 | 1.00 | 0.00 | H |
| ATOM | 709 | N | VAL | A | 41 | 23.238 | 21.230 | −3.329 | 1.00 | 0.00 | N |
| ATOM | 710 | CA | VAL | A | 41 | 24.395 | 20.511 | −2.796 | 1.00 | 0.00 | C |
| ATOM | 711 | C | VAL | A | 41 | 25.462 | 20.658 | −3.884 | 1.00 | 0.00 | C |
| ATOM | 712 | O | VAL | A | 41 | 25.237 | 20.298 | −5.034 | 1.00 | 0.00 | O |
| ATOM | 713 | CB | VAL | A | 41 | 24.055 | 19.044 | −2.529 | 1.00 | 0.00 | C |
| ATOM | 714 | CG1 | VAL | A | 41 | 25.203 | 18.305 | −1.806 | 1.00 | 0.00 | C |
| ATOM | 715 | CG2 | VAL | A | 41 | 22.753 | 18.864 | −1.717 | 1.00 | 0.00 | C |
| ATOM | 716 | H | VAL | A | 41 | 22.942 | 20.996 | −4.265 | 1.00 | 0.00 | H |
| ATOM | 717 | HA | VAL | A | 41 | 24.684 | 20.952 | −1.846 | 1.00 | 0.00 | H |
| ATOM | 718 | HB | VAL | A | 41 | 23.903 | 18.548 | −3.486 | 1.00 | 0.00 | H |
| ATOM | 719 | 1HG1 | VAL | A | 41 | 24.928 | 17.276 | −1.577 | 1.00 | 0.00 | H |
| ATOM | 720 | 2HG1 | VAL | A | 41 | 26.110 | 18.264 | −2.409 | 1.00 | 0.00 | H |
| ATOM | 721 | 3HG1 | VAL | A | 41 | 25.451 | 18.779 | −0.855 | 1.00 | 0.00 | H |
| ATOM | 722 | 1HG2 | VAL | A | 41 | 22.561 | 17.812 | −1.505 | 1.00 | 0.00 | H |
| ATOM | 723 | 2HG2 | VAL | A | 41 | 22.802 | 19.389 | −0.761 | 1.00 | 0.00 | H |
| ATOM | 724 | 3HG2 | VAL | A | 41 | 21.880 | 19.238 | −2.252 | 1.00 | 0.00 | H |
| ATOM | 725 | N | PRO | A | 42 | 26.651 | 21.161 | −3.515 | 1.00 | 0.00 | N |
| ATOM | 726 | CA | PRO | A | 42 | 27.762 | 21.397 | −4.447 | 1.00 | 0.00 | C |
| ATOM | 727 | C | PRO | A | 42 | 28.454 | 20.299 | −5.248 | 1.00 | 0.00 | C |
| ATOM | 728 | O | PRO | A | 42 | 28.887 | 20.560 | −6.372 | 1.00 | 0.00 | O |
| ATOM | 729 | CB | PRO | A | 42 | 28.759 | 22.190 | −3.606 | 1.00 | 0.00 | C |
| ATOM | 730 | CG | PRO | A | 42 | 28.547 | 21.756 | −2.177 | 1.00 | 0.00 | C |
| ATOM | 731 | CD | PRO | A | 42 | 27.058 | 21.453 | −2.132 | 1.00 | 0.00 | C |
| ATOM | 732 | HA | PRO | A | 42 | 27.371 | 22.077 | −5.206 | 1.00 | 0.00 | H |
| ATOM | 733 | 1HB | PRO | A | 42 | 29.797 | 22.063 | −3.920 | 1.00 | 0.00 | H |
| ATOM | 734 | 2HB | PRO | A | 42 | 28.535 | 23.254 | −3.693 | 1.00 | 0.00 | H |
| ATOM | 735 | 1HG | PRO | A | 42 | 28.863 | 22.501 | −1.444 | 1.00 | 0.00 | H |
| ATOM | 736 | 2HG | PRO | A | 42 | 29.124 | 20.850 | −1.992 | 1.00 | 0.00 | H |
| ATOM | 737 | 1HD | PRO | A | 42 | 26.515 | 22.333 | −1.780 | 1.00 | 0.00 | H |
| ATOM | 738 | 2HD | PRO | A | 42 | 26.856 | 20.633 | −1.444 | 1.00 | 0.00 | H |
| ATOM | 739 | N | GLU | A | 43 | 28.565 | 19.090 | −4.709 | 1.00 | 0.00 | N |
| ATOM | 740 | CA | GLU | A | 43 | 29.265 | 18.023 | −5.425 | 1.00 | 0.00 | C |
| ATOM | 741 | C | GLU | A | 43 | 28.487 | 16.715 | −5.515 | 1.00 | 0.00 | C |
| ATOM | 742 | O | GLU | A | 43 | 27.365 | 16.618 | −5.047 | 1.00 | 0.00 | O |
| ATOM | 743 | CB | GLU | A | 43 | 30.602 | 17.748 | −4.729 | 1.00 | 0.00 | C |
| ATOM | 744 | CG | GLU | A | 43 | 31.771 | 18.643 | −5.200 | 1.00 | 0.00 | C |
| ATOM | 745 | CD | GLU | A | 43 | 32.327 | 18.245 | −6.562 | 1.00 | 0.00 | C |
| ATOM | 746 | OE1 | GLU | A | 43 | 31.603 | 17.548 | −7.301 | 1.00 | 0.00 | O |
| ATOM | 747 | OE2 | GLU | A | 43 | 33.479 | 18.620 | −6.857 | 1.00 | 0.00 | O |
| ATOM | 748 | H | GLU | A | 43 | 28.109 | 18.856 | −3.841 | 1.00 | 0.00 | H |
| ATOM | 749 | HA | GLU | A | 43 | 29.452 | 18.274 | −6.470 | 1.00 | 0.00 | H |
| ATOM | 750 | 1HB | GLU | A | 43 | 30.479 | 17.903 | −3.661 | 1.00 | 0.00 | H |
| ATOM | 751 | 2HB | GLU | A | 43 | 30.943 | 16.715 | −4.809 | 1.00 | 0.00 | H |
| ATOM | 752 | 1HG | GLU | A | 43 | 31.468 | 19.689 | −5.234 | 1.00 | 0.00 | H |
| ATOM | 753 | 2HG | GLU | A | 43 | 32.588 | 18.578 | −4.483 | 1.00 | 0.00 | H |
| ATOM | 754 | N | THR | A | 44 | 29.110 | 15.716 | −6.133 | 1.00 | 0.00 | N |
| ATOM | 755 | CA | THR | A | 44 | 28.529 | 14.384 | −6.258 | 1.00 | 0.00 | C |
| ATOM | 756 | C | THR | A | 44 | 29.664 | 13.400 | −6.035 | 1.00 | 0.00 | C |
| ATOM | 757 | O | THR | A | 44 | 30.840 | 13.742 | −6.217 | 1.00 | 0.00 | O |
| ATOM | 758 | CB | THR | A | 44 | 27.943 | 14.114 | −7.657 | 1.00 | 0.00 | C |
| ATOM | 759 | OG1 | THR | A | 44 | 28.903 | 14.337 | −8.681 | 1.00 | 0.00 | O |
| ATOM | 760 | CG2 | THR | A | 44 | 26.700 | 14.950 | −7.975 | 1.00 | 0.00 | C |
| ATOM | 761 | H | THR | A | 44 | 30.088 | 15.820 | −6.382 | 1.00 | 0.00 | H |
| ATOM | 762 | HA | THR | A | 44 | 27.802 | 14.204 | −5.482 | 1.00 | 0.00 | H |
| ATOM | 763 | HB | THR | A | 44 | 27.639 | 13.066 | −7.714 | 1.00 | 0.00 | H |
| ATOM | 764 | HG1 | THR | A | 44 | 29.129 | 15.258 | −8.642 | 1.00 | 0.00 | H |
| ATOM | 765 | 1HG2 | THR | A | 44 | 26.280 | 14.681 | −8.945 | 1.00 | 0.00 | H |
| ATOM | 766 | 2HG2 | THR | A | 44 | 25.925 | 14.773 | −7.230 | 1.00 | 0.00 | H |
| ATOM | 767 | 3HG2 | THR | A | 44 | 26.911 | 16.020 | −7.984 | 1.00 | 0.00 | H |
| ATOM | 768 | N | VAL | A | 45 | 29.320 | 12.185 | −5.633 | 1.00 | 0.00 | N |
| ATOM | 769 | CA | VAL | A | 45 | 30.330 | 11.154 | −5.402 | 1.00 | 0.00 | C |
| ATOM | 770 | C | VAL | A | 45 | 31.026 | 10.877 | −6.734 | 1.00 | 0.00 | C |
| ATOM | 771 | O | VAL | A | 45 | 32.207 | 10.524 | −6.777 | 1.00 | 0.00 | O |
| ATOM | 772 | CB | VAL | A | 45 | 29.675 | 9.852 | −4.873 | 1.00 | 0.00 | C |
| ATOM | 773 | CG1 | VAL | A | 45 | 30.622 | 8.639 | −4.770 | 1.00 | 0.00 | C |
| ATOM | 774 | CG2 | VAL | A | 45 | 29.043 | 10.071 | −3.487 | 1.00 | 0.00 | C |
| ATOM | 775 | H | VAL | A | 45 | 28.338 | 11.960 | −5.473 | 1.00 | 0.00 | H |
| ATOM | 776 | HA | VAL | A | 45 | 31.082 | 11.514 | −4.696 | 1.00 | 0.00 | H |
| ATOM | 777 | HB | VAL | A | 45 | 28.876 | 9.572 | −5.561 | 1.00 | 0.00 | H |
| ATOM | 778 | 1HG1 | VAL | A | 45 | 30.120 | 7.781 | −4.319 | 1.00 | 0.00 | H |
| ATOM | 779 | 2HG1 | VAL | A | 45 | 30.985 | 8.307 | −5.743 | 1.00 | 0.00 | H |
| ATOM | 780 | 3HG1 | VAL | A | 45 | 31.489 | 8.872 | −4.150 | 1.00 | 0.00 | H |
| ATOM | 781 | 1HG2 | VAL | A | 45 | 28.490 | 9.188 | −3.168 | 1.00 | 0.00 | H |
| ATOM | 782 | 2HG2 | VAL | A | 45 | 29.809 | 10.248 | −2.735 | 1.00 | 0.00 | H |
| ATOM | 783 | 3HG2 | VAL | A | 45 | 28.354 | 10.914 | −3.465 | 1.00 | 0.00 | H |
| ATOM | 784 | N | TYR | A | 46 | 30.284 | 11.046 | −7.820 | 1.00 | 0.00 | N |
| ATOM | 785 | CA | TYR | A | 46 | 30.834 | 10.821 | −9.148 | 1.00 | 0.00 | C |
| ATOM | 786 | C | TYR | A | 46 | 32.026 | 11.748 | −9.420 | 1.00 | 0.00 | C |
| ATOM | 787 | O | TYR | A | 46 | 33.112 | 11.291 | −9.784 | 1.00 | 0.00 | O |
| ATOM | 788 | CB | TYR | A | 46 | 29.753 | 11.056 | −10.201 | 1.00 | 0.00 | C |
| ATOM | 789 | CG | TYR | A | 46 | 30.217 | 10.908 | −11.644 | 1.00 | 0.00 | C |
| ATOM | 790 | CD1 | TYR | A | 46 | 30.710 | 9.670 | −12.107 | 1.00 | 0.00 | C |
| ATOM | 791 | CD2 | TYR | A | 46 | 30.168 | 12.012 | −12.522 | 1.00 | 0.00 | C |
| ATOM | 792 | CE1 | TYR | A | 46 | 31.149 | 9.537 | −13.438 | 1.00 | 0.00 | C |
| ATOM | 793 | CE2 | TYR | A | 46 | 30.600 | 11.876 | −13.855 | 1.00 | 0.00 | C |
| ATOM | 794 | CZ | TYR | A | 46 | 31.089 | 10.637 | −14.314 | 1.00 | 0.00 | C |
| ATOM | 795 | OH | TYR | A | 46 | 31.499 | 10.494 | −15.605 | 1.00 | 0.00 | O |
| ATOM | 796 | H | TYR | A | 46 | 29.320 | 11.326 | −7.708 | 1.00 | 0.00 | H |
| ATOM | 797 | HA | TYR | A | 46 | 31.188 | 9.790 | −9.220 | 1.00 | 0.00 | H |
| ATOM | 798 | 1HB | TYR | A | 46 | 28.938 | 10.357 | −10.062 | 1.00 | 0.00 | H |
| ATOM | 799 | 2HB | TYR | A | 46 | 29.309 | 12.044 | −10.072 | 1.00 | 0.00 | H |
| ATOM | 800 | HD1 | TYR | A | 46 | 30.756 | 8.813 | −11.450 | 1.00 | 0.00 | H |
| ATOM | 801 | HD2 | TYR | A | 46 | 29.797 | 12.968 | −12.179 | 1.00 | 0.00 | H |
| ATOM | 802 | HE1 | TYR | A | 46 | 31.524 | 8.585 | −13.786 | 1.00 | 0.00 | H |
| ATOM | 803 | HE2 | TYR | A | 46 | 30.552 | 12.729 | −14.517 | 1.00 | 0.00 | H |
| ATOM | 804 | HH | TYR | A | 46 | 31.367 | 11.278 | −16.126 | 1.00 | 0.00 | H |
| ATOM | 805 | N | ARG | A | 47 | 31.818 | 13.049 | −9.243 | 1.00 | 0.00 | N |
| ATOM | 806 | CA | ARG | A | 47 | 32.872 | 14.028 | −9.497 | 1.00 | 0.00 | C |
| ATOM | 807 | C | ARG | A | 47 | 34.063 | 13.855 | −8.564 | 1.00 | 0.00 | C |
| ATOM | 808 | O | ARG | A | 47 | 35.212 | 13.881 | −9.001 | 1.00 | 0.00 | O |
| ATOM | 809 | CB | ARG | A | 47 | 32.326 | 15.444 | −9.356 | 1.00 | 0.00 | C |
| ATOM | 810 | CG | ARG | A | 47 | 31.410 | 15.861 | −10.519 | 1.00 | 0.00 | C |
| ATOM | 811 | CD | ARG | A | 47 | 30.767 | 17.237 | −10.294 | 1.00 | 0.00 | C |
| ATOM | 812 | NE | ARG | A | 47 | 29.918 | 17.623 | −11.433 | 1.00 | 0.00 | N |
| ATOM | 813 | CZ | ARG | A | 47 | 29.094 | 18.678 | −11.479 | 1.00 | 0.00 | C |
| ATOM | 814 | NH1 | ARG | A | 47 | 28.980 | 19.504 | −10.440 | 1.00 | 0.00 | N |
| ATOM | 815 | NH2 | ARG | A | 47 | 28.380 | 18.901 | −12.578 | 1.00 | 0.00 | N |
| ATOM | 816 | H | ARG | A | 47 | 30.921 | 13.351 | −8.878 | 1.00 | 0.00 | H |
| ATOM | 817 | HA | ARG | A | 47 | 33.236 | 13.890 | −10.518 | 1.00 | 0.00 | H |
| ATOM | 818 | 1HB | ARG | A | 47 | 31.764 | 15.480 | −8.425 | 1.00 | 0.00 | H |
| ATOM | 819 | 2HB | ARG | A | 47 | 33.133 | 16.174 | −9.259 | 1.00 | 0.00 | H |
| ATOM | 820 | 1HG | ARG | A | 47 | 31.990 | 15.874 | −11.442 | 1.00 | 0.00 | H |
| ATOM | 821 | 2HG | ARG | A | 47 | 30.627 | 15.114 | −10.660 | 1.00 | 0.00 | H |
| ATOM | 822 | 1HD | ARG | A | 47 | 30.153 | 17.214 | −9.393 | 1.00 | 0.00 | H |
| ATOM | 823 | 2HD | ARG | A | 47 | 31.533 | 17.998 | −10.138 | 1.00 | 0.00 | H |
| ATOM | 824 | HE | ARG | A | 47 | 30.003 | 17.030 | −12.249 | 1.00 | 0.00 | H |
| ATOM | 825 | 1HH1 | ARG | A | 47 | 28.360 | 20.300 | −10.448 | 1.00 | 0.00 | H |
| ATOM | 826 | 2HH1 | ARG | A | 47 | 29.514 | 19.361 | −9.596 | 1.00 | 0.00 | H |
| ATOM | 827 | 1HH2 | ARG | A | 47 | 27.746 | 19.681 | −12.656 | 1.00 | 0.00 | H |
| ATOM | 828 | 2HH2 | ARG | A | 47 | 28.433 | 18.295 | −13.383 | 1.00 | 0.00 | H |
| ATOM | 829 | N | VAL | A | 48 | 33.795 | 13.677 | −7.278 | 1.00 | 0.00 | N |
| ATOM | 830 | CA | VAL | A | 48 | 34.873 | 13.502 | −6.314 | 1.00 | 0.00 | C |
| ATOM | 831 | C | VAL | A | 48 | 35.700 | 12.257 | −6.638 | 1.00 | 0.00 | C |
| ATOM | 832 | O | VAL | A | 48 | 36.928 | 12.306 | −6.613 | 1.00 | 0.00 | O |
| ATOM | 833 | CB | VAL | A | 48 | 34.322 | 13.407 | −4.871 | 1.00 | 0.00 | C |
| ATOM | 834 | CG1 | VAL | A | 48 | 35.376 | 13.096 | −3.786 | 1.00 | 0.00 | C |
| ATOM | 835 | CG2 | VAL | A | 48 | 33.585 | 14.705 | −4.477 | 1.00 | 0.00 | C |
| ATOM | 836 | H | VAL | A | 48 | 32.834 | 13.677 | −6.953 | 1.00 | 0.00 | H |
| ATOM | 837 | HA | VAL | A | 48 | 35.545 | 14.361 | −6.382 | 1.00 | 0.00 | H |
| ATOM | 838 | HB | VAL | A | 48 | 33.587 | 12.599 | −4.844 | 1.00 | 0.00 | H |
| ATOM | 839 | 1HG1 | VAL | A | 48 | 34.931 | 13.109 | −2.790 | 1.00 | 0.00 | H |
| ATOM | 840 | 2HG1 | VAL | A | 48 | 35.819 | 12.107 | −3.911 | 1.00 | 0.00 | H |
| ATOM | 841 | 3HG1 | VAL | A | 48 | 36.182 | 13.830 | −3.798 | 1.00 | 0.00 | H |
| ATOM | 842 | 1HG2 | VAL | A | 48 | 33.096 | 14.604 | −3.507 | 1.00 | 0.00 | H |
| ATOM | 843 | 2HG2 | VAL | A | 48 | 34.274 | 15.548 | −4.415 | 1.00 | 0.00 | H |
| ATOM | 844 | 3HG2 | VAL | A | 48 | 32.812 | 14.983 | −5.191 | 1.00 | 0.00 | H |
| ATOM | 845 | N | ALA | A | 49 | 35.032 | 11.148 | −6.947 | 1.00 | 0.00 | N |
| ATOM | 846 | CA | ALA | A | 49 | 35.738 | 9.909 | −7.285 | 1.00 | 0.00 | C |
| ATOM | 847 | C | ALA | A | 49 | 36.557 | 10.084 | −8.568 | 1.00 | 0.00 | C |
| ATOM | 848 | O | ALA | A | 49 | 37.637 | 9.502 | −8.715 | 1.00 | 0.00 | O |
| ATOM | 849 | CB | ALA | A | 49 | 34.740 | 8.753 | −7.455 | 1.00 | 0.00 | C |
| ATOM | 850 | H | ALA | A | 49 | 34.015 | 11.138 | −6.938 | 1.00 | 0.00 | H |
| ATOM | 851 | HA | ALA | A | 49 | 36.425 | 9.660 | −6.474 | 1.00 | 0.00 | H |
| ATOM | 852 | 1HB | ALA | A | 49 | 35.255 | 7.823 | −7.698 | 1.00 | 0.00 | H |
| ATOM | 853 | 2HB | ALA | A | 49 | 34.176 | 8.584 | −6.538 | 1.00 | 0.00 | H |
| ATOM | 854 | 3HB | ALA | A | 49 | 34.024 | 8.953 | −8.253 | 1.00 | 0.00 | H |
| ATOM | 855 | N | ARG | A | 50 | 36.039 | 10.883 | −9.496 | 1.00 | 0.00 | N |
| ATOM | 856 | CA | ARG | A | 50 | 36.728 | 11.123 | −10.757 | 1.00 | 0.00 | C |
| ATOM | 857 | C | ARG | A | 50 | 38.019 | 11.902 | −10.528 | 1.00 | 0.00 | C |
| ATOM | 858 | O | ARG | A | 50 | 39.014 | 11.695 | −11.226 | 1.00 | 0.00 | O |
| ATOM | 859 | CB | ARG | A | 50 | 35.822 | 11.892 | −11.714 | 1.00 | 0.00 | C |
| ATOM | 860 | CG | ARG | A | 50 | 36.340 | 11.992 | −13.160 | 1.00 | 0.00 | C |
| ATOM | 861 | CD | ARG | A | 50 | 35.425 | 12.838 | −14.055 | 1.00 | 0.00 | C |
| ATOM | 862 | NE | ARG | A | 50 | 35.672 | 14.278 | −13.852 | 1.00 | 0.00 | N |
| ATOM | 863 | CZ | ARG | A | 50 | 35.446 | 15.263 | −14.729 | 1.00 | 0.00 | C |
| ATOM | 864 | NH1 | ARG | A | 50 | 35.010 | 15.003 | −15.959 | 1.00 | 0.00 | N |
| ATOM | 865 | NH2 | ARG | A | 50 | 35.730 | 16.513 | −14.384 | 1.00 | 0.00 | N |
| ATOM | 866 | H | ARG | A | 50 | 35.105 | 11.256 | −9.354 | 1.00 | 0.00 | H |
| ATOM | 867 | HA | ARG | A | 50 | 36.962 | 10.153 | −11.199 | 1.00 | 0.00 | H |
| ATOM | 868 | 1HB | ARG | A | 50 | 34.847 | 11.404 | −11.745 | 1.00 | 0.00 | H |
| ATOM | 869 | 2HB | ARG | A | 50 | 35.638 | 12.893 | −11.327 | 1.00 | 0.00 | H |
| ATOM | 870 | 1HG | ARG | A | 50 | 37.347 | 12.406 | −13.178 | 1.00 | 0.00 | H |
| ATOM | 871 | 2HG | ARG | A | 50 | 36.425 | 10.988 | −13.580 | 1.00 | 0.00 | H |
| ATOM | 872 | 1HD | ARG | A | 50 | 35.635 | 12.587 | −15.095 | 1.00 | 0.00 | H |
| ATOM | 873 | 2HD | ARG | A | 50 | 34.372 | 12.608 | −13.884 | 1.00 | 0.00 | H |
| ATOM | 874 | HE | ARG | A | 50 | 35.876 | 14.520 | −12.889 | 1.00 | 0.00 | H |
| ATOM | 875 | 1HH1 | ARG | A | 50 | 34.793 | 15.737 | −16.619 | 1.00 | 0.00 | H |
| ATOM | 876 | 2HH1 | ARG | A | 50 | 34.711 | 14.076 | −16.223 | 1.00 | 0.00 | H |
| ATOM | 877 | 1HH2 | ARG | A | 50 | 35.553 | 17.290 | −15.005 | 1.00 | 0.00 | H |
| ATOM | 878 | 2HH2 | ARG | A | 50 | 36.022 | 16.769 | −13.451 | 1.00 | 0.00 | H |
| ATOM | 879 | N | HIS | A | 51 | 38.002 | 12.800 | −9.549 | 1.00 | 0.00 | N |
| ATOM | 880 | CA | HIS | A | 51 | 39.185 | 13.588 | −9.236 | 1.00 | 0.00 | C |
| ATOM | 881 | C | HIS | A | 51 | 40.309 | 12.658 | −8.783 | 1.00 | 0.00 | C |
| ATOM | 882 | O | HIS | A | 51 | 41.435 | 12.741 | −9.281 | 1.00 | 0.00 | O |
| ATOM | 883 | CB | HIS | A | 51 | 38.885 | 14.612 | −8.132 | 1.00 | 0.00 | C |
| ATOM | 884 | CG | HIS | A | 51 | 38.217 | 15.893 | −8.571 | 1.00 | 0.00 | C |
| ATOM | 885 | ND1 | HIS | A | 51 | 36.881 | 15.980 | −8.914 | 1.00 | 0.00 | N |
| ATOM | 886 | CD2 | HIS | A | 51 | 38.708 | 17.172 | −8.706 | 1.00 | 0.00 | C |
| ATOM | 887 | CE1 | HIS | A | 51 | 36.604 | 17.259 | −9.169 | 1.00 | 0.00 | C |
| ATOM | 888 | NE2 | HIS | A | 51 | 37.679 | 18.036 | −9.088 | 1.00 | 0.00 | N |
| ATOM | 889 | H | HIS | A | 51 | 37.149 | 12.934 | −9.016 | 1.00 | 0.00 | H |
| ATOM | 890 | HA | HIS | A | 51 | 39.525 | 14.118 | −10.128 | 1.00 | 0.00 | H |
| ATOM | 891 | 1HB | HIS | A | 51 | 38.319 | 14.179 | −7.312 | 1.00 | 0.00 | H |
| ATOM | 892 | 2HB | HIS | A | 51 | 39.829 | 14.925 | −7.681 | 1.00 | 0.00 | H |
| ATOM | 893 | HD1 | HIS | A | 51 | 36.202 | 15.209 | −8.922 | 1.00 | 0.00 | H |
| ATOM | 894 | HD2 | HIS | A | 51 | 39.712 | 17.538 | −8.533 | 1.00 | 0.00 | H |
| ATOM | 895 | HE1 | HIS | A | 51 | 35.615 | 17.623 | −9.421 | 1.00 | 0.00 | H |
| ATOM | 896 | N | PHE | A | 52 | 40.005 | 11.777 | −7.835 | 1.00 | 0.00 | N |
| ATOM | 897 | CA | PHE | A | 52 | 41.000 | 10.848 | −7.328 | 1.00 | 0.00 | C |
| ATOM | 898 | C | PHE | A | 52 | 41.427 | 9.869 | −8.415 | 1.00 | 0.00 | C |
| ATOM | 899 | O | PHE | A | 52 | 42.587 | 9.469 | −8.484 | 1.00 | 0.00 | O |
| ATOM | 900 | CB | PHE | A | 52 | 40.453 | 10.091 | −6.118 | 1.00 | 0.00 | C |
| ATOM | 901 | CG | PHE | A | 52 | 40.352 | 10.915 | −4.840 | 1.00 | 0.00 | C |
| ATOM | 902 | CD1 | PHE | A | 52 | 41.496 | 11.129 | −4.048 | 1.00 | 0.00 | C |
| ATOM | 903 | CD2 | PHE | A | 52 | 39.119 | 11.447 | −4.412 | 1.00 | 0.00 | C |
| ATOM | 904 | CE1 | PHE | A | 52 | 41.424 | 11.894 | −2.869 | 1.00 | 0.00 | C |
| ATOM | 905 | CE2 | PHE | A | 52 | 39.041 | 12.219 | −3.237 | 1.00 | 0.00 | C |
| ATOM | 906 | CZ | PHE | A | 52 | 40.195 | 12.447 | −2.465 | 1.00 | 0.00 | C |
| ATOM | 907 | H | PHE | A | 52 | 39.068 | 11.764 | −7.450 | 1.00 | 0.00 | H |
| ATOM | 908 | HA | PHE | A | 52 | 41.888 | 11.405 | −7.022 | 1.00 | 0.00 | H |
| ATOM | 909 | 1HB | PHE | A | 52 | 39.476 | 9.674 | −6.369 | 1.00 | 0.00 | H |
| ATOM | 910 | 2HB | PHE | A | 52 | 41.075 | 9.234 | −5.880 | 1.00 | 0.00 | H |
| ATOM | 911 | HD1 | PHE | A | 52 | 42.433 | 10.690 | −4.339 | 1.00 | 0.00 | H |
| ATOM | 912 | HD2 | PHE | A | 52 | 38.221 | 11.243 | −4.968 | 1.00 | 0.00 | H |
| ATOM | 913 | HE1 | PHE | A | 52 | 42.311 | 12.050 | −2.272 | 1.00 | 0.00 | H |
| ATOM | 914 | HE2 | PHE | A | 52 | 38.093 | 12.628 | −2.922 | 1.00 | 0.00 | H |
| ATOM | 915 | HZ | PHE | A | 52 | 40.135 | 13.035 | −1.561 | 1.00 | 0.00 | H |
| ATOM | 916 | N | THR | A | 53 | 40.485 | 9.485 | −9.265 | 1.00 | 0.00 | N |
| ATOM | 917 | CA | THR | A | 53 | 40.786 | 8.565 | −10.349 | 1.00 | 0.00 | C |
| ATOM | 918 | C | THR | A | 53 | 41.823 | 9.192 | −11.269 | 1.00 | 0.00 | C |
| ATOM | 919 | O | THR | A | 53 | 42.846 | 8.585 | −11.581 | 1.00 | 0.00 | O |
| ATOM | 920 | CB | THR | A | 53 | 39.516 | 8.260 | −11.143 | 1.00 | 0.00 | C |
| ATOM | 921 | OG1 | THR | A | 53 | 38.689 | 7.395 | −10.385 | 1.00 | 0.00 | O |
| ATOM | 922 | CG2 | THR | A | 53 | 39.705 | 7.517 | −12.482 | 1.00 | 0.00 | C |
| ATOM | 923 | H | THR | A | 53 | 39.507 | 9.710 | −9.099 | 1.00 | 0.00 | H |
| ATOM | 924 | HA | THR | A | 53 | 41.205 | 7.642 | −9.942 | 1.00 | 0.00 | H |
| ATOM | 925 | HB | THR | A | 53 | 38.966 | 9.175 | −11.339 | 1.00 | 0.00 | H |
| ATOM | 926 | HG1 | THR | A | 53 | 38.977 | 6.494 | −10.489 | 1.00 | 0.00 | H |
| ATOM | 927 | 1HG2 | THR | A | 53 | 38.742 | 7.246 | −12.917 | 1.00 | 0.00 | H |
| ATOM | 928 | 2HG2 | THR | A | 53 | 40.214 | 8.133 | −13.223 | 1.00 | 0.00 | H |
| ATOM | 929 | 3HG2 | THR | A | 53 | 40.281 | 6.600 | −12.356 | 1.00 | 0.00 | H |
| ATOM | 930 | N | LYS | A | 54 | 41.550 | 10.420 | −11.691 | 1.00 | 0.00 | N |
| ATOM | 931 | CA | LYS | A | 54 | 42.440 | 11.147 | −12.583 | 1.00 | 0.00 | C |
| ATOM | 932 | C | LYS | A | 54 | 43.810 | 11.393 | −11.951 | 1.00 | 0.00 | C |
| ATOM | 933 | O | LYS | A | 54 | 44.840 | 11.248 | −12.612 | 1.00 | 0.00 | O |
| ATOM | 934 | CB | LYS | A | 54 | 41.790 | 12.475 | −12.984 | 1.00 | 0.00 | C |
| ATOM | 935 | CG | LYS | A | 54 | 40.687 | 12.319 | −14.045 | 1.00 | 0.00 | C |
| ATOM | 936 | CD | LYS | A | 54 | 39.943 | 13.637 | −14.310 | 1.00 | 0.00 | C |
| ATOM | 937 | CE | LYS | A | 54 | 38.985 | 13.547 | −15.508 | 1.00 | 0.00 | C |
| ATOM | 938 | NZ | LYS | A | 54 | 38.217 | 14.788 | −15.678 | 1.00 | 0.00 | N |
| ATOM | 939 | H | LYS | A | 54 | 40.698 | 10.880 | −11.377 | 1.00 | 0.00 | H |
| ATOM | 940 | HA | LYS | A | 54 | 42.604 | 10.548 | −13.481 | 1.00 | 0.00 | H |
| ATOM | 941 | 1HB | LYS | A | 54 | 41.392 | 12.974 | −12.098 | 1.00 | 0.00 | H |
| ATOM | 942 | 2HB | LYS | A | 54 | 42.548 | 13.150 | −13.385 | 1.00 | 0.00 | H |
| ATOM | 943 | 1HG | LYS | A | 54 | 41.133 | 11.954 | −14.971 | 1.00 | 0.00 | H |
| ATOM | 944 | 2HG | LYS | A | 54 | 39.969 | 11.559 | −13.732 | 1.00 | 0.00 | H |
| ATOM | 945 | 1HD | LYS | A | 54 | 39.395 | 13.918 | −13.409 | 1.00 | 0.00 | H |
| ATOM | 946 | 2HD | LYS | A | 54 | 40.668 | 14.432 | −14.490 | 1.00 | 0.00 | H |
| ATOM | 947 | 1HE | LYS | A | 54 | 39.544 | 13.353 | −16.423 | 1.00 | 0.00 | H |
| ATOM | 948 | 2HE | LYS | A | 54 | 38.288 | 12.719 | −15.381 | 1.00 | 0.00 | H |
| ATOM | 949 | 1HZ | LYS | A | 54 | 37.616 | 14.716 | −16.481 | 1.00 | 0.00 | H |
| ATOM | 950 | 2HZ | LYS | A | 54 | 37.636 | 14.949 | −14.870 | 1.00 | 0.00 | H |
| ATOM | 951 | 3HZ | LYS | A | 54 | 38.835 | 15.571 | −15.803 | 1.00 | 0.00 | H |
| ATOM | 952 | N | ALA | A | 55 | 43.822 | 11.763 | −10.674 | 1.00 | 0.00 | N |
| ATOM | 953 | CA | ALA | A | 55 | 45.070 | 12.022 | −9.968 | 1.00 | 0.00 | C |
| ATOM | 954 | C | ALA | A | 55 | 45.754 | 10.711 | −9.610 | 1.00 | 0.00 | C |
| ATOM | 955 | O | ALA | A | 55 | 46.723 | 10.699 | −8.850 | 1.00 | 0.00 | O |
| ATOM | 956 | CB | ALA | A | 55 | 44.803 | 12.828 | −8.700 | 1.00 | 0.00 | C |
| ATOM | 957 | H | ALA | A | 55 | 42.943 | 11.909 | −10.184 | 1.00 | 0.00 | H |
| ATOM | 958 | HA | ALA | A | 55 | 45.748 | 12.595 | −10.604 | 1.00 | 0.00 | H |
| ATOM | 959 | 1HB | ALA | A | 55 | 45.729 | 13.078 | −8.181 | 1.00 | 0.00 | H |
| ATOM | 960 | 2HB | ALA | A | 55 | 44.297 | 13.766 | −8.930 | 1.00 | 0.00 | H |
| ATOM | 961 | 3HB | ALA | A | 55 | 44.171 | 12.277 | −8.002 | 1.00 | 0.00 | H |
| ATOM | 962 | N | LYS | A | 56 | 45.251 | 9.609 | −10.160 | 1.00 | 0.00 | N |
| ATOM | 963 | CA | LYS | A | 56 | 45.815 | 8.293 | −9.876 | 1.00 | 0.00 | C |
| ATOM | 964 | C | LYS | A | 56 | 45.988 | 8.144 | −8.364 | 1.00 | 0.00 | C |
| ATOM | 965 | O | LYS | A | 56 | 47.002 | 7.644 | −7.879 | 1.00 | 0.00 | O |
| ATOM | 966 | CB | LYS | A | 56 | 47.163 | 8.136 | −10.590 | 1.00 | 0.00 | C |
| ATOM | 967 | CG | LYS | A | 56 | 47.105 | 8.340 | −12.117 | 1.00 | 0.00 | C |
| ATOM | 968 | CD | LYS | A | 56 | 46.323 | 7.239 | −12.861 | 1.00 | 0.00 | C |
| ATOM | 969 | CE | LYS | A | 56 | 45.689 | 7.725 | −14.169 | 1.00 | 0.00 | C |
| ATOM | 970 | NZ | LYS | A | 56 | 44.610 | 8.689 | −13.906 | 1.00 | 0.00 | N |
| ATOM | 971 | H | LYS | A | 56 | 44.425 | 9.664 | −10.741 | 1.00 | 0.00 | H |
| ATOM | 972 | HA | LYS | A | 56 | 45.110 | 7.541 | −10.232 | 1.00 | 0.00 | H |
| ATOM | 973 | 1HB | LYS | A | 56 | 47.900 | 8.826 | −10.175 | 1.00 | 0.00 | H |
| ATOM | 974 | 2HB | LYS | A | 56 | 47.564 | 7.141 | −10.391 | 1.00 | 0.00 | H |
| ATOM | 975 | 1HG | LYS | A | 56 | 46.700 | 9.328 | −12.329 | 1.00 | 0.00 | H |
| ATOM | 976 | 2HG | LYS | A | 56 | 48.122 | 8.368 | −12.512 | 1.00 | 0.00 | H |
| ATOM | 977 | 1HD | LYS | A | 56 | 47.000 | 6.410 | −13.073 | 1.00 | 0.00 | H |
| ATOM | 978 | 2HD | LYS | A | 56 | 45.537 | 6.813 | −12.239 | 1.00 | 0.00 | H |
| ATOM | 979 | 1HE | LYS | A | 56 | 46.435 | 8.178 | −14.823 | 1.00 | 0.00 | H |
| ATOM | 980 | 2HE | LYS | A | 56 | 45.266 | 6.877 | −14.709 | 1.00 | 0.00 | H |
| ATOM | 981 | 1HZ | LYS | A | 56 | 44.122 | 8.894 | −14.761 | 1.00 | 0.00 | H |
| ATOM | 982 | 2HZ | LYS | A | 56 | 43.959 | 8.321 | −13.222 | 1.00 | 0.00 | H |
| ATOM | 983 | 3HZ | LYS | A | 56 | 44.993 | 9.551 | −13.533 | 1.00 | 0.00 | H |
| ATOM | 984 | N | LEU | A | 57 | 44.979 | 8.597 | −7.629 | 1.00 | 0.00 | N |
| ATOM | 985 | CA | LEU | A | 57 | 44.973 | 8.548 | −6.174 | 1.00 | 0.00 | C |
| ATOM | 986 | C | LEU | A | 57 | 43.761 | 7.753 | −5.709 | 1.00 | 0.00 | C |
| ATOM | 987 | O | LEU | A | 57 | 42.768 | 7.639 | −6.429 | 1.00 | 0.00 | O |
| ATOM | 988 | CB | LEU | A | 57 | 44.879 | 9.967 | −5.607 | 1.00 | 0.00 | C |
| ATOM | 989 | CG | LEU | A | 57 | 45.125 | 10.165 | −4.089 | 1.00 | 0.00 | C |
| ATOM | 990 | CD1 | LEU | A | 57 | 46.436 | 9.531 | −3.594 | 1.00 | 0.00 | C |
| ATOM | 991 | CD2 | LEU | A | 57 | 45.119 | 11.664 | −3.745 | 1.00 | 0.00 | C |
| ATOM | 992 | H | LEU | A | 57 | 44.118 | 8.912 | −8.083 | 1.00 | 0.00 | H |
| ATOM | 993 | HA | LEU | A | 57 | 45.870 | 8.057 | −5.798 | 1.00 | 0.00 | H |
| ATOM | 994 | 1HB | LEU | A | 57 | 45.628 | 10.566 | −6.129 | 1.00 | 0.00 | H |
| ATOM | 995 | 2HB | LEU | A | 57 | 43.927 | 10.410 | −5.899 | 1.00 | 0.00 | H |
| ATOM | 996 | HG | LEU | A | 57 | 44.308 | 9.707 | −3.532 | 1.00 | 0.00 | H |
| ATOM | 997 | 1HD1 | LEU | A | 57 | 46.617 | 9.775 | −2.546 | 1.00 | 0.00 | H |
| ATOM | 998 | 2HD1 | LEU | A | 57 | 46.420 | 8.443 | −3.658 | 1.00 | 0.00 | H |
| ATOM | 999 | 3HD1 | LEU | A | 57 | 47.293 | 9.888 | −4.167 | 1.00 | 0.00 | H |
| ATOM | 1000 | 1HD2 | LEU | A | 57 | 45.176 | 11.822 | −2.667 | 1.00 | 0.00 | H |
| ATOM | 1001 | 2HD2 | LEU | A | 57 | 45.966 | 12.179 | −4.199 | 1.00 | 0.00 | H |
| ATOM | 1002 | 3HD2 | LEU | A | 57 | 44.215 | 12.159 | −4.098 | 1.00 | 0.00 | H |
| ATOM | 1003 | N | THR | A | 58 | 43.846 | 7.207 | −4.504 | 1.00 | 0.00 | N |
| ATOM | 1004 | CA | THR | A | 58 | 42.744 | 6.445 | −3.927 | 1.00 | 0.00 | C |
| ATOM | 1005 | C | THR | A | 58 | 41.960 | 7.365 | −2.996 | 1.00 | 0.00 | C |
| ATOM | 1006 | O | THR | A | 58 | 42.554 | 8.156 | −2.261 | 1.00 | 0.00 | O |
| ATOM | 1007 | CB | THR | A | 58 | 43.260 | 5.264 | −3.081 | 1.00 | 0.00 | C |
| ATOM | 1008 | OG1 | THR | A | 58 | 44.299 | 5.676 | −2.202 | 1.00 | 0.00 | O |
| ATOM | 1009 | CG2 | THR | A | 58 | 43.812 | 4.126 | −3.950 | 1.00 | 0.00 | C |
| ATOM | 1010 | H | THR | A | 58 | 44.620 | 7.446 | −3.904 | 1.00 | 0.00 | H |
| ATOM | 1011 | HA | THR | A | 58 | 42.077 | 6.058 | −4.701 | 1.00 | 0.00 | H |
| ATOM | 1012 | HB | THR | A | 58 | 42.443 | 4.860 | −2.477 | 1.00 | 0.00 | H |
| ATOM | 1013 | HG1 | THR | A | 58 | 43.972 | 6.406 | −1.685 | 1.00 | 0.00 | H |
| ATOM | 1014 | 1HG2 | THR | A | 58 | 44.150 | 3.291 | −3.336 | 1.00 | 0.00 | H |
| ATOM | 1015 | 2HG2 | THR | A | 58 | 43.043 | 3.752 | −4.625 | 1.00 | 0.00 | H |
| ATOM | 1016 | 3HG2 | THR | A | 58 | 44.655 | 4.459 | −4.556 | 1.00 | 0.00 | H |
| ATOM | 1017 | N | ILE | A | 59 | 40.634 | 7.282 | −3.025 | 1.00 | 0.00 | N |
| ATOM | 1018 | CA | ILE | A | 59 | 39.844 | 8.107 | −2.125 | 1.00 | 0.00 | C |
| ATOM | 1019 | C | ILE | A | 59 | 40.100 | 7.542 | −0.725 | 1.00 | 0.00 | C |
| ATOM | 1020 | O | ILE | A | 59 | 39.935 | 6.337 | −0.496 | 1.00 | 0.00 | O |
| ATOM | 1021 | CB | ILE | A | 59 | 38.347 | 8.024 | −2.466 | 1.00 | 0.00 | C |
| ATOM | 1022 | CG1 | ILE | A | 59 | 38.034 | 8.195 | −3.973 | 1.00 | 0.00 | C |
| ATOM | 1023 | CG2 | ILE | A | 59 | 37.543 | 9.056 | −1.658 | 1.00 | 0.00 | C |
| ATOM | 1024 | CD1 | ILE | A | 59 | 37.737 | 6.882 | −4.704 | 1.00 | 0.00 | C |
| ATOM | 1025 | H | ILE | A | 59 | 40.153 | 6.617 | −3.611 | 1.00 | 0.00 | H |
| ATOM | 1026 | HA | ILE | A | 59 | 40.155 | 9.143 | −2.254 | 1.00 | 0.00 | H |
| ATOM | 1027 | HB | ILE | A | 59 | 37.973 | 7.042 | −2.174 | 1.00 | 0.00 | H |
| ATOM | 1028 | 1HG1 | ILE | A | 59 | 37.172 | 8.849 | −4.111 | 1.00 | 0.00 | H |
| ATOM | 1029 | 2HG1 | ILE | A | 59 | 38.859 | 8.696 | −4.464 | 1.00 | 0.00 | H |
| ATOM | 1030 | 1HG2 | ILE | A | 59 | 36.477 | 8.990 | −1.876 | 1.00 | 0.00 | H |
| ATOM | 1031 | 2HG2 | ILE | A | 59 | 37.661 | 8.906 | −0.586 | 1.00 | 0.00 | H |
| ATOM | 1032 | 3HG2 | ILE | A | 59 | 37.870 | 10.063 | −1.905 | 1.00 | 0.00 | H |
| ATOM | 1033 | 1HD1 | ILE | A | 59 | 37.501 | 7.064 | −5.753 | 1.00 | 0.00 | H |
| ATOM | 1034 | 2HD1 | ILE | A | 59 | 38.582 | 6.195 | −4.669 | 1.00 | 0.00 | H |
| ATOM | 1035 | 3HD1 | ILE | A | 59 | 36.880 | 6.381 | −4.255 | 1.00 | 0.00 | H |
| ATOM | 1036 | N | PRO | A | 60 | 40.552 | 8.394 | 0.218 | 1.00 | 0.00 | N |
| ATOM | 1037 | CA | PRO | A | 60 | 40.819 | 7.926 | 1.582 | 1.00 | 0.00 | C |
| ATOM | 1038 | C | PRO | A | 60 | 39.640 | 7.122 | 2.132 | 1.00 | 0.00 | C |
| ATOM | 1039 | O | PRO | A | 60 | 38.481 | 7.524 | 1.995 | 1.00 | 0.00 | O |
| ATOM | 1040 | CB | PRO | A | 60 | 41.067 | 9.217 | 2.361 | 1.00 | 0.00 | C |
| ATOM | 1041 | CG | PRO | A | 60 | 41.661 | 10.173 | 1.347 | 1.00 | 0.00 | C |
| ATOM | 1042 | CD | PRO | A | 60 | 40.966 | 9.789 | 0.043 | 1.00 | 0.00 | C |
| ATOM | 1043 | HA | PRO | A | 60 | 41.723 | 7.315 | 1.558 | 1.00 | 0.00 | H |
| ATOM | 1044 | 1HB | PRO | A | 60 | 40.162 | 9.695 | 2.666 | 1.00 | 0.00 | H |
| ATOM | 1045 | 2HB | PRO | A | 60 | 41.696 | 9.074 | 3.240 | 1.00 | 0.00 | H |
| ATOM | 1046 | 1HG | PRO | A | 60 | 42.733 | 9.989 | 1.259 | 1.00 | 0.00 | H |
| ATOM | 1047 | 2HG | PRO | A | 60 | 41.526 | 11.223 | 1.612 | 1.00 | 0.00 | H |
| ATOM | 1048 | 1HD | PRO | A | 60 | 41.646 | 9.937 | −0.795 | 1.00 | 0.00 | H |
| ATOM | 1049 | 2HD | PRO | A | 60 | 40.081 | 10.401 | −0.130 | 1.00 | 0.00 | H |
| ATOM | 1050 | N | ILE | A | 61 | 39.928 | 6.008 | 2.826 | 1.00 | 0.00 | N |
| ATOM | 1051 | CA | ILE | A | 61 | 38.999 | 4.943 | 3.257 | 1.00 | 0.00 | C |
| ATOM | 1052 | C | ILE | A | 61 | 37.632 | 5.045 | 2.538 | 1.00 | 0.00 | C |
| ATOM | 1053 | O | ILE | A | 61 | 36.587 | 5.303 | 3.135 | 1.00 | 0.00 | O |
| ATOM | 1054 | CB | ILE | A | 61 | 38.934 | 4.842 | 4.811 | 1.00 | 0.00 | C |
| ATOM | 1055 | CG1 | ILE | A | 61 | 40.316 | 4.980 | 5.505 | 1.00 | 0.00 | C |
| ATOM | 1056 | CG2 | ILE | A | 61 | 38.224 | 3.551 | 5.275 | 1.00 | 0.00 | C |
| ATOM | 1057 | CD1 | ILE | A | 61 | 40.243 | 5.116 | 7.034 | 1.00 | 0.00 | C |
| ATOM | 1058 | H | ILE | A | 61 | 40.905 | 5.784 | 2.928 | 1.00 | 0.00 | H |
| ATOM | 1059 | HA | ILE | A | 61 | 39.441 | 4.012 | 2.902 | 1.00 | 0.00 | H |
| ATOM | 1060 | HB | ILE | A | 61 | 38.332 | 5.679 | 5.154 | 1.00 | 0.00 | H |
| ATOM | 1061 | 1HG1 | ILE | A | 61 | 40.953 | 4.134 | 5.240 | 1.00 | 0.00 | H |
| ATOM | 1062 | 2HG1 | ILE | A | 61 | 40.833 | 5.870 | 5.151 | 1.00 | 0.00 | H |
| ATOM | 1063 | 1HG2 | ILE | A | 61 | 38.099 | 3.521 | 6.357 | 1.00 | 0.00 | H |
| ATOM | 1064 | 2HG2 | ILE | A | 61 | 37.219 | 3.467 | 4.862 | 1.00 | 0.00 | H |
| ATOM | 1065 | 3HG2 | ILE | A | 61 | 38.779 | 2.660 | 4.979 | 1.00 | 0.00 | H |
| ATOM | 1066 | 1HD1 | ILE | A | 61 | 41.225 | 5.337 | 7.452 | 1.00 | 0.00 | H |
| ATOM | 1067 | 2HD1 | ILE | A | 61 | 39.573 | 5.922 | 7.336 | 1.00 | 0.00 | H |
| ATOM | 1068 | 3HD1 | ILE | A | 61 | 39.894 | 4.203 | 7.517 | 1.00 | 0.00 | H |
| ATOM | 1069 | N | LEU | A | 62 | 37.767 | 4.959 | 1.202 | 1.00 | 0.00 | N |
| ATOM | 1070 | CA | LEU | A | 62 | 36.866 | 5.047 | 0.062 | 1.00 | 0.00 | C |
| ATOM | 1071 | C | LEU | A | 62 | 35.877 | 6.225 | −0.019 | 1.00 | 0.00 | C |
| ATOM | 1072 | O | LEU | A | 62 | 35.543 | 6.643 | −1.125 | 1.00 | 0.00 | O |
| ATOM | 1073 | CB | LEU | A | 62 | 36.254 | 3.685 | −0.292 | 1.00 | 0.00 | C |
| ATOM | 1074 | CG | LEU | A | 62 | 35.535 | 2.795 | 0.751 | 1.00 | 0.00 | C |
| ATOM | 1075 | CD1 | LEU | A | 62 | 36.436 | 2.145 | 1.812 | 1.00 | 0.00 | C |
| ATOM | 1076 | CD2 | LEU | A | 62 | 34.279 | 3.441 | 1.338 | 1.00 | 0.00 | C |
| ATOM | 1077 | H | LEU | A | 62 | 38.732 | 4.937 | 0.909 | 1.00 | 0.00 | H |
| ATOM | 1078 | HA | LEU | A | 62 | 37.544 | 5.266 | −0.763 | 1.00 | 0.00 | H |
| ATOM | 1079 | 1HB | LEU | A | 62 | 35.516 | 3.840 | −1.060 | 1.00 | 0.00 | H |
| ATOM | 1080 | 2HB | LEU | A | 62 | 37.027 | 3.089 | −0.777 | 1.00 | 0.00 | H |
| ATOM | 1081 | HG | LEU | A | 62 | 35.164 | 1.954 | 0.164 | 1.00 | 0.00 | H |
| ATOM | 1082 | 1HD1 | LEU | A | 62 | 36.057 | 1.162 | 2.091 | 1.00 | 0.00 | H |
| ATOM | 1083 | 2HD1 | LEU | A | 62 | 37.464 | 2.036 | 1.475 | 1.00 | 0.00 | H |
| ATOM | 1084 | 3HD1 | LEU | A | 62 | 36.480 | 2.723 | 2.725 | 1.00 | 0.00 | H |
| ATOM | 1085 | 1HD2 | LEU | A | 62 | 33.705 | 2.728 | 1.929 | 1.00 | 0.00 | H |
| ATOM | 1086 | 2HD2 | LEU | A | 62 | 34.514 | 4.287 | 1.980 | 1.00 | 0.00 | H |
| ATOM | 1087 | 3HD2 | LEU | A | 62 | 33.630 | 3.792 | 0.535 | 1.00 | 0.00 | H |
| ATOM | 1088 | N | TYR | A | 63 | 35.472 | 6.822 | 1.096 | 1.00 | 0.00 | N |
| ATOM | 1089 | CA | TYR | A | 63 | 34.709 | 8.063 | 1.161 | 1.00 | 0.00 | C |
| ATOM | 1090 | C | TYR | A | 63 | 35.558 | 8.982 | 2.045 | 1.00 | 0.00 | C |
| ATOM | 1091 | O | TYR | A | 63 | 35.584 | 8.751 | 3.243 | 1.00 | 0.00 | O |
| ATOM | 1092 | CB | TYR | A | 63 | 33.293 | 7.816 | 1.757 | 1.00 | 0.00 | C |
| ATOM | 1093 | CG | TYR | A | 63 | 32.216 | 7.263 | 0.834 | 1.00 | 0.00 | C |
| ATOM | 1094 | CD1 | TYR | A | 63 | 31.519 | 8.121 | −0.043 | 1.00 | 0.00 | C |
| ATOM | 1095 | CD2 | TYR | A | 63 | 31.824 | 5.912 | 0.930 | 1.00 | 0.00 | C |
| ATOM | 1096 | CE1 | TYR | A | 63 | 30.413 | 7.643 | −0.772 | 1.00 | 0.00 | C |
| ATOM | 1097 | CE2 | TYR | A | 63 | 30.721 | 5.431 | 0.197 | 1.00 | 0.00 | C |
| ATOM | 1098 | CZ | TYR | A | 63 | 30.002 | 6.304 | −0.642 | 1.00 | 0.00 | C |
| ATOM | 1099 | OH | TYR | A | 63 | 28.910 | 5.865 | −1.331 | 1.00 | 0.00 | O |
| ATOM | 1100 | H | TYR | A | 63 | 35.791 | 6.413 | 1.973 | 1.00 | 0.00 | H |
| ATOM | 1101 | HA | TYR | A | 63 | 34.603 | 8.518 | 0.175 | 1.00 | 0.00 | H |
| ATOM | 1102 | 1HB | TYR | A | 63 | 33.369 | 7.180 | 2.640 | 1.00 | 0.00 | H |
| ATOM | 1103 | 2HB | TYR | A | 63 | 32.900 | 8.755 | 2.132 | 1.00 | 0.00 | H |
| ATOM | 1104 | HD1 | TYR | A | 63 | 31.800 | 9.159 | −0.136 | 1.00 | 0.00 | H |
| ATOM | 1105 | HD2 | TYR | A | 63 | 32.324 | 5.255 | 1.624 | 1.00 | 0.00 | H |
| ATOM | 1106 | HE1 | TYR | A | 63 | 29.867 | 8.309 | −1.420 | 1.00 | 0.00 | H |
| ATOM | 1107 | HE2 | TYR | A | 63 | 30.422 | 4.399 | 0.310 | 1.00 | 0.00 | H |
| ATOM | 1108 | HH | TYR | A | 63 | 28.748 | 4.940 | −1.223 | 1.00 | 0.00 | H |
| ATOM | 1109 | N | VAL | A | 64 | 36.252 | 9.985 | 1.487 | 1.00 | 0.00 | N |
| ATOM | 1110 | CA | VAL | A | 64 | 37.390 | 10.780 | 2.001 | 1.00 | 0.00 | C |
| ATOM | 1111 | C | VAL | A | 64 | 37.796 | 10.661 | 3.501 | 1.00 | 0.00 | C |
| ATOM | 1112 | O | VAL | A | 64 | 37.805 | 11.641 | 4.236 | 1.00 | 0.00 | O |
| ATOM | 1113 | CB | VAL | A | 64 | 37.382 | 12.233 | 1.427 | 1.00 | 0.00 | C |
| ATOM | 1114 | CG1 | VAL | A | 64 | 38.764 | 12.917 | 1.477 | 1.00 | 0.00 | C |
| ATOM | 1115 | CG2 | VAL | A | 64 | 36.822 | 12.356 | −0.005 | 1.00 | 0.00 | C |
| ATOM | 1116 | H | VAL | A | 64 | 36.071 | 10.121 | 0.505 | 1.00 | 0.00 | H |
| ATOM | 1117 | HA | VAL | A | 64 | 38.223 | 10.298 | 1.492 | 1.00 | 0.00 | H |
| ATOM | 1118 | HB | VAL | A | 64 | 36.731 | 12.818 | 2.070 | 1.00 | 0.00 | H |
| ATOM | 1119 | 1HG1 | VAL | A | 64 | 38.698 | 13.951 | 1.137 | 1.00 | 0.00 | H |
| ATOM | 1120 | 2HG1 | VAL | A | 64 | 39.177 | 12.947 | 2.485 | 1.00 | 0.00 | H |
| ATOM | 1121 | 3HG1 | VAL | A | 64 | 39.485 | 12.411 | 0.839 | 1.00 | 0.00 | H |
| ATOM | 1122 | 1HG2 | VAL | A | 64 | 36.789 | 13.400 | −0.319 | 1.00 | 0.00 | H |
| ATOM | 1123 | 2HG2 | VAL | A | 64 | 37.450 | 11.828 | −0.716 | 1.00 | 0.00 | H |
| ATOM | 1124 | 3HG2 | VAL | A | 64 | 35.805 | 11.978 | −0.095 | 1.00 | 0.00 | H |
| ATOM | 1125 | N | LYS | A | 65 | 38.219 | 9.460 | 3.913 | 1.00 | 0.00 | N |
| ATOM | 1126 | CA | LYS | A | 65 | 38.852 | 9.025 | 5.167 | 1.00 | 0.00 | C |
| ATOM | 1127 | C | LYS | A | 65 | 37.818 | 8.418 | 6.107 | 1.00 | 0.00 | C |
| ATOM | 1128 | O | LYS | A | 65 | 37.863 | 8.615 | 7.317 | 1.00 | 0.00 | O |
| ATOM | 1129 | CB | LYS | A | 65 | 39.826 | 10.055 | 5.786 | 1.00 | 0.00 | C |
| ATOM | 1130 | CG | LYS | A | 65 | 40.764 | 9.420 | 6.853 | 1.00 | 0.00 | C |
| ATOM | 1131 | CD | LYS | A | 65 | 41.004 | 10.231 | 8.150 | 1.00 | 0.00 | C |
| ATOM | 1132 | CE | LYS | A | 65 | 39.793 | 10.460 | 9.091 | 1.00 | 0.00 | C |
| ATOM | 1133 | NZ | LYS | A | 65 | 39.103 | 9.230 | 9.524 | 1.00 | 0.00 | N |
| ATOM | 1134 | H | LYS | A | 65 | 38.120 | 8.731 | 3.206 | 1.00 | 0.00 | H |
| ATOM | 1135 | HA | LYS | A | 65 | 39.457 | 8.173 | 4.866 | 1.00 | 0.00 | H |
| ATOM | 1136 | 1HB | LYS | A | 65 | 40.431 | 10.528 | 5.012 | 1.00 | 0.00 | H |
| ATOM | 1137 | 2HB | LYS | A | 65 | 39.244 | 10.858 | 6.237 | 1.00 | 0.00 | H |
| ATOM | 1138 | 1HG | LYS | A | 65 | 40.428 | 8.422 | 7.132 | 1.00 | 0.00 | H |
| ATOM | 1139 | 2HG | LYS | A | 65 | 41.730 | 9.245 | 6.377 | 1.00 | 0.00 | H |
| ATOM | 1140 | 1HD | LYS | A | 65 | 41.787 | 9.731 | 8.721 | 1.00 | 0.00 | H |
| ATOM | 1141 | 2HD | LYS | A | 65 | 41.420 | 11.203 | 7.881 | 1.00 | 0.00 | H |
| ATOM | 1142 | 1HE | LYS | A | 65 | 40.124 | 10.995 | 9.982 | 1.00 | 0.00 | H |
| ATOM | 1143 | 2HE | LYS | A | 65 | 39.057 | 11.104 | 8.609 | 1.00 | 0.00 | H |
| ATOM | 1144 | 1HZ | LYS | A | 65 | 38.379 | 9.437 | 10.190 | 1.00 | 0.00 | H |
| ATOM | 1145 | 2HZ | LYS | A | 65 | 38.628 | 8.819 | 8.716 | 1.00 | 0.00 | H |
| ATOM | 1146 | 3HZ | LYS | A | 65 | 39.652 | 8.481 | 9.942 | 1.00 | 0.00 | H |
| ATOM | 1147 | N | VAL | A | 66 | 36.824 | 7.781 | 5.483 | 1.00 | 0.00 | N |
| ATOM | 1148 | CA | VAL | A | 66 | 35.530 | 7.394 | 6.000 | 1.00 | 0.00 | C |
| ATOM | 1149 | C | VAL | A | 66 | 34.843 | 8.675 | 6.543 | 1.00 | 0.00 | C |
| ATOM | 1150 | O | VAL | A | 66 | 34.370 | 8.743 | 7.668 | 1.00 | 0.00 | O |
| ATOM | 1151 | CB | VAL | A | 66 | 35.626 | 6.096 | 6.858 | 1.00 | 0.00 | C |
| ATOM | 1152 | CG1 | VAL | A | 66 | 36.229 | 6.214 | 8.270 | 1.00 | 0.00 | C |
| ATOM | 1153 | CG2 | VAL | A | 66 | 34.330 | 5.260 | 6.777 | 1.00 | 0.00 | C |
| ATOM | 1154 | H | VAL | A | 66 | 36.860 | 7.792 | 4.472 | 1.00 | 0.00 | H |
| ATOM | 1155 | HA | VAL | A | 66 | 34.966 | 7.128 | 5.111 | 1.00 | 0.00 | H |
| ATOM | 1156 | HB | VAL | A | 66 | 36.338 | 5.455 | 6.339 | 1.00 | 0.00 | H |
| ATOM | 1157 | 1HG1 | VAL | A | 66 | 36.035 | 5.319 | 8.861 | 1.00 | 0.00 | H |
| ATOM | 1158 | 2HG1 | VAL | A | 66 | 37.313 | 6.305 | 8.218 | 1.00 | 0.00 | H |
| ATOM | 1159 | 3HG1 | VAL | A | 66 | 35.867 | 7.074 | 8.828 | 1.00 | 0.00 | H |
| ATOM | 1160 | 1HG2 | VAL | A | 66 | 34.311 | 4.459 | 7.517 | 1.00 | 0.00 | H |
| ATOM | 1161 | 2HG2 | VAL | A | 66 | 33.428 | 5.858 | 6.889 | 1.00 | 0.00 | H |
| ATOM | 1162 | 3HG2 | VAL | A | 66 | 34.251 | 4.785 | 5.798 | 1.00 | 0.00 | H |
| ATOM | 1163 | N | TYR | A | 67 | 34.848 | 9.756 | 5.738 | 1.00 | 0.00 | N |
| ATOM | 1164 | CA | TYR | A | 67 | 34.322 | 11.069 | 6.132 | 1.00 | 0.00 | C |
| ATOM | 1165 | C | TYR | A | 67 | 32.819 | 11.051 | 6.417 | 1.00 | 0.00 | C |
| ATOM | 1166 | O | TYR | A | 67 | 32.326 | 11.885 | 7.165 | 1.00 | 0.00 | O |
| ATOM | 1167 | CB | TYR | A | 67 | 34.697 | 12.196 | 5.139 | 1.00 | 0.00 | C |
| ATOM | 1168 | CG | TYR | A | 67 | 34.164 | 12.335 | 3.716 | 1.00 | 0.00 | C |
| ATOM | 1169 | CD1 | TYR | A | 67 | 33.388 | 11.353 | 3.067 | 1.00 | 0.00 | C |
| ATOM | 1170 | CD2 | TYR | A | 67 | 34.458 | 13.533 | 3.028 | 1.00 | 0.00 | C |
| ATOM | 1171 | CE1 | TYR | A | 67 | 32.949 | 11.553 | 1.744 | 1.00 | 0.00 | C |
| ATOM | 1172 | CE2 | TYR | A | 67 | 34.039 | 13.724 | 1.698 | 1.00 | 0.00 | C |
| ATOM | 1173 | CZ | TYR | A | 67 | 33.296 | 12.724 | 1.049 | 1.00 | 0.00 | C |
| ATOM | 1174 | OH | TYR | A | 67 | 32.935 | 12.871 | −0.256 | 1.00 | 0.00 | O |
| ATOM | 1175 | H | TYR | A | 67 | 35.262 | 9.646 | 4.817 | 1.00 | 0.00 | H |
| ATOM | 1176 | HA | TYR | A | 67 | 34.801 | 11.316 | 7.082 | 1.00 | 0.00 | H |
| ATOM | 1177 | 1HB | TYR | A | 67 | 34.412 | 13.124 | 5.626 | 1.00 | 0.00 | H |
| ATOM | 1178 | 2HB | TYR | A | 67 | 35.772 | 12.280 | 5.112 | 1.00 | 0.00 | H |
| ATOM | 1179 | HD1 | TYR | A | 67 | 33.130 | 10.434 | 3.560 | 1.00 | 0.00 | H |
| ATOM | 1180 | HD2 | TYR | A | 67 | 35.042 | 14.303 | 3.510 | 1.00 | 0.00 | H |
| ATOM | 1181 | HE1 | TYR | A | 67 | 32.350 | 10.800 | 1.256 | 1.00 | 0.00 | H |
| ATOM | 1182 | HE2 | TYR | A | 67 | 34.319 | 14.627 | 1.178 | 1.00 | 0.00 | H |
| ATOM | 1183 | HH | TYR | A | 67 | 33.104 | 13.736 | −0.596 | 1.00 | 0.00 | H |
| ATOM | 1184 | N | MET | A | 68 | 32.124 | 10.065 | 5.852 | 1.00 | 0.00 | N |
| ATOM | 1185 | CA | MET | A | 68 | 30.744 | 9.690 | 6.035 | 1.00 | 0.00 | C |
| ATOM | 1186 | C | MET | A | 68 | 30.441 | 8.723 | 4.888 | 1.00 | 0.00 | C |
| ATOM | 1187 | O | MET | A | 68 | 30.898 | 8.930 | 3.764 | 1.00 | 0.00 | O |
| ATOM | 1188 | CB | MET | A | 68 | 29.784 | 10.892 | 5.880 | 1.00 | 0.00 | C |
| ATOM | 1189 | CG | MET | A | 68 | 28.370 | 10.646 | 6.439 | 1.00 | 0.00 | C |
| ATOM | 1190 | SD | MET | A | 68 | 27.014 | 11.604 | 5.695 | 1.00 | 0.00 | S |
| ATOM | 1191 | CE | MET | A | 68 | 25.911 | 11.825 | 7.122 | 1.00 | 0.00 | C |
| ATOM | 1192 | H | MET | A | 68 | 32.636 | 9.473 | 5.216 | 1.00 | 0.00 | H |
| ATOM | 1193 | HA | MET | A | 68 | 30.650 | 9.207 | 7.010 | 1.00 | 0.00 | H |
| ATOM | 1194 | 1HB | MET | A | 68 | 30.137 | 11.805 | 6.347 | 1.00 | 0.00 | H |
| ATOM | 1195 | 2HB | MET | A | 68 | 29.699 | 11.117 | 4.817 | 1.00 | 0.00 | H |
| ATOM | 1196 | 1HG | MET | A | 68 | 28.103 | 9.601 | 6.331 | 1.00 | 0.00 | H |
| ATOM | 1197 | 2HG | MET | A | 68 | 28.390 | 10.861 | 7.503 | 1.00 | 0.00 | H |
| ATOM | 1198 | 1HE | MET | A | 68 | 25.096 | 12.506 | 6.879 | 1.00 | 0.00 | H |
| ATOM | 1199 | 2HE | MET | A | 68 | 25.479 | 10.879 | 7.443 | 1.00 | 0.00 | H |
| ATOM | 1200 | 3HE | MET | A | 68 | 26.445 | 12.251 | 7.967 | 1.00 | 0.00 | H |
| ATOM | 1201 | N | TYR | A | 69 | 29.594 | 7.729 | 5.137 | 1.00 | 0.00 | N |
| ATOM | 1202 | CA | TYR | A | 69 | 28.804 | 7.093 | 4.098 | 1.00 | 0.00 | C |
| ATOM | 1203 | C | TYR | A | 69 | 27.544 | 6.609 | 4.753 | 1.00 | 0.00 | C |
| ATOM | 1204 | O | TYR | A | 69 | 27.522 | 5.618 | 5.476 | 1.00 | 0.00 | O |
| ATOM | 1205 | CB | TYR | A | 69 | 29.525 | 5.998 | 3.347 | 1.00 | 0.00 | C |
| ATOM | 1206 | CG | TYR | A | 69 | 29.772 | 4.655 | 4.032 | 1.00 | 0.00 | C |
| ATOM | 1207 | CD1 | TYR | A | 69 | 30.607 | 4.559 | 5.168 | 1.00 | 0.00 | C |
| ATOM | 1208 | CD2 | TYR | A | 69 | 29.092 | 3.507 | 3.573 | 1.00 | 0.00 | C |
| ATOM | 1209 | CE1 | TYR | A | 69 | 30.686 | 3.349 | 5.888 | 1.00 | 0.00 | C |
| ATOM | 1210 | CE2 | TYR | A | 69 | 29.176 | 2.300 | 4.292 | 1.00 | 0.00 | C |
| ATOM | 1211 | CZ | TYR | A | 69 | 29.938 | 2.231 | 5.469 | 1.00 | 0.00 | C |
| ATOM | 1212 | OH | TYR | A | 69 | 29.930 | 1.082 | 6.203 | 1.00 | 0.00 | O |
| ATOM | 1213 | H | TYR | A | 69 | 29.306 | 7.547 | 6.087 | 1.00 | 0.00 | H |
| ATOM | 1214 | HA | TYR | A | 69 | 28.545 | 7.859 | 3.361 | 1.00 | 0.00 | H |
| ATOM | 1215 | 1HB | TYR | A | 69 | 28.993 | 5.815 | 2.413 | 1.00 | 0.00 | H |
| ATOM | 1216 | 2HB | TYR | A | 69 | 30.497 | 6.385 | 3.056 | 1.00 | 0.00 | H |
| ATOM | 1217 | HD1 | TYR | A | 69 | 31.150 | 5.423 | 5.520 | 1.00 | 0.00 | H |
| ATOM | 1218 | HD2 | TYR | A | 69 | 28.463 | 3.557 | 2.698 | 1.00 | 0.00 | H |
| ATOM | 1219 | HE1 | TYR | A | 69 | 31.298 | 3.290 | 6.775 | 1.00 | 0.00 | H |
| ATOM | 1220 | HE2 | TYR | A | 69 | 28.630 | 1.433 | 3.956 | 1.00 | 0.00 | H |
| ATOM | 1221 | HH | TYR | A | 69 | 29.218 | 0.504 | 5.969 | 1.00 | 0.00 | H |
| ATOM | 1222 | N | GLN | A | 70 | 26.495 | 7.372 | 4.518 | 1.00 | 0.00 | N |
| ATOM | 1223 | CA | GLN | A | 70 | 25.164 | 7.149 | 5.013 | 1.00 | 0.00 | C |
| ATOM | 1224 | C | GLN | A | 70 | 24.261 | 7.124 | 3.781 | 1.00 | 0.00 | C |
| ATOM | 1225 | O | GLN | A | 70 | 23.098 | 7.517 | 3.842 | 1.00 | 0.00 | O |
| ATOM | 1226 | CB | GLN | A | 70 | 24.855 | 8.198 | 6.092 | 1.00 | 0.00 | C |
| ATOM | 1227 | CG | GLN | A | 70 | 25.633 | 8.030 | 7.416 | 1.00 | 0.00 | C |
| ATOM | 1228 | CD | GLN | A | 70 | 25.685 | 6.608 | 7.969 | 1.00 | 0.00 | C |
| ATOM | 1229 | OE1 | GLN | A | 70 | 24.700 | 5.893 | 7.959 | 1.00 | 0.00 | O |
| ATOM | 1230 | NE2 | GLN | A | 70 | 26.843 | 6.149 | 8.410 | 1.00 | 0.00 | N |
| ATOM | 1231 | H | GLN | A | 70 | 26.610 | 8.152 | 3.889 | 1.00 | 0.00 | H |
| ATOM | 1232 | HA | GLN | A | 70 | 25.061 | 6.154 | 5.448 | 1.00 | 0.00 | H |
| ATOM | 1233 | 1HB | GLN | A | 70 | 25.062 | 9.187 | 5.690 | 1.00 | 0.00 | H |
| ATOM | 1234 | 2HB | GLN | A | 70 | 23.795 | 8.215 | 6.320 | 1.00 | 0.00 | H |
| ATOM | 1235 | 1HG | GLN | A | 70 | 26.651 | 8.371 | 7.271 | 1.00 | 0.00 | H |
| ATOM | 1236 | 2HG | GLN | A | 70 | 25.182 | 8.667 | 8.177 | 1.00 | 0.00 | H |
| ATOM | 1237 | 1HE2 | GLN | A | 70 | 26.883 | 5.182 | 8.700 | 1.00 | 0.00 | H |
| ATOM | 1238 | 2HE2 | GLN | A | 70 | 27.682 | 6.691 | 8.331 | 1.00 | 0.00 | H |
| ATOM | 1239 | N | LEU | A | 71 | 24.798 | 6.556 | 2.679 | 1.00 | 0.00 | N |
| ATOM | 1240 | CA | LEU | A | 71 | 23.976 | 6.013 | 1.599 | 1.00 | 0.00 | C |
| ATOM | 1241 | C | LEU | A | 71 | 23.086 | 4.897 | 2.171 | 1.00 | 0.00 | C |
| ATOM | 1242 | O | LEU | A | 71 | 21.887 | 4.842 | 1.894 | 1.00 | 0.00 | O |
| ATOM | 1243 | CB | LEU | A | 71 | 24.849 | 5.432 | 0.480 | 1.00 | 0.00 | C |
| ATOM | 1244 | CG | LEU | A | 71 | 24.220 | 4.443 | −0.539 | 1.00 | 0.00 | C |
| ATOM | 1245 | CD1 | LEU | A | 71 | 22.997 | 5.019 | −1.264 | 1.00 | 0.00 | C |
| ATOM | 1246 | CD2 | LEU | A | 71 | 25.278 | 3.979 | −1.553 | 1.00 | 0.00 | C |
| ATOM | 1247 | H | LEU | A | 71 | 25.763 | 6.267 | 2.715 | 1.00 | 0.00 | H |
| ATOM | 1248 | HA | LEU | A | 71 | 23.343 | 6.818 | 1.223 | 1.00 | 0.00 | H |
| ATOM | 1249 | 1HB | LEU | A | 71 | 25.285 | 6.268 | −0.069 | 1.00 | 0.00 | H |
| ATOM | 1250 | 2HB | LEU | A | 71 | 25.697 | 4.918 | 0.936 | 1.00 | 0.00 | H |
| ATOM | 1251 | HG | LEU | A | 71 | 23.894 | 3.542 | −0.016 | 1.00 | 0.00 | H |
| ATOM | 1252 | 1HD1 | LEU | A | 71 | 22.648 | 4.332 | −2.032 | 1.00 | 0.00 | H |
| ATOM | 1253 | 2HD1 | LEU | A | 71 | 22.159 | 5.197 | −0.593 | 1.00 | 0.00 | H |
| ATOM | 1254 | 3HD1 | LEU | A | 71 | 23.227 | 5.967 | −1.735 | 1.00 | 0.00 | H |
| ATOM | 1255 | 1HD2 | LEU | A | 71 | 24.850 | 3.291 | −2.282 | 1.00 | 0.00 | H |
| ATOM | 1256 | 2HD2 | LEU | A | 71 | 25.698 | 4.821 | −2.101 | 1.00 | 0.00 | H |
| ATOM | 1257 | 3HD2 | LEU | A | 71 | 26.100 | 3.461 | −1.058 | 1.00 | 0.00 | H |
| ATOM | 1258 | N | PHE | A | 72 | 23.669 | 4.014 | 2.978 | 1.00 | 0.00 | N |
| ATOM | 1259 | CA | PHE | A | 72 | 22.887 | 2.929 | 3.550 | 1.00 | 0.00 | C |
| ATOM | 1260 | C | PHE | A | 72 | 21.783 | 3.448 | 4.475 | 1.00 | 0.00 | C |
| ATOM | 1261 | O | PHE | A | 72 | 20.694 | 2.875 | 4.527 | 1.00 | 0.00 | O |
| ATOM | 1262 | CB | PHE | A | 72 | 23.811 | 1.940 | 4.261 | 1.00 | 0.00 | C |
| ATOM | 1263 | CG | PHE | A | 72 | 24.631 | 1.149 | 3.255 | 1.00 | 0.00 | C |
| ATOM | 1264 | CD1 | PHE | A | 72 | 25.983 | 1.471 | 3.022 | 1.00 | 0.00 | C |
| ATOM | 1265 | CD2 | PHE | A | 72 | 24.018 | 0.127 | 2.498 | 1.00 | 0.00 | C |
| ATOM | 1266 | CE1 | PHE | A | 72 | 26.716 | 0.775 | 2.039 | 1.00 | 0.00 | C |
| ATOM | 1267 | CE2 | PHE | A | 72 | 24.753 | −0.576 | 1.524 | 1.00 | 0.00 | C |
| ATOM | 1268 | CZ | PHE | A | 72 | 26.103 | −0.249 | 1.294 | 1.00 | 0.00 | C |
| ATOM | 1269 | H | PHE | A | 72 | 24.669 | 4.023 | 3.100 | 1.00 | 0.00 | H |
| ATOM | 1270 | HA | PHE | A | 72 | 22.361 | 2.425 | 2.738 | 1.00 | 0.00 | H |
| ATOM | 1271 | 1HB | PHE | A | 72 | 24.464 | 2.448 | 4.972 | 1.00 | 0.00 | H |
| ATOM | 1272 | 2HB | PHE | A | 72 | 23.221 | 1.225 | 4.836 | 1.00 | 0.00 | H |
| ATOM | 1273 | HD1 | PHE | A | 72 | 26.452 | 2.262 | 3.587 | 1.00 | 0.00 | H |
| ATOM | 1274 | HD2 | PHE | A | 72 | 22.977 | −0.111 | 2.652 | 1.00 | 0.00 | H |
| ATOM | 1275 | HE1 | PHE | A | 72 | 27.748 | 1.029 | 1.847 | 1.00 | 0.00 | H |
| ATOM | 1276 | HE2 | PHE | A | 72 | 24.278 | −1.354 | 0.945 | 1.00 | 0.00 | H |
| ATOM | 1277 | HZ | PHE | A | 72 | 26.665 | −0.774 | 0.536 | 1.00 | 0.00 | H |
| ATOM | 1278 | N | ARG | A | 73 | 22.042 | 4.543 | 5.190 | 1.00 | 0.00 | N |
| ATOM | 1279 | CA | ARG | A | 73 | 21.023 | 5.123 | 6.065 | 1.00 | 0.00 | C |
| ATOM | 1280 | C | ARG | A | 73 | 19.863 | 5.618 | 5.211 | 1.00 | 0.00 | C |
| ATOM | 1281 | O | ARG | A | 73 | 18.701 | 5.395 | 5.551 | 1.00 | 0.00 | O |
| ATOM | 1282 | CB | ARG | A | 73 | 21.600 | 6.290 | 6.862 | 1.00 | 0.00 | C |
| ATOM | 1283 | CG | ARG | A | 73 | 21.762 | 5.931 | 8.354 | 1.00 | 0.00 | C |
| ATOM | 1284 | CD | ARG | A | 73 | 22.007 | 7.156 | 9.258 | 1.00 | 0.00 | C |
| ATOM | 1285 | NE | ARG | A | 73 | 20.729 | 7.639 | 9.796 | 1.00 | 0.00 | N |
| ATOM | 1286 | CZ | ARG | A | 73 | 20.446 | 8.720 | 10.525 | 1.00 | 0.00 | C |
| ATOM | 1287 | NH1 | ARG | A | 73 | 21.407 | 9.585 | 10.854 | 1.00 | 0.00 | N |
| ATOM | 1288 | NH2 | ARG | A | 73 | 19.187 | 8.918 | 10.915 | 1.00 | 0.00 | N |
| ATOM | 1289 | H | ARG | A | 73 | 22.927 | 5.004 | 5.078 | 1.00 | 0.00 | H |
| ATOM | 1290 | HA | ARG | A | 73 | 20.630 | 4.363 | 6.744 | 1.00 | 0.00 | H |
| ATOM | 1291 | 1HB | ARG | A | 73 | 22.556 | 6.581 | 6.450 | 1.00 | 0.00 | H |
| ATOM | 1292 | 2HB | ARG | A | 73 | 20.964 | 7.175 | 6.800 | 1.00 | 0.00 | H |
| ATOM | 1293 | 1HG | ARG | A | 73 | 20.877 | 5.404 | 8.698 | 1.00 | 0.00 | H |
| ATOM | 1294 | 2HG | ARG | A | 73 | 22.535 | 5.180 | 8.490 | 1.00 | 0.00 | H |
| ATOM | 1295 | 1HD | ARG | A | 73 | 22.639 | 6.873 | 10.101 | 1.00 | 0.00 | H |
| ATOM | 1296 | 2HD | ARG | A | 73 | 22.524 | 7.948 | 8.715 | 1.00 | 0.00 | H |
| ATOM | 1297 | HE | ARG | A | 73 | 19.912 | 7.024 | 9.514 | 1.00 | 0.00 | H |
| ATOM | 1298 | 1HH1 | ARG | A | 73 | 21.231 | 10.453 | 11.362 | 1.00 | 0.00 | H |
| ATOM | 1299 | 2HH1 | ARG | A | 73 | 22.333 | 9.436 | 10.491 | 1.00 | 0.00 | H |
| ATOM | 1300 | 1HH2 | ARG | A | 73 | 18.885 | 9.645 | 11.548 | 1.00 | 0.00 | H |
| ATOM | 1301 | 2HH2 | ARG | A | 73 | 18.447 | 8.208 | 10.670 | 1.00 | 0.00 | H |
| ATOM | 1302 | N | SER | A | 74 | 20.164 | 6.293 | 4.099 | 1.00 | 0.00 | N |
| ATOM | 1303 | CA | SER | A | 74 | 19.100 | 6.791 | 3.232 | 1.00 | 0.00 | C |
| ATOM | 1304 | C | SER | A | 74 | 18.268 | 5.637 | 2.653 | 1.00 | 0.00 | C |
| ATOM | 1305 | O | SER | A | 74 | 17.046 | 5.765 | 2.520 | 1.00 | 0.00 | O |
| ATOM | 1306 | CB | SER | A | 74 | 19.666 | 7.677 | 2.099 | 1.00 | 0.00 | C |
| ATOM | 1307 | OG | SER | A | 74 | 20.397 | 7.033 | 1.078 | 1.00 | 0.00 | O |
| ATOM | 1308 | H | SER | A | 74 | 21.123 | 6.405 | 3.799 | 1.00 | 0.00 | H |
| ATOM | 1309 | HA | SER | A | 74 | 18.439 | 7.409 | 3.844 | 1.00 | 0.00 | H |
| ATOM | 1310 | 1HB | SER | A | 74 | 18.833 | 8.185 | 1.615 | 1.00 | 0.00 | H |
| ATOM | 1311 | 2HB | SER | A | 74 | 20.291 | 8.459 | 2.527 | 1.00 | 0.00 | H |
| ATOM | 1312 | HG | SER | A | 74 | 20.710 | 7.685 | 0.470 | 1.00 | 0.00 | H |
| ATOM | 1313 | N | LEU | A | 75 | 18.922 | 4.519 | 2.322 | 1.00 | 0.00 | N |
| ATOM | 1314 | CA | LEU | A | 75 | 18.229 | 3.346 | 1.778 | 1.00 | 0.00 | C |
| ATOM | 1315 | C | LEU | A | 75 | 17.383 | 2.665 | 2.868 | 1.00 | 0.00 | C |
| ATOM | 1316 | O | LEU | A | 75 | 16.260 | 2.226 | 2.602 | 1.00 | 0.00 | O |
| ATOM | 1317 | CB | LEU | A | 75 | 19.240 | 2.343 | 1.202 | 1.00 | 0.00 | C |
| ATOM | 1318 | CG | LEU | A | 75 | 19.900 | 2.767 | −0.124 | 1.00 | 0.00 | C |
| ATOM | 1319 | CD1 | LEU | A | 75 | 21.031 | 1.794 | −0.501 | 1.00 | 0.00 | C |
| ATOM | 1320 | CD2 | LEU | A | 75 | 18.871 | 2.866 | −1.261 | 1.00 | 0.00 | C |
| ATOM | 1321 | H | LEU | A | 75 | 19.937 | 4.556 | 2.305 | 1.00 | 0.00 | H |
| ATOM | 1322 | HA | LEU | A | 75 | 17.556 | 3.645 | 0.984 | 1.00 | 0.00 | H |
| ATOM | 1323 | 1HB | LEU | A | 75 | 20.016 | 2.161 | 1.945 | 1.00 | 0.00 | H |
| ATOM | 1324 | 2HB | LEU | A | 75 | 18.749 | 1.381 | 1.045 | 1.00 | 0.00 | H |
| ATOM | 1325 | HG | LEU | A | 75 | 20.349 | 3.752 | 0.007 | 1.00 | 0.00 | H |
| ATOM | 1326 | 1HD1 | LEU | A | 75 | 21.518 | 2.101 | −1.427 | 1.00 | 0.00 | H |
| ATOM | 1327 | 2HD1 | LEU | A | 75 | 21.799 | 1.762 | 0.273 | 1.00 | 0.00 | H |
| ATOM | 1328 | 3HD1 | LEU | A | 75 | 20.656 | 0.781 | −0.647 | 1.00 | 0.00 | H |
| ATOM | 1329 | 1HD2 | LEU | A | 75 | 19.361 | 2.908 | −2.231 | 1.00 | 0.00 | H |
| ATOM | 1330 | 2HD2 | LEU | A | 75 | 18.200 | 2.007 | −1.279 | 1.00 | 0.00 | H |
| ATOM | 1331 | 3HD2 | LEU | A | 75 | 18.259 | 3.763 | −1.174 | 1.00 | 0.00 | H |
| ATOM | 1332 | N | ALA | A | 76 | 17.925 | 2.563 | 4.082 | 1.00 | 0.00 | N |
| ATOM | 1333 | CA | ALA | A | 76 | 17.172 | 1.977 | 5.193 | 1.00 | 0.00 | C |
| ATOM | 1334 | C | ALA | A | 76 | 15.882 | 2.789 | 5.350 | 1.00 | 0.00 | C |
| ATOM | 1335 | O | ALA | A | 76 | 14.789 | 2.238 | 5.498 | 1.00 | 0.00 | O |
| ATOM | 1336 | CB | ALA | A | 76 | 17.992 | 2.036 | 6.488 | 1.00 | 0.00 | C |
| ATOM | 1337 | H | ALA | A | 76 | 18.867 | 2.913 | 4.241 | 1.00 | 0.00 | H |
| ATOM | 1338 | HA | ALA | A | 76 | 16.916 | 0.942 | 4.958 | 1.00 | 0.00 | H |
| ATOM | 1339 | 1HB | ALA | A | 76 | 17.440 | 1.590 | 7.317 | 1.00 | 0.00 | H |
| ATOM | 1340 | 2HB | ALA | A | 76 | 18.934 | 1.497 | 6.392 | 1.00 | 0.00 | H |
| ATOM | 1341 | 3HB | ALA | A | 76 | 18.232 | 3.061 | 6.775 | 1.00 | 0.00 | H |
| ATOM | 1342 | N | TYR | A | 77 | 16.011 | 4.107 | 5.296 | 1.00 | 0.00 | N |
| ATOM | 1343 | CA | TYR | A | 77 | 14.859 | 4.969 | 5.441 | 1.00 | 0.00 | C |
| ATOM | 1344 | C | TYR | A | 77 | 13.813 | 4.783 | 4.320 | 1.00 | 0.00 | C |
| ATOM | 1345 | O | TYR | A | 77 | 12.643 | 4.536 | 4.605 | 1.00 | 0.00 | O |
| ATOM | 1346 | CB | TYR | A | 77 | 15.312 | 6.433 | 5.515 | 1.00 | 0.00 | C |
| ATOM | 1347 | CG | TYR | A | 77 | 14.207 | 7.469 | 5.627 | 1.00 | 0.00 | C |
| ATOM | 1348 | CD1 | TYR | A | 77 | 13.896 | 8.285 | 4.520 | 1.00 | 0.00 | C |
| ATOM | 1349 | CD2 | TYR | A | 77 | 13.479 | 7.603 | 6.827 | 1.00 | 0.00 | C |
| ATOM | 1350 | CE1 | TYR | A | 77 | 12.852 | 9.222 | 4.611 | 1.00 | 0.00 | C |
| ATOM | 1351 | CE2 | TYR | A | 77 | 12.425 | 8.533 | 6.911 | 1.00 | 0.00 | C |
| ATOM | 1352 | CZ | TYR | A | 77 | 12.107 | 9.335 | 5.798 | 1.00 | 0.00 | C |
| ATOM | 1353 | OH | TYR | A | 77 | 11.072 | 10.217 | 5.857 | 1.00 | 0.00 | O |
| ATOM | 1354 | H | TYR | A | 77 | 16.940 | 4.516 | 5.225 | 1.00 | 0.00 | H |
| ATOM | 1355 | HA | TYR | A | 77 | 14.372 | 4.724 | 6.388 | 1.00 | 0.00 | H |
| ATOM | 1356 | 1HB | TYR | A | 77 | 15.973 | 6.564 | 6.374 | 1.00 | 0.00 | H |
| ATOM | 1357 | 2HB | TYR | A | 77 | 15.930 | 6.678 | 4.652 | 1.00 | 0.00 | H |
| ATOM | 1358 | HD1 | TYR | A | 77 | 14.451 | 8.192 | 3.597 | 1.00 | 0.00 | H |
| ATOM | 1359 | HD2 | TYR | A | 77 | 13.719 | 6.987 | 7.682 | 1.00 | 0.00 | H |
| ATOM | 1360 | HE1 | TYR | A | 77 | 12.623 | 9.848 | 3.765 | 1.00 | 0.00 | H |
| ATOM | 1361 | HE2 | TYR | A | 77 | 11.867 | 8.620 | 7.832 | 1.00 | 0.00 | H |
| ATOM | 1362 | HH | TYR | A | 77 | 10.640 | 10.215 | 6.701 | 1.00 | 0.00 | H |
| ATOM | 1363 | N | ILE | A | 78 | 14.204 | 4.890 | 3.052 | 1.00 | 0.00 | N |
| ATOM | 1364 | CA | ILE | A | 78 | 13.190 | 4.728 | 2.010 | 1.00 | 0.00 | C |
| ATOM | 1365 | C | ILE | A | 78 | 12.623 | 3.314 | 1.931 | 1.00 | 0.00 | C |
| ATOM | 1366 | O | ILE | A | 78 | 11.437 | 3.144 | 1.666 | 1.00 | 0.00 | O |
| ATOM | 1367 | CB | ILE | A | 78 | 13.693 | 5.151 | 0.601 | 1.00 | 0.00 | C |
| ATOM | 1368 | CG1 | ILE | A | 78 | 14.912 | 4.378 | 0.051 | 1.00 | 0.00 | C |
| ATOM | 1369 | CG2 | ILE | A | 78 | 13.987 | 6.665 | 0.608 | 1.00 | 0.00 | C |
| ATOM | 1370 | CD1 | ILE | A | 78 | 15.084 | 4.492 | −1.469 | 1.00 | 0.00 | C |
| ATOM | 1371 | H | ILE | A | 78 | 15.173 | 5.076 | 2.809 | 1.00 | 0.00 | H |
| ATOM | 1372 | HA | ILE | A | 78 | 12.345 | 5.380 | 2.249 | 1.00 | 0.00 | H |
| ATOM | 1373 | HB | ILE | A | 78 | 12.870 | 4.986 | −0.096 | 1.00 | 0.00 | H |
| ATOM | 1374 | 1HG1 | ILE | A | 78 | 15.809 | 4.743 | 0.539 | 1.00 | 0.00 | H |
| ATOM | 1375 | 2HG1 | ILE | A | 78 | 14.849 | 3.315 | 0.275 | 1.00 | 0.00 | H |
| ATOM | 1376 | 1HG2 | ILE | A | 78 | 14.321 | 7.017 | −0.363 | 1.00 | 0.00 | H |
| ATOM | 1377 | 2HG2 | ILE | A | 78 | 13.099 | 7.237 | 0.874 | 1.00 | 0.00 | H |
| ATOM | 1378 | 3HG2 | ILE | A | 78 | 14.771 | 6.920 | 1.321 | 1.00 | 0.00 | H |
| ATOM | 1379 | 1HD1 | ILE | A | 78 | 15.883 | 3.830 | −1.804 | 1.00 | 0.00 | H |
| ATOM | 1380 | 2HD1 | ILE | A | 78 | 14.178 | 4.190 | −1.995 | 1.00 | 0.00 | H |
| ATOM | 1381 | 3HD1 | ILE | A | 78 | 15.340 | 5.505 | −1.779 | 1.00 | 0.00 | H |
| ATOM | 1382 | N | HIS | A | 79 | 13.451 | 2.305 | 2.175 | 1.00 | 0.00 | N |
| ATOM | 1383 | CA | HIS | A | 79 | 12.987 | 0.922 | 2.108 | 1.00 | 0.00 | C |
| ATOM | 1384 | C | HIS | A | 79 | 11.953 | 0.606 | 3.189 | 1.00 | 0.00 | C |
| ATOM | 1385 | O | HIS | A | 79 | 11.100 | −0.275 | 3.001 | 1.00 | 0.00 | O |
| ATOM | 1386 | CB | HIS | A | 79 | 14.161 | −0.047 | 2.236 | 1.00 | 0.00 | C |
| ATOM | 1387 | CG | HIS | A | 79 | 13.864 | −1.526 | 2.385 | 1.00 | 0.00 | C |
| ATOM | 1388 | ND1 | HIS | A | 79 | 13.122 | −2.271 | 1.479 | 1.00 | 0.00 | N |
| ATOM | 1389 | CD2 | HIS | A | 79 | 14.220 | −2.413 | 3.378 | 1.00 | 0.00 | C |
| ATOM | 1390 | CE1 | HIS | A | 79 | 13.040 | −3.514 | 1.955 | 1.00 | 0.00 | C |
| ATOM | 1391 | NE2 | HIS | A | 79 | 13.693 | −3.675 | 3.099 | 1.00 | 0.00 | N |
| ATOM | 1392 | H | HIS | A | 79 | 14.443 | 2.476 | 2.335 | 1.00 | 0.00 | H |
| ATOM | 1393 | HA | HIS | A | 79 | 12.495 | 0.756 | 1.146 | 1.00 | 0.00 | H |
| ATOM | 1394 | 1HB | HIS | A | 79 | 14.823 | 0.076 | 1.378 | 1.00 | 0.00 | H |
| ATOM | 1395 | 2HB | HIS | A | 79 | 14.751 | 0.228 | 3.112 | 1.00 | 0.00 | H |
| ATOM | 1396 | HD1 | HIS | A | 79 | 12.707 | −1.975 | 0.598 | 1.00 | 0.00 | H |
| ATOM | 1397 | HD2 | HIS | A | 79 | 14.804 | −2.238 | 4.271 | 1.00 | 0.00 | H |
| ATOM | 1398 | HE1 | HIS | A | 79 | 12.511 | −4.311 | 1.450 | 1.00 | 0.00 | H |
| ATOM | 1399 | N | SER | A | 80 | 12.023 | 1.318 | 4.312 | 1.00 | 0.00 | N |
| ATOM | 1400 | CA | SER | A | 80 | 11.084 | 1.084 | 5.402 | 1.00 | 0.00 | C |
| ATOM | 1401 | C | SER | A | 80 | 9.676 | 1.506 | 4.987 | 1.00 | 0.00 | C |
| ATOM | 1402 | O | SER | A | 80 | 8.695 | 1.131 | 5.630 | 1.00 | 0.00 | O |
| ATOM | 1403 | CB | SER | A | 80 | 11.533 | 1.823 | 6.677 | 1.00 | 0.00 | C |
| ATOM | 1404 | OG | SER | A | 80 | 10.782 | 1.346 | 7.780 | 1.00 | 0.00 | O |
| ATOM | 1405 | H | SER | A | 80 | 12.773 | 1.990 | 4.436 | 1.00 | 0.00 | H |
| ATOM | 1406 | HA | SER | A | 80 | 11.068 | 0.014 | 5.623 | 1.00 | 0.00 | H |
| ATOM | 1407 | 1HB | SER | A | 80 | 12.587 | 1.639 | 6.884 | 1.00 | 0.00 | H |
| ATOM | 1408 | 2HB | SER | A | 80 | 11.411 | 2.903 | 6.578 | 1.00 | 0.00 | H |
| ATOM | 1409 | HG | SER | A | 80 | 9.860 | 1.489 | 7.601 | 1.00 | 0.00 | H |
| ATOM | 1410 | N | GLN | A | 81 | 9.573 | 2.298 | 3.922 | 1.00 | 0.00 | N |
| ATOM | 1411 | CA | GLN | A | 81 | 8.269 | 2.719 | 3.408 | 1.00 | 0.00 | C |
| ATOM | 1412 | C | GLN | A | 81 | 7.948 | 1.872 | 2.179 | 1.00 | 0.00 | C |
| ATOM | 1413 | O | GLN | A | 81 | 6.979 | 2.141 | 1.477 | 1.00 | 0.00 | O |
| ATOM | 1414 | CB | GLN | A | 81 | 8.280 | 4.188 | 2.980 | 1.00 | 0.00 | C |
| ATOM | 1415 | CG | GLN | A | 81 | 8.735 | 5.190 | 4.058 | 1.00 | 0.00 | C |
| ATOM | 1416 | CD | GLN | A | 81 | 8.640 | 6.629 | 3.549 | 1.00 | 0.00 | C |
| ATOM | 1417 | OE1 | GLN | A | 81 | 8.018 | 6.885 | 2.519 | 1.00 | 0.00 | O |
| ATOM | 1418 | NE2 | GLN | A | 81 | 9.245 | 7.575 | 4.253 | 1.00 | 0.00 | N |
| ATOM | 1419 | H | GLN | A | 81 | 10.407 | 2.601 | 3.433 | 1.00 | 0.00 | H |
| ATOM | 1420 | HA | GLN | A | 81 | 7.465 | 2.581 | 4.133 | 1.00 | 0.00 | H |
| ATOM | 1421 | 1HB | GLN | A | 81 | 8.930 | 4.309 | 2.111 | 1.00 | 0.00 | H |
| ATOM | 1422 | 2HB | GLN | A | 81 | 7.273 | 4.450 | 2.651 | 1.00 | 0.00 | H |
| ATOM | 1423 | 1HG | GLN | A | 81 | 8.119 | 5.089 | 4.952 | 1.00 | 0.00 | H |
| ATOM | 1424 | 2HG | GLN | A | 81 | 9.766 | 4.987 | 4.352 | 1.00 | 0.00 | H |
| ATOM | 1425 | 1HE2 | GLN | A | 81 | 9.181 | 8.540 | 3.963 | 1.00 | 0.00 | H |
| ATOM | 1426 | 2HE2 | GLN | A | 81 | 9.791 | 7.343 | 5.069 | 1.00 | 0.00 | H |
| ATOM | 1427 | N | GLY | A | 82 | 8.772 | 0.858 | 1.920 | 1.00 | 0.00 | N |
| ATOM | 1428 | CA | GLY | A | 82 | 8.566 | 0.001 | 0.764 | 1.00 | 0.00 | C |
| ATOM | 1429 | C | GLY | A | 82 | 8.995 | 0.655 | −0.543 | 1.00 | 0.00 | C |
| ATOM | 1430 | O | GLY | A | 82 | 8.752 | 0.112 | −1.624 | 1.00 | 0.00 | O |
| ATOM | 1431 | H | GLY | A | 82 | 9.551 | 0.652 | 2.537 | 1.00 | 0.00 | H |
| ATOM | 1432 | 1HA | GLY | A | 82 | 9.153 | −0.908 | 0.898 | 1.00 | 0.00 | H |
| ATOM | 1433 | 2HA | GLY | A | 82 | 7.522 | −0.309 | 0.687 | 1.00 | 0.00 | H |
| ATOM | 1434 | N | VAL | A | 83 | 9.643 | 1.820 | −0.450 | 1.00 | 0.00 | N |
| ATOM | 1435 | CA | VAL | A | 83 | 10.092 | 2.552 | −1.638 | 1.00 | 0.00 | C |
| ATOM | 1436 | C | VAL | A | 83 | 11.506 | 2.148 | −2.052 | 1.00 | 0.00 | C |
| ATOM | 1437 | O | VAL | A | 83 | 12.435 | 2.147 | −1.237 | 1.00 | 0.00 | O |
| ATOM | 1438 | CB | VAL | A | 83 | 10.061 | 4.087 | −1.404 | 1.00 | 0.00 | C |
| ATOM | 1439 | CG1 | VAL | A | 83 | 10.542 | 4.928 | −2.611 | 1.00 | 0.00 | C |
| ATOM | 1440 | CG2 | VAL | A | 83 | 8.651 | 4.565 | −1.012 | 1.00 | 0.00 | C |
| ATOM | 1441 | H | VAL | A | 83 | 9.926 | 2.176 | 0.454 | 1.00 | 0.00 | H |
| ATOM | 1442 | HA | VAL | A | 83 | 9.429 | 2.357 | −2.474 | 1.00 | 0.00 | H |
| ATOM | 1443 | HB | VAL | A | 83 | 10.720 | 4.315 | −0.567 | 1.00 | 0.00 | H |
| ATOM | 1444 | 1HG1 | VAL | A | 83 | 10.440 | 5.994 | −2.412 | 1.00 | 0.00 | H |
| ATOM | 1445 | 2HG1 | VAL | A | 83 | 11.595 | 4.759 | −2.839 | 1.00 | 0.00 | H |
| ATOM | 1446 | 3HG1 | VAL | A | 83 | 9.967 | 4.702 | −3.510 | 1.00 | 0.00 | H |
| ATOM | 1447 | 1HG2 | VAL | A | 83 | 8.657 | 5.629 | −0.779 | 1.00 | 0.00 | H |
| ATOM | 1448 | 2HG2 | VAL | A | 83 | 7.944 | 4.399 | −1.825 | 1.00 | 0.00 | H |
| ATOM | 1449 | 3HG2 | VAL | A | 83 | 8.271 | 4.040 | −0.136 | 1.00 | 0.00 | H |
| ATOM | 1450 | N | CYS | A | 84 | 11.646 | 1.803 | −3.328 | 1.00 | 0.00 | N |
| ATOM | 1451 | CA | CYS | A | 84 | 12.929 | 1.405 | −3.912 | 1.00 | 0.00 | C |
| ATOM | 1452 | C | CYS | A | 84 | 13.357 | 2.555 | −4.830 | 1.00 | 0.00 | C |
| ATOM | 1453 | O | CYS | A | 84 | 12.552 | 3.065 | −5.601 | 1.00 | 0.00 | O |
| ATOM | 1454 | CB | CYS | A | 84 | 12.756 | 0.107 | −4.717 | 1.00 | 0.00 | C |
| ATOM | 1455 | SG | CYS | A | 84 | 14.299 | −0.572 | −5.377 | 1.00 | 0.00 | S |
| ATOM | 1456 | H | CYS | A | 84 | 10.849 | 1.850 | −3.955 | 1.00 | 0.00 | H |
| ATOM | 1457 | HA | CYS | A | 84 | 13.697 | 1.244 | −3.150 | 1.00 | 0.00 | H |
| ATOM | 1458 | 1HB | CYS | A | 84 | 12.315 | −0.652 | −4.073 | 1.00 | 0.00 | H |
| ATOM | 1459 | 2HB | CYS | A | 84 | 12.061 | 0.259 | −5.545 | 1.00 | 0.00 | H |
| ATOM | 1460 | HG | CYS | A | 84 | 13.776 | −1.680 | −5.909 | 1.00 | 0.00 | H |
| ATOM | 1461 | N | HIS | A | 85 | 14.624 | 2.959 | −4.743 | 1.00 | 0.00 | N |
| ATOM | 1462 | CA | HIS | A | 85 | 15.132 | 4.059 | −5.555 | 1.00 | 0.00 | C |
| ATOM | 1463 | C | HIS | A | 85 | 15.257 | 3.656 | −7.029 | 1.00 | 0.00 | C |
| ATOM | 1464 | O | HIS | A | 85 | 14.853 | 4.407 | −7.911 | 1.00 | 0.00 | O |
| ATOM | 1465 | CB | HIS | A | 85 | 16.485 | 4.523 | −5.001 | 1.00 | 0.00 | C |
| ATOM | 1466 | CG | HIS | A | 85 | 16.989 | 5.841 | −5.485 | 1.00 | 0.00 | C |
| ATOM | 1467 | ND1 | HIS | A | 85 | 16.412 | 7.048 | −5.137 | 1.00 | 0.00 | N |
| ATOM | 1468 | CD2 | HIS | A | 85 | 18.067 | 6.145 | −6.275 | 1.00 | 0.00 | C |
| ATOM | 1469 | CE1 | HIS | A | 85 | 17.156 | 7.998 | −5.697 | 1.00 | 0.00 | C |
| ATOM | 1470 | NE2 | HIS | A | 85 | 18.174 | 7.521 | −6.407 | 1.00 | 0.00 | N |
| ATOM | 1471 | H | HIS | A | 85 | 15.297 | 2.385 | −4.209 | 1.00 | 0.00 | H |
| ATOM | 1472 | HA | HIS | A | 85 | 14.436 | 4.898 | −5.492 | 1.00 | 0.00 | H |
| ATOM | 1473 | 1HB | HIS | A | 85 | 16.439 | 4.588 | −3.916 | 1.00 | 0.00 | H |
| ATOM | 1474 | 2HB | HIS | A | 85 | 17.245 | 3.766 | −5.202 | 1.00 | 0.00 | H |
| ATOM | 1475 | HD1 | HIS | A | 85 | 15.620 | 7.227 | −4.537 | 1.00 | 0.00 | H |
| ATOM | 1476 | HD2 | HIS | A | 85 | 18.790 | 5.485 | −6.720 | 1.00 | 0.00 | H |
| ATOM | 1477 | HE1 | HIS | A | 85 | 16.933 | 9.046 | −5.573 | 1.00 | 0.00 | H |
| ATOM | 1478 | N | ARG | A | 86 | 15.826 | 2.474 | −7.271 | 1.00 | 0.00 | N |
| ATOM | 1479 | CA | ARG | A | 86 | 15.990 | 1.909 | −8.614 | 1.00 | 0.00 | C |
| ATOM | 1480 | C | ARG | A | 86 | 17.013 | 2.578 | −9.543 | 1.00 | 0.00 | C |
| ATOM | 1481 | O | ARG | A | 86 | 17.134 | 2.176 | −10.706 | 1.00 | 0.00 | O |
| ATOM | 1482 | CB | ARG | A | 86 | 14.624 | 1.825 | −9.320 | 1.00 | 0.00 | C |
| ATOM | 1483 | CG | ARG | A | 86 | 13.550 | 1.105 | −8.483 | 1.00 | 0.00 | C |
| ATOM | 1484 | CD | ARG | A | 86 | 12.422 | 0.467 | −9.290 | 1.00 | 0.00 | C |
| ATOM | 1485 | NE | ARG | A | 86 | 11.665 | 1.478 | −10.035 | 1.00 | 0.00 | N |
| ATOM | 1486 | CZ | ARG | A | 86 | 10.744 | 1.261 | −10.974 | 1.00 | 0.00 | C |
| ATOM | 1487 | NH1 | ARG | A | 86 | 10.449 | 0.031 | −11.393 | 1.00 | 0.00 | N |
| ATOM | 1488 | NH2 | ARG | A | 86 | 10.115 | 2.313 | −11.492 | 1.00 | 0.00 | N |
| ATOM | 1489 | H | ARG | A | 86 | 16.227 | 1.936 | −6.492 | 1.00 | 0.00 | H |
| ATOM | 1490 | HA | ARG | A | 86 | 16.362 | 0.896 | −8.456 | 1.00 | 0.00 | H |
| ATOM | 1491 | 1HB | ARG | A | 86 | 14.264 | 2.797 | −9.653 | 1.00 | 0.00 | H |
| ATOM | 1492 | 2HB | ARG | A | 86 | 14.762 | 1.257 | −10.240 | 1.00 | 0.00 | H |
| ATOM | 1493 | 1HG | ARG | A | 86 | 14.033 | 0.307 | −7.929 | 1.00 | 0.00 | H |
| ATOM | 1494 | 2HG | ARG | A | 86 | 13.136 | 1.781 | −7.738 | 1.00 | 0.00 | H |
| ATOM | 1495 | 1HD | ARG | A | 86 | 12.827 | −0.292 | −9.957 | 1.00 | 0.00 | H |
| ATOM | 1496 | 2HD | ARG | A | 86 | 11.737 | −0.048 | −8.618 | 1.00 | 0.00 | H |
| ATOM | 1497 | HE | ARG | A | 86 | 11.896 | 2.448 | −9.790 | 1.00 | 0.00 | H |
| ATOM | 1498 | 1HH1 | ARG | A | 86 | 9.764 | −0.137 | −12.109 | 1.00 | 0.00 | H |
| ATOM | 1499 | 2HH1 | ARG | A | 86 | 10.982 | −0.786 | −11.077 | 1.00 | 0.00 | H |
| ATOM | 1500 | 1HH2 | ARG | A | 86 | 9.405 | 2.241 | −12.203 | 1.00 | 0.00 | H |
| ATOM | 1501 | 2HH2 | ARG | A | 86 | 10.358 | 3.238 | −11.160 | 1.00 | 0.00 | H |
| ATOM | 1502 | N | ASP | A | 87 | 17.745 | 3.580 | −9.046 | 1.00 | 0.00 | N |
| ATOM | 1503 | CA | ASP | A | 87 | 18.783 | 4.240 | −9.852 | 1.00 | 0.00 | C |
| ATOM | 1504 | C | ASP | A | 87 | 19.949 | 4.701 | −8.976 | 1.00 | 0.00 | C |
| ATOM | 1505 | O | ASP | A | 87 | 20.403 | 5.848 | −9.076 | 1.00 | 0.00 | O |
| ATOM | 1506 | CB | ASP | A | 87 | 18.214 | 5.444 | −10.610 | 1.00 | 0.00 | C |
| ATOM | 1507 | CG | ASP | A | 87 | 19.060 | 6.043 | −11.732 | 1.00 | 0.00 | C |
| ATOM | 1508 | OD1 | ASP | A | 87 | 19.735 | 5.304 | −12.486 | 1.00 | 0.00 | O |
| ATOM | 1509 | OD2 | ASP | A | 87 | 19.023 | 7.279 | −11.916 | 1.00 | 0.00 | O |
| ATOM | 1510 | H | ASP | A | 87 | 17.565 | 3.885 | −8.103 | 1.00 | 0.00 | H |
| ATOM | 1511 | HA | ASP | A | 87 | 19.206 | 3.508 | −10.541 | 1.00 | 0.00 | H |
| ATOM | 1512 | 1HB | ASP | A | 87 | 17.333 | 5.100 | −11.141 | 1.00 | 0.00 | H |
| ATOM | 1513 | 2HB | ASP | A | 87 | 17.887 | 6.231 | −9.928 | 1.00 | 0.00 | H |
| ATOM | 1514 | N | ILE | A | 88 | 20.425 | 3.810 | −8.110 | 1.00 | 0.00 | N |
| ATOM | 1515 | CA | ILE | A | 88 | 21.543 | 4.138 | −7.232 | 1.00 | 0.00 | C |
| ATOM | 1516 | C | ILE | A | 88 | 22.832 | 4.120 | −8.058 | 1.00 | 0.00 | C |
| ATOM | 1517 | O | ILE | A | 88 | 23.188 | 3.100 | −8.662 | 1.00 | 0.00 | O |
| ATOM | 1518 | CB | ILE | A | 88 | 21.658 | 3.132 | −6.054 | 1.00 | 0.00 | C |
| ATOM | 1519 | CG1 | ILE | A | 88 | 20.408 | 3.167 | −5.143 | 1.00 | 0.00 | C |
| ATOM | 1520 | CG2 | ILE | A | 88 | 22.949 | 3.219 | −5.205 | 1.00 | 0.00 | C |
| ATOM | 1521 | CD1 | ILE | A | 88 | 20.262 | 4.451 | −4.312 | 1.00 | 0.00 | C |
| ATOM | 1522 | H | ILE | A | 88 | 20.069 | 2.866 | −8.115 | 1.00 | 0.00 | H |
| ATOM | 1523 | HA | ILE | A | 88 | 21.414 | 5.148 | −6.843 | 1.00 | 0.00 | H |
| ATOM | 1524 | HB | ILE | A | 88 | 21.633 | 2.155 | −6.516 | 1.00 | 0.00 | H |
| ATOM | 1525 | 1HG1 | ILE | A | 88 | 19.508 | 3.026 | −5.743 | 1.00 | 0.00 | H |
| ATOM | 1526 | 2HG1 | ILE | A | 88 | 20.426 | 2.310 | −4.468 | 1.00 | 0.00 | H |
| ATOM | 1527 | 1HG2 | ILE | A | 88 | 22.905 | 2.529 | −4.361 | 1.00 | 0.00 | H |
| ATOM | 1528 | 2HG2 | ILE | A | 88 | 23.837 | 2.952 | −5.778 | 1.00 | 0.00 | H |
| ATOM | 1529 | 3HG2 | ILE | A | 88 | 23.100 | 4.222 | −4.804 | 1.00 | 0.00 | H |
| ATOM | 1530 | 1HD1 | ILE | A | 88 | 19.282 | 4.501 | −3.838 | 1.00 | 0.00 | H |
| ATOM | 1531 | 2HD1 | ILE | A | 88 | 21.007 | 4.506 | −3.523 | 1.00 | 0.00 | H |
| ATOM | 1532 | 3HD1 | ILE | A | 88 | 20.364 | 5.345 | −4.921 | 1.00 | 0.00 | H |
| ATOM | 1533 | N | LYS | A | 89 | 23.518 | 5.260 | −8.084 | 1.00 | 0.00 | N |
| ATOM | 1534 | CA | LYS | A | 89 | 24.763 | 5.411 | −8.836 | 1.00 | 0.00 | C |
| ATOM | 1535 | C | LYS | A | 89 | 25.483 | 6.664 | −8.328 | 1.00 | 0.00 | C |
| ATOM | 1536 | O | LYS | A | 89 | 24.857 | 7.554 | −7.744 | 1.00 | 0.00 | O |
| ATOM | 1537 | CB | LYS | A | 89 | 24.462 | 5.537 | −10.336 | 1.00 | 0.00 | C |
| ATOM | 1538 | CG | LYS | A | 89 | 23.567 | 6.731 | −10.738 | 1.00 | 0.00 | C |
| ATOM | 1539 | CD | LYS | A | 89 | 22.958 | 6.499 | −12.118 | 1.00 | 0.00 | C |
| ATOM | 1540 | CE | LYS | A | 89 | 22.078 | 7.633 | −12.630 | 1.00 | 0.00 | C |
| ATOM | 1541 | NZ | LYS | A | 89 | 21.182 | 7.162 | −13.689 | 1.00 | 0.00 | N |
| ATOM | 1542 | H | LYS | A | 89 | 23.173 | 6.073 | −7.592 | 1.00 | 0.00 | H |
| ATOM | 1543 | HA | LYS | A | 89 | 25.372 | 4.526 | −8.645 | 1.00 | 0.00 | H |
| ATOM | 1544 | 1HB | LYS | A | 89 | 25.383 | 5.577 | −10.916 | 1.00 | 0.00 | H |
| ATOM | 1545 | 2HB | LYS | A | 89 | 23.986 | 4.611 | −10.661 | 1.00 | 0.00 | H |
| ATOM | 1546 | 1HG | LYS | A | 89 | 22.749 | 6.848 | −10.027 | 1.00 | 0.00 | H |
| ATOM | 1547 | 2HG | LYS | A | 89 | 24.127 | 7.666 | −10.716 | 1.00 | 0.00 | H |
| ATOM | 1548 | 1HD | LYS | A | 89 | 23.741 | 6.276 | −12.844 | 1.00 | 0.00 | H |
| ATOM | 1549 | 2HD | LYS | A | 89 | 22.329 | 5.615 | −12.020 | 1.00 | 0.00 | H |
| ATOM | 1550 | 1HE | LYS | A | 89 | 21.427 | 7.956 | −11.831 | 1.00 | 0.00 | H |
| ATOM | 1551 | 2HE | LYS | A | 89 | 22.666 | 8.492 | −12.954 | 1.00 | 0.00 | H |
| ATOM | 1552 | 1HZ | LYS | A | 89 | 20.536 | 7.890 | −13.937 | 1.00 | 0.00 | H |
| ATOM | 1553 | 2HZ | LYS | A | 89 | 21.667 | 6.786 | −14.479 | 1.00 | 0.00 | H |
| ATOM | 1554 | 3HZ | LYS | A | 89 | 20.593 | 6.430 | −13.246 | 1.00 | 0.00 | H |
| ATOM | 1555 | N | PRO | A | 90 | 26.801 | 6.768 | −8.572 | 1.00 | 0.00 | N |
| ATOM | 1556 | CA | PRO | A | 90 | 27.573 | 7.930 | −8.106 | 1.00 | 0.00 | C |
| ATOM | 1557 | C | PRO | A | 90 | 27.009 | 9.304 | −8.415 | 1.00 | 0.00 | C |
| ATOM | 1558 | O | PRO | A | 90 | 27.101 | 10.206 | −7.585 | 1.00 | 0.00 | O |
| ATOM | 1559 | CB | PRO | A | 90 | 28.959 | 7.716 | −8.724 | 1.00 | 0.00 | C |
| ATOM | 1560 | CG | PRO | A | 90 | 29.095 | 6.226 | −8.938 | 1.00 | 0.00 | C |
| ATOM | 1561 | CD | PRO | A | 90 | 27.666 | 5.765 | −9.193 | 1.00 | 0.00 | C |
| ATOM | 1562 | HA | PRO | A | 90 | 27.653 | 7.838 | −7.020 | 1.00 | 0.00 | H |
| ATOM | 1563 | 1HB | PRO | A | 90 | 29.024 | 8.191 | −9.702 | 1.00 | 0.00 | H |
| ATOM | 1564 | 2HB | PRO | A | 90 | 29.759 | 8.118 | −8.102 | 1.00 | 0.00 | H |
| ATOM | 1565 | 1HG | PRO | A | 90 | 29.477 | 5.754 | −8.032 | 1.00 | 0.00 | H |
| ATOM | 1566 | 2HG | PRO | A | 90 | 29.778 | 5.969 | −9.750 | 1.00 | 0.00 | H |
| ATOM | 1567 | 1HD | PRO | A | 90 | 27.514 | 4.761 | −8.811 | 1.00 | 0.00 | H |
| ATOM | 1568 | 2HD | PRO | A | 90 | 27.485 | 5.752 | −10.268 | 1.00 | 0.00 | H |
| ATOM | 1569 | N | GLN | A | 91 | 26.427 | 9.465 | −9.596 | 1.00 | 0.00 | N |
| ATOM | 1570 | CA | GLN | A | 91 | 25.866 | 10.748 | −9.998 | 1.00 | 0.00 | C |
| ATOM | 1571 | C | GLN | A | 91 | 24.618 | 11.143 | −9.213 | 1.00 | 0.00 | C |
| ATOM | 1572 | O | GLN | A | 91 | 24.219 | 12.310 | −9.215 | 1.00 | 0.00 | O |
| ATOM | 1573 | CB | GLN | A | 91 | 25.532 | 10.731 | −11.487 | 1.00 | 0.00 | C |
| ATOM | 1574 | CG | GLN | A | 91 | 26.679 | 10.379 | −12.464 | 1.00 | 0.00 | C |
| ATOM | 1575 | CD | GLN | A | 91 | 26.976 | 8.881 | −12.620 | 1.00 | 0.00 | C |
| ATOM | 1576 | OE1 | GLN | A | 91 | 26.369 | 8.035 | −11.970 | 1.00 | 0.00 | O |
| ATOM | 1577 | NE2 | GLN | A | 91 | 27.924 | 8.532 | −13.474 | 1.00 | 0.00 | N |
| ATOM | 1578 | H | GLN | A | 91 | 26.372 | 8.686 | −10.231 | 1.00 | 0.00 | H |
| ATOM | 1579 | HA | GLN | A | 91 | 26.605 | 11.533 | −9.820 | 1.00 | 0.00 | H |
| ATOM | 1580 | 1HB | GLN | A | 91 | 24.687 | 10.063 | −11.667 | 1.00 | 0.00 | H |
| ATOM | 1581 | 2HB | GLN | A | 91 | 25.164 | 11.721 | −11.761 | 1.00 | 0.00 | H |
| ATOM | 1582 | 1HG | GLN | A | 91 | 26.413 | 10.751 | −13.454 | 1.00 | 0.00 | H |
| ATOM | 1583 | 2HG | GLN | A | 91 | 27.582 | 10.909 | −12.176 | 1.00 | 0.00 | H |
| ATOM | 1584 | 1HE2 | GLN | A | 91 | 28.147 | 7.554 | −13.582 | 1.00 | 0.00 | H |
| ATOM | 1585 | 2HE2 | GLN | A | 91 | 28.466 | 9.228 | −13.962 | 1.00 | 0.00 | H |
| ATOM | 1586 | N | ASN | A | 92 | 23.966 | 10.187 | −8.535 | 1.00 | 0.00 | N |
| ATOM | 1587 | CA | ASN | A | 92 | 22.717 | 10.385 | −7.779 | 1.00 | 0.00 | C |
| ATOM | 1588 | C | ASN | A | 92 | 23.000 | 10.449 | −6.268 | 1.00 | 0.00 | C |
| ATOM | 1589 | O | ASN | A | 92 | 22.083 | 10.630 | −5.466 | 1.00 | 0.00 | O |
| ATOM | 1590 | CB | ASN | A | 92 | 21.717 | 9.236 | −8.079 | 1.00 | 0.00 | C |
| ATOM | 1591 | CG | ASN | A | 92 | 21.078 | 9.284 | −9.466 | 1.00 | 0.00 | C |
| ATOM | 1592 | OD1 | ASN | A | 92 | 21.634 | 9.848 | −10.406 | 1.00 | 0.00 | O |
| ATOM | 1593 | ND2 | ASN | A | 92 | 19.925 | 8.643 | −9.615 | 1.00 | 0.00 | N |
| ATOM | 1594 | H | ASN | A | 92 | 24.362 | 9.255 | −8.500 | 1.00 | 0.00 | H |
| ATOM | 1595 | HA | ASN | A | 92 | 22.245 | 11.330 | −8.050 | 1.00 | 0.00 | H |
| ATOM | 1596 | 1HB | ASN | A | 92 | 22.192 | 8.264 | −7.945 | 1.00 | 0.00 | H |
| ATOM | 1597 | 2HB | ASN | A | 92 | 20.898 | 9.272 | −7.357 | 1.00 | 0.00 | H |
| ATOM | 1598 | 1HD2 | ASN | A | 92 | 19.539 | 8.437 | −10.542 | 1.00 | 0.00 | H |
| ATOM | 1599 | 2HD2 | ASN | A | 92 | 19.499 | 8.178 | −8.822 | 1.00 | 0.00 | H |
| ATOM | 1600 | N | LEU | A | 93 | 24.273 | 10.330 | −5.867 | 1.00 | 0.00 | N |
| ATOM | 1601 | CA | LEU | A | 93 | 24.744 | 10.578 | −4.516 | 1.00 | 0.00 | C |
| ATOM | 1602 | C | LEU | A | 93 | 25.438 | 11.942 | −4.521 | 1.00 | 0.00 | C |
| ATOM | 1603 | O | LEU | A | 93 | 26.553 | 12.076 | −5.034 | 1.00 | 0.00 | O |
| ATOM | 1604 | CB | LEU | A | 93 | 25.782 | 9.499 | −4.115 | 1.00 | 0.00 | C |
| ATOM | 1605 | CG | LEU | A | 93 | 25.321 | 8.040 | −4.294 | 1.00 | 0.00 | C |
| ATOM | 1606 | CD1 | LEU | A | 93 | 26.416 | 7.058 | −3.860 | 1.00 | 0.00 | C |
| ATOM | 1607 | CD2 | LEU | A | 93 | 23.994 | 7.737 | −3.595 | 1.00 | 0.00 | C |
| ATOM | 1608 | H | LEU | A | 93 | 24.979 | 10.238 | −6.584 | 1.00 | 0.00 | H |
| ATOM | 1609 | HA | LEU | A | 93 | 23.915 | 10.631 | −3.812 | 1.00 | 0.00 | H |
| ATOM | 1610 | 1HB | LEU | A | 93 | 26.677 | 9.614 | −4.725 | 1.00 | 0.00 | H |
| ATOM | 1611 | 2HB | LEU | A | 93 | 26.081 | 9.669 | −3.081 | 1.00 | 0.00 | H |
| ATOM | 1612 | HG | LEU | A | 93 | 25.162 | 7.874 | −5.354 | 1.00 | 0.00 | H |
| ATOM | 1613 | 1HD1 | LEU | A | 93 | 26.122 | 6.032 | −4.077 | 1.00 | 0.00 | H |
| ATOM | 1614 | 2HD1 | LEU | A | 93 | 27.350 | 7.247 | −4.391 | 1.00 | 0.00 | H |
| ATOM | 1615 | 3HD1 | LEU | A | 93 | 26.623 | 7.130 | −2.791 | 1.00 | 0.00 | H |
| ATOM | 1616 | 1HD2 | LEU | A | 93 | 23.689 | 6.709 | −3.786 | 1.00 | 0.00 | H |
| ATOM | 1617 | 2HD2 | LEU | A | 93 | 24.069 | 7.886 | −2.519 | 1.00 | 0.00 | H |
| ATOM | 1618 | 3HD2 | LEU | A | 93 | 23.190 | 8.372 | −3.965 | 1.00 | 0.00 | H |
| ATOM | 1619 | N | LEU | A | 94 | 24.784 | 12.954 | −3.946 | 1.00 | 0.00 | N |
| ATOM | 1620 | CA | LEU | A | 94 | 25.299 | 14.311 | −3.840 | 1.00 | 0.00 | C |
| ATOM | 1621 | C | LEU | A | 94 | 25.982 | 14.415 | −2.477 | 1.00 | 0.00 | C |
| ATOM | 1622 | O | LEU | A | 94 | 25.314 | 14.622 | −1.466 | 1.00 | 0.00 | O |
| ATOM | 1623 | CB | LEU | A | 94 | 24.138 | 15.320 | −3.984 | 1.00 | 0.00 | C |
| ATOM | 1624 | CG | LEU | A | 94 | 23.564 | 15.523 | −5.413 | 1.00 | 0.00 | C |
| ATOM | 1625 | CD1 | LEU | A | 94 | 23.111 | 14.231 | −6.118 | 1.00 | 0.00 | C |
| ATOM | 1626 | CD2 | LEU | A | 94 | 22.401 | 16.528 | −5.387 | 1.00 | 0.00 | C |
| ATOM | 1627 | H | LEU | A | 94 | 23.879 | 12.785 | −3.515 | 1.00 | 0.00 | H |
| ATOM | 1628 | HA | LEU | A | 94 | 26.012 | 14.535 | −4.618 | 1.00 | 0.00 | H |
| ATOM | 1629 | 1HB | LEU | A | 94 | 23.333 | 15.068 | −3.294 | 1.00 | 0.00 | H |
| ATOM | 1630 | 2HB | LEU | A | 94 | 24.505 | 16.290 | −3.653 | 1.00 | 0.00 | H |
| ATOM | 1631 | HG | LEU | A | 94 | 24.351 | 15.967 | −6.023 | 1.00 | 0.00 | H |
| ATOM | 1632 | 1HD1 | LEU | A | 94 | 22.564 | 14.443 | −7.038 | 1.00 | 0.00 | H |
| ATOM | 1633 | 2HD1 | LEU | A | 94 | 23.958 | 13.612 | −6.410 | 1.00 | 0.00 | H |
| ATOM | 1634 | 3HD1 | LEU | A | 94 | 22.465 | 13.629 | −5.480 | 1.00 | 0.00 | H |
| ATOM | 1635 | 1HD2 | LEU | A | 94 | 21.983 | 16.677 | −6.384 | 1.00 | 0.00 | H |
| ATOM | 1636 | 2HD2 | LEU | A | 94 | 21.594 | 16.189 | −4.737 | 1.00 | 0.00 | H |
| ATOM | 1637 | 3HD2 | LEU | A | 94 | 22.724 | 17.505 | −5.033 | 1.00 | 0.00 | H |
| ATOM | 1638 | N | VAL | A | 95 | 27.303 | 14.206 | −2.445 | 1.00 | 0.00 | N |
| ATOM | 1639 | CA | VAL | A | 95 | 28.135 | 14.399 | −1.266 | 1.00 | 0.00 | C |
| ATOM | 1640 | C | VAL | A | 95 | 28.903 | 15.695 | −1.466 | 1.00 | 0.00 | C |
| ATOM | 1641 | O | VAL | A | 95 | 29.496 | 15.888 | −2.524 | 1.00 | 0.00 | O |
| ATOM | 1642 | CB | VAL | A | 95 | 29.073 | 13.177 | −1.038 | 1.00 | 0.00 | C |
| ATOM | 1643 | CG1 | VAL | A | 95 | 30.182 | 12.994 | −2.093 | 1.00 | 0.00 | C |
| ATOM | 1644 | CG2 | VAL | A | 95 | 29.695 | 13.172 | 0.370 | 1.00 | 0.00 | C |
| ATOM | 1645 | H | VAL | A | 95 | 27.794 | 14.112 | −3.319 | 1.00 | 0.00 | H |
| ATOM | 1646 | HA | VAL | A | 95 | 27.512 | 14.555 | −0.405 | 1.00 | 0.00 | H |
| ATOM | 1647 | HB | VAL | A | 95 | 28.440 | 12.292 | −1.091 | 1.00 | 0.00 | H |
| ATOM | 1648 | 1HG1 | VAL | A | 95 | 30.739 | 12.074 | −1.921 | 1.00 | 0.00 | H |
| ATOM | 1649 | 2HG1 | VAL | A | 95 | 29.770 | 12.962 | −3.097 | 1.00 | 0.00 | H |
| ATOM | 1650 | 3HG1 | VAL | A | 95 | 30.916 | 13.799 | −2.063 | 1.00 | 0.00 | H |
| ATOM | 1651 | 1HG2 | VAL | A | 95 | 30.266 | 12.260 | 0.544 | 1.00 | 0.00 | H |
| ATOM | 1652 | 2HG2 | VAL | A | 95 | 30.370 | 14.015 | 0.520 | 1.00 | 0.00 | H |
| ATOM | 1653 | 3HG2 | VAL | A | 95 | 28.937 | 13.223 | 1.148 | 1.00 | 0.00 | H |
| ATOM | 1654 | N | ASP | A | 96 | 28.902 | 16.568 | −0.458 | 1.00 | 0.00 | N |
| ATOM | 1655 | CA | ASP | A | 96 | 29.760 | 17.745 | −0.423 | 1.00 | 0.00 | C |
| ATOM | 1656 | C | ASP | A | 96 | 31.022 | 17.364 | 0.385 | 1.00 | 0.00 | C |
| ATOM | 1657 | O | ASP | A | 96 | 30.925 | 16.670 | 1.400 | 1.00 | 0.00 | O |
| ATOM | 1658 | CB | ASP | A | 96 | 29.009 | 18.951 | 0.204 | 1.00 | 0.00 | C |
| ATOM | 1659 | CG | ASP | A | 96 | 28.638 | 18.887 | 1.683 | 1.00 | 0.00 | C |
| ATOM | 1660 | OD1 | ASP | A | 96 | 29.551 | 18.980 | 2.536 | 1.00 | 0.00 | O |
| ATOM | 1661 | OD2 | ASP | A | 96 | 27.429 | 18.819 | 1.969 | 1.00 | 0.00 | O |
| ATOM | 1662 | H | ASP | A | 96 | 28.425 | 16.310 | 0.405 | 1.00 | 0.00 | H |
| ATOM | 1663 | HA | ASP | A | 96 | 30.011 | 18.034 | −1.440 | 1.00 | 0.00 | H |
| ATOM | 1664 | 1HB | ASP | A | 96 | 29.612 | 19.848 | 0.077 | 1.00 | 0.00 | H |
| ATOM | 1665 | 2HB | ASP | A | 96 | 28.096 | 19.123 | −0.360 | 1.00 | 0.00 | H |
| ATOM | 1666 | N | PRO | A | 97 | 32.225 | 17.816 | −0.031 | 1.00 | 0.00 | N |
| ATOM | 1667 | CA | PRO | A | 97 | 33.453 | 17.689 | 0.767 | 1.00 | 0.00 | C |
| ATOM | 1668 | C | PRO | A | 97 | 33.586 | 18.805 | 1.829 | 1.00 | 0.00 | C |
| ATOM | 1669 | O | PRO | A | 97 | 34.646 | 18.941 | 2.436 | 1.00 | 0.00 | O |
| ATOM | 1670 | CB | PRO | A | 97 | 34.573 | 17.803 | −0.282 | 1.00 | 0.00 | C |
| ATOM | 1671 | CG | PRO | A | 97 | 34.005 | 18.761 | −1.322 | 1.00 | 0.00 | C |
| ATOM | 1672 | CD | PRO | A | 97 | 32.509 | 18.440 | −1.328 | 1.00 | 0.00 | C |
| ATOM | 1673 | HA | PRO | A | 97 | 33.505 | 16.727 | 1.277 | 1.00 | 0.00 | H |
| ATOM | 1674 | 1HB | PRO | A | 97 | 35.528 | 18.142 | 0.122 | 1.00 | 0.00 | H |
| ATOM | 1675 | 2HB | PRO | A | 97 | 34.745 | 16.827 | −0.737 | 1.00 | 0.00 | H |
| ATOM | 1676 | 1HG | PRO | A | 97 | 34.465 | 18.649 | −2.305 | 1.00 | 0.00 | H |
| ATOM | 1677 | 2HG | PRO | A | 97 | 34.157 | 19.792 | −0.998 | 1.00 | 0.00 | H |
| ATOM | 1678 | 1HD | PRO | A | 97 | 32.279 | 17.730 | −2.123 | 1.00 | 0.00 | H |
| ATOM | 1679 | 2HD | PRO | A | 97 | 31.919 | 19.346 | −1.485 | 1.00 | 0.00 | H |
| ATOM | 1680 | N | ASP | A | 98 | 32.544 | 19.624 | 2.029 | 1.00 | 0.00 | N |
| ATOM | 1681 | CA | ASP | A | 98 | 32.540 | 20.812 | 2.884 | 1.00 | 0.00 | C |
| ATOM | 1682 | C | ASP | A | 98 | 32.192 | 20.409 | 4.327 | 1.00 | 0.00 | C |
| ATOM | 1683 | O | ASP | A | 98 | 32.784 | 20.907 | 5.280 | 1.00 | 0.00 | O |
| ATOM | 1684 | CB | ASP | A | 98 | 31.458 | 21.776 | 2.332 | 1.00 | 0.00 | C |
| ATOM | 1685 | CG | ASP | A | 98 | 31.767 | 22.341 | 0.948 | 1.00 | 0.00 | C |
| ATOM | 1686 | OD1 | ASP | A | 98 | 32.396 | 23.417 | 0.893 | 1.00 | 0.00 | O |
| ATOM | 1687 | OD2 | ASP | A | 98 | 31.367 | 21.709 | −0.050 | 1.00 | 0.00 | O |
| ATOM | 1688 | H | ASP | A | 98 | 31.645 | 19.367 | 1.643 | 1.00 | 0.00 | H |
| ATOM | 1689 | HA | ASP | A | 98 | 33.523 | 21.289 | 2.861 | 1.00 | 0.00 | H |
| ATOM | 1690 | 1HB | ASP | A | 98 | 30.478 | 21.303 | 2.299 | 1.00 | 0.00 | H |
| ATOM | 1691 | 2HB | ASP | A | 98 | 31.350 | 22.622 | 3.012 | 1.00 | 0.00 | H |
| ATOM | 1692 | N | THR | A | 99 | 31.266 | 19.456 | 4.480 | 1.00 | 0.00 | N |
| ATOM | 1693 | CA | THR | A | 99 | 30.768 | 18.913 | 5.736 | 1.00 | 0.00 | C |
| ATOM | 1694 | C | THR | A | 99 | 30.887 | 17.374 | 5.793 | 1.00 | 0.00 | C |
| ATOM | 1695 | O | THR | A | 99 | 30.665 | 16.798 | 6.857 | 1.00 | 0.00 | O |
| ATOM | 1696 | CB | THR | A | 99 | 29.316 | 19.420 | 5.951 | 1.00 | 0.00 | C |
| ATOM | 1697 | OG1 | THR | A | 99 | 28.409 | 18.931 | 4.984 | 1.00 | 0.00 | O |
| ATOM | 1698 | CG2 | THR | A | 99 | 29.178 | 20.948 | 6.012 | 1.00 | 0.00 | C |
| ATOM | 1699 | H | THR | A | 99 | 30.700 | 19.230 | 3.648 | 1.00 | 0.00 | H |
| ATOM | 1700 | HA | THR | A | 99 | 31.378 | 19.282 | 6.562 | 1.00 | 0.00 | H |
| ATOM | 1701 | HB | THR | A | 99 | 28.973 | 19.028 | 6.911 | 1.00 | 0.00 | H |
| ATOM | 1702 | HG1 | THR | A | 99 | 28.715 | 19.099 | 4.080 | 1.00 | 0.00 | H |
| ATOM | 1703 | 1HG2 | THR | A | 99 | 28.153 | 21.236 | 6.248 | 1.00 | 0.00 | H |
| ATOM | 1704 | 2HG2 | THR | A | 99 | 29.820 | 21.362 | 6.789 | 1.00 | 0.00 | H |
| ATOM | 1705 | 3HG2 | THR | A | 99 | 29.441 | 21.420 | 5.066 | 1.00 | 0.00 | H |
| ATOM | 1706 | N | ALA | A | 100 | 31.207 | 16.721 | 4.657 | 1.00 | 0.00 | N |
| ATOM | 1707 | CA | ALA | A | 100 | 31.220 | 15.289 | 4.380 | 1.00 | 0.00 | C |
| ATOM | 1708 | C | ALA | A | 100 | 29.804 | 14.694 | 4.410 | 1.00 | 0.00 | C |
| ATOM | 1709 | O | ALA | A | 100 | 29.622 | 13.484 | 4.560 | 1.00 | 0.00 | O |
| ATOM | 1710 | CB | ALA | A | 100 | 32.124 | 14.569 | 5.366 | 1.00 | 0.00 | C |
| ATOM | 1711 | H | ALA | A | 100 | 31.293 | 17.307 | 3.837 | 1.00 | 0.00 | H |
| ATOM | 1712 | HA | ALA | A | 100 | 31.614 | 15.141 | 3.375 | 1.00 | 0.00 | H |
| ATOM | 1713 | 1HB | ALA | A | 100 | 32.203 | 13.525 | 5.074 | 1.00 | 0.00 | H |
| ATOM | 1714 | 2HB | ALA | A | 100 | 33.121 | 15.007 | 5.385 | 1.00 | 0.00 | H |
| ATOM | 1715 | 3HB | ALA | A | 100 | 31.727 | 14.571 | 6.380 | 1.00 | 0.00 | H |
| ATOM | 1716 | N | VAL | A | 101 | 28.802 | 15.550 | 4.248 | 1.00 | 0.00 | N |
| ATOM | 1717 | CA | VAL | A | 101 | 27.421 | 15.096 | 4.233 | 1.00 | 0.00 | C |
| ATOM | 1718 | C | VAL | A | 101 | 27.048 | 14.606 | 2.837 | 1.00 | 0.00 | C |
| ATOM | 1719 | O | VAL | A | 101 | 27.361 | 15.253 | 1.834 | 1.00 | 0.00 | O |
| ATOM | 1720 | CB | VAL | A | 101 | 26.446 | 16.230 | 4.634 | 1.00 | 0.00 | C |
| ATOM | 1721 | CG1 | VAL | A | 101 | 24.944 | 15.906 | 4.451 | 1.00 | 0.00 | C |
| ATOM | 1722 | CG2 | VAL | A | 101 | 26.654 | 16.645 | 6.097 | 1.00 | 0.00 | C |
| ATOM | 1723 | H | VAL | A | 101 | 28.988 | 16.526 | 4.061 | 1.00 | 0.00 | H |
| ATOM | 1724 | HA | VAL | A | 101 | 27.295 | 14.278 | 4.945 | 1.00 | 0.00 | H |
| ATOM | 1725 | HB | VAL | A | 101 | 26.667 | 17.096 | 4.005 | 1.00 | 0.00 | H |
| ATOM | 1726 | 1HG1 | VAL | A | 101 | 24.321 | 16.709 | 4.846 | 1.00 | 0.00 | H |
| ATOM | 1727 | 2HG1 | VAL | A | 101 | 24.675 | 15.794 | 3.402 | 1.00 | 0.00 | H |
| ATOM | 1728 | 3HG1 | VAL | A | 101 | 24.663 | 14.986 | 4.967 | 1.00 | 0.00 | H |
| ATOM | 1729 | 1HG2 | VAL | A | 101 | 26.131 | 17.575 | 6.320 | 1.00 | 0.00 | H |
| ATOM | 1730 | 2HG2 | VAL | A | 101 | 26.274 | 15.879 | 6.769 | 1.00 | 0.00 | H |
| ATOM | 1731 | 3HG2 | VAL | A | 101 | 27.707 | 16.805 | 6.324 | 1.00 | 0.00 | H |
| ATOM | 1732 | N | LEU | A | 102 | 26.401 | 13.444 | 2.785 | 1.00 | 0.00 | N |
| ATOM | 1733 | CA | LEU | A | 102 | 25.948 | 12.860 | 1.525 | 1.00 | 0.00 | C |
| ATOM | 1734 | C | LEU | A | 102 | 24.420 | 12.923 | 1.528 | 1.00 | 0.00 | C |
| ATOM | 1735 | O | LEU | A | 102 | 23.780 | 12.689 | 2.564 | 1.00 | 0.00 | O |
| ATOM | 1736 | CB | LEU | A | 102 | 26.400 | 11.393 | 1.409 | 1.00 | 0.00 | C |
| ATOM | 1737 | CG | LEU | A | 102 | 26.117 | 10.626 | 0.091 | 1.00 | 0.00 | C |
| ATOM | 1738 | CD1 | LEU | A | 102 | 27.240 | 9.606 | −0.175 | 1.00 | 0.00 | C |
| ATOM | 1739 | CD2 | LEU | A | 102 | 24.768 | 9.881 | 0.108 | 1.00 | 0.00 | C |
| ATOM | 1740 | H | LEU | A | 102 | 26.149 | 12.992 | 3.649 | 1.00 | 0.00 | H |
| ATOM | 1741 | HA | LEU | A | 102 | 26.297 | 13.392 | 0.656 | 1.00 | 0.00 | H |
| ATOM | 1742 | 1HB | LEU | A | 102 | 27.480 | 11.402 | 1.565 | 1.00 | 0.00 | H |
| ATOM | 1743 | 2HB | LEU | A | 102 | 26.013 | 10.818 | 2.252 | 1.00 | 0.00 | H |
| ATOM | 1744 | HG | LEU | A | 102 | 26.116 | 11.336 | −0.738 | 1.00 | 0.00 | H |
| ATOM | 1745 | 1HD1 | LEU | A | 102 | 27.067 | 9.042 | −1.090 | 1.00 | 0.00 | H |
| ATOM | 1746 | 2HD1 | LEU | A | 102 | 28.212 | 10.091 | −0.274 | 1.00 | 0.00 | H |
| ATOM | 1747 | 3HD1 | LEU | A | 102 | 27.321 | 8.882 | 0.637 | 1.00 | 0.00 | H |
| ATOM | 1748 | 1HD2 | LEU | A | 102 | 24.616 | 9.337 | −0.824 | 1.00 | 0.00 | H |
| ATOM | 1749 | 2HD2 | LEU | A | 102 | 24.724 | 9.154 | 0.920 | 1.00 | 0.00 | H |
| ATOM | 1750 | 3HD2 | LEU | A | 102 | 23.916 | 10.546 | 0.221 | 1.00 | 0.00 | H |
| ATOM | 1751 | N | LYS | A | 103 | 23.845 | 13.255 | 0.379 | 1.00 | 0.00 | N |
| ATOM | 1752 | CA | LYS | A | 103 | 22.397 | 13.326 | 0.231 | 1.00 | 0.00 | C |
| ATOM | 1753 | C | LYS | A | 103 | 21.974 | 12.636 | −1.054 | 1.00 | 0.00 | C |
| ATOM | 1754 | O | LYS | A | 103 | 22.411 | 13.002 | −2.147 | 1.00 | 0.00 | O |
| ATOM | 1755 | CB | LYS | A | 103 | 21.926 | 14.782 | 0.214 | 1.00 | 0.00 | C |
| ATOM | 1756 | CG | LYS | A | 103 | 22.214 | 15.468 | 1.563 | 1.00 | 0.00 | C |
| ATOM | 1757 | CD | LYS | A | 103 | 21.270 | 16.619 | 1.894 | 1.00 | 0.00 | C |
| ATOM | 1758 | CE | LYS | A | 103 | 21.633 | 17.282 | 3.231 | 1.00 | 0.00 | C |
| ATOM | 1759 | NZ | LYS | A | 103 | 20.596 | 18.227 | 3.650 | 1.00 | 0.00 | N |
| ATOM | 1760 | H | LYS | A | 103 | 24.412 | 13.563 | −0.412 | 1.00 | 0.00 | H |
| ATOM | 1761 | HA | LYS | A | 103 | 21.901 | 12.812 | 1.049 | 1.00 | 0.00 | H |
| ATOM | 1762 | 1HB | LYS | A | 103 | 22.393 | 15.339 | −0.601 | 1.00 | 0.00 | H |
| ATOM | 1763 | 2HB | LYS | A | 103 | 20.855 | 14.800 | 0.015 | 1.00 | 0.00 | H |
| ATOM | 1764 | 1HG | LYS | A | 103 | 22.133 | 14.741 | 2.372 | 1.00 | 0.00 | H |
| ATOM | 1765 | 2HG | LYS | A | 103 | 23.247 | 15.817 | 1.566 | 1.00 | 0.00 | H |
| ATOM | 1766 | 1HD | LYS | A | 103 | 21.299 | 17.352 | 1.087 | 1.00 | 0.00 | H |
| ATOM | 1767 | 2HD | LYS | A | 103 | 20.248 | 16.242 | 1.934 | 1.00 | 0.00 | H |
| ATOM | 1768 | 1HE | LYS | A | 103 | 21.770 | 16.541 | 4.017 | 1.00 | 0.00 | H |
| ATOM | 1769 | 2HE | LYS | A | 103 | 22.577 | 17.821 | 3.130 | 1.00 | 0.00 | H |
| ATOM | 1770 | 1HZ | LYS | A | 103 | 20.892 | 18.815 | 4.408 | 1.00 | 0.00 | H |
| ATOM | 1771 | 2HZ | LYS | A | 103 | 20.432 | 18.854 | 2.831 | 1.00 | 0.00 | H |
| ATOM | 1772 | 3HZ | LYS | A | 103 | 19.706 | 17.779 | 3.839 | 1.00 | 0.00 | H |
| ATOM | 1773 | N | LEU | A | 104 | 21.130 | 11.623 | −0.909 | 1.00 | 0.00 | N |
| ATOM | 1774 | CA | LEU | A | 104 | 20.608 | 10.871 | −2.042 | 1.00 | 0.00 | C |
| ATOM | 1775 | C | LEU | A | 104 | 19.635 | 11.758 | −2.823 | 1.00 | 0.00 | C |
| ATOM | 1776 | O | LEU | A | 104 | 18.832 | 12.480 | −2.228 | 1.00 | 0.00 | O |
| ATOM | 1777 | CB | LEU | A | 104 | 19.886 | 9.616 | −1.530 | 1.00 | 0.00 | C |
| ATOM | 1778 | CG | LEU | A | 104 | 19.251 | 8.673 | −2.579 | 1.00 | 0.00 | C |
| ATOM | 1779 | CD1 | LEU | A | 104 | 20.294 | 8.080 | −3.534 | 1.00 | 0.00 | C |
| ATOM | 1780 | CD2 | LEU | A | 104 | 18.466 | 7.541 | −1.895 | 1.00 | 0.00 | C |
| ATOM | 1781 | H | LEU | A | 104 | 20.762 | 11.400 | 0.011 | 1.00 | 0.00 | H |
| ATOM | 1782 | HA | LEU | A | 104 | 21.438 | 10.578 | −2.689 | 1.00 | 0.00 | H |
| ATOM | 1783 | 1HB | LEU | A | 104 | 20.589 | 9.040 | −0.929 | 1.00 | 0.00 | H |
| ATOM | 1784 | 2HB | LEU | A | 104 | 19.101 | 9.933 | −0.842 | 1.00 | 0.00 | H |
| ATOM | 1785 | HG | LEU | A | 104 | 18.533 | 9.235 | −3.178 | 1.00 | 0.00 | H |
| ATOM | 1786 | 1HD1 | LEU | A | 104 | 19.814 | 7.422 | −4.255 | 1.00 | 0.00 | H |
| ATOM | 1787 | 2HD1 | LEU | A | 104 | 20.797 | 8.850 | −4.114 | 1.00 | 0.00 | H |
| ATOM | 1788 | 3HD1 | LEU | A | 104 | 21.047 | 7.499 | −3.003 | 1.00 | 0.00 | H |
| ATOM | 1789 | 1HD2 | LEU | A | 104 | 17.942 | 6.930 | −2.631 | 1.00 | 0.00 | H |
| ATOM | 1790 | 2HD2 | LEU | A | 104 | 19.123 | 6.876 | −1.333 | 1.00 | 0.00 | H |
| ATOM | 1791 | 3HD2 | LEU | A | 104 | 17.719 | 7.930 | −1.203 | 1.00 | 0.00 | H |
| ATOM | 1792 | N | CYS | A | 105 | 19.715 | 11.720 | −4.147 | 1.00 | 0.00 | N |
| ATOM | 1793 | CA | CYS | A | 105 | 18.831 | 12.528 | −4.973 | 1.00 | 0.00 | C |
| ATOM | 1794 | C | CYS | A | 105 | 18.285 | 11.753 | −6.164 | 1.00 | 0.00 | C |
| ATOM | 1795 | O | CYS | A | 105 | 18.566 | 10.566 | −6.332 | 1.00 | 0.00 | O |
| ATOM | 1796 | CB | CYS | A | 105 | 19.553 | 13.786 | −5.472 | 1.00 | 0.00 | C |
| ATOM | 1797 | SG | CYS | A | 105 | 18.437 | 15.198 | −5.756 | 1.00 | 0.00 | S |
| ATOM | 1798 | H | CYS | A | 105 | 20.392 | 11.132 | −4.628 | 1.00 | 0.00 | H |
| ATOM | 1799 | HA | CYS | A | 105 | 17.950 | 12.842 | −4.411 | 1.00 | 0.00 | H |
| ATOM | 1800 | 1HB | CYS | A | 105 | 20.271 | 14.115 | −4.721 | 1.00 | 0.00 | H |
| ATOM | 1801 | 2HB | CYS | A | 105 | 20.127 | 13.584 | −6.379 | 1.00 | 0.00 | H |
| ATOM | 1802 | HG | CYS | A | 105 | 19.393 | 16.041 | −6.161 | 1.00 | 0.00 | H |
| ATOM | 1803 | N | ASP | A | 106 | 17.488 | 12.445 | −6.971 | 1.00 | 0.00 | N |
| ATOM | 1804 | CA | ASP | A | 106 | 16.865 | 11.892 | −8.169 | 1.00 | 0.00 | C |
| ATOM | 1805 | C | ASP | A | 106 | 15.924 | 10.736 | −7.897 | 1.00 | 0.00 | C |
| ATOM | 1806 | O | ASP | A | 106 | 16.297 | 9.565 | −8.029 | 1.00 | 0.00 | O |
| ATOM | 1807 | CB | ASP | A | 106 | 17.922 | 11.443 | −9.175 | 1.00 | 0.00 | C |
| ATOM | 1808 | CG | ASP | A | 106 | 18.745 | 12.603 | −9.737 | 1.00 | 0.00 | C |
| ATOM | 1809 | OD1 | ASP | A | 106 | 19.717 | 13.025 | −9.076 | 1.00 | 0.00 | O |
| ATOM | 1810 | OD2 | ASP | A | 106 | 18.358 | 13.109 | −10.812 | 1.00 | 0.00 | O |
| ATOM | 1811 | H | ASP | A | 106 | 17.288 | 13.407 | −6.750 | 1.00 | 0.00 | H |
| ATOM | 1812 | HA | ASP | A | 106 | 16.286 | 12.703 | −8.616 | 1.00 | 0.00 | H |
| ATOM | 1813 | 1HB | ASP | A | 106 | 18.607 | 10.711 | −8.760 | 1.00 | 0.00 | H |
| ATOM | 1814 | 2HB | ASP | A | 106 | 17.451 | 10.945 | −10.024 | 1.00 | 0.00 | H |
| ATOM | 1815 | N | PHE | A | 107 | 14.691 | 11.063 | −7.536 | 1.00 | 0.00 | N |
| ATOM | 1816 | CA | PHE | A | 107 | 13.708 | 10.027 | −7.267 | 1.00 | 0.00 | C |
| ATOM | 1817 | C | PHE | A | 107 | 12.814 | 9.787 | −8.483 | 1.00 | 0.00 | C |
| ATOM | 1818 | O | PHE | A | 107 | 11.692 | 9.290 | −8.361 | 1.00 | 0.00 | O |
| ATOM | 1819 | CB | PHE | A | 107 | 12.900 | 10.406 | −6.027 | 1.00 | 0.00 | C |
| ATOM | 1820 | CG | PHE | A | 107 | 13.663 | 10.119 | −4.742 | 1.00 | 0.00 | C |
| ATOM | 1821 | CD1 | PHE | A | 107 | 14.669 | 10.998 | −4.283 | 1.00 | 0.00 | C |
| ATOM | 1822 | CD2 | PHE | A | 107 | 13.408 | 8.928 | −4.032 | 1.00 | 0.00 | C |
| ATOM | 1823 | CE1 | PHE | A | 107 | 15.419 | 10.682 | −3.133 | 1.00 | 0.00 | C |
| ATOM | 1824 | CE2 | PHE | A | 107 | 14.162 | 8.607 | −2.890 | 1.00 | 0.00 | C |
| ATOM | 1825 | CZ | PHE | A | 107 | 15.167 | 9.484 | −2.437 | 1.00 | 0.00 | C |
| ATOM | 1826 | H | PHE | A | 107 | 14.399 | 12.030 | −7.559 | 1.00 | 0.00 | H |
| ATOM | 1827 | HA | PHE | A | 107 | 14.171 | 9.061 | −7.053 | 1.00 | 0.00 | H |
| ATOM | 1828 | 1HB | PHE | A | 107 | 12.606 | 11.456 | −6.055 | 1.00 | 0.00 | H |
| ATOM | 1829 | 2HB | PHE | A | 107 | 11.965 | 9.846 | −5.974 | 1.00 | 0.00 | H |
| ATOM | 1830 | HD1 | PHE | A | 107 | 14.888 | 11.907 | −4.824 | 1.00 | 0.00 | H |
| ATOM | 1831 | HD2 | PHE | A | 107 | 12.639 | 8.248 | −4.364 | 1.00 | 0.00 | H |
| ATOM | 1832 | HE1 | PHE | A | 107 | 16.195 | 11.352 | −2.789 | 1.00 | 0.00 | H |
| ATOM | 1833 | HE2 | PHE | A | 107 | 13.958 | 7.687 | −2.365 | 1.00 | 0.00 | H |
| ATOM | 1834 | HZ | PHE | A | 107 | 15.742 | 9.242 | −1.555 | 1.00 | 0.00 | H |
| ATOM | 1835 | N | GLY | A | 108 | 13.353 | 10.111 | −9.655 | 1.00 | 0.00 | N |
| ATOM | 1836 | CA | GLY | A | 108 | 12.622 | 9.942 | −10.900 | 1.00 | 0.00 | C |
| ATOM | 1837 | C | GLY | A | 108 | 12.367 | 8.504 | −11.323 | 1.00 | 0.00 | C |
| ATOM | 1838 | O | GLY | A | 108 | 11.568 | 8.261 | −12.221 | 1.00 | 0.00 | O |
| ATOM | 1839 | H | GLY | A | 108 | 14.296 | 10.500 | −9.713 | 1.00 | 0.00 | H |
| ATOM | 1840 | 1HA | GLY | A | 108 | 11.669 | 10.471 | −10.843 | 1.00 | 0.00 | H |
| ATOM | 1841 | 2HA | GLY | A | 108 | 13.193 | 10.433 | −11.688 | 1.00 | 0.00 | H |
| ATOM | 1842 | N | SER | A | 109 | 13.037 | 7.546 | −10.698 | 1.00 | 0.00 | N |
| ATOM | 1843 | CA | SER | A | 109 | 12.837 | 6.142 | −11.046 | 1.00 | 0.00 | C |
| ATOM | 1844 | C | SER | A | 109 | 12.257 | 5.397 | −9.861 | 1.00 | 0.00 | C |
| ATOM | 1845 | O | SER | A | 109 | 11.930 | 4.220 | −9.966 | 1.00 | 0.00 | O |
| ATOM | 1846 | CB | SER | A | 109 | 14.167 | 5.475 | −11.421 | 1.00 | 0.00 | C |
| ATOM | 1847 | OG | SER | A | 109 | 15.119 | 5.735 | −10.399 | 1.00 | 0.00 | O |
| ATOM | 1848 | H | SER | A | 109 | 13.737 | 7.796 | −10.014 | 1.00 | 0.00 | H |
| ATOM | 1849 | HA | SER | A | 109 | 12.140 | 5.988 | −11.872 | 1.00 | 0.00 | H |
| ATOM | 1850 | 1HB | SER | A | 109 | 14.064 | 4.399 | −11.575 | 1.00 | 0.00 | H |
| ATOM | 1851 | 2HB | SER | A | 109 | 14.540 | 5.891 | −12.357 | 1.00 | 0.00 | H |
| ATOM | 1852 | HG | SER | A | 109 | 14.928 | 5.239 | −9.602 | 1.00 | 0.00 | H |
| ATOM | 1853 | N | ALA | A | 110 | 12.141 | 6.094 | −8.733 | 1.00 | 0.00 | N |
| ATOM | 1854 | CA | ALA | A | 110 | 11.648 | 5.505 | −7.496 | 1.00 | 0.00 | C |
| ATOM | 1855 | C | ALA | A | 110 | 10.208 | 5.000 | −7.570 | 1.00 | 0.00 | C |
| ATOM | 1856 | O | ALA | A | 110 | 9.368 | 5.559 | −8.269 | 1.00 | 0.00 | O |
| ATOM | 1857 | CB | ALA | A | 110 | 11.804 | 6.508 | −6.351 | 1.00 | 0.00 | C |
| ATOM | 1858 | H | ALA | A | 110 | 12.283 | 7.093 | −8.766 | 1.00 | 0.00 | H |
| ATOM | 1859 | HA | ALA | A | 110 | 12.303 | 4.660 | −7.311 | 1.00 | 0.00 | H |
| ATOM | 1860 | 1HB | ALA | A | 110 | 11.533 | 6.058 | −5.395 | 1.00 | 0.00 | H |
| ATOM | 1861 | 2HB | ALA | A | 110 | 12.840 | 6.838 | −6.269 | 1.00 | 0.00 | H |
| ATOM | 1862 | 3HB | ALA | A | 110 | 11.181 | 7.390 | −6.501 | 1.00 | 0.00 | H |
| ATOM | 1863 | N | LYS | A | 111 | 9.935 | 3.933 | −6.833 | 1.00 | 0.00 | N |
| ATOM | 1864 | CA | LYS | A | 111 | 8.606 | 3.337 | −6.826 | 1.00 | 0.00 | C |
| ATOM | 1865 | C | LYS | A | 111 | 8.391 | 2.415 | −5.637 | 1.00 | 0.00 | C |
| ATOM | 1866 | O | LYS | A | 111 | 9.299 | 1.693 | −5.207 | 1.00 | 0.00 | O |
| ATOM | 1867 | CB | LYS | A | 111 | 8.395 | 2.545 | −8.122 | 1.00 | 0.00 | C |
| ATOM | 1868 | CG | LYS | A | 111 | 6.938 | 2.059 | −8.325 | 1.00 | 0.00 | C |
| ATOM | 1869 | CD | LYS | A | 111 | 6.746 | 1.035 | −9.452 | 1.00 | 0.00 | C |
| ATOM | 1870 | CE | LYS | A | 111 | 5.374 | 0.331 | −9.368 | 1.00 | 0.00 | C |
| ATOM | 1871 | NZ | LYS | A | 111 | 5.328 | −0.677 | −8.296 | 1.00 | 0.00 | N |
| ATOM | 1872 | H | LYS | A | 111 | 10.674 | 3.493 | −6.288 | 1.00 | 0.00 | H |
| ATOM | 1873 | HA | LYS | A | 111 | 7.866 | 4.138 | −6.766 | 1.00 | 0.00 | H |
| ATOM | 1874 | 1HB | LYS | A | 111 | 8.670 | 3.138 | −8.994 | 1.00 | 0.00 | H |
| ATOM | 1875 | 2HB | LYS | A | 111 | 9.071 | 1.687 | −8.129 | 1.00 | 0.00 | H |
| ATOM | 1876 | 1HG | LYS | A | 111 | 6.572 | 1.587 | −7.420 | 1.00 | 0.00 | H |
| ATOM | 1877 | 2HG | LYS | A | 111 | 6.287 | 2.918 | −8.494 | 1.00 | 0.00 | H |
| ATOM | 1878 | 1HD | LYS | A | 111 | 6.837 | 1.547 | −10.411 | 1.00 | 0.00 | H |
| ATOM | 1879 | 2HD | LYS | A | 111 | 7.544 | 0.290 | −9.433 | 1.00 | 0.00 | H |
| ATOM | 1880 | 1HE | LYS | A | 111 | 4.570 | 1.053 | −9.226 | 1.00 | 0.00 | H |
| ATOM | 1881 | 2HE | LYS | A | 111 | 5.170 | −0.187 | −10.306 | 1.00 | 0.00 | H |
| ATOM | 1882 | 1HZ | LYS | A | 111 | 4.399 | −1.150 | −8.295 | 1.00 | 0.00 | H |
| ATOM | 1883 | 2HZ | LYS | A | 111 | 6.002 | −1.406 | −8.529 | 1.00 | 0.00 | H |
| ATOM | 1884 | 3HZ | LYS | A | 111 | 5.502 | −0.309 | −7.368 | 1.00 | 0.00 | H |
| ATOM | 1885 | N | GLN | A | 112 | 7.183 | 2.438 | −5.096 | 1.00 | 0.00 | N |
| ATOM | 1886 | CA | GLN | A | 112 | 6.886 | 1.558 | −3.988 | 1.00 | 0.00 | C |
| ATOM | 1887 | C | GLN | A | 112 | 6.695 | 0.168 | −4.590 | 1.00 | 0.00 | C |
| ATOM | 1888 | O | GLN | A | 112 | 5.870 | −0.018 | −5.491 | 1.00 | 0.00 | O |
| ATOM | 1889 | CB | GLN | A | 112 | 5.611 | 2.005 | −3.278 | 1.00 | 0.00 | C |
| ATOM | 1890 | CG | GLN | A | 112 | 5.581 | 1.766 | −1.751 | 1.00 | 0.00 | C |
| ATOM | 1891 | CD | GLN | A | 112 | 4.911 | 2.911 | −0.985 | 1.00 | 0.00 | C |
| ATOM | 1892 | OE1 | GLN | A | 112 | 5.374 | 4.054 | −1.009 | 1.00 | 0.00 | O |
| ATOM | 1893 | NE2 | GLN | A | 112 | 3.824 | 2.624 | −0.284 | 1.00 | 0.00 | N |
| ATOM | 1894 | H | GLN | A | 112 | 6.444 | 2.986 | −5.505 | 1.00 | 0.00 | H |
| ATOM | 1895 | HA | GLN | A | 112 | 7.689 | 1.543 | −3.264 | 1.00 | 0.00 | H |
| ATOM | 1896 | 1HB | GLN | A | 112 | 5.500 | 3.077 | −3.451 | 1.00 | 0.00 | H |
| ATOM | 1897 | 2HB | GLN | A | 112 | 4.717 | 1.571 | −3.730 | 1.00 | 0.00 | H |
| ATOM | 1898 | 1HG | GLN | A | 112 | 5.125 | 0.803 | −1.519 | 1.00 | 0.00 | H |
| ATOM | 1899 | 2HG | GLN | A | 112 | 6.591 | 1.724 | −1.354 | 1.00 | 0.00 | H |
| ATOM | 1900 | 1HE2 | GLN | A | 112 | 3.349 | 3.344 | 0.237 | 1.00 | 0.00 | H |
| ATOM | 1901 | 2HE2 | GLN | A | 112 | 3.468 | 1.682 | −0.259 | 1.00 | 0.00 | H |
| ATOM | 1902 | N | LEU | A | 113 | 7.470 | −0.800 | −4.113 | 1.00 | 0.00 | N |
| ATOM | 1903 | CA | LEU | A | 113 | 7.376 | −2.162 | −4.621 | 1.00 | 0.00 | C |
| ATOM | 1904 | C | LEU | A | 113 | 6.431 | −2.974 | −3.760 | 1.00 | 0.00 | C |
| ATOM | 1905 | O | LEU | A | 113 | 6.610 | −3.071 | −2.546 | 1.00 | 0.00 | O |
| ATOM | 1906 | CB | LEU | A | 113 | 8.751 | −2.824 | −4.639 | 1.00 | 0.00 | C |
| ATOM | 1907 | CG | LEU | A | 113 | 9.841 | −2.077 | −5.444 | 1.00 | 0.00 | C |
| ATOM | 1908 | CD1 | LEU | A | 113 | 11.142 | −2.893 | −5.444 | 1.00 | 0.00 | C |
| ATOM | 1909 | CD2 | LEU | A | 113 | 9.429 | −1.762 | −6.889 | 1.00 | 0.00 | C |
| ATOM | 1910 | H | LEU | A | 113 | 8.081 | −0.610 | −3.323 | 1.00 | 0.00 | H |
| ATOM | 1911 | HA | LEU | A | 113 | 6.983 | −2.178 | −5.639 | 1.00 | 0.00 | H |
| ATOM | 1912 | 1HB | LEU | A | 113 | 9.106 | −2.954 | −3.614 | 1.00 | 0.00 | H |
| ATOM | 1913 | 2HB | LEU | A | 113 | 8.645 | −3.833 | −5.043 | 1.00 | 0.00 | H |
| ATOM | 1914 | HG | LEU | A | 113 | 10.038 | −1.127 | −4.946 | 1.00 | 0.00 | H |
| ATOM | 1915 | 1HD1 | LEU | A | 113 | 11.931 | −2.393 | −6.005 | 1.00 | 0.00 | H |
| ATOM | 1916 | 2HD1 | LEU | A | 113 | 11.501 | −3.047 | −4.427 | 1.00 | 0.00 | H |
| ATOM | 1917 | 3HD1 | LEU | A | 113 | 10.996 | −3.873 | −5.901 | 1.00 | 0.00 | H |
| ATOM | 1918 | 1HD2 | LEU | A | 113 | 10.232 | −1.259 | −7.423 | 1.00 | 0.00 | H |
| ATOM | 1919 | 2HD2 | LEU | A | 113 | 9.199 | −2.676 | −7.433 | 1.00 | 0.00 | H |
| ATOM | 1920 | 3HD2 | LEU | A | 113 | 8.561 | −1.104 | −6.936 | 1.00 | 0.00 | H |
| ATOM | 1921 | N | VAL | A | 114 | 5.424 | −3.556 | −4.401 | 1.00 | 0.00 | N |
| ATOM | 1922 | CA | VAL | A | 114 | 4.430 | −4.360 | −3.704 | 1.00 | 0.00 | C |
| ATOM | 1923 | C | VAL | A | 114 | 4.592 | −5.822 | −4.088 | 1.00 | 0.00 | C |
| ATOM | 1924 | O | VAL | A | 114 | 4.523 | −6.174 | −5.266 | 1.00 | 0.00 | O |
| ATOM | 1925 | CB | VAL | A | 114 | 3.003 | −3.902 | −4.060 | 1.00 | 0.00 | C |
| ATOM | 1926 | CG1 | VAL | A | 114 | 1.909 | −4.625 | −3.246 | 1.00 | 0.00 | C |
| ATOM | 1927 | CG2 | VAL | A | 114 | 2.825 | −2.377 | −3.903 | 1.00 | 0.00 | C |
| ATOM | 1928 | H | VAL | A | 114 | 5.325 | −3.461 | −5.400 | 1.00 | 0.00 | H |
| ATOM | 1929 | HA | VAL | A | 114 | 4.538 | −4.271 | −2.621 | 1.00 | 0.00 | H |
| ATOM | 1930 | HB | VAL | A | 114 | 2.816 | −4.122 | −5.113 | 1.00 | 0.00 | H |
| ATOM | 1931 | 1HG1 | VAL | A | 114 | 0.917 | −4.261 | −3.515 | 1.00 | 0.00 | H |
| ATOM | 1932 | 2HG1 | VAL | A | 114 | 1.905 | −5.701 | −3.427 | 1.00 | 0.00 | H |
| ATOM | 1933 | 3HG1 | VAL | A | 114 | 2.039 | −4.468 | −2.175 | 1.00 | 0.00 | H |
| ATOM | 1934 | 1HG2 | VAL | A | 114 | 1.802 | −2.075 | −4.131 | 1.00 | 0.00 | H |
| ATOM | 1935 | 2HG2 | VAL | A | 114 | 3.052 | −2.051 | −2.887 | 1.00 | 0.00 | H |
| ATOM | 1936 | 3HG2 | VAL | A | 114 | 3.469 | −1.818 | −4.584 | 1.00 | 0.00 | H |
| ATOM | 1937 | N | ARG | A | 115 | 4.821 | −6.667 | −3.091 | 1.00 | 0.00 | N |
| ATOM | 1938 | CA | ARG | A | 115 | 4.993 | −8.091 | −3.329 | 1.00 | 0.00 | C |
| ATOM | 1939 | C | ARG | A | 115 | 3.896 | −8.592 | −4.271 | 1.00 | 0.00 | C |
| ATOM | 1940 | O | ARG | A | 115 | 2.715 | −8.312 | −4.066 | 1.00 | 0.00 | O |
| ATOM | 1941 | CB | ARG | A | 115 | 4.941 | −8.841 | −1.996 | 1.00 | 0.00 | C |
| ATOM | 1942 | CG | ARG | A | 115 | 6.096 | −8.472 | −1.047 | 1.00 | 0.00 | C |
| ATOM | 1943 | CD | ARG | A | 115 | 6.089 | −9.300 | 0.245 | 1.00 | 0.00 | C |
| ATOM | 1944 | NE | ARG | A | 115 | 7.214 | −8.930 | 1.116 | 1.00 | 0.00 | N |
| ATOM | 1945 | CZ | ARG | A | 115 | 7.641 | −9.603 | 2.192 | 1.00 | 0.00 | C |
| ATOM | 1946 | NH1 | ARG | A | 115 | 7.042 | −10.729 | 2.576 | 1.00 | 0.00 | N |
| ATOM | 1947 | NH2 | ARG | A | 115 | 8.678 | −9.139 | 2.883 | 1.00 | 0.00 | N |
| ATOM | 1948 | H | ARG | A | 115 | 4.857 | −6.319 | −2.146 | 1.00 | 0.00 | H |
| ATOM | 1949 | HA | ARG | A | 115 | 5.969 | −8.234 | −3.797 | 1.00 | 0.00 | H |
| ATOM | 1950 | 1HB | ARG | A | 115 | 3.984 | −8.662 | −1.502 | 1.00 | 0.00 | H |
| ATOM | 1951 | 2HB | ARG | A | 115 | 4.980 | −9.914 | −2.193 | 1.00 | 0.00 | H |
| ATOM | 1952 | 1HG | ARG | A | 115 | 7.045 | −8.619 | −1.563 | 1.00 | 0.00 | H |
| ATOM | 1953 | 2HG | ARG | A | 115 | 6.044 | −7.413 | −0.794 | 1.00 | 0.00 | H |
| ATOM | 1954 | 1HD | ARG | A | 115 | 5.156 | −9.155 | 0.790 | 1.00 | 0.00 | H |
| ATOM | 1955 | 2HD | ARG | A | 115 | 6.161 | −10.360 | 0.002 | 1.00 | 0.00 | H |
| ATOM | 1956 | HE | ARG | A | 115 | 7.670 | −8.059 | 0.871 | 1.00 | 0.00 | H |
| ATOM | 1957 | 1HH1 | ARG | A | 115 | 7.347 | −11.255 | 3.382 | 1.00 | 0.00 | H |
| ATOM | 1958 | 2HH1 | ARG | A | 115 | 6.252 | −11.101 | 2.073 | 1.00 | 0.00 | H |
| ATOM | 1959 | 1HH2 | ARG | A | 115 | 9.034 | −9.613 | 3.700 | 1.00 | 0.00 | H |
| ATOM | 1960 | 2HH2 | ARG | A | 115 | 9.160 | −8.291 | 2.623 | 1.00 | 0.00 | H |
| ATOM | 1961 | N | GLY | A | 116 | 4.288 | −9.302 | −5.324 | 1.00 | 0.00 | N |
| ATOM | 1962 | CA | GLY | A | 116 | 3.303 | −9.817 | −6.261 | 1.00 | 0.00 | C |
| ATOM | 1963 | C | GLY | A | 116 | 2.967 | −8.922 | −7.442 | 1.00 | 0.00 | C |
| ATOM | 1964 | O | GLY | A | 116 | 2.395 | −9.393 | −8.421 | 1.00 | 0.00 | O |
| ATOM | 1965 | H | GLY | A | 116 | 5.268 | −9.440 | −5.517 | 1.00 | 0.00 | H |
| ATOM | 1966 | 1HA | GLY | A | 116 | 3.706 | −10.746 | −6.666 | 1.00 | 0.00 | H |
| ATOM | 1967 | 2HA | GLY | A | 116 | 2.374 | −10.103 | −5.764 | 1.00 | 0.00 | H |
| ATOM | 1968 | N | GLU | A | 117 | 3.296 | −7.635 | −7.354 | 1.00 | 0.00 | N |
| ATOM | 1969 | CA | GLU | A | 117 | 3.030 | −6.700 | −8.450 | 1.00 | 0.00 | C |
| ATOM | 1970 | C | GLU | A | 117 | 4.276 | −6.607 | −9.331 | 1.00 | 0.00 | C |
| ATOM | 1971 | O | GLU | A | 117 | 5.365 | −6.317 | −8.840 | 1.00 | 0.00 | O |
| ATOM | 1972 | CB | GLU | A | 117 | 2.702 | −5.315 | −7.907 | 1.00 | 0.00 | C |
| ATOM | 1973 | CG | GLU | A | 117 | 2.474 | −4.180 | −8.935 | 1.00 | 0.00 | C |
| ATOM | 1974 | CD | GLU | A | 117 | 2.234 | −2.842 | −8.245 | 1.00 | 0.00 | C |
| ATOM | 1975 | OE1 | GLU | A | 117 | 3.132 | −1.974 | −8.315 | 1.00 | 0.00 | O |
| ATOM | 1976 | OE2 | GLU | A | 117 | 1.165 | −2.692 | −7.624 | 1.00 | 0.00 | O |
| ATOM | 1977 | H | GLU | A | 117 | 3.780 | −7.277 | −6.532 | 1.00 | 0.00 | H |
| ATOM | 1978 | HA | GLU | A | 117 | 2.157 | −7.030 | −9.009 | 1.00 | 0.00 | H |
| ATOM | 1979 | 1HB | GLU | A | 117 | 1.823 | −5.387 | −7.265 | 1.00 | 0.00 | H |
| ATOM | 1980 | 2HB | GLU | A | 117 | 3.513 | −4.992 | −7.254 | 1.00 | 0.00 | H |
| ATOM | 1981 | 1HG | GLU | A | 117 | 3.330 | −4.062 | −9.600 | 1.00 | 0.00 | H |
| ATOM | 1982 | 2HG | GLU | A | 117 | 1.614 | −4.406 | −9.565 | 1.00 | 0.00 | H |
| ATOM | 1983 | N | PRO | A | 118 | 4.130 | −6.840 | −10.645 | 1.00 | 0.00 | N |
| ATOM | 1984 | CA | PRO | A | 118 | 5.292 | −6.771 | −11.541 | 1.00 | 0.00 | C |
| ATOM | 1985 | C | PRO | A | 118 | 5.830 | −5.359 | −11.760 | 1.00 | 0.00 | C |
| ATOM | 1986 | O | PRO | A | 118 | 5.084 | −4.381 | −11.718 | 1.00 | 0.00 | O |
| ATOM | 1987 | CB | PRO | A | 118 | 4.780 | −7.395 | −12.839 | 1.00 | 0.00 | C |
| ATOM | 1988 | CG | PRO | A | 118 | 3.281 | −7.148 | −12.853 | 1.00 | 0.00 | C |
| ATOM | 1989 | CD | PRO | A | 118 | 2.894 | −7.133 | −11.376 | 1.00 | 0.00 | C |
| ATOM | 1990 | HA | PRO | A | 118 | 6.087 | −7.398 | −11.135 | 1.00 | 0.00 | H |
| ATOM | 1991 | 1HB | PRO | A | 118 | 5.273 | −7.006 | −13.732 | 1.00 | 0.00 | H |
| ATOM | 1992 | 2HB | PRO | A | 118 | 4.969 | −8.469 | −12.816 | 1.00 | 0.00 | H |
| ATOM | 1993 | 1HG | PRO | A | 118 | 2.729 | −7.891 | −13.430 | 1.00 | 0.00 | H |
| ATOM | 1994 | 2HG | PRO | A | 118 | 3.079 | −6.169 | −13.292 | 1.00 | 0.00 | H |
| ATOM | 1995 | 1HD | PRO | A | 118 | 2.517 | −8.110 | −11.071 | 1.00 | 0.00 | H |
| ATOM | 1996 | 2HD | PRO | A | 118 | 2.119 | −6.387 | −11.189 | 1.00 | 0.00 | H |
| ATOM | 1997 | N | ASN | A | 119 | 7.133 | −5.268 | −12.001 | 1.00 | 0.00 | N |
| ATOM | 1998 | CA | ASN | A | 119 | 7.777 | −3.987 | −12.246 | 1.00 | 0.00 | C |
| ATOM | 1999 | C | ASN | A | 119 | 8.767 | −4.100 | −13.411 | 1.00 | 0.00 | C |
| ATOM | 2000 | O | ASN | A | 119 | 9.305 | −5.178 | −13.662 | 1.00 | 0.00 | O |
| ATOM | 2001 | CB | ASN | A | 119 | 8.471 | −3.526 | −10.971 | 1.00 | 0.00 | C |
| ATOM | 2002 | CG | ASN | A | 119 | 7.649 | −3.443 | −9.686 | 1.00 | 0.00 | C |
| ATOM | 2003 | OD1 | ASN | A | 119 | 7.014 | −2.431 | −9.401 | 1.00 | 0.00 | O |
| ATOM | 2004 | ND2 | ASN | A | 119 | 7.692 | −4.487 | −8.869 | 1.00 | 0.00 | N |
| ATOM | 2005 | H | ASN | A | 119 | 7.720 | −6.097 | −11.961 | 1.00 | 0.00 | H |
| ATOM | 2006 | HA | ASN | A | 119 | 7.050 | −3.230 | −12.550 | 1.00 | 0.00 | H |
| ATOM | 2007 | 1HB | ASN | A | 119 | 9.326 | −4.176 | −10.776 | 1.00 | 0.00 | H |
| ATOM | 2008 | 2HB | ASN | A | 119 | 8.901 | −2.538 | −11.138 | 1.00 | 0.00 | H |
| ATOM | 2009 | 1HD2 | ASN | A | 119 | 7.071 | −4.539 | −8.078 | 1.00 | 0.00 | H |
| ATOM | 2010 | 2HD2 | ASN | A | 119 | 8.205 | −5.315 | −9.133 | 1.00 | 0.00 | H |
| ATOM | 2011 | N | VAL | A | 120 | 8.995 | −2.996 | −14.121 | 1.00 | 0.00 | N |
| ATOM | 2012 | CA | VAL | A | 120 | 9.900 | −3.010 | −15.270 | 1.00 | 0.00 | C |
| ATOM | 2013 | C | VAL | A | 120 | 11.346 | −3.303 | −14.916 | 1.00 | 0.00 | C |
| ATOM | 2014 | O | VAL | A | 120 | 11.876 | −2.811 | −13.918 | 1.00 | 0.00 | O |
| ATOM | 2015 | CB | VAL | A | 120 | 9.852 | −1.693 | −16.053 | 1.00 | 0.00 | C |
| ATOM | 2016 | CG1 | VAL | A | 120 | 8.483 | −1.528 | −16.740 | 1.00 | 0.00 | C |
| ATOM | 2017 | CG2 | VAL | A | 120 | 10.164 | −0.430 | −15.219 | 1.00 | 0.00 | C |
| ATOM | 2018 | H | VAL | A | 120 | 8.529 | −2.134 | −13.893 | 1.00 | 0.00 | H |
| ATOM | 2019 | HA | VAL | A | 120 | 9.553 | −3.805 | −15.935 | 1.00 | 0.00 | H |
| ATOM | 2020 | HB | VAL | A | 120 | 10.596 | −1.740 | −16.849 | 1.00 | 0.00 | H |
| ATOM | 2021 | 1HG1 | VAL | A | 120 | 8.443 | −0.609 | −17.323 | 1.00 | 0.00 | H |
| ATOM | 2022 | 2HG1 | VAL | A | 120 | 8.280 | −2.355 | −17.421 | 1.00 | 0.00 | H |
| ATOM | 2023 | 3HG1 | VAL | A | 120 | 7.668 | −1.488 | −16.017 | 1.00 | 0.00 | H |
| ATOM | 2024 | 1HG2 | VAL | A | 120 | 10.139 | 0.461 | −15.849 | 1.00 | 0.00 | H |
| ATOM | 2025 | 2HG2 | VAL | A | 120 | 9.442 | −0.281 | −14.418 | 1.00 | 0.00 | H |
| ATOM | 2026 | 3HG2 | VAL | A | 120 | 11.158 | −0.465 | −14.773 | 1.00 | 0.00 | H |
| ATOM | 2027 | N | SER | A | 121 | 11.990 | −4.103 | −15.757 | 1.00 | 0.00 | N |
| ATOM | 2028 | CA | SER | A | 121 | 13.365 | −4.485 | −15.514 | 1.00 | 0.00 | C |
| ATOM | 2029 | C | SER | A | 121 | 14.373 | −3.582 | −16.219 | 1.00 | 0.00 | C |
| ATOM | 2030 | O | SER | A | 121 | 15.573 | −3.825 | −16.131 | 1.00 | 0.00 | O |
| ATOM | 2031 | CB | SER | A | 121 | 13.577 | −5.940 | −15.939 | 1.00 | 0.00 | C |
| ATOM | 2032 | OG | SER | A | 121 | 13.357 | −6.117 | −17.333 | 1.00 | 0.00 | O |
| ATOM | 2033 | H | SER | A | 121 | 11.535 | −4.518 | −16.557 | 1.00 | 0.00 | H |
| ATOM | 2034 | HA | SER | A | 121 | 13.596 | −4.449 | −14.447 | 1.00 | 0.00 | H |
| ATOM | 2035 | 1HB | SER | A | 121 | 14.547 | −6.336 | −15.633 | 1.00 | 0.00 | H |
| ATOM | 2036 | 2HB | SER | A | 121 | 12.801 | −6.532 | −15.482 | 1.00 | 0.00 | H |
| ATOM | 2037 | HG | SER | A | 121 | 13.161 | −7.033 | −17.484 | 1.00 | 0.00 | H |
| ATOM | 2038 | N | TYR | A | 122 | 13.903 | −2.539 | −16.899 | 1.00 | 0.00 | N |
| ATOM | 2039 | CA | TYR | A | 122 | 14.836 | −1.659 | −17.590 | 1.00 | 0.00 | C |
| ATOM | 2040 | C | TYR | A | 122 | 15.225 | −0.394 | −16.843 | 1.00 | 0.00 | C |
| ATOM | 2041 | O | TYR | A | 122 | 15.728 | 0.555 | −17.450 | 1.00 | 0.00 | O |
| ATOM | 2042 | CB | TYR | A | 122 | 14.325 | −1.284 | −18.987 | 1.00 | 0.00 | C |
| ATOM | 2043 | CG | TYR | A | 122 | 12.921 | −0.697 | −19.081 | 1.00 | 0.00 | C |
| ATOM | 2044 | CD1 | TYR | A | 122 | 12.695 | 0.676 | −18.842 | 1.00 | 0.00 | C |
| ATOM | 2045 | CD2 | TYR | A | 122 | 11.838 | −1.523 | −19.453 | 1.00 | 0.00 | C |
| ATOM | 2046 | CE1 | TYR | A | 122 | 11.402 | 1.216 | −18.970 | 1.00 | 0.00 | C |
| ATOM | 2047 | CE2 | TYR | A | 122 | 10.546 | −0.980 | −19.594 | 1.00 | 0.00 | C |
| ATOM | 2048 | CZ | TYR | A | 122 | 10.328 | 0.391 | −19.353 | 1.00 | 0.00 | C |
| ATOM | 2049 | OH | TYR | A | 122 | 9.083 | 0.926 | −19.493 | 1.00 | 0.00 | O |
| ATOM | 2050 | H | TYR | A | 122 | 12.912 | −2.365 | −16.960 | 1.00 | 0.00 | H |
| ATOM | 2051 | HA | TYR | A | 122 | 15.790 | −2.154 | −17.783 | 1.00 | 0.00 | H |
| ATOM | 2052 | 1HB | TYR | A | 122 | 15.016 | −0.612 | −19.499 | 1.00 | 0.00 | H |
| ATOM | 2053 | 2HB | TYR | A | 122 | 14.345 | −2.187 | −19.599 | 1.00 | 0.00 | H |
| ATOM | 2054 | HD1 | TYR | A | 122 | 13.513 | 1.328 | −18.569 | 1.00 | 0.00 | H |
| ATOM | 2055 | HD2 | TYR | A | 122 | 11.994 | −2.575 | −19.647 | 1.00 | 0.00 | H |
| ATOM | 2056 | HE1 | TYR | A | 122 | 11.238 | 2.268 | −18.787 | 1.00 | 0.00 | H |
| ATOM | 2057 | HE2 | TYR | A | 122 | 9.730 | −1.624 | −19.886 | 1.00 | 0.00 | H |
| ATOM | 2058 | HH | TYR | A | 122 | 8.435 | 0.290 | −19.774 | 1.00 | 0.00 | H |
| ATOM | 2059 | N | ILE | A | 123 | 14.990 | −0.360 | −15.534 | 1.00 | 0.00 | N |
| ATOM | 2060 | CA | ILE | A | 123 | 15.405 | 0.802 | −14.766 | 1.00 | 0.00 | C |
| ATOM | 2061 | C | ILE | A | 123 | 16.845 | 0.517 | −14.327 | 1.00 | 0.00 | C |
| ATOM | 2062 | O | ILE | A | 123 | 17.373 | −0.573 | −14.585 | 1.00 | 0.00 | O |
| ATOM | 2063 | CB | ILE | A | 123 | 14.505 | 1.052 | −13.502 | 1.00 | 0.00 | C |
| ATOM | 2064 | CG1 | ILE | A | 123 | 13.964 | −0.227 | −12.816 | 1.00 | 0.00 | C |
| ATOM | 2065 | CG2 | ILE | A | 123 | 13.316 | 1.950 | −13.886 | 1.00 | 0.00 | C |
| ATOM | 2066 | CD1 | ILE | A | 123 | 15.013 | −1.121 | −12.136 | 1.00 | 0.00 | C |
| ATOM | 2067 | H | ILE | A | 123 | 14.637 | −1.169 | −15.050 | 1.00 | 0.00 | H |
| ATOM | 2068 | HA | ILE | A | 123 | 15.428 | 1.717 | −15.363 | 1.00 | 0.00 | H |
| ATOM | 2069 | HB | ILE | A | 123 | 15.062 | 1.615 | −12.751 | 1.00 | 0.00 | H |
| ATOM | 2070 | 1HG1 | ILE | A | 123 | 13.253 | 0.084 | −12.056 | 1.00 | 0.00 | H |
| ATOM | 2071 | 2HG1 | ILE | A | 123 | 13.379 | −0.817 | −13.517 | 1.00 | 0.00 | H |
| ATOM | 2072 | 1HG2 | ILE | A | 123 | 12.688 | 2.171 | −13.022 | 1.00 | 0.00 | H |
| ATOM | 2073 | 2HG2 | ILE | A | 123 | 13.654 | 2.904 | −14.290 | 1.00 | 0.00 | H |
| ATOM | 2074 | 3HG2 | ILE | A | 123 | 12.689 | 1.476 | −14.642 | 1.00 | 0.00 | H |
| ATOM | 2075 | 1HD1 | ILE | A | 123 | 14.533 | −1.890 | −11.529 | 1.00 | 0.00 | H |
| ATOM | 2076 | 2HD1 | ILE | A | 123 | 15.635 | −1.640 | −12.864 | 1.00 | 0.00 | H |
| ATOM | 2077 | 3HD1 | ILE | A | 123 | 15.669 | −0.548 | −11.480 | 1.00 | 0.00 | H |
| ATOM | 2078 | N | CYS | A | 124 | 17.473 | 1.497 | −13.684 | 1.00 | 0.00 | N |
| ATOM | 2079 | CA | CYS | A | 124 | 18.845 | 1.387 | −13.183 | 1.00 | 0.00 | C |
| ATOM | 2080 | C | CYS | A | 124 | 19.926 | 1.483 | −14.259 | 1.00 | 0.00 | C |
| ATOM | 2081 | O | CYS | A | 124 | 19.852 | 0.842 | −15.310 | 1.00 | 0.00 | O |
| ATOM | 2082 | CB | CYS | A | 124 | 19.031 | 0.101 | −12.385 | 1.00 | 0.00 | C |
| ATOM | 2083 | SG | CYS | A | 124 | 20.413 | 0.270 | −11.211 | 1.00 | 0.00 | S |
| ATOM | 2084 | H | CYS | A | 124 | 16.978 | 2.349 | −13.468 | 1.00 | 0.00 | H |
| ATOM | 2085 | HA | CYS | A | 124 | 18.963 | 2.237 | −12.510 | 1.00 | 0.00 | H |
| ATOM | 2086 | 1HB | CYS | A | 124 | 18.142 | −0.113 | −11.792 | 1.00 | 0.00 | H |
| ATOM | 2087 | 2HB | CYS | A | 124 | 19.222 | −0.773 | −13.007 | 1.00 | 0.00 | H |
| ATOM | 2088 | HG | CYS | A | 124 | 20.064 | −0.735 | −10.400 | 1.00 | 0.00 | H |
| ATOM | 2089 | N | SER | A | 125 | 20.945 | 2.287 | −13.977 | 1.00 | 0.00 | N |
| ATOM | 2090 | CA | SER | A | 125 | 22.031 | 2.502 | −14.928 | 1.00 | 0.00 | C |
| ATOM | 2091 | C | SER | A | 125 | 23.022 | 1.363 | −15.039 | 1.00 | 0.00 | C |
| ATOM | 2092 | O | SER | A | 125 | 23.304 | 0.662 | −14.068 | 1.00 | 0.00 | O |
| ATOM | 2093 | CB | SER | A | 125 | 22.772 | 3.788 | −14.579 | 1.00 | 0.00 | C |
| ATOM | 2094 | OG | SER | A | 125 | 23.257 | 3.717 | −13.255 | 1.00 | 0.00 | O |
| ATOM | 2095 | H | SER | A | 125 | 20.955 | 2.782 | −13.095 | 1.00 | 0.00 | H |
| ATOM | 2096 | HA | SER | A | 125 | 21.580 | 2.652 | −15.912 | 1.00 | 0.00 | H |
| ATOM | 2097 | 1HB | SER | A | 125 | 23.609 | 3.957 | −15.260 | 1.00 | 0.00 | H |
| ATOM | 2098 | 2HB | SER | A | 125 | 22.108 | 4.646 | −14.673 | 1.00 | 0.00 | H |
| ATOM | 2099 | HG | SER | A | 125 | 23.626 | 2.857 | −13.105 | 1.00 | 0.00 | H |
| ATOM | 2100 | N | ARG | A | 126 | 23.552 | 1.201 | −16.246 | 1.00 | 0.00 | N |
| ATOM | 2101 | CA | ARG | A | 126 | 24.539 | 0.171 | −16.539 | 1.00 | 0.00 | C |
| ATOM | 2102 | C | ARG | A | 126 | 25.742 | 0.314 | −15.608 | 1.00 | 0.00 | C |
| ATOM | 2103 | O | ARG | A | 126 | 26.188 | 1.426 | −15.308 | 1.00 | 0.00 | O |
| ATOM | 2104 | CB | ARG | A | 126 | 24.990 | 0.289 | −18.000 | 1.00 | 0.00 | C |
| ATOM | 2105 | CG | ARG | A | 126 | 25.891 | −0.840 | −18.559 | 1.00 | 0.00 | C |
| ATOM | 2106 | CD | ARG | A | 126 | 25.284 | −2.253 | −18.504 | 1.00 | 0.00 | C |
| ATOM | 2107 | NE | ARG | A | 126 | 24.013 | −2.336 | −19.242 | 1.00 | 0.00 | N |
| ATOM | 2108 | CZ | ARG | A | 126 | 23.154 | −3.364 | −19.257 | 1.00 | 0.00 | C |
| ATOM | 2109 | NH1 | ARG | A | 126 | 23.341 | −4.449 | −18.503 | 1.00 | 0.00 | N |
| ATOM | 2110 | NH2 | ARG | A | 126 | 22.080 | −3.295 | −20.038 | 1.00 | 0.00 | N |
| ATOM | 2111 | H | ARG | A | 126 | 23.289 | 1.831 | −16.986 | 1.00 | 0.00 | H |
| ATOM | 2112 | HA | ARG | A | 126 | 24.072 | −0.803 | −16.386 | 1.00 | 0.00 | H |
| ATOM | 2113 | 1HB | ARG | A | 126 | 24.105 | 0.356 | −18.633 | 1.00 | 0.00 | H |
| ATOM | 2114 | 2HB | ARG | A | 126 | 25.511 | 1.238 | −18.137 | 1.00 | 0.00 | H |
| ATOM | 2115 | 1HG | ARG | A | 126 | 26.148 | −0.609 | −19.593 | 1.00 | 0.00 | H |
| ATOM | 2116 | 2HG | ARG | A | 126 | 26.845 | −0.848 | −18.036 | 1.00 | 0.00 | H |
| ATOM | 2117 | 1HD | ARG | A | 126 | 25.983 | −2.978 | −18.926 | 1.00 | 0.00 | H |
| ATOM | 2118 | 2HD | ARG | A | 126 | 25.121 | −2.539 | −17.469 | 1.00 | 0.00 | H |
| ATOM | 2119 | HE | ARG | A | 126 | 23.854 | −1.565 | −19.877 | 1.00 | 0.00 | H |
| ATOM | 2120 | 1HH1 | ARG | A | 126 | 22.735 | −5.248 | −18.560 | 1.00 | 0.00 | H |
| ATOM | 2121 | 2HH1 | ARG | A | 126 | 24.085 | −4.501 | −17.793 | 1.00 | 0.00 | H |
| ATOM | 2122 | 1HH2 | ARG | A | 126 | 21.383 | −4.020 | −20.075 | 1.00 | 0.00 | H |
| ATOM | 2123 | 2HH2 | ARG | A | 126 | 21.903 | −2.492 | −20.622 | 1.00 | 0.00 | H |
| ATOM | 2124 | N | TYR | A | 127 | 26.264 | −0.828 | −15.183 | 1.00 | 0.00 | N |
| ATOM | 2125 | CA | TYR | A | 127 | 27.387 | −0.943 | −14.255 | 1.00 | 0.00 | C |
| ATOM | 2126 | C | TYR | A | 127 | 26.830 | −1.191 | −12.862 | 1.00 | 0.00 | C |
| ATOM | 2127 | O | TYR | A | 127 | 27.443 | −1.915 | −12.087 | 1.00 | 0.00 | O |
| ATOM | 2128 | CB | TYR | A | 127 | 28.258 | 0.323 | −14.190 | 1.00 | 0.00 | C |
| ATOM | 2129 | CG | TYR | A | 127 | 29.059 | 0.757 | −15.417 | 1.00 | 0.00 | C |
| ATOM | 2130 | CD1 | TYR | A | 127 | 29.123 | −0.026 | −16.591 | 1.00 | 0.00 | C |
| ATOM | 2131 | CD2 | TYR | A | 127 | 29.754 | 1.983 | −15.369 | 1.00 | 0.00 | C |
| ATOM | 2132 | CE1 | TYR | A | 127 | 29.855 | 0.423 | −17.707 | 1.00 | 0.00 | C |
| ATOM | 2133 | CE2 | TYR | A | 127 | 30.503 | 2.424 | −16.477 | 1.00 | 0.00 | C |
| ATOM | 2134 | CZ | TYR | A | 127 | 30.554 | 1.642 | −17.646 | 1.00 | 0.00 | C |
| ATOM | 2135 | OH | TYR | A | 127 | 31.280 | 2.055 | −18.722 | 1.00 | 0.00 | O |
| ATOM | 2136 | H | TYR | A | 127 | 25.854 | −1.692 | −15.503 | 1.00 | 0.00 | H |
| ATOM | 2137 | HA | TYR | A | 127 | 27.989 | −1.810 | −14.529 | 1.00 | 0.00 | H |
| ATOM | 2138 | 1HB | TYR | A | 127 | 27.790 | 1.168 | −13.694 | 1.00 | 0.00 | H |
| ATOM | 2139 | 2HB | TYR | A | 127 | 29.046 | 0.084 | −13.473 | 1.00 | 0.00 | H |
| ATOM | 2140 | HD1 | TYR | A | 127 | 28.618 | −0.978 | −16.649 | 1.00 | 0.00 | H |
| ATOM | 2141 | HD2 | TYR | A | 127 | 29.717 | 2.592 | −14.475 | 1.00 | 0.00 | H |
| ATOM | 2142 | HE1 | TYR | A | 127 | 29.883 | −0.170 | −18.609 | 1.00 | 0.00 | H |
| ATOM | 2143 | HE2 | TYR | A | 127 | 31.033 | 3.363 | −16.416 | 1.00 | 0.00 | H |
| ATOM | 2144 | HH | TYR | A | 127 | 31.707 | 2.891 | −18.580 | 1.00 | 0.00 | H |
| ATOM | 2145 | N | TYR | A | 128 | 25.662 | −0.608 | −12.561 | 1.00 | 0.00 | N |
| ATOM | 2146 | CA | TYR | A | 128 | 25.046 | −0.732 | −11.227 | 1.00 | 0.00 | C |
| ATOM | 2147 | C | TYR | A | 128 | 23.802 | −1.618 | −11.142 | 1.00 | 0.00 | C |
| ATOM | 2148 | O | TYR | A | 128 | 23.233 | −1.787 | −10.063 | 1.00 | 0.00 | O |
| ATOM | 2149 | CB | TYR | A | 128 | 24.720 | 0.672 | −10.680 | 1.00 | 0.00 | C |
| ATOM | 2150 | CG | TYR | A | 128 | 25.891 | 1.644 | −10.800 | 1.00 | 0.00 | C |
| ATOM | 2151 | CD1 | TYR | A | 128 | 26.886 | 1.705 | −9.803 | 1.00 | 0.00 | C |
| ATOM | 2152 | CD2 | TYR | A | 128 | 26.043 | 2.423 | −11.966 | 1.00 | 0.00 | C |
| ATOM | 2153 | CE1 | TYR | A | 128 | 28.067 | 2.443 | −10.020 | 1.00 | 0.00 | C |
| ATOM | 2154 | CE2 | TYR | A | 128 | 27.194 | 3.205 | −12.161 | 1.00 | 0.00 | C |
| ATOM | 2155 | CZ | TYR | A | 128 | 28.226 | 3.183 | −11.209 | 1.00 | 0.00 | C |
| ATOM | 2156 | OH | TYR | A | 128 | 29.364 | 3.887 | −11.456 | 1.00 | 0.00 | O |
| ATOM | 2157 | H | TYR | A | 128 | 25.220 | 0.012 | −13.229 | 1.00 | 0.00 | H |
| ATOM | 2158 | HA | TYR | A | 128 | 25.754 | −1.171 | −10.523 | 1.00 | 0.00 | H |
| ATOM | 2159 | 1HB | TYR | A | 128 | 23.861 | 1.094 | −11.205 | 1.00 | 0.00 | H |
| ATOM | 2160 | 2HB | TYR | A | 128 | 24.420 | 0.615 | −9.633 | 1.00 | 0.00 | H |
| ATOM | 2161 | HD1 | TYR | A | 128 | 26.769 | 1.157 | −8.884 | 1.00 | 0.00 | H |
| ATOM | 2162 | HD2 | TYR | A | 128 | 25.315 | 2.374 | −12.757 | 1.00 | 0.00 | H |
| ATOM | 2163 | HE1 | TYR | A | 128 | 28.852 | 2.440 | −9.275 | 1.00 | 0.00 | H |
| ATOM | 2164 | HE2 | TYR | A | 128 | 27.310 | 3.773 | −13.073 | 1.00 | 0.00 | H |
| ATOM | 2165 | HH | TYR | A | 128 | 30.079 | 3.747 | −10.799 | 1.00 | 0.00 | H |
| ATOM | 2166 | N | ARG | A | 129 | 23.398 | −2.192 | −12.274 | 1.00 | 0.00 | N |
| ATOM | 2167 | CA | ARG | A | 129 | 22.216 | −3.061 | −12.341 | 1.00 | 0.00 | C |
| ATOM | 2168 | C | ARG | A | 129 | 22.404 | −4.423 | −11.673 | 1.00 | 0.00 | C |
| ATOM | 2169 | O | ARG | A | 129 | 23.375 | −5.133 | −11.964 | 1.00 | 0.00 | O |
| ATOM | 2170 | CB | ARG | A | 129 | 21.838 | −3.301 | −13.802 | 1.00 | 0.00 | C |
| ATOM | 2171 | CG | ARG | A | 129 | 21.787 | −2.094 | −14.744 | 1.00 | 0.00 | C |
| ATOM | 2172 | CD | ARG | A | 129 | 21.640 | −2.488 | −16.223 | 1.00 | 0.00 | C |
| ATOM | 2173 | NE | ARG | A | 129 | 20.529 | −3.432 | −16.437 | 1.00 | 0.00 | N |
| ATOM | 2174 | CZ | ARG | A | 129 | 19.234 | −3.100 | −16.427 | 1.00 | 0.00 | C |
| ATOM | 2175 | NH1 | ARG | A | 129 | 18.892 | −1.822 | −16.310 | 1.00 | 0.00 | N |
| ATOM | 2176 | NH2 | ARG | A | 129 | 18.296 | −4.046 | −16.509 | 1.00 | 0.00 | N |
| ATOM | 2177 | H | ARG | A | 129 | 23.941 | −2.026 | −13.104 | 1.00 | 0.00 | H |
| ATOM | 2178 | HA | ARG | A | 129 | 21.386 | −2.553 | −11.845 | 1.00 | 0.00 | H |
| ATOM | 2179 | 1HB | ARG | A | 129 | 22.542 | −4.016 | −14.233 | 1.00 | 0.00 | H |
| ATOM | 2180 | 2HB | ARG | A | 129 | 20.870 | −3.805 | −13.820 | 1.00 | 0.00 | H |
| ATOM | 2181 | 1HG | ARG | A | 129 | 21.014 | −1.394 | −14.432 | 1.00 | 0.00 | H |
| ATOM | 2182 | 2HG | ARG | A | 129 | 22.731 | −1.570 | −14.667 | 1.00 | 0.00 | H |
| ATOM | 2183 | 1HD | ARG | A | 129 | 21.525 | −1.607 | −16.855 | 1.00 | 0.00 | H |
| ATOM | 2184 | 2HD | ARG | A | 129 | 22.563 | −2.969 | −16.540 | 1.00 | 0.00 | H |
| ATOM | 2185 | HE | ARG | A | 129 | 20.803 | −4.403 | −16.477 | 1.00 | 0.00 | H |
| ATOM | 2186 | 1HH1 | ARG | A | 129 | 18.017 | −1.524 | −15.868 | 1.00 | 0.00 | H |
| ATOM | 2187 | 2HH1 | ARG | A | 129 | 19.581 | −1.079 | −16.269 | 1.00 | 0.00 | H |
| ATOM | 2188 | 1HH2 | ARG | A | 129 | 17.293 | −3.836 | −16.421 | 1.00 | 0.00 | H |
| ATOM | 2189 | 2HH2 | ARG | A | 129 | 18.502 | −5.026 | −16.635 | 1.00 | 0.00 | H |
| ATOM | 2190 | N | ALA | A | 130 | 21.469 | −4.802 | −10.799 | 1.00 | 0.00 | N |
| ATOM | 2191 | CA | ALA | A | 130 | 21.544 | −6.106 | −10.134 | 1.00 | 0.00 | C |
| ATOM | 2192 | C | ALA | A | 130 | 21.393 | −7.176 | −11.214 | 1.00 | 0.00 | C |
| ATOM | 2193 | O | ALA | A | 130 | 20.741 | −6.942 | −12.235 | 1.00 | 0.00 | O |
| ATOM | 2194 | CB | ALA | A | 130 | 20.434 | −6.242 | −9.092 | 1.00 | 0.00 | C |
| ATOM | 2195 | H | ALA | A | 130 | 20.709 | −4.182 | −10.575 | 1.00 | 0.00 | H |
| ATOM | 2196 | HA | ALA | A | 130 | 22.514 | −6.205 | −9.668 | 1.00 | 0.00 | H |
| ATOM | 2197 | 1HB | ALA | A | 130 | 20.554 | −7.150 | −8.502 | 1.00 | 0.00 | H |
| ATOM | 2198 | 2HB | ALA | A | 130 | 20.450 | −5.406 | −8.395 | 1.00 | 0.00 | H |
| ATOM | 2199 | 3HB | ALA | A | 130 | 19.444 | −6.272 | −9.548 | 1.00 | 0.00 | H |
| ATOM | 2200 | N | PRO | A | 131 | 22.013 | −8.352 | −11.018 | 1.00 | 0.00 | N |
| ATOM | 2201 | CA | PRO | A | 131 | 21.917 | −9.427 | −12.014 | 1.00 | 0.00 | C |
| ATOM | 2202 | C | PRO | A | 131 | 20.494 | −9.844 | −12.370 | 1.00 | 0.00 | C |
| ATOM | 2203 | O | PRO | A | 131 | 20.205 | −10.135 | −13.531 | 1.00 | 0.00 | O |
| ATOM | 2204 | CB | PRO | A | 131 | 22.734 | −10.571 | −11.404 | 1.00 | 0.00 | C |
| ATOM | 2205 | CG | PRO | A | 131 | 22.809 | −10.318 | −9.912 | 1.00 | 0.00 | C |
| ATOM | 2206 | CD | PRO | A | 131 | 22.654 | −8.809 | −9.784 | 1.00 | 0.00 | C |
| ATOM | 2207 | HA | PRO | A | 131 | 22.396 | −9.096 | −12.934 | 1.00 | 0.00 | H |
| ATOM | 2208 | 1HB | PRO | A | 131 | 22.355 | −11.553 | −11.673 | 1.00 | 0.00 | H |
| ATOM | 2209 | 2HB | PRO | A | 131 | 23.745 | −10.523 | −11.810 | 1.00 | 0.00 | H |
| ATOM | 2210 | 1HG | PRO | A | 131 | 23.724 | −10.695 | −9.454 | 1.00 | 0.00 | H |
| ATOM | 2211 | 2HG | PRO | A | 131 | 21.966 | −10.802 | −9.416 | 1.00 | 0.00 | H |
| ATOM | 2212 | 1HD | PRO | A | 131 | 23.637 | −8.353 | −9.753 | 1.00 | 0.00 | H |
| ATOM | 2213 | 2HD | PRO | A | 131 | 22.125 | −8.552 | −8.869 | 1.00 | 0.00 | H |
| ATOM | 2214 | N | GLU | A | 132 | 19.595 | −9.865 | −11.389 | 1.00 | 0.00 | N |
| ATOM | 2215 | CA | GLU | A | 132 | 18.226 | −10.251 | −11.677 | 1.00 | 0.00 | C |
| ATOM | 2216 | C | GLU | A | 132 | 17.604 | −9.299 | −12.695 | 1.00 | 0.00 | C |
| ATOM | 2217 | O | GLU | A | 132 | 16.727 | −9.701 | −13.466 | 1.00 | 0.00 | O |
| ATOM | 2218 | CB | GLU | A | 132 | 17.381 | −10.309 | −10.392 | 1.00 | 0.00 | C |
| ATOM | 2219 | CG | GLU | A | 132 | 17.348 | −9.062 | −9.468 | 1.00 | 0.00 | C |
| ATOM | 2220 | CD | GLU | A | 132 | 18.467 | −9.009 | −8.433 | 1.00 | 0.00 | C |
| ATOM | 2221 | OE1 | GLU | A | 132 | 19.551 | −9.582 | −8.671 | 1.00 | 0.00 | O |
| ATOM | 2222 | OE2 | GLU | A | 132 | 18.281 | −8.366 | −7.381 | 1.00 | 0.00 | O |
| ATOM | 2223 | H | GLU | A | 132 | 19.831 | −9.686 | −10.409 | 1.00 | 0.00 | H |
| ATOM | 2224 | HA | GLU | A | 132 | 18.228 | −11.240 | −12.135 | 1.00 | 0.00 | H |
| ATOM | 2225 | 1HB | GLU | A | 132 | 16.354 | −10.550 | −10.667 | 1.00 | 0.00 | H |
| ATOM | 2226 | 2HB | GLU | A | 132 | 17.710 | −11.162 | −9.800 | 1.00 | 0.00 | H |
| ATOM | 2227 | 1HG | GLU | A | 132 | 17.342 | −8.133 | −10.036 | 1.00 | 0.00 | H |
| ATOM | 2228 | 2HG | GLU | A | 132 | 16.414 | −9.075 | −8.907 | 1.00 | 0.00 | H |
| ATOM | 2229 | N | LEU | A | 133 | 18.064 | −8.045 | −12.707 | 1.00 | 0.00 | N |
| ATOM | 2230 | CA | LEU | A | 133 | 17.564 | −7.051 | −13.658 | 1.00 | 0.00 | C |
| ATOM | 2231 | C | LEU | A | 133 | 18.122 | −7.371 | −15.053 | 1.00 | 0.00 | C |
| ATOM | 2232 | O | LEU | A | 133 | 17.405 | −7.297 | −16.054 | 1.00 | 0.00 | O |
| ATOM | 2233 | CB | LEU | A | 133 | 17.999 | −5.632 | −13.254 | 1.00 | 0.00 | C |
| ATOM | 2234 | CG | LEU | A | 133 | 17.449 | −5.119 | −11.908 | 1.00 | 0.00 | C |
| ATOM | 2235 | CD1 | LEU | A | 133 | 18.024 | −3.727 | −11.602 | 1.00 | 0.00 | C |
| ATOM | 2236 | CD2 | LEU | A | 133 | 15.912 | −5.069 | −11.888 | 1.00 | 0.00 | C |
| ATOM | 2237 | H | LEU | A | 133 | 18.837 | −7.799 | −12.103 | 1.00 | 0.00 | H |
| ATOM | 2238 | HA | LEU | A | 133 | 16.476 | −7.107 | −13.720 | 1.00 | 0.00 | H |
| ATOM | 2239 | 1HB | LEU | A | 133 | 19.082 | −5.538 | −13.273 | 1.00 | 0.00 | H |
| ATOM | 2240 | 2HB | LEU | A | 133 | 17.652 | −4.943 | −14.025 | 1.00 | 0.00 | H |
| ATOM | 2241 | HG | LEU | A | 133 | 17.776 | −5.794 | −11.117 | 1.00 | 0.00 | H |
| ATOM | 2242 | 1HD1 | LEU | A | 133 | 17.726 | −3.390 | −10.611 | 1.00 | 0.00 | H |
| ATOM | 2243 | 2HD1 | LEU | A | 133 | 19.112 | −3.730 | −11.632 | 1.00 | 0.00 | H |
| ATOM | 2244 | 3HD1 | LEU | A | 133 | 17.681 | −2.985 | −12.324 | 1.00 | 0.00 | H |
| ATOM | 2245 | 1HD2 | LEU | A | 133 | 15.541 | −4.637 | −10.960 | 1.00 | 0.00 | H |
| ATOM | 2246 | 2HD2 | LEU | A | 133 | 15.518 | −4.465 | −12.707 | 1.00 | 0.00 | H |
| ATOM | 2247 | 3HD2 | LEU | A | 133 | 15.472 | −6.064 | −11.964 | 1.00 | 0.00 | H |
| ATOM | 2248 | N | ILE | A | 134 | 19.403 | −7.720 | −15.116 | 1.00 | 0.00 | N |
| ATOM | 2249 | CA | ILE | A | 134 | 20.013 | −8.058 | −16.394 | 1.00 | 0.00 | C |
| ATOM | 2250 | C | ILE | A | 134 | 19.288 | −9.291 | −16.948 | 1.00 | 0.00 | C |
| ATOM | 2251 | O | ILE | A | 134 | 19.051 | −9.396 | −18.151 | 1.00 | 0.00 | O |
| ATOM | 2252 | CB | ILE | A | 134 | 21.519 | −8.346 | −16.237 | 1.00 | 0.00 | C |
| ATOM | 2253 | CG1 | ILE | A | 134 | 22.284 | −7.309 | −15.379 | 1.00 | 0.00 | C |
| ATOM | 2254 | CG2 | ILE | A | 134 | 22.176 | −8.425 | −17.636 | 1.00 | 0.00 | C |
| ATOM | 2255 | CD1 | ILE | A | 134 | 23.710 | −7.756 | −15.023 | 1.00 | 0.00 | C |
| ATOM | 2256 | H | ILE | A | 134 | 19.937 | −7.811 | −14.261 | 1.00 | 0.00 | H |
| ATOM | 2257 | HA | ILE | A | 134 | 19.868 | −7.233 | −17.092 | 1.00 | 0.00 | H |
| ATOM | 2258 | HB | ILE | A | 134 | 21.637 | −9.317 | −15.750 | 1.00 | 0.00 | H |
| ATOM | 2259 | 1HG1 | ILE | A | 134 | 22.311 | −6.336 | −15.869 | 1.00 | 0.00 | H |
| ATOM | 2260 | 2HG1 | ILE | A | 134 | 21.772 | −7.149 | −14.437 | 1.00 | 0.00 | H |
| ATOM | 2261 | 1HG2 | ILE | A | 134 | 23.252 | −8.564 | −17.585 | 1.00 | 0.00 | H |
| ATOM | 2262 | 2HG2 | ILE | A | 134 | 21.786 | −9.257 | −18.223 | 1.00 | 0.00 | H |
| ATOM | 2263 | 3HG2 | ILE | A | 134 | 22.001 | −7.514 | −18.206 | 1.00 | 0.00 | H |
| ATOM | 2264 | 1HD1 | ILE | A | 134 | 24.099 | −7.167 | −14.194 | 1.00 | 0.00 | H |
| ATOM | 2265 | 2HD1 | ILE | A | 134 | 23.743 | −8.799 | −14.713 | 1.00 | 0.00 | H |
| ATOM | 2266 | 3HD1 | ILE | A | 134 | 24.396 | −7.646 | −15.862 | 1.00 | 0.00 | H |
| ATOM | 2267 | N | PHE | A | 135 | 18.925 | −10.209 | −16.059 | 1.00 | 0.00 | N |
| ATOM | 2268 | CA | PHE | A | 135 | 18.211 | −11.423 | −16.445 | 1.00 | 0.00 | C |
| ATOM | 2269 | C | PHE | A | 135 | 16.736 | −11.149 | −16.726 | 1.00 | 0.00 | C |
| ATOM | 2270 | O | PHE | A | 135 | 15.972 | −12.080 | −16.945 | 1.00 | 0.00 | O |
| ATOM | 2271 | CB | PHE | A | 135 | 18.314 | −12.482 | −15.343 | 1.00 | 0.00 | C |
| ATOM | 2272 | CG | PHE | A | 135 | 19.664 | −13.185 | −15.271 | 1.00 | 0.00 | C |
| ATOM | 2273 | CD1 | PHE | A | 135 | 20.163 | −13.870 | −16.401 | 1.00 | 0.00 | C |
| ATOM | 2274 | CD2 | PHE | A | 135 | 20.425 | −13.174 | −14.082 | 1.00 | 0.00 | C |
| ATOM | 2275 | CE1 | PHE | A | 135 | 21.415 | −14.514 | −16.352 | 1.00 | 0.00 | C |
| ATOM | 2276 | CE2 | PHE | A | 135 | 21.684 | −13.803 | −14.038 | 1.00 | 0.00 | C |
| ATOM | 2277 | CZ | PHE | A | 135 | 22.178 | −14.477 | −15.170 | 1.00 | 0.00 | C |
| ATOM | 2278 | H | PHE | A | 135 | 19.233 | −10.110 | −15.094 | 1.00 | 0.00 | H |
| ATOM | 2279 | HA | PHE | A | 135 | 18.627 | −11.812 | −17.374 | 1.00 | 0.00 | H |
| ATOM | 2280 | 1HB | PHE | A | 135 | 18.063 | −12.047 | −14.376 | 1.00 | 0.00 | H |
| ATOM | 2281 | 2HB | PHE | A | 135 | 17.591 | −13.283 | −15.499 | 1.00 | 0.00 | H |
| ATOM | 2282 | HD1 | PHE | A | 135 | 19.592 | −13.902 | −17.316 | 1.00 | 0.00 | H |
| ATOM | 2283 | HD2 | PHE | A | 135 | 20.060 | −12.671 | −13.199 | 1.00 | 0.00 | H |
| ATOM | 2284 | HE1 | PHE | A | 135 | 21.793 | −15.031 | −17.223 | 1.00 | 0.00 | H |
| ATOM | 2285 | HE2 | PHE | A | 135 | 22.274 | −13.761 | −13.135 | 1.00 | 0.00 | H |
| ATOM | 2286 | HZ | PHE | A | 135 | 23.145 | −14.960 | −15.135 | 1.00 | 0.00 | H |
| ATOM | 2287 | N | GLY | A | 136 | 16.328 | −9.884 | −16.690 | 1.00 | 0.00 | N |
| ATOM | 2288 | CA | GLY | A | 136 | 14.946 | −9.530 | −16.976 | 1.00 | 0.00 | C |
| ATOM | 2289 | C | GLY | A | 136 | 13.847 | −9.801 | −15.950 | 1.00 | 0.00 | C |
| ATOM | 2290 | O | GLY | A | 136 | 12.667 | −9.835 | −16.310 | 1.00 | 0.00 | O |
| ATOM | 2291 | H | GLY | A | 136 | 16.987 | −9.140 | −16.494 | 1.00 | 0.00 | H |
| ATOM | 2292 | 1HA | GLY | A | 136 | 14.937 | −8.460 | −17.179 | 1.00 | 0.00 | H |
| ATOM | 2293 | 2HA | GLY | A | 136 | 14.632 | −10.001 | −17.906 | 1.00 | 0.00 | H |
| ATOM | 2294 | N | ALA | A | 137 | 14.200 | −9.984 | −14.681 | 1.00 | 0.00 | N |
| ATOM | 2295 | CA | ALA | A | 137 | 13.182 | −10.235 | −13.662 | 1.00 | 0.00 | C |
| ATOM | 2296 | C | ALA | A | 137 | 12.242 | −9.041 | −13.502 | 1.00 | 0.00 | C |
| ATOM | 2297 | O | ALA | A | 137 | 12.640 | −7.893 | −13.698 | 1.00 | 0.00 | O |
| ATOM | 2298 | CB | ALA | A | 137 | 13.841 | −10.557 | −12.332 | 1.00 | 0.00 | C |
| ATOM | 2299 | H | ALA | A | 137 | 15.175 | −9.904 | −14.402 | 1.00 | 0.00 | H |
| ATOM | 2300 | HA | ALA | A | 137 | 12.595 | −11.092 | −13.987 | 1.00 | 0.00 | H |
| ATOM | 2301 | 1HB | ALA | A | 137 | 13.179 | −11.133 | −11.686 | 1.00 | 0.00 | H |
| ATOM | 2302 | 2HB | ALA | A | 137 | 14.763 | −11.112 | −12.442 | 1.00 | 0.00 | H |
| ATOM | 2303 | 3HB | ALA | A | 137 | 14.111 | −9.649 | −11.790 | 1.00 | 0.00 | H |
| ATOM | 2304 | N | THR | A | 138 | 10.987 | −9.320 | −13.158 | 1.00 | 0.00 | N |
| ATOM | 2305 | CA | THR | A | 138 | 9.990 | −8.267 | −12.952 | 1.00 | 0.00 | C |
| ATOM | 2306 | C | THR | A | 138 | 9.436 | −8.384 | −11.530 | 1.00 | 0.00 | C |
| ATOM | 2307 | O | THR | A | 138 | 8.595 | −7.592 | −11.106 | 1.00 | 0.00 | O |
| ATOM | 2308 | CB | THR | A | 138 | 8.821 | −8.389 | −13.950 | 1.00 | 0.00 | C |
| ATOM | 2309 | OG1 | THR | A | 138 | 8.236 | −9.683 | −13.923 | 1.00 | 0.00 | O |
| ATOM | 2310 | CG2 | THR | A | 138 | 9.267 | −8.112 | −15.394 | 1.00 | 0.00 | C |
| ATOM | 2311 | H | THR | A | 138 | 10.654 | −10.271 | −13.117 | 1.00 | 0.00 | H |
| ATOM | 2312 | HA | THR | A | 138 | 10.426 | −7.271 | −13.019 | 1.00 | 0.00 | H |
| ATOM | 2313 | HB | THR | A | 138 | 8.048 | −7.661 | −13.695 | 1.00 | 0.00 | H |
| ATOM | 2314 | HG1 | THR | A | 138 | 7.510 | −9.695 | −14.534 | 1.00 | 0.00 | H |
| ATOM | 2315 | 1HG2 | THR | A | 138 | 8.422 | −8.125 | −16.083 | 1.00 | 0.00 | H |
| ATOM | 2316 | 2HG2 | THR | A | 138 | 9.739 | −7.132 | −15.473 | 1.00 | 0.00 | H |
| ATOM | 2317 | 3HG2 | THR | A | 138 | 9.993 | −8.850 | −15.740 | 1.00 | 0.00 | H |
| ATOM | 2318 | N | ASP | A | 139 | 9.921 | −9.385 | −10.806 | 1.00 | 0.00 | N |
| ATOM | 2319 | CA | ASP | A | 139 | 9.501 | −9.638 | −9.434 | 1.00 | 0.00 | C |
| ATOM | 2320 | C | ASP | A | 139 | 10.620 | −9.297 | −8.443 | 1.00 | 0.00 | C |
| ATOM | 2321 | O | ASP | A | 139 | 10.674 | −9.839 | −7.335 | 1.00 | 0.00 | O |
| ATOM | 2322 | CB | ASP | A | 139 | 9.114 | −11.111 | −9.273 | 1.00 | 0.00 | C |
| ATOM | 2323 | CG | ASP | A | 139 | 10.200 | −12.167 | −9.523 | 1.00 | 0.00 | C |
| ATOM | 2324 | OD1 | ASP | A | 139 | 11.089 | −11.912 | −10.363 | 1.00 | 0.00 | O |
| ATOM | 2325 | OD2 | ASP | A | 139 | 10.094 | −13.253 | −8.917 | 1.00 | 0.00 | O |
| ATOM | 2326 | H | ASP | A | 139 | 10.653 | −9.996 | −11.154 | 1.00 | 0.00 | H |
| ATOM | 2327 | HA | ASP | A | 139 | 8.635 | −9.037 | −9.153 | 1.00 | 0.00 | H |
| ATOM | 2328 | 1HB | ASP | A | 139 | 8.713 | −11.275 | −8.272 | 1.00 | 0.00 | H |
| ATOM | 2329 | 2HB | ASP | A | 139 | 8.291 | −11.336 | −9.951 | 1.00 | 0.00 | H |
| ATOM | 2330 | N | TYR | A | 140 | 11.517 | −8.404 | −8.847 | 1.00 | 0.00 | N |
| ATOM | 2331 | CA | TYR | A | 140 | 12.622 | −8.013 | −7.979 | 1.00 | 0.00 | C |
| ATOM | 2332 | C | TYR | A | 140 | 12.144 | −7.198 | −6.772 | 1.00 | 0.00 | C |
| ATOM | 2333 | O | TYR | A | 140 | 11.022 | −6.696 | −6.742 | 1.00 | 0.00 | O |
| ATOM | 2334 | CB | TYR | A | 140 | 13.661 | −7.219 | −8.780 | 1.00 | 0.00 | C |
| ATOM | 2335 | CG | TYR | A | 140 | 13.155 | −5.943 | −9.444 | 1.00 | 0.00 | C |
| ATOM | 2336 | CD1 | TYR | A | 140 | 13.140 | −4.717 | −8.741 | 1.00 | 0.00 | C |
| ATOM | 2337 | CD2 | TYR | A | 140 | 12.685 | −5.983 | −10.772 | 1.00 | 0.00 | C |
| ATOM | 2338 | CE1 | TYR | A | 140 | 12.649 | −3.549 | −9.356 | 1.00 | 0.00 | C |
| ATOM | 2339 | CE2 | TYR | A | 140 | 12.206 | −4.815 | −11.391 | 1.00 | 0.00 | C |
| ATOM | 2340 | CZ | TYR | A | 140 | 12.191 | −3.594 | −10.686 | 1.00 | 0.00 | C |
| ATOM | 2341 | OH | TYR | A | 140 | 11.752 | −2.458 | −11.302 | 1.00 | 0.00 | O |
| ATOM | 2342 | H | TYR | A | 140 | 11.388 | −7.936 | −9.728 | 1.00 | 0.00 | H |
| ATOM | 2343 | HA | TYR | A | 140 | 13.106 | −8.918 | −7.604 | 1.00 | 0.00 | H |
| ATOM | 2344 | 1HB | TYR | A | 140 | 14.504 | −6.965 | −8.138 | 1.00 | 0.00 | H |
| ATOM | 2345 | 2HB | TYR | A | 140 | 14.086 | −7.864 | −9.551 | 1.00 | 0.00 | H |
| ATOM | 2346 | HD1 | TYR | A | 140 | 13.501 | −4.668 | −7.723 | 1.00 | 0.00 | H |
| ATOM | 2347 | HD2 | TYR | A | 140 | 12.699 | −6.908 | −11.328 | 1.00 | 0.00 | H |
| ATOM | 2348 | HE1 | TYR | A | 140 | 12.644 | −2.621 | −8.805 | 1.00 | 0.00 | H |
| ATOM | 2349 | HE2 | TYR | A | 140 | 11.852 | −4.879 | −12.409 | 1.00 | 0.00 | H |
| ATOM | 2350 | HH | TYR | A | 140 | 11.816 | −2.592 | −12.257 | 1.00 | 0.00 | H |
| ATOM | 2351 | N | THR | A | 141 | 13.017 | −7.068 | −5.781 | 1.00 | 0.00 | N |
| ATOM | 2352 | CA | THR | A | 141 | 12.702 | −6.336 | −4.558 | 1.00 | 0.00 | C |
| ATOM | 2353 | C | THR | A | 141 | 13.648 | −5.148 | −4.359 | 1.00 | 0.00 | C |
| ATOM | 2354 | O | THR | A | 141 | 14.468 | −4.834 | −5.225 | 1.00 | 0.00 | O |
| ATOM | 2355 | CB | THR | A | 141 | 12.847 | −7.243 | −3.344 | 1.00 | 0.00 | C |
| ATOM | 2356 | OG1 | THR | A | 141 | 14.186 | −7.687 | −3.146 | 1.00 | 0.00 | O |
| ATOM | 2357 | CG2 | THR | A | 141 | 11.948 | −8.489 | −3.427 | 1.00 | 0.00 | C |
| ATOM | 2358 | H | THR | A | 141 | 13.951 | −7.448 | −5.836 | 1.00 | 0.00 | H |
| ATOM | 2359 | HA | THR | A | 141 | 11.687 | −5.934 | −4.568 | 1.00 | 0.00 | H |
| ATOM | 2360 | HB | THR | A | 141 | 12.538 | −6.716 | −2.440 | 1.00 | 0.00 | H |
| ATOM | 2361 | HG1 | THR | A | 141 | 14.169 | −8.389 | −2.508 | 1.00 | 0.00 | H |
| ATOM | 2362 | 1HG2 | THR | A | 141 | 11.984 | −9.068 | −2.504 | 1.00 | 0.00 | H |
| ATOM | 2363 | 2HG2 | THR | A | 141 | 10.908 | −8.206 | −3.594 | 1.00 | 0.00 | H |
| ATOM | 2364 | 3HG2 | THR | A | 141 | 12.240 | −9.151 | −4.243 | 1.00 | 0.00 | H |
| ATOM | 2365 | N | SER | A | 142 | 13.536 | −4.508 | −3.200 | 1.00 | 0.00 | N |
| ATOM | 2366 | CA | SER | A | 142 | 14.365 | −3.353 | −2.867 | 1.00 | 0.00 | C |
| ATOM | 2367 | C | SER | A | 142 | 15.839 | −3.722 | −2.768 | 1.00 | 0.00 | C |
| ATOM | 2368 | O | SER | A | 142 | 16.705 | −2.839 | −2.746 | 1.00 | 0.00 | O |
| ATOM | 2369 | CB | SER | A | 142 | 13.894 | −2.741 | −1.544 | 1.00 | 0.00 | C |
| ATOM | 2370 | OG | SER | A | 142 | 12.493 | −2.497 | −1.523 | 1.00 | 0.00 | O |
| ATOM | 2371 | H | SER | A | 142 | 12.819 | −4.758 | −2.538 | 1.00 | 0.00 | H |
| ATOM | 2372 | HA | SER | A | 142 | 14.278 | −2.615 | −3.655 | 1.00 | 0.00 | H |
| ATOM | 2373 | 1HB | SER | A | 142 | 14.147 | −3.414 | −0.723 | 1.00 | 0.00 | H |
| ATOM | 2374 | 2HB | SER | A | 142 | 14.425 | −1.805 | −1.355 | 1.00 | 0.00 | H |
| ATOM | 2375 | HG | SER | A | 142 | 12.290 | −1.858 | −2.193 | 1.00 | 0.00 | H |
| ATOM | 2376 | N | SER | A | 143 | 16.128 | −5.019 | −2.694 | 1.00 | 0.00 | N |
| ATOM | 2377 | CA | SER | A | 143 | 17.507 | −5.471 | −2.602 | 1.00 | 0.00 | C |
| ATOM | 2378 | C | SER | A | 143 | 18.304 | −5.148 | −3.871 | 1.00 | 0.00 | C |
| ATOM | 2379 | O | SER | A | 143 | 19.513 | −5.360 | −3.904 | 1.00 | 0.00 | O |
| ATOM | 2380 | CB | SER | A | 143 | 17.574 | −6.971 | −2.310 | 1.00 | 0.00 | C |
| ATOM | 2381 | OG | SER | A | 143 | 18.817 | −7.427 | −1.812 | 1.00 | 0.00 | O |
| ATOM | 2382 | H | SER | A | 143 | 15.413 | −5.723 | −2.852 | 1.00 | 0.00 | H |
| ATOM | 2383 | HA | SER | A | 143 | 17.985 | −4.946 | −1.772 | 1.00 | 0.00 | H |
| ATOM | 2384 | 1HB | SER | A | 143 | 16.827 | −7.226 | −1.557 | 1.00 | 0.00 | H |
| ATOM | 2385 | 2HB | SER | A | 143 | 17.313 | −7.549 | −3.200 | 1.00 | 0.00 | H |
| ATOM | 2386 | HG | SER | A | 143 | 18.748 | −8.358 | −1.609 | 1.00 | 0.00 | H |
| ATOM | 2387 | N | ILE | A | 144 | 17.641 | −4.657 | −4.918 | 1.00 | 0.00 | N |
| ATOM | 2388 | CA | ILE | A | 144 | 18.393 | −4.287 | −6.118 | 1.00 | 0.00 | C |
| ATOM | 2389 | C | ILE | A | 144 | 19.191 | −3.011 | −5.782 | 1.00 | 0.00 | C |
| ATOM | 2390 | O | ILE | A | 144 | 20.240 | −2.750 | −6.373 | 1.00 | 0.00 | O |
| ATOM | 2391 | CB | ILE | A | 144 | 17.481 | −4.013 | −7.358 | 1.00 | 0.00 | C |
| ATOM | 2392 | CG1 | ILE | A | 144 | 16.453 | −2.857 | −7.221 | 1.00 | 0.00 | C |
| ATOM | 2393 | CG2 | ILE | A | 144 | 16.802 | −5.312 | −7.819 | 1.00 | 0.00 | C |
| ATOM | 2394 | CD1 | ILE | A | 144 | 15.970 | −2.300 | −8.568 | 1.00 | 0.00 | C |
| ATOM | 2395 | H | ILE | A | 144 | 16.629 | −4.600 | −4.918 | 1.00 | 0.00 | H |
| ATOM | 2396 | HA | ILE | A | 144 | 19.095 | −5.081 | −6.380 | 1.00 | 0.00 | H |
| ATOM | 2397 | HB | ILE | A | 144 | 18.163 | −3.723 | −8.160 | 1.00 | 0.00 | H |
| ATOM | 2398 | 1HG1 | ILE | A | 144 | 15.596 | −3.180 | −6.636 | 1.00 | 0.00 | H |
| ATOM | 2399 | 2HG1 | ILE | A | 144 | 16.867 | −2.008 | −6.678 | 1.00 | 0.00 | H |
| ATOM | 2400 | 1HG2 | ILE | A | 144 | 16.206 | −5.169 | −8.718 | 1.00 | 0.00 | H |
| ATOM | 2401 | 2HG2 | ILE | A | 144 | 17.545 | −6.070 | −8.053 | 1.00 | 0.00 | H |
| ATOM | 2402 | 3HG2 | ILE | A | 144 | 16.152 | −5.719 | −7.047 | 1.00 | 0.00 | H |
| ATOM | 2403 | 1HD1 | ILE | A | 144 | 15.211 | −1.537 | −8.415 | 1.00 | 0.00 | H |
| ATOM | 2404 | 2HD1 | ILE | A | 144 | 16.790 | −1.835 | −9.116 | 1.00 | 0.00 | H |
| ATOM | 2405 | 3HD1 | ILE | A | 144 | 15.532 | −3.069 | −9.201 | 1.00 | 0.00 | H |
| ATOM | 2406 | N | ASP | A | 145 | 18.698 | −2.229 | −4.821 | 1.00 | 0.00 | N |
| ATOM | 2407 | CA | ASP | A | 145 | 19.378 | −0.988 | −4.414 | 1.00 | 0.00 | C |
| ATOM | 2408 | C | ASP | A | 145 | 20.646 | −1.344 | −3.648 | 1.00 | 0.00 | C |
| ATOM | 2409 | O | ASP | A | 145 | 21.638 | −0.609 | −3.675 | 1.00 | 0.00 | O |
| ATOM | 2410 | CB | ASP | A | 145 | 18.496 | −0.141 | −3.485 | 1.00 | 0.00 | C |
| ATOM | 2411 | CG | ASP | A | 145 | 17.444 | 0.756 | −4.188 | 1.00 | 0.00 | C |
| ATOM | 2412 | OD1 | ASP | A | 145 | 17.527 | 0.986 | −5.417 | 1.00 | 0.00 | O |
| ATOM | 2413 | OD2 | ASP | A | 145 | 16.551 | 1.288 | −3.489 | 1.00 | 0.00 | O |
| ATOM | 2414 | H | ASP | A | 145 | 17.810 | −2.450 | −4.385 | 1.00 | 0.00 | H |
| ATOM | 2415 | HA | ASP | A | 145 | 19.678 | −0.412 | −5.293 | 1.00 | 0.00 | H |
| ATOM | 2416 | 1HB | ASP | A | 145 | 18.009 | −0.740 | −2.720 | 1.00 | 0.00 | H |
| ATOM | 2417 | 2HB | ASP | A | 145 | 19.126 | 0.563 | −2.941 | 1.00 | 0.00 | H |
| ATOM | 2418 | N | VAL | A | 146 | 20.601 | −2.469 | −2.944 | 1.00 | 0.00 | N |
| ATOM | 2419 | CA | VAL | A | 146 | 21.752 | −2.913 | −2.159 | 1.00 | 0.00 | C |
| ATOM | 2420 | C | VAL | A | 146 | 22.867 | −3.409 | −3.093 | 1.00 | 0.00 | C |
| ATOM | 2421 | O | VAL | A | 146 | 24.057 | −3.197 | −2.832 | 1.00 | 0.00 | O |
| ATOM | 2422 | CB | VAL | A | 146 | 21.338 | −4.019 | −1.166 | 1.00 | 0.00 | C |
| ATOM | 2423 | CG1 | VAL | A | 146 | 22.495 | −4.582 | −0.322 | 1.00 | 0.00 | C |
| ATOM | 2424 | CG2 | VAL | A | 146 | 20.201 | −3.567 | −0.231 | 1.00 | 0.00 | C |
| ATOM | 2425 | H | VAL | A | 146 | 19.758 | −3.021 | −2.985 | 1.00 | 0.00 | H |
| ATOM | 2426 | HA | VAL | A | 146 | 22.155 | −2.067 | −1.597 | 1.00 | 0.00 | H |
| ATOM | 2427 | HB | VAL | A | 146 | 20.955 | −4.858 | −1.750 | 1.00 | 0.00 | H |
| ATOM | 2428 | 1HG1 | VAL | A | 146 | 22.180 | −5.490 | 0.182 | 1.00 | 0.00 | H |
| ATOM | 2429 | 2HG1 | VAL | A | 146 | 23.366 | −4.870 | −0.910 | 1.00 | 0.00 | H |
| ATOM | 2430 | 3HG1 | VAL | A | 146 | 22.811 | −3.868 | 0.437 | 1.00 | 0.00 | H |
| ATOM | 2431 | 1HG2 | VAL | A | 146 | 19.894 | −4.384 | 0.424 | 1.00 | 0.00 | H |
| ATOM | 2432 | 2HG2 | VAL | A | 146 | 20.522 | −2.738 | 0.400 | 1.00 | 0.00 | H |
| ATOM | 2433 | 3HG2 | VAL | A | 146 | 19.315 | −3.242 | −0.776 | 1.00 | 0.00 | H |
| ATOM | 2434 | N | TRP | A | 147 | 22.489 | −4.066 | −4.186 | 1.00 | 0.00 | N |
| ATOM | 2435 | CA | TRP | A | 147 | 23.491 | −4.527 | −5.136 | 1.00 | 0.00 | C |
| ATOM | 2436 | C | TRP | A | 147 | 24.174 | −3.276 | −5.698 | 1.00 | 0.00 | C |
| ATOM | 2437 | O | TRP | A | 147 | 25.404 | −3.200 | −5.725 | 1.00 | 0.00 | O |
| ATOM | 2438 | CB | TRP | A | 147 | 22.845 | −5.334 | −6.276 | 1.00 | 0.00 | C |
| ATOM | 2439 | CG | TRP | A | 147 | 23.821 | −5.694 | −7.368 | 1.00 | 0.00 | C |
| ATOM | 2440 | CD1 | TRP | A | 147 | 24.183 | −4.879 | −8.389 | 1.00 | 0.00 | C |
| ATOM | 2441 | CD2 | TRP | A | 147 | 24.584 | −6.924 | −7.560 | 1.00 | 0.00 | C |
| ATOM | 2442 | NE1 | TRP | A | 147 | 25.107 | −5.511 | −9.190 | 1.00 | 0.00 | N |
| ATOM | 2443 | CE3 | TRP | A | 147 | 24.659 | −8.167 | −6.887 | 1.00 | 0.00 | C |
| ATOM | 2444 | CE2 | TRP | A | 147 | 25.407 | −6.768 | −8.720 | 1.00 | 0.00 | C |
| ATOM | 2445 | CZ3 | TRP | A | 147 | 25.504 | −9.197 | −7.350 | 1.00 | 0.00 | C |
| ATOM | 2446 | CZ2 | TRP | A | 147 | 26.260 | −7.782 | −9.182 | 1.00 | 0.00 | C |
| ATOM | 2447 | CH2 | TRP | A | 147 | 26.305 | −9.004 | −8.492 | 1.00 | 0.00 | C |
| ATOM | 2448 | H | TRP | A | 147 | 21.505 | −4.188 | −4.381 | 1.00 | 0.00 | H |
| ATOM | 2449 | HA | TRP | A | 147 | 24.236 | −5.146 | −4.631 | 1.00 | 0.00 | H |
| ATOM | 2450 | 1HB | TRP | A | 147 | 22.396 | −6.250 | −5.893 | 1.00 | 0.00 | H |
| ATOM | 2451 | 2HB | TRP | A | 147 | 22.036 | −4.761 | −6.729 | 1.00 | 0.00 | H |
| ATOM | 2452 | HD1 | TRP | A | 147 | 23.786 | −3.885 | −8.548 | 1.00 | 0.00 | H |
| ATOM | 2453 | HE1 | TRP | A | 147 | 25.446 | −5.110 | −10.055 | 1.00 | 0.00 | H |
| ATOM | 2454 | HE3 | TRP | A | 147 | 24.047 | −8.332 | −6.014 | 1.00 | 0.00 | H |
| ATOM | 2455 | HZ3 | TRP | A | 147 | 25.538 | −10.144 | −6.833 | 1.00 | 0.00 | H |
| ATOM | 2456 | HZ2 | TRP | A | 147 | 26.861 | −7.634 | −10.066 | 1.00 | 0.00 | H |
| ATOM | 2457 | HH2 | TRP | A | 147 | 26.944 | −9.796 | −8.847 | 1.00 | 0.00 | H |
| ATOM | 2458 | N | SER | A | 148 | 23.368 | −2.299 | −6.127 | 1.00 | 0.00 | N |
| ATOM | 2459 | CA | SER | A | 148 | 23.882 | −1.049 | −6.686 | 1.00 | 0.00 | C |
| ATOM | 2460 | C | SER | A | 148 | 24.817 | −0.360 | −5.700 | 1.00 | 0.00 | C |
| ATOM | 2461 | O | SER | A | 148 | 25.881 | 0.131 | −6.086 | 1.00 | 0.00 | O |
| ATOM | 2462 | CB | SER | A | 148 | 22.739 | −0.099 | −7.046 | 1.00 | 0.00 | C |
| ATOM | 2463 | OG | SER | A | 148 | 21.858 | −0.709 | −7.972 | 1.00 | 0.00 | O |
| ATOM | 2464 | H | SER | A | 148 | 22.360 | −2.426 | −6.140 | 1.00 | 0.00 | H |
| ATOM | 2465 | HA | SER | A | 148 | 24.457 | −1.285 | −7.584 | 1.00 | 0.00 | H |
| ATOM | 2466 | 1HB | SER | A | 148 | 22.171 | 0.151 | −6.151 | 1.00 | 0.00 | H |
| ATOM | 2467 | 2HB | SER | A | 148 | 23.138 | 0.818 | −7.478 | 1.00 | 0.00 | H |
| ATOM | 2468 | HG | SER | A | 148 | 22.352 | −1.025 | −8.730 | 1.00 | 0.00 | H |
| ATOM | 2469 | N | ALA | A | 149 | 24.414 | −0.314 | −4.431 | 1.00 | 0.00 | N |
| ATOM | 2470 | CA | ALA | A | 149 | 25.255 | 0.293 | −3.399 | 1.00 | 0.00 | C |
| ATOM | 2471 | C | ALA | A | 149 | 26.590 | −0.470 | −3.313 | 1.00 | 0.00 | C |
| ATOM | 2472 | O | ALA | A | 149 | 27.660 | 0.128 | −3.168 | 1.00 | 0.00 | O |
| ATOM | 2473 | CB | ALA | A | 149 | 24.531 | 0.269 | −2.046 | 1.00 | 0.00 | C |
| ATOM | 2474 | H | ALA | A | 149 | 23.490 | −0.658 | −4.180 | 1.00 | 0.00 | H |
| ATOM | 2475 | HA | ALA | A | 149 | 25.464 | 1.329 | −3.673 | 1.00 | 0.00 | H |
| ATOM | 2476 | 1HB | ALA | A | 149 | 25.136 | 0.741 | −1.271 | 1.00 | 0.00 | H |
| ATOM | 2477 | 2HB | ALA | A | 149 | 23.587 | 0.811 | −2.097 | 1.00 | 0.00 | H |
| ATOM | 2478 | 3HB | ALA | A | 149 | 24.314 | −0.748 | −1.722 | 1.00 | 0.00 | H |
| ATOM | 2479 | N | GLY | A | 150 | 26.525 | −1.795 | −3.407 | 1.00 | 0.00 | N |
| ATOM | 2480 | CA | GLY | A | 150 | 27.734 | −2.600 | −3.365 | 1.00 | 0.00 | C |
| ATOM | 2481 | C | GLY | A | 150 | 28.645 | −2.289 | −4.542 | 1.00 | 0.00 | C |
| ATOM | 2482 | O | GLY | A | 150 | 29.867 | −2.357 | −4.427 | 1.00 | 0.00 | O |
| ATOM | 2483 | H | GLY | A | 150 | 25.625 | −2.257 | −3.511 | 1.00 | 0.00 | H |
| ATOM | 2484 | 1HA | GLY | A | 150 | 28.283 | −2.426 | −2.440 | 1.00 | 0.00 | H |
| ATOM | 2485 | 2HA | GLY | A | 150 | 27.464 | −3.656 | −3.394 | 1.00 | 0.00 | H |
| ATOM | 2486 | N | CYS | A | 151 | 28.060 | −1.947 | −5.685 | 1.00 | 0.00 | N |
| ATOM | 2487 | CA | CYS | A | 151 | 28.848 | −1.622 | −6.873 | 1.00 | 0.00 | C |
| ATOM | 2488 | C | CYS | A | 151 | 29.543 | −0.288 | −6.651 | 1.00 | 0.00 | C |
| ATOM | 2489 | O | CYS | A | 151 | 30.663 | −0.077 | −7.115 | 1.00 | 0.00 | O |
| ATOM | 2490 | CB | CYS | A | 151 | 27.948 | −1.533 | −8.103 | 1.00 | 0.00 | C |
| ATOM | 2491 | SG | CYS | A | 151 | 28.992 | −1.462 | −9.594 | 1.00 | 0.00 | S |
| ATOM | 2492 | H | CYS | A | 151 | 27.051 | −2.029 | −5.761 | 1.00 | 0.00 | H |
| ATOM | 2493 | HA | CYS | A | 151 | 29.612 | −2.384 | −7.040 | 1.00 | 0.00 | H |
| ATOM | 2494 | 1HB | CYS | A | 151 | 27.327 | −2.427 | −8.175 | 1.00 | 0.00 | H |
| ATOM | 2495 | 2HB | CYS | A | 151 | 27.274 | −0.685 | −8.061 | 1.00 | 0.00 | H |
| ATOM | 2496 | HG | CYS | A | 151 | 28.031 | −1.642 | −10.518 | 1.00 | 0.00 | H |
| ATOM | 2497 | N | VAL | A | 152 | 28.875 | 0.621 | −5.945 | 1.00 | 0.00 | N |
| ATOM | 2498 | CA | VAL | A | 152 | 29.463 | 1.925 | −5.654 | 1.00 | 0.00 | C |
| ATOM | 2499 | C | VAL | A | 152 | 30.643 | 1.749 | −4.691 | 1.00 | 0.00 | C |
| ATOM | 2500 | O | VAL | A | 152 | 31.707 | 2.336 | −4.895 | 1.00 | 0.00 | O |
| ATOM | 2501 | CB | VAL | A | 152 | 28.423 | 2.906 | −5.031 | 1.00 | 0.00 | C |
| ATOM | 2502 | CG1 | VAL | A | 152 | 29.025 | 4.235 | −4.521 | 1.00 | 0.00 | C |
| ATOM | 2503 | CG2 | VAL | A | 152 | 27.305 | 3.242 | −6.033 | 1.00 | 0.00 | C |
| ATOM | 2504 | H | VAL | A | 152 | 27.948 | 0.402 | −5.600 | 1.00 | 0.00 | H |
| ATOM | 2505 | HA | VAL | A | 152 | 29.849 | 2.358 | −6.580 | 1.00 | 0.00 | H |
| ATOM | 2506 | HB | VAL | A | 152 | 27.958 | 2.423 | −4.173 | 1.00 | 0.00 | H |
| ATOM | 2507 | 1HG1 | VAL | A | 152 | 28.249 | 4.906 | −4.157 | 1.00 | 0.00 | H |
| ATOM | 2508 | 2HG1 | VAL | A | 152 | 29.712 | 4.088 | −3.687 | 1.00 | 0.00 | H |
| ATOM | 2509 | 3HG1 | VAL | A | 152 | 29.570 | 4.755 | −5.310 | 1.00 | 0.00 | H |
| ATOM | 2510 | 1HG2 | VAL | A | 152 | 26.514 | 3.831 | −5.568 | 1.00 | 0.00 | H |
| ATOM | 2511 | 2HG2 | VAL | A | 152 | 27.708 | 3.819 | −6.862 | 1.00 | 0.00 | H |
| ATOM | 2512 | 3HG2 | VAL | A | 152 | 26.844 | 2.355 | −6.459 | 1.00 | 0.00 | H |
| ATOM | 2513 | N | LEU | A | 153 | 30.461 | 0.942 | −3.647 | 1.00 | 0.00 | N |
| ATOM | 2514 | CA | LEU | A | 153 | 31.534 | 0.693 | −2.685 | 1.00 | 0.00 | C |
| ATOM | 2515 | C | LEU | A | 153 | 32.768 | 0.125 | −3.396 | 1.00 | 0.00 | C |
| ATOM | 2516 | O | LEU | A | 153 | 33.873 | 0.630 | −3.229 | 1.00 | 0.00 | O |
| ATOM | 2517 | CB | LEU | A | 153 | 31.073 | −0.295 | −1.600 | 1.00 | 0.00 | C |
| ATOM | 2518 | CG | LEU | A | 153 | 32.083 | −0.656 | −0.482 | 1.00 | 0.00 | C |
| ATOM | 2519 | CD1 | LEU | A | 153 | 32.383 | 0.539 | 0.435 | 1.00 | 0.00 | C |
| ATOM | 2520 | CD2 | LEU | A | 153 | 31.581 | −1.855 | 0.341 | 1.00 | 0.00 | C |
| ATOM | 2521 | H | LEU | A | 153 | 29.551 | 0.510 | −3.516 | 1.00 | 0.00 | H |
| ATOM | 2522 | HA | LEU | A | 153 | 31.809 | 1.643 | −2.222 | 1.00 | 0.00 | H |
| ATOM | 2523 | 1HB | LEU | A | 153 | 30.168 | 0.097 | −1.134 | 1.00 | 0.00 | H |
| ATOM | 2524 | 2HB | LEU | A | 153 | 30.758 | −1.219 | −2.088 | 1.00 | 0.00 | H |
| ATOM | 2525 | HG | LEU | A | 153 | 33.023 | −0.971 | −0.935 | 1.00 | 0.00 | H |
| ATOM | 2526 | 1HD1 | LEU | A | 153 | 33.108 | 0.272 | 1.205 | 1.00 | 0.00 | H |
| ATOM | 2527 | 2HD1 | LEU | A | 153 | 32.803 | 1.372 | −0.127 | 1.00 | 0.00 | H |
| ATOM | 2528 | 3HD1 | LEU | A | 153 | 31.484 | 0.896 | 0.938 | 1.00 | 0.00 | H |
| ATOM | 2529 | 1HD2 | LEU | A | 153 | 32.303 | −2.138 | 1.108 | 1.00 | 0.00 | H |
| ATOM | 2530 | 2HD2 | LEU | A | 153 | 30.638 | −1.630 | 0.839 | 1.00 | 0.00 | H |
| ATOM | 2531 | 3HD2 | LEU | A | 153 | 31.424 | −2.730 | −0.290 | 1.00 | 0.00 | H |
| ATOM | 2532 | N | ALA | A | 154 | 32.575 | −0.923 | −4.190 | 1.00 | 0.00 | N |
| ATOM | 2533 | CA | ALA | A | 154 | 33.688 | −1.548 | −4.909 | 1.00 | 0.00 | C |
| ATOM | 2534 | C | ALA | A | 154 | 34.400 | −0.541 | −5.802 | 1.00 | 0.00 | C |
| ATOM | 2535 | O | ALA | A | 154 | 35.629 | −0.524 | −5.876 | 1.00 | 0.00 | O |
| ATOM | 2536 | CB | ALA | A | 154 | 33.178 | −2.734 | −5.747 | 1.00 | 0.00 | C |
| ATOM | 2537 | H | ALA | A | 154 | 31.642 | −1.314 | −4.279 | 1.00 | 0.00 | H |
| ATOM | 2538 | HA | ALA | A | 154 | 34.402 | −1.918 | −4.171 | 1.00 | 0.00 | H |
| ATOM | 2539 | 1HB | ALA | A | 154 | 34.004 | −3.201 | −6.278 | 1.00 | 0.00 | H |
| ATOM | 2540 | 2HB | ALA | A | 154 | 32.707 | −3.494 | −5.124 | 1.00 | 0.00 | H |
| ATOM | 2541 | 3HB | ALA | A | 154 | 32.443 | −2.412 | −6.485 | 1.00 | 0.00 | H |
| ATOM | 2542 | N | GLU | A | 155 | 33.624 | 0.310 | −6.468 | 1.00 | 0.00 | N |
| ATOM | 2543 | CA | GLU | A | 155 | 34.177 | 1.330 | −7.358 | 1.00 | 0.00 | C |
| ATOM | 2544 | C | GLU | A | 155 | 35.027 | 2.363 | −6.608 | 1.00 | 0.00 | C |
| ATOM | 2545 | O | GLU | A | 155 | 36.045 | 2.826 | −7.119 | 1.00 | 0.00 | O |
| ATOM | 2546 | CB | GLU | A | 155 | 33.043 | 2.032 | −8.103 | 1.00 | 0.00 | C |
| ATOM | 2547 | CG | GLU | A | 155 | 33.431 | 3.309 | −8.888 | 1.00 | 0.00 | C |
| ATOM | 2548 | CD | GLU | A | 155 | 32.411 | 3.716 | −9.941 | 1.00 | 0.00 | C |
| ATOM | 2549 | OE1 | GLU | A | 155 | 31.200 | 3.510 | −9.713 | 1.00 | 0.00 | O |
| ATOM | 2550 | OE2 | GLU | A | 155 | 32.856 | 4.239 | −10.980 | 1.00 | 0.00 | O |
| ATOM | 2551 | H | GLU | A | 155 | 32.615 | 0.248 | −6.376 | 1.00 | 0.00 | H |
| ATOM | 2552 | HA | GLU | A | 155 | 34.823 | 0.829 | −8.074 | 1.00 | 0.00 | H |
| ATOM | 2553 | 1HB | GLU | A | 155 | 32.587 | 1.315 | −8.787 | 1.00 | 0.00 | H |
| ATOM | 2554 | 2HB | GLU | A | 155 | 32.248 | 2.307 | −7.411 | 1.00 | 0.00 | H |
| ATOM | 2555 | 1HG | GLU | A | 155 | 33.564 | 4.154 | −8.213 | 1.00 | 0.00 | H |
| ATOM | 2556 | 2HG | GLU | A | 155 | 34.383 | 3.159 | −9.395 | 1.00 | 0.00 | H |
| ATOM | 2557 | N | LEU | A | 156 | 34.602 | 2.738 | −5.404 | 1.00 | 0.00 | N |
| ATOM | 2558 | CA | LEU | A | 156 | 35.364 | 3.709 | −4.612 | 1.00 | 0.00 | C |
| ATOM | 2559 | C | LEU | A | 156 | 36.662 | 3.074 | −4.120 | 1.00 | 0.00 | C |
| ATOM | 2560 | O | LEU | A | 156 | 37.673 | 3.756 | −3.954 | 1.00 | 0.00 | O |
| ATOM | 2561 | CB | LEU | A | 156 | 34.541 | 4.191 | −3.415 | 1.00 | 0.00 | C |
| ATOM | 2562 | CG | LEU | A | 156 | 33.344 | 5.068 | −3.838 | 1.00 | 0.00 | C |
| ATOM | 2563 | CD1 | LEU | A | 156 | 32.360 | 5.221 | −2.680 | 1.00 | 0.00 | C |
| ATOM | 2564 | CD2 | LEU | A | 156 | 33.784 | 6.453 | −4.346 | 1.00 | 0.00 | C |
| ATOM | 2565 | H | LEU | A | 156 | 33.712 | 2.392 | −5.055 | 1.00 | 0.00 | H |
| ATOM | 2566 | HA | LEU | A | 156 | 35.656 | 4.549 | −5.241 | 1.00 | 0.00 | H |
| ATOM | 2567 | 1HB | LEU | A | 156 | 34.209 | 3.342 | −2.816 | 1.00 | 0.00 | H |
| ATOM | 2568 | 2HB | LEU | A | 156 | 35.183 | 4.821 | −2.807 | 1.00 | 0.00 | H |
| ATOM | 2569 | HG | LEU | A | 156 | 32.807 | 4.580 | −4.650 | 1.00 | 0.00 | H |
| ATOM | 2570 | 1HD1 | LEU | A | 156 | 31.497 | 5.815 | −2.979 | 1.00 | 0.00 | H |
| ATOM | 2571 | 2HD1 | LEU | A | 156 | 31.992 | 4.253 | −2.341 | 1.00 | 0.00 | H |
| ATOM | 2572 | 3HD1 | LEU | A | 156 | 32.831 | 5.720 | −1.831 | 1.00 | 0.00 | H |
| ATOM | 2573 | 1HD2 | LEU | A | 156 | 32.921 | 7.051 | −4.631 | 1.00 | 0.00 | H |
| ATOM | 2574 | 2HD2 | LEU | A | 156 | 34.328 | 7.006 | −3.579 | 1.00 | 0.00 | H |
| ATOM | 2575 | 3HD2 | LEU | A | 156 | 34.420 | 6.387 | −5.227 | 1.00 | 0.00 | H |
| ATOM | 2576 | N | LEU | A | 157 | 36.627 | 1.765 | −3.883 | 1.00 | 0.00 | N |
| ATOM | 2577 | CA | LEU | A | 157 | 37.800 | 1.026 | −3.421 | 1.00 | 0.00 | C |
| ATOM | 2578 | C | LEU | A | 157 | 38.755 | 0.746 | −4.582 | 1.00 | 0.00 | C |
| ATOM | 2579 | O | LEU | A | 157 | 39.975 | 0.845 | −4.429 | 1.00 | 0.00 | O |
| ATOM | 2580 | CB | LEU | A | 157 | 37.378 | −0.307 | −2.790 | 1.00 | 0.00 | C |
| ATOM | 2581 | CG | LEU | A | 157 | 36.679 | −0.236 | −1.430 | 1.00 | 0.00 | C |
| ATOM | 2582 | CD1 | LEU | A | 157 | 35.936 | −1.550 | −1.138 | 1.00 | 0.00 | C |
| ATOM | 2583 | CD2 | LEU | A | 157 | 37.715 | 0.056 | −0.345 | 1.00 | 0.00 | C |
| ATOM | 2584 | H | LEU | A | 157 | 35.735 | 1.286 | −3.966 | 1.00 | 0.00 | H |
| ATOM | 2585 | HA | LEU | A | 157 | 38.348 | 1.620 | −2.688 | 1.00 | 0.00 | H |
| ATOM | 2586 | 1HB | LEU | A | 157 | 36.714 | −0.810 | −3.492 | 1.00 | 0.00 | H |
| ATOM | 2587 | 2HB | LEU | A | 157 | 38.241 | −0.970 | −2.706 | 1.00 | 0.00 | H |
| ATOM | 2588 | HG | LEU | A | 157 | 35.936 | 0.561 | −1.452 | 1.00 | 0.00 | H |
| ATOM | 2589 | 1HD1 | LEU | A | 157 | 35.368 | −1.485 | −0.211 | 1.00 | 0.00 | H |
| ATOM | 2590 | 2HD1 | LEU | A | 157 | 35.220 | −1.783 | −1.926 | 1.00 | 0.00 | H |
| ATOM | 2591 | 3HD1 | LEU | A | 157 | 36.622 | −2.395 | −1.056 | 1.00 | 0.00 | H |
| ATOM | 2592 | 1HD2 | LEU | A | 157 | 37.312 | −0.112 | 0.651 | 1.00 | 0.00 | H |
| ATOM | 2593 | 2HD2 | LEU | A | 157 | 38.576 | −0.589 | −0.458 | 1.00 | 0.00 | H |
| ATOM | 2594 | 3HD2 | LEU | A | 157 | 38.085 | 1.076 | −0.413 | 1.00 | 0.00 | H |
| ATOM | 2595 | N | LEU | A | 158 | 38.192 | 0.404 | −5.738 | 1.00 | 0.00 | N |
| ATOM | 2596 | CA | LEU | A | 158 | 38.969 | 0.082 | −6.935 | 1.00 | 0.00 | C |
| ATOM | 2597 | C | LEU | A | 158 | 39.458 | 1.280 | −7.726 | 1.00 | 0.00 | C |
| ATOM | 2598 | O | LEU | A | 158 | 40.559 | 1.256 | −8.276 | 1.00 | 0.00 | O |
| ATOM | 2599 | CB | LEU | A | 158 | 38.143 | −0.785 | −7.880 | 1.00 | 0.00 | C |
| ATOM | 2600 | CG | LEU | A | 158 | 37.913 | −2.231 | −7.417 | 1.00 | 0.00 | C |
| ATOM | 2601 | CD1 | LEU | A | 158 | 36.810 | −2.880 | −8.257 | 1.00 | 0.00 | C |
| ATOM | 2602 | CD2 | LEU | A | 158 | 39.189 | −3.084 | −7.462 | 1.00 | 0.00 | C |
| ATOM | 2603 | H | LEU | A | 158 | 37.182 | 0.258 | −5.765 | 1.00 | 0.00 | H |
| ATOM | 2604 | HA | LEU | A | 158 | 39.861 | −0.469 | −6.637 | 1.00 | 0.00 | H |
| ATOM | 2605 | 1HB | LEU | A | 158 | 37.180 | −0.299 | −8.050 | 1.00 | 0.00 | H |
| ATOM | 2606 | 2HB | LEU | A | 158 | 38.611 | −0.827 | −8.866 | 1.00 | 0.00 | H |
| ATOM | 2607 | HG | LEU | A | 158 | 37.557 | −2.202 | −6.391 | 1.00 | 0.00 | H |
| ATOM | 2608 | 1HD1 | LEU | A | 158 | 36.602 | −3.894 | −7.919 | 1.00 | 0.00 | H |
| ATOM | 2609 | 2HD1 | LEU | A | 158 | 35.892 | −2.301 | −8.172 | 1.00 | 0.00 | H |
| ATOM | 2610 | 3HD1 | LEU | A | 158 | 37.078 | −2.925 | −9.313 | 1.00 | 0.00 | H |
| ATOM | 2611 | 1HD2 | LEU | A | 158 | 38.972 | −4.100 | −7.132 | 1.00 | 0.00 | H |
| ATOM | 2612 | 2HD2 | LEU | A | 158 | 39.598 | −3.148 | −8.470 | 1.00 | 0.00 | H |
| ATOM | 2613 | 3HD2 | LEU | A | 158 | 39.968 | −2.700 | −6.806 | 1.00 | 0.00 | H |
| ATOM | 2614 | N | GLY | A | 159 | 38.634 | 2.317 | −7.808 | 1.00 | 0.00 | N |
| ATOM | 2615 | CA | GLY | A | 159 | 39.017 | 3.490 | −8.569 | 1.00 | 0.00 | C |
| ATOM | 2616 | C | GLY | A | 159 | 38.383 | 3.461 | −9.944 | 1.00 | 0.00 | C |
| ATOM | 2617 | O | GLY | A | 159 | 38.647 | 4.326 | −10.780 | 1.00 | 0.00 | O |
| ATOM | 2618 | H | GLY | A | 159 | 37.719 | 2.319 | −7.356 | 1.00 | 0.00 | H |
| ATOM | 2619 | 1HA | GLY | A | 159 | 38.639 | 4.369 | −8.045 | 1.00 | 0.00 | H |
| ATOM | 2620 | 2HA | GLY | A | 159 | 40.095 | 3.631 | −8.661 | 1.00 | 0.00 | H |
| ATOM | 2621 | N | GLN | A | 160 | 37.540 | 2.460 | −10.179 | 1.00 | 0.00 | N |
| ATOM | 2622 | CA | GLN | A | 160 | 36.837 | 2.318 | −11.454 | 1.00 | 0.00 | C |
| ATOM | 2623 | C | GLN | A | 160 | 35.677 | 1.338 | −11.270 | 1.00 | 0.00 | C |
| ATOM | 2624 | O | GLN | A | 160 | 35.674 | 0.549 | −10.326 | 1.00 | 0.00 | O |
| ATOM | 2625 | CB | GLN | A | 160 | 37.795 | 1.790 | −12.525 | 1.00 | 0.00 | C |
| ATOM | 2626 | CG | GLN | A | 160 | 38.411 | 0.396 | −12.243 | 1.00 | 0.00 | C |
| ATOM | 2627 | CD | GLN | A | 160 | 39.469 | 0.003 | −13.272 | 1.00 | 0.00 | C |
| ATOM | 2628 | OE1 | GLN | A | 160 | 39.436 | 0.441 | −14.419 | 1.00 | 0.00 | O |
| ATOM | 2629 | NE2 | GLN | A | 160 | 40.430 | −0.819 | −12.873 | 1.00 | 0.00 | N |
| ATOM | 2630 | H | GLN | A | 160 | 37.304 | 1.805 | −9.447 | 1.00 | 0.00 | H |
| ATOM | 2631 | HA | GLN | A | 160 | 36.443 | 3.295 | −11.737 | 1.00 | 0.00 | H |
| ATOM | 2632 | 1HB | GLN | A | 160 | 37.286 | 1.751 | −13.487 | 1.00 | 0.00 | H |
| ATOM | 2633 | 2HB | GLN | A | 160 | 38.601 | 2.510 | −12.674 | 1.00 | 0.00 | H |
| ATOM | 2634 | 1HG | GLN | A | 160 | 38.864 | 0.384 | −11.252 | 1.00 | 0.00 | H |
| ATOM | 2635 | 2HG | GLN | A | 160 | 37.641 | −0.377 | −12.247 | 1.00 | 0.00 | H |
| ATOM | 2636 | 1HE2 | GLN | A | 160 | 41.149 | −1.101 | −13.521 | 1.00 | 0.00 | H |
| ATOM | 2637 | 2HE2 | GLN | A | 160 | 40.445 | −1.173 | −11.930 | 1.00 | 0.00 | H |
| ATOM | 2638 | N | PRO | A | 161 | 34.679 | 1.368 | −12.175 | 1.00 | 0.00 | N |
| ATOM | 2639 | CA | PRO | A | 161 | 33.539 | 0.443 | −12.040 | 1.00 | 0.00 | C |
| ATOM | 2640 | C | PRO | A | 161 | 33.990 | −1.016 | −11.944 | 1.00 | 0.00 | C |
| ATOM | 2641 | O | PRO | A | 161 | 34.923 | −1.431 | −12.638 | 1.00 | 0.00 | O |
| ATOM | 2642 | CB | PRO | A | 161 | 32.699 | 0.706 | −13.297 | 1.00 | 0.00 | C |
| ATOM | 2643 | CG | PRO | A | 161 | 33.051 | 2.111 | −13.753 | 1.00 | 0.00 | C |
| ATOM | 2644 | CD | PRO | A | 161 | 34.469 | 2.350 | −13.243 | 1.00 | 0.00 | C |
| ATOM | 2645 | HA | PRO | A | 161 | 32.975 | 0.728 | −11.150 | 1.00 | 0.00 | H |
| ATOM | 2646 | 1HB | PRO | A | 161 | 32.959 | 0.003 | −14.091 | 1.00 | 0.00 | H |
| ATOM | 2647 | 2HB | PRO | A | 161 | 31.631 | 0.595 | −13.107 | 1.00 | 0.00 | H |
| ATOM | 2648 | 1HG | PRO | A | 161 | 32.373 | 2.820 | −13.272 | 1.00 | 0.00 | H |
| ATOM | 2649 | 2HG | PRO | A | 161 | 32.958 | 2.246 | −14.831 | 1.00 | 0.00 | H |
| ATOM | 2650 | 1HD | PRO | A | 161 | 34.570 | 3.378 | −12.892 | 1.00 | 0.00 | H |
| ATOM | 2651 | 2HD | PRO | A | 161 | 35.184 | 2.184 | −14.048 | 1.00 | 0.00 | H |
| ATOM | 2652 | N | ILE | A | 162 | 33.330 | −1.797 | −11.094 | 1.00 | 0.00 | N |
| ATOM | 2653 | CA | ILE | A | 162 | 33.710 | −3.197 | −10.933 | 1.00 | 0.00 | C |
| ATOM | 2654 | C | ILE | A | 162 | 33.090 | −4.150 | −11.962 | 1.00 | 0.00 | C |
| ATOM | 2655 | O | ILE | A | 162 | 33.675 | −5.186 | −12.269 | 1.00 | 0.00 | O |
| ATOM | 2656 | CB | ILE | A | 162 | 33.395 | −3.705 | −9.500 | 1.00 | 0.00 | C |
| ATOM | 2657 | CG1 | ILE | A | 162 | 33.832 | −5.177 | −9.257 | 1.00 | 0.00 | C |
| ATOM | 2658 | CG2 | ILE | A | 162 | 31.925 | −3.475 | −9.086 | 1.00 | 0.00 | C |
| ATOM | 2659 | CD1 | ILE | A | 162 | 33.945 | −5.614 | −7.792 | 1.00 | 0.00 | C |
| ATOM | 2660 | H | ILE | A | 162 | 32.603 | −1.414 | −10.509 | 1.00 | 0.00 | H |
| ATOM | 2661 | HA | ILE | A | 162 | 34.793 | −3.278 | −11.051 | 1.00 | 0.00 | H |
| ATOM | 2662 | HB | ILE | A | 162 | 33.989 | −3.095 | −8.825 | 1.00 | 0.00 | H |
| ATOM | 2663 | 1HG1 | ILE | A | 162 | 33.137 | −5.854 | −9.755 | 1.00 | 0.00 | H |
| ATOM | 2664 | 2HG1 | ILE | A | 162 | 34.801 | −5.347 | −9.727 | 1.00 | 0.00 | H |
| ATOM | 2665 | 1HG2 | ILE | A | 162 | 31.704 | −3.886 | −8.103 | 1.00 | 0.00 | H |
| ATOM | 2666 | 2HG2 | ILE | A | 162 | 31.688 | −2.413 | −9.037 | 1.00 | 0.00 | H |
| ATOM | 2667 | 3HG2 | ILE | A | 162 | 31.227 | −3.942 | −9.779 | 1.00 | 0.00 | H |
| ATOM | 2668 | 1HD1 | ILE | A | 162 | 34.204 | −6.672 | −7.742 | 1.00 | 0.00 | H |
| ATOM | 2669 | 2HD1 | ILE | A | 162 | 34.726 | −5.068 | −7.266 | 1.00 | 0.00 | H |
| ATOM | 2670 | 3HD1 | ILE | A | 162 | 33.009 | −5.486 | −7.249 | 1.00 | 0.00 | H |
| ATOM | 2671 | N | PHE | A | 163 | 31.930 | −3.805 | −12.507 | 1.00 | 0.00 | N |
| ATOM | 2672 | CA | PHE | A | 163 | 31.277 | −4.663 | −13.498 | 1.00 | 0.00 | C |
| ATOM | 2673 | C | PHE | A | 163 | 30.937 | −3.883 | −14.768 | 1.00 | 0.00 | C |
| ATOM | 2674 | O | PHE | A | 163 | 29.770 | −3.686 | −15.092 | 1.00 | 0.00 | O |
| ATOM | 2675 | CB | PHE | A | 163 | 29.988 | −5.282 | −12.932 | 1.00 | 0.00 | C |
| ATOM | 2676 | CG | PHE | A | 163 | 30.137 | −6.077 | −11.644 | 1.00 | 0.00 | C |
| ATOM | 2677 | CD1 | PHE | A | 163 | 31.104 | −7.100 | −11.532 | 1.00 | 0.00 | C |
| ATOM | 2678 | CD2 | PHE | A | 163 | 29.301 | −5.791 | −10.543 | 1.00 | 0.00 | C |
| ATOM | 2679 | CE1 | PHE | A | 163 | 31.238 | −7.822 | −10.331 | 1.00 | 0.00 | C |
| ATOM | 2680 | CE2 | PHE | A | 163 | 29.435 | −6.518 | −9.346 | 1.00 | 0.00 | C |
| ATOM | 2681 | CZ | PHE | A | 163 | 30.399 | −7.538 | −9.238 | 1.00 | 0.00 | C |
| ATOM | 2682 | H | PHE | A | 163 | 31.479 | −2.951 | −12.218 | 1.00 | 0.00 | H |
| ATOM | 2683 | HA | PHE | A | 163 | 31.911 | −5.482 | −13.836 | 1.00 | 0.00 | H |
| ATOM | 2684 | 1HB | PHE | A | 163 | 29.248 | −4.499 | −12.756 | 1.00 | 0.00 | H |
| ATOM | 2685 | 2HB | PHE | A | 163 | 29.547 | −5.952 | −13.673 | 1.00 | 0.00 | H |
| ATOM | 2686 | HD1 | PHE | A | 163 | 31.760 | −7.332 | −12.359 | 1.00 | 0.00 | H |
| ATOM | 2687 | HD2 | PHE | A | 163 | 28.558 | −5.008 | −10.605 | 1.00 | 0.00 | H |
| ATOM | 2688 | HE1 | PHE | A | 163 | 31.985 | −8.598 | −10.254 | 1.00 | 0.00 | H |
| ATOM | 2689 | HE2 | PHE | A | 163 | 28.792 | −6.290 | −8.510 | 1.00 | 0.00 | H |
| ATOM | 2690 | HZ | PHE | A | 163 | 30.497 | −8.098 | −8.320 | 1.00 | 0.00 | H |
| ATOM | 2691 | N | PRO | A | 164 | 31.960 | −3.433 | −15.511 | 1.00 | 0.00 | N |
| ATOM | 2692 | CA | PRO | A | 164 | 31.705 | −2.680 | −16.741 | 1.00 | 0.00 | C |
| ATOM | 2693 | C | PRO | A | 164 | 31.240 | −3.600 | −17.863 | 1.00 | 0.00 | C |
| ATOM | 2694 | O | PRO | A | 164 | 31.330 | −4.819 | −17.751 | 1.00 | 0.00 | O |
| ATOM | 2695 | CB | PRO | A | 164 | 33.054 | −2.045 | −17.047 | 1.00 | 0.00 | C |
| ATOM | 2696 | CG | PRO | A | 164 | 34.088 | −3.020 | −16.506 | 1.00 | 0.00 | C |
| ATOM | 2697 | CD | PRO | A | 164 | 33.399 | −3.615 | −15.283 | 1.00 | 0.00 | C |
| ATOM | 2698 | HA | PRO | A | 164 | 30.959 | −1.909 | −16.564 | 1.00 | 0.00 | H |
| ATOM | 2699 | 1HB | PRO | A | 164 | 33.204 | −1.826 | −18.105 | 1.00 | 0.00 | H |
| ATOM | 2700 | 2HB | PRO | A | 164 | 33.133 | −1.097 | −16.512 | 1.00 | 0.00 | H |
| ATOM | 2701 | 1HG | PRO | A | 164 | 35.041 | −2.547 | −16.268 | 1.00 | 0.00 | H |
| ATOM | 2702 | 2HG | PRO | A | 164 | 34.276 | −3.805 | −17.241 | 1.00 | 0.00 | H |
| ATOM | 2703 | 1HD | PRO | A | 164 | 33.704 | −3.048 | −14.406 | 1.00 | 0.00 | H |
| ATOM | 2704 | 2HD | PRO | A | 164 | 33.676 | −4.661 | −15.137 | 1.00 | 0.00 | H |
| ATOM | 2705 | N | GLY | A | 165 | 30.745 | −3.009 | −18.946 | 1.00 | 0.00 | N |
| ATOM | 2706 | CA | GLY | A | 165 | 30.272 | −3.794 | −20.072 | 1.00 | 0.00 | C |
| ATOM | 2707 | C | GLY | A | 165 | 29.275 | −3.010 | −20.902 | 1.00 | 0.00 | C |
| ATOM | 2708 | O | GLY | A | 165 | 28.410 | −2.325 | −20.353 | 1.00 | 0.00 | O |
| ATOM | 2709 | H | GLY | A | 165 | 30.641 | −2.006 | −18.990 | 1.00 | 0.00 | H |
| ATOM | 2710 | 1HA | GLY | A | 165 | 31.129 | −4.086 | −20.680 | 1.00 | 0.00 | H |
| ATOM | 2711 | 2HA | GLY | A | 165 | 29.787 | −4.716 | −19.748 | 1.00 | 0.00 | H |
| ATOM | 2712 | N | ASP | A | 166 | 29.388 | −3.107 | −22.224 | 1.00 | 0.00 | N |
| ATOM | 2713 | CA | ASP | A | 166 | 28.481 | −2.383 | −23.109 | 1.00 | 0.00 | C |
| ATOM | 2714 | C | ASP | A | 166 | 27.170 | −3.126 | −23.350 | 1.00 | 0.00 | C |
| ATOM | 2715 | O | ASP | A | 166 | 26.191 | −2.536 | −23.805 | 1.00 | 0.00 | O |
| ATOM | 2716 | CB | ASP | A | 166 | 29.172 | −2.076 | −24.441 | 1.00 | 0.00 | C |
| ATOM | 2717 | CG | ASP | A | 166 | 30.266 | −1.018 | −24.359 | 1.00 | 0.00 | C |
| ATOM | 2718 | OD1 | ASP | A | 166 | 29.927 | 0.154 | −24.106 | 1.00 | 0.00 | O |
| ATOM | 2719 | OD2 | ASP | A | 166 | 31.442 | −1.364 | −24.586 | 1.00 | 0.00 | O |
| ATOM | 2720 | H | ASP | A | 166 | 30.100 | −3.694 | −22.635 | 1.00 | 0.00 | H |
| ATOM | 2721 | HA | ASP | A | 166 | 28.164 | −1.447 | −22.644 | 1.00 | 0.00 | H |
| ATOM | 2722 | 1HB | ASP | A | 166 | 29.583 | −2.990 | −24.873 | 1.00 | 0.00 | H |
| ATOM | 2723 | 2HB | ASP | A | 166 | 28.442 | −1.708 | −25.164 | 1.00 | 0.00 | H |
| ATOM | 2724 | N | SER | A | 167 | 27.154 | −4.417 | −23.047 | 1.00 | 0.00 | N |
| ATOM | 2725 | CA | SER | A | 167 | 25.948 | −5.220 | −23.205 | 1.00 | 0.00 | C |
| ATOM | 2726 | C | SER | A | 167 | 25.707 | −5.999 | −21.918 | 1.00 | 0.00 | C |
| ATOM | 2727 | O | SER | A | 167 | 26.609 | −6.125 | −21.090 | 1.00 | 0.00 | O |
| ATOM | 2728 | CB | SER | A | 167 | 26.097 | −6.201 | −24.370 | 1.00 | 0.00 | C |
| ATOM | 2729 | OG | SER | A | 167 | 24.876 | −6.796 | −24.795 | 1.00 | 0.00 | O |
| ATOM | 2730 | H | SER | A | 167 | 27.956 | −4.846 | −22.612 | 1.00 | 0.00 | H |
| ATOM | 2731 | HA | SER | A | 167 | 25.065 | −4.601 | −23.379 | 1.00 | 0.00 | H |
| ATOM | 2732 | 1HB | SER | A | 167 | 26.499 | −5.663 | −25.229 | 1.00 | 0.00 | H |
| ATOM | 2733 | 2HB | SER | A | 167 | 26.829 | −6.979 | −24.143 | 1.00 | 0.00 | H |
| ATOM | 2734 | HG | SER | A | 167 | 24.668 | −7.527 | −24.232 | 1.00 | 0.00 | H |
| ATOM | 2735 | N | GLY | A | 168 | 24.493 | −6.519 | −21.762 | 1.00 | 0.00 | N |
| ATOM | 2736 | CA | GLY | A | 168 | 24.166 | −7.294 | −20.581 | 1.00 | 0.00 | C |
| ATOM | 2737 | C | GLY | A | 168 | 25.054 | −8.513 | −20.455 | 1.00 | 0.00 | C |
| ATOM | 2738 | O | GLY | A | 168 | 25.481 | −8.872 | −19.364 | 1.00 | 0.00 | O |
| ATOM | 2739 | H | GLY | A | 168 | 23.789 | −6.387 | −22.472 | 1.00 | 0.00 | H |
| ATOM | 2740 | 1HA | GLY | A | 168 | 24.265 | −6.674 | −19.688 | 1.00 | 0.00 | H |
| ATOM | 2741 | 2HA | GLY | A | 168 | 23.126 | −7.617 | −20.633 | 1.00 | 0.00 | H |
| ATOM | 2742 | N | VAL | A | 169 | 25.336 | −9.161 | −21.580 | 1.00 | 0.00 | N |
| ATOM | 2743 | CA | VAL | A | 169 | 26.189 | −10.347 | −21.566 | 1.00 | 0.00 | C |
| ATOM | 2744 | C | VAL | A | 169 | 27.579 | −10.018 | −21.016 | 1.00 | 0.00 | C |
| ATOM | 2745 | O | VAL | A | 169 | 28.121 | −10.769 | −20.203 | 1.00 | 0.00 | O |
| ATOM | 2746 | CB | VAL | A | 169 | 26.352 | −10.948 | −22.987 | 1.00 | 0.00 | C |
| ATOM | 2747 | CG1 | VAL | A | 169 | 27.086 | −12.306 | −22.987 | 1.00 | 0.00 | C |
| ATOM | 2748 | CG2 | VAL | A | 169 | 24.995 | −11.112 | −23.702 | 1.00 | 0.00 | C |
| ATOM | 2749 | H | VAL | A | 169 | 25.036 | −8.780 | −22.465 | 1.00 | 0.00 | H |
| ATOM | 2750 | HA | VAL | A | 169 | 25.737 | −11.092 | −20.907 | 1.00 | 0.00 | H |
| ATOM | 2751 | HB | VAL | A | 169 | 26.946 | −10.261 | −23.594 | 1.00 | 0.00 | H |
| ATOM | 2752 | 1HG1 | VAL | A | 169 | 27.160 | −12.715 | −23.995 | 1.00 | 0.00 | H |
| ATOM | 2753 | 2HG1 | VAL | A | 169 | 28.105 | −12.225 | −22.607 | 1.00 | 0.00 | H |
| ATOM | 2754 | 3HG1 | VAL | A | 169 | 26.563 | −13.040 | −22.371 | 1.00 | 0.00 | H |
| ATOM | 2755 | 1HG2 | VAL | A | 169 | 25.120 | −11.574 | −24.682 | 1.00 | 0.00 | H |
| ATOM | 2756 | 2HG2 | VAL | A | 169 | 24.318 | −11.743 | −23.125 | 1.00 | 0.00 | H |
| ATOM | 2757 | 3HG2 | VAL | A | 169 | 24.497 | −10.157 | −23.869 | 1.00 | 0.00 | H |
| ATOM | 2758 | N | ASP | A | 170 | 28.147 | −8.896 | −21.457 | 1.00 | 0.00 | N |
| ATOM | 2759 | CA | ASP | A | 170 | 29.472 | −8.481 | −21.003 | 1.00 | 0.00 | C |
| ATOM | 2760 | C | ASP | A | 170 | 29.478 | −8.219 | −19.506 | 1.00 | 0.00 | C |
| ATOM | 2761 | O | ASP | A | 170 | 30.381 | −8.658 | −18.797 | 1.00 | 0.00 | O |
| ATOM | 2762 | CB | ASP | A | 170 | 29.920 | −7.220 | −21.736 | 1.00 | 0.00 | C |
| ATOM | 2763 | CG | ASP | A | 170 | 30.002 | −7.305 | −23.252 | 1.00 | 0.00 | C |
| ATOM | 2764 | OD1 | ASP | A | 170 | 30.527 | −8.310 | −23.768 | 1.00 | 0.00 | O |
| ATOM | 2765 | OD2 | ASP | A | 170 | 29.533 | −6.339 | −23.886 | 1.00 | 0.00 | O |
| ATOM | 2766 | H | ASP | A | 170 | 27.643 | −8.329 | −22.127 | 1.00 | 0.00 | H |
| ATOM | 2767 | HA | ASP | A | 170 | 30.181 | −9.291 | −21.190 | 1.00 | 0.00 | H |
| ATOM | 2768 | 1HB | ASP | A | 170 | 29.262 | −6.388 | −21.484 | 1.00 | 0.00 | H |
| ATOM | 2769 | 2HB | ASP | A | 170 | 30.911 | −6.932 | −21.384 | 1.00 | 0.00 | H |
| ATOM | 2770 | N | GLN | A | 171 | 28.473 | −7.486 | −19.039 | 1.00 | 0.00 | N |
| ATOM | 2771 | CA | GLN | A | 171 | 28.334 | −7.179 | −17.621 | 1.00 | 0.00 | C |
| ATOM | 2772 | C | GLN | A | 171 | 28.253 | −8.476 | −16.811 | 1.00 | 0.00 | C |
| ATOM | 2773 | O | GLN | A | 171 | 28.926 | −8.619 | −15.795 | 1.00 | 0.00 | O |
| ATOM | 2774 | CB | GLN | A | 171 | 27.068 | −6.353 | −17.384 | 1.00 | 0.00 | C |
| ATOM | 2775 | CG | GLN | A | 171 | 27.139 | −5.569 | −16.050 | 1.00 | 0.00 | C |
| ATOM | 2776 | CD | GLN | A | 171 | 25.799 | −5.043 | −15.540 | 1.00 | 0.00 | C |
| ATOM | 2777 | OE1 | GLN | A | 171 | 24.905 | −4.722 | −16.320 | 1.00 | 0.00 | O |
| ATOM | 2778 | NE2 | GLN | A | 171 | 25.637 | −4.959 | −14.224 | 1.00 | 0.00 | N |
| ATOM | 2779 | H | GLN | A | 171 | 27.771 | −7.154 | −19.692 | 1.00 | 0.00 | H |
| ATOM | 2780 | HA | GLN | A | 171 | 29.211 | −6.623 | −17.281 | 1.00 | 0.00 | H |
| ATOM | 2781 | 1HB | GLN | A | 171 | 26.940 | −5.630 | −18.191 | 1.00 | 0.00 | H |
| ATOM | 2782 | 2HB | GLN | A | 171 | 26.178 | −6.984 | −17.413 | 1.00 | 0.00 | H |
| ATOM | 2783 | 1HG | GLN | A | 171 | 27.578 | −6.194 | −15.271 | 1.00 | 0.00 | H |
| ATOM | 2784 | 2HG | GLN | A | 171 | 27.807 | −4.720 | −16.180 | 1.00 | 0.00 | H |
| ATOM | 2785 | 1HE2 | GLN | A | 171 | 24.729 | −4.776 | −13.815 | 1.00 | 0.00 | H |
| ATOM | 2786 | 2HE2 | GLN | A | 171 | 26.388 | −5.233 | −13.610 | 1.00 | 0.00 | H |
| ATOM | 2787 | N | LEU | A | 172 | 27.430 | −9.418 | −17.269 | 1.00 | 0.00 | N |
| ATOM | 2788 | CA | LEU | A | 172 | 27.269 | −10.706 | −16.589 | 1.00 | 0.00 | C |
| ATOM | 2789 | C | LEU | A | 172 | 28.566 | −11.495 | −16.556 | 1.00 | 0.00 | C |
| ATOM | 2790 | O | LEU | A | 172 | 28.867 | −12.168 | −15.574 | 1.00 | 0.00 | O |
| ATOM | 2791 | CB | LEU | A | 172 | 26.187 | −11.544 | −17.270 | 1.00 | 0.00 | C |
| ATOM | 2792 | CG | LEU | A | 172 | 24.747 | −11.145 | −16.892 | 1.00 | 0.00 | C |
| ATOM | 2793 | CD1 | LEU | A | 172 | 23.739 | −11.736 | −17.889 | 1.00 | 0.00 | C |
| ATOM | 2794 | CD2 | LEU | A | 172 | 24.379 | −11.564 | −15.457 | 1.00 | 0.00 | C |
| ATOM | 2795 | H | LEU | A | 172 | 26.884 | −9.240 | −18.108 | 1.00 | 0.00 | H |
| ATOM | 2796 | HA | LEU | A | 172 | 27.019 | −10.512 | −15.548 | 1.00 | 0.00 | H |
| ATOM | 2797 | 1HB | LEU | A | 172 | 26.317 | −11.482 | −18.351 | 1.00 | 0.00 | H |
| ATOM | 2798 | 2HB | LEU | A | 172 | 26.315 | −12.602 | −17.029 | 1.00 | 0.00 | H |
| ATOM | 2799 | HG | LEU | A | 172 | 24.680 | −10.060 | −16.951 | 1.00 | 0.00 | H |
| ATOM | 2800 | 1HD1 | LEU | A | 172 | 22.716 | −11.465 | −17.625 | 1.00 | 0.00 | H |
| ATOM | 2801 | 2HD1 | LEU | A | 172 | 23.924 | −11.371 | −18.900 | 1.00 | 0.00 | H |
| ATOM | 2802 | 3HD1 | LEU | A | 172 | 23.793 | −12.825 | −17.910 | 1.00 | 0.00 | H |
| ATOM | 2803 | 1HD2 | LEU | A | 172 | 23.344 | −11.307 | −15.225 | 1.00 | 0.00 | H |
| ATOM | 2804 | 2HD2 | LEU | A | 172 | 24.485 | −12.640 | −15.318 | 1.00 | 0.00 | H |
| ATOM | 2805 | 3HD2 | LEU | A | 172 | 25.003 | −11.072 | −14.711 | 1.00 | 0.00 | H |
| ATOM | 2806 | N | VAL | A | 173 | 29.338 | −11.420 | −17.631 | 1.00 | 0.00 | N |
| ATOM | 2807 | CA | VAL | A | 173 | 30.609 | −12.131 | −17.676 | 1.00 | 0.00 | C |
| ATOM | 2808 | C | VAL | A | 173 | 31.558 | −11.541 | −16.636 | 1.00 | 0.00 | C |
| ATOM | 2809 | O | VAL | A | 173 | 32.305 | −12.269 | −15.987 | 1.00 | 0.00 | O |
| ATOM | 2810 | CB | VAL | A | 173 | 31.261 | −12.040 | −19.082 | 1.00 | 0.00 | C |
| ATOM | 2811 | CG1 | VAL | A | 173 | 32.772 | −12.358 | −19.164 | 1.00 | 0.00 | C |
| ATOM | 2812 | CG2 | VAL | A | 173 | 30.520 | −12.972 | −20.060 | 1.00 | 0.00 | C |
| ATOM | 2813 | H | VAL | A | 173 | 29.019 | −10.910 | −18.450 | 1.00 | 0.00 | H |
| ATOM | 2814 | HA | VAL | A | 173 | 30.447 | −13.181 | −17.423 | 1.00 | 0.00 | H |
| ATOM | 2815 | HB | VAL | A | 173 | 31.141 | −11.020 | −19.448 | 1.00 | 0.00 | H |
| ATOM | 2816 | 1HG1 | VAL | A | 173 | 33.109 | −12.402 | −20.200 | 1.00 | 0.00 | H |
| ATOM | 2817 | 2HG1 | VAL | A | 173 | 33.375 | −11.590 | −18.679 | 1.00 | 0.00 | H |
| ATOM | 2818 | 3HG1 | VAL | A | 173 | 33.006 | −13.317 | −18.699 | 1.00 | 0.00 | H |
| ATOM | 2819 | 1HG2 | VAL | A | 173 | 30.816 | −12.780 | −21.092 | 1.00 | 0.00 | H |
| ATOM | 2820 | 2HG2 | VAL | A | 173 | 30.733 | −14.020 | −19.843 | 1.00 | 0.00 | H |
| ATOM | 2821 | 3HG2 | VAL | A | 173 | 29.439 | −12.854 | −20.006 | 1.00 | 0.00 | H |
| ATOM | 2822 | N | GLU | A | 174 | 31.526 | −10.219 | −16.484 | 1.00 | 0.00 | N |
| ATOM | 2823 | CA | GLU | A | 174 | 32.376 | −9.535 | −15.513 | 1.00 | 0.00 | C |
| ATOM | 2824 | C | GLU | A | 174 | 31.950 | −9.904 | −14.092 | 1.00 | 0.00 | C |
| ATOM | 2825 | O | GLU | A | 174 | 32.772 | −9.950 | −13.179 | 1.00 | 0.00 | O |
| ATOM | 2826 | CB | GLU | A | 174 | 32.277 | −8.018 | −15.688 | 1.00 | 0.00 | C |
| ATOM | 2827 | CG | GLU | A | 174 | 33.034 | −7.479 | −16.927 | 1.00 | 0.00 | C |
| ATOM | 2828 | CD | GLU | A | 174 | 34.469 | −7.976 | −17.039 | 1.00 | 0.00 | C |
| ATOM | 2829 | OE1 | GLU | A | 174 | 35.284 | −7.701 | −16.140 | 1.00 | 0.00 | O |
| ATOM | 2830 | OE2 | GLU | A | 174 | 34.774 | −8.750 | −17.968 | 1.00 | 0.00 | O |
| ATOM | 2831 | H | GLU | A | 174 | 30.930 | −9.660 | −17.088 | 1.00 | 0.00 | H |
| ATOM | 2832 | HA | GLU | A | 174 | 33.403 | −9.884 | −15.627 | 1.00 | 0.00 | H |
| ATOM | 2833 | 1HB | GLU | A | 174 | 31.243 | −7.680 | −15.712 | 1.00 | 0.00 | H |
| ATOM | 2834 | 2HB | GLU | A | 174 | 32.724 | −7.537 | −14.816 | 1.00 | 0.00 | H |
| ATOM | 2835 | 1HG | GLU | A | 174 | 32.506 | −7.737 | −17.842 | 1.00 | 0.00 | H |
| ATOM | 2836 | 2HG | GLU | A | 174 | 33.071 | −6.393 | −16.888 | 1.00 | 0.00 | H |
| ATOM | 2837 | N | ILE | A | 175 | 30.659 | −10.161 | −13.908 | 1.00 | 0.00 | N |
| ATOM | 2838 | CA | ILE | A | 175 | 30.147 | −10.534 | −12.596 | 1.00 | 0.00 | C |
| ATOM | 2839 | C | ILE | A | 175 | 30.566 | −11.973 | −12.312 | 1.00 | 0.00 | C |
| ATOM | 2840 | O | ILE | A | 175 | 31.012 | −12.300 | −11.214 | 1.00 | 0.00 | O |
| ATOM | 2841 | CB | ILE | A | 175 | 28.605 | −10.432 | −12.537 | 1.00 | 0.00 | C |
| ATOM | 2842 | CG1 | ILE | A | 175 | 28.182 | −8.941 | −12.582 | 1.00 | 0.00 | C |
| ATOM | 2843 | CG2 | ILE | A | 175 | 27.977 | −11.083 | −11.281 | 1.00 | 0.00 | C |
| ATOM | 2844 | CD1 | ILE | A | 175 | 26.703 | −8.682 | −12.916 | 1.00 | 0.00 | C |
| ATOM | 2845 | H | ILE | A | 175 | 30.015 | −10.008 | −14.675 | 1.00 | 0.00 | H |
| ATOM | 2846 | HA | ILE | A | 175 | 30.579 | −9.901 | −11.823 | 1.00 | 0.00 | H |
| ATOM | 2847 | HB | ILE | A | 175 | 28.186 | −10.935 | −13.408 | 1.00 | 0.00 | H |
| ATOM | 2848 | 1HG1 | ILE | A | 175 | 28.421 | −8.475 | −11.626 | 1.00 | 0.00 | H |
| ATOM | 2849 | 2HG1 | ILE | A | 175 | 28.785 | −8.400 | −13.309 | 1.00 | 0.00 | H |
| ATOM | 2850 | 1HG2 | ILE | A | 175 | 26.903 | −10.913 | −11.219 | 1.00 | 0.00 | H |
| ATOM | 2851 | 2HG2 | ILE | A | 175 | 28.106 | −12.165 | −11.266 | 1.00 | 0.00 | H |
| ATOM | 2852 | 3HG2 | ILE | A | 175 | 28.432 | −10.685 | −10.374 | 1.00 | 0.00 | H |
| ATOM | 2853 | 1HD1 | ILE | A | 175 | 26.505 | −7.611 | −12.956 | 1.00 | 0.00 | H |
| ATOM | 2854 | 2HD1 | ILE | A | 175 | 26.433 | −9.097 | −13.885 | 1.00 | 0.00 | H |
| ATOM | 2855 | 3HD1 | ILE | A | 175 | 26.029 | −9.100 | −12.169 | 1.00 | 0.00 | H |
| ATOM | 2856 | N | ILE | A | 176 | 30.432 | −12.823 | −13.324 | 1.00 | 0.00 | N |
| ATOM | 2857 | CA | ILE | A | 176 | 30.792 | −14.227 | −13.191 | 1.00 | 0.00 | C |
| ATOM | 2858 | C | ILE | A | 176 | 32.285 | −14.402 | −12.915 | 1.00 | 0.00 | C |
| ATOM | 2859 | O | ILE | A | 176 | 32.684 | −15.301 | −12.176 | 1.00 | 0.00 | O |
| ATOM | 2860 | CB | ILE | A | 176 | 30.380 | −15.012 | −14.460 | 1.00 | 0.00 | C |
| ATOM | 2861 | CG1 | ILE | A | 176 | 28.839 | −15.135 | −14.534 | 1.00 | 0.00 | C |
| ATOM | 2862 | CG2 | ILE | A | 176 | 30.998 | −16.426 | −14.603 | 1.00 | 0.00 | C |
| ATOM | 2863 | CD1 | ILE | A | 176 | 28.288 | −15.453 | −15.933 | 1.00 | 0.00 | C |
| ATOM | 2864 | H | ILE | A | 176 | 29.987 | −12.509 | −14.184 | 1.00 | 0.00 | H |
| ATOM | 2865 | HA | ILE | A | 176 | 30.282 | −14.646 | −12.326 | 1.00 | 0.00 | H |
| ATOM | 2866 | HB | ILE | A | 176 | 30.715 | −14.434 | −15.322 | 1.00 | 0.00 | H |
| ATOM | 2867 | 1HG1 | ILE | A | 176 | 28.513 | −15.898 | −13.832 | 1.00 | 0.00 | H |
| ATOM | 2868 | 2HG1 | ILE | A | 176 | 28.363 | −14.218 | −14.185 | 1.00 | 0.00 | H |
| ATOM | 2869 | 1HG2 | ILE | A | 176 | 30.624 | −16.956 | −15.478 | 1.00 | 0.00 | H |
| ATOM | 2870 | 2HG2 | ILE | A | 176 | 32.078 | −16.389 | −14.723 | 1.00 | 0.00 | H |
| ATOM | 2871 | 3HG2 | ILE | A | 176 | 30.787 | −17.040 | −13.732 | 1.00 | 0.00 | H |
| ATOM | 2872 | 1HD1 | ILE | A | 176 | 27.197 | −15.463 | −15.922 | 1.00 | 0.00 | H |
| ATOM | 2873 | 2HD1 | ILE | A | 176 | 28.598 | −14.705 | −16.662 | 1.00 | 0.00 | H |
| ATOM | 2874 | 3HD1 | ILE | A | 176 | 28.616 | −16.427 | −16.289 | 1.00 | 0.00 | H |
| ATOM | 2875 | N | LYS | A | 177 | 33.110 | −13.538 | −13.490 | 1.00 | 0.00 | N |
| ATOM | 2876 | CA | LYS | A | 177 | 34.544 | −13.631 | −13.268 | 1.00 | 0.00 | C |
| ATOM | 2877 | C | LYS | A | 177 | 34.888 | −13.423 | −11.795 | 1.00 | 0.00 | C |
| ATOM | 2878 | O | LYS | A | 177 | 35.833 | −14.026 | −11.278 | 1.00 | 0.00 | O |
| ATOM | 2879 | CB | LYS | A | 177 | 35.277 | −12.600 | −14.122 | 1.00 | 0.00 | C |
| ATOM | 2880 | CG | LYS | A | 177 | 35.489 | −13.037 | −15.585 | 1.00 | 0.00 | C |
| ATOM | 2881 | CD | LYS | A | 177 | 35.711 | −11.874 | −16.573 | 1.00 | 0.00 | C |
| ATOM | 2882 | CE | LYS | A | 177 | 36.788 | −10.859 | −16.151 | 1.00 | 0.00 | C |
| ATOM | 2883 | NZ | LYS | A | 177 | 37.003 | −9.838 | −17.184 | 1.00 | 0.00 | N |
| ATOM | 2884 | H | LYS | A | 177 | 32.741 | −12.864 | −14.155 | 1.00 | 0.00 | H |
| ATOM | 2885 | HA | LYS | A | 177 | 34.881 | −14.624 | −13.551 | 1.00 | 0.00 | H |
| ATOM | 2886 | 1HB | LYS | A | 177 | 34.726 | −11.658 | −14.088 | 1.00 | 0.00 | H |
| ATOM | 2887 | 2HB | LYS | A | 177 | 36.257 | −12.381 | −13.692 | 1.00 | 0.00 | H |
| ATOM | 2888 | 1HG | LYS | A | 177 | 36.327 | −13.733 | −15.633 | 1.00 | 0.00 | H |
| ATOM | 2889 | 2HG | LYS | A | 177 | 34.619 | −13.598 | −15.928 | 1.00 | 0.00 | H |
| ATOM | 2890 | 1HD | LYS | A | 177 | 35.961 | −12.293 | −17.548 | 1.00 | 0.00 | H |
| ATOM | 2891 | 2HD | LYS | A | 177 | 34.769 | −11.346 | −16.714 | 1.00 | 0.00 | H |
| ATOM | 2892 | 1HE | LYS | A | 177 | 36.486 | −10.344 | −15.238 | 1.00 | 0.00 | H |
| ATOM | 2893 | 2HE | LYS | A | 177 | 37.734 | −11.360 | −15.945 | 1.00 | 0.00 | H |
| ATOM | 2894 | 1HZ | LYS | A | 177 | 37.588 | −9.101 | −16.834 | 1.00 | 0.00 | H |
| ATOM | 2895 | 2HZ | LYS | A | 177 | 37.385 | −10.225 | −18.026 | 1.00 | 0.00 | H |
| ATOM | 2896 | 3HZ | LYS | A | 177 | 36.100 | −9.385 | −17.421 | 1.00 | 0.00 | H |
| ATOM | 2897 | N | VAL | A | 178 | 34.118 | −12.577 | −11.116 | 1.00 | 0.00 | N |
| ATOM | 2898 | CA | VAL | A | 178 | 34.367 | −12.307 | −9.707 | 1.00 | 0.00 | C |
| ATOM | 2899 | C | VAL | A | 178 | 33.604 | −13.270 | −8.805 | 1.00 | 0.00 | C |
| ATOM | 2900 | O | VAL | A | 178 | 34.195 | −13.912 | −7.940 | 1.00 | 0.00 | O |
| ATOM | 2901 | CB | VAL | A | 178 | 33.966 | −10.861 | −9.320 | 1.00 | 0.00 | C |
| ATOM | 2902 | CG1 | VAL | A | 178 | 34.209 | −10.515 | −7.835 | 1.00 | 0.00 | C |
| ATOM | 2903 | CG2 | VAL | A | 178 | 34.711 | −9.827 | −10.189 | 1.00 | 0.00 | C |
| ATOM | 2904 | H | VAL | A | 178 | 33.339 | −12.127 | −11.579 | 1.00 | 0.00 | H |
| ATOM | 2905 | HA | VAL | A | 178 | 35.424 | −12.383 | −9.506 | 1.00 | 0.00 | H |
| ATOM | 2906 | HB | VAL | A | 178 | 32.899 | −10.730 | −9.514 | 1.00 | 0.00 | H |
| ATOM | 2907 | 1HG1 | VAL | A | 178 | 34.025 | −9.459 | −7.639 | 1.00 | 0.00 | H |
| ATOM | 2908 | 2HG1 | VAL | A | 178 | 33.556 | −11.072 | −7.163 | 1.00 | 0.00 | H |
| ATOM | 2909 | 3HG1 | VAL | A | 178 | 35.236 | −10.732 | −7.543 | 1.00 | 0.00 | H |
| ATOM | 2910 | 1HG2 | VAL | A | 178 | 34.416 | −8.808 | −9.936 | 1.00 | 0.00 | H |
| ATOM | 2911 | 2HG2 | VAL | A | 178 | 35.790 | −9.900 | −10.054 | 1.00 | 0.00 | H |
| ATOM | 2912 | 3HG2 | VAL | A | 178 | 34.504 | −9.958 | −11.251 | 1.00 | 0.00 | H |
| ATOM | 2913 | N | LEU | A | 179 | 32.296 | −13.379 | −9.014 | 1.00 | 0.00 | N |
| ATOM | 2914 | CA | LEU | A | 179 | 31.458 | −14.247 | −8.189 | 1.00 | 0.00 | C |
| ATOM | 2915 | C | LEU | A | 179 | 31.440 | −15.713 | −8.598 | 1.00 | 0.00 | C |
| ATOM | 2916 | O | LEU | A | 179 | 30.957 | −16.551 | −7.848 | 1.00 | 0.00 | O |
| ATOM | 2917 | CB | LEU | A | 179 | 30.010 | −13.738 | −8.173 | 1.00 | 0.00 | C |
| ATOM | 2918 | CG | LEU | A | 179 | 29.791 | −12.307 | −7.640 | 1.00 | 0.00 | C |
| ATOM | 2919 | CD1 | LEU | A | 179 | 28.287 | −11.999 | −7.575 | 1.00 | 0.00 | C |
| ATOM | 2920 | CD2 | LEU | A | 179 | 30.424 | −12.075 | −6.259 | 1.00 | 0.00 | C |
| ATOM | 2921 | H | LEU | A | 179 | 31.854 | −12.868 | −9.778 | 1.00 | 0.00 | H |
| ATOM | 2922 | HA | LEU | A | 179 | 31.827 | −14.238 | −7.161 | 1.00 | 0.00 | H |
| ATOM | 2923 | 1HB | LEU | A | 179 | 29.600 | −13.799 | −9.183 | 1.00 | 0.00 | H |
| ATOM | 2924 | 2HB | LEU | A | 179 | 29.405 | −14.416 | −7.567 | 1.00 | 0.00 | H |
| ATOM | 2925 | HG | LEU | A | 179 | 30.245 | −11.604 | −8.341 | 1.00 | 0.00 | H |
| ATOM | 2926 | 1HD1 | LEU | A | 179 | 28.115 | −10.973 | −7.249 | 1.00 | 0.00 | H |
| ATOM | 2927 | 2HD1 | LEU | A | 179 | 27.806 | −12.134 | −8.543 | 1.00 | 0.00 | H |
| ATOM | 2928 | 3HD1 | LEU | A | 179 | 27.778 | −12.652 | −6.865 | 1.00 | 0.00 | H |
| ATOM | 2929 | 1HD2 | LEU | A | 179 | 30.169 | −11.085 | −5.886 | 1.00 | 0.00 | H |
| ATOM | 2930 | 2HD2 | LEU | A | 179 | 30.062 | −12.798 | −5.527 | 1.00 | 0.00 | H |
| ATOM | 2931 | 3HD2 | LEU | A | 179 | 31.512 | −12.135 | −6.288 | 1.00 | 0.00 | H |
| ATOM | 2932 | N | GLY | A | 180 | 31.963 | −16.030 | −9.775 | 1.00 | 0.00 | N |
| ATOM | 2933 | CA | GLY | A | 180 | 31.930 | −17.408 | −10.224 | 1.00 | 0.00 | C |
| ATOM | 2934 | C | GLY | A | 180 | 30.571 | −17.677 | −10.846 | 1.00 | 0.00 | C |
| ATOM | 2935 | O | GLY | A | 180 | 29.648 | −16.882 | −10.689 | 1.00 | 0.00 | O |
| ATOM | 2936 | H | GLY | A | 180 | 32.301 | −15.331 | −10.429 | 1.00 | 0.00 | H |
| ATOM | 2937 | 1HA | GLY | A | 180 | 32.706 | −17.523 | −10.973 | 1.00 | 0.00 | H |
| ATOM | 2938 | 2HA | GLY | A | 180 | 32.138 | −18.126 | −9.429 | 1.00 | 0.00 | H |
| ATOM | 2939 | N | THR | A | 181 | 30.436 | −18.793 | −11.554 | 1.00 | 0.00 | N |
| ATOM | 2940 | CA | THR | A | 181 | 29.174 | −19.134 | −12.207 | 1.00 | 0.00 | C |
| ATOM | 2941 | C | THR | A | 181 | 28.030 | −19.316 | −11.212 | 1.00 | 0.00 | C |
| ATOM | 2942 | O | THR | A | 181 | 28.186 | −19.962 | −10.179 | 1.00 | 0.00 | O |
| ATOM | 2943 | CB | THR | A | 181 | 29.329 | −20.418 | −13.049 | 1.00 | 0.00 | C |
| ATOM | 2944 | OG1 | THR | A | 181 | 28.149 | −20.796 | −13.741 | 1.00 | 0.00 | O |
| ATOM | 2945 | CG2 | THR | A | 181 | 29.852 | −21.648 | −12.280 | 1.00 | 0.00 | C |
| ATOM | 2946 | H | THR | A | 181 | 31.236 | −19.401 | −11.694 | 1.00 | 0.00 | H |
| ATOM | 2947 | HA | THR | A | 181 | 28.977 | −18.307 | −12.888 | 1.00 | 0.00 | H |
| ATOM | 2948 | HB | THR | A | 181 | 30.056 | −20.189 | −13.830 | 1.00 | 0.00 | H |
| ATOM | 2949 | HG1 | THR | A | 181 | 27.491 | −21.081 | −13.118 | 1.00 | 0.00 | H |
| ATOM | 2950 | 1HG2 | THR | A | 181 | 30.048 | −22.479 | −12.953 | 1.00 | 0.00 | H |
| ATOM | 2951 | 2HG2 | THR | A | 181 | 30.789 | −21.436 | −11.765 | 1.00 | 0.00 | H |
| ATOM | 2952 | 3HG2 | THR | A | 181 | 29.141 | −22.003 | −11.534 | 1.00 | 0.00 | H |
| ATOM | 2953 | N | PRO | A | 182 | 26.859 | −18.737 | −11.509 | 1.00 | 0.00 | N |
| ATOM | 2954 | CA | PRO | A | 182 | 25.715 | −18.863 | −10.605 | 1.00 | 0.00 | C |
| ATOM | 2955 | C | PRO | A | 182 | 25.081 | −20.253 | −10.612 | 1.00 | 0.00 | C |
| ATOM | 2956 | O | PRO | A | 182 | 24.840 | −20.841 | −11.665 | 1.00 | 0.00 | O |
| ATOM | 2957 | CB | PRO | A | 182 | 24.746 | −17.799 | −11.103 | 1.00 | 0.00 | C |
| ATOM | 2958 | CG | PRO | A | 182 | 25.028 | −17.638 | −12.585 | 1.00 | 0.00 | C |
| ATOM | 2959 | CD | PRO | A | 182 | 26.520 | −17.937 | −12.692 | 1.00 | 0.00 | C |
| ATOM | 2960 | HA | PRO | A | 182 | 26.030 | −18.601 | −9.594 | 1.00 | 0.00 | H |
| ATOM | 2961 | 1HB | PRO | A | 182 | 23.704 | −18.023 | −10.883 | 1.00 | 0.00 | H |
| ATOM | 2962 | 2HB | PRO | A | 182 | 24.965 | −16.859 | −10.597 | 1.00 | 0.00 | H |
| ATOM | 2963 | 1HG | PRO | A | 182 | 24.763 | −16.652 | −12.967 | 1.00 | 0.00 | H |
| ATOM | 2964 | 2HG | PRO | A | 182 | 24.464 | −18.380 | −13.151 | 1.00 | 0.00 | H |
| ATOM | 2965 | 1HD | PRO | A | 182 | 27.073 | −17.001 | −12.642 | 1.00 | 0.00 | H |
| ATOM | 2966 | 2HD | PRO | A | 182 | 26.758 | −18.439 | −13.630 | 1.00 | 0.00 | H |
| ATOM | 2967 | N | THR | A | 183 | 24.813 | −20.758 | −9.415 | 1.00 | 0.00 | N |
| ATOM | 2968 | CA | THR | A | 183 | 24.192 | −22.059 | −9.215 | 1.00 | 0.00 | C |
| ATOM | 2969 | C | THR | A | 183 | 22.843 | −22.066 | −9.913 | 1.00 | 0.00 | C |
| ATOM | 2970 | O | THR | A | 183 | 22.316 | −21.011 | −10.258 | 1.00 | 0.00 | O |
| ATOM | 2971 | CB | THR | A | 183 | 23.998 | −22.305 | −7.706 | 1.00 | 0.00 | C |
| ATOM | 2972 | OG1 | THR | A | 183 | 25.208 | −22.034 | −7.015 | 1.00 | 0.00 | O |
| ATOM | 2973 | CG2 | THR | A | 183 | 23.672 | −23.755 | −7.322 | 1.00 | 0.00 | C |
| ATOM | 2974 | H | THR | A | 183 | 25.032 | −20.197 | −8.590 | 1.00 | 0.00 | H |
| ATOM | 2975 | HA | THR | A | 183 | 24.815 | −22.835 | −9.665 | 1.00 | 0.00 | H |
| ATOM | 2976 | HB | THR | A | 183 | 23.227 | −21.646 | −7.305 | 1.00 | 0.00 | H |
| ATOM | 2977 | HG1 | THR | A | 183 | 25.358 | −21.088 | −7.013 | 1.00 | 0.00 | H |
| ATOM | 2978 | 1HG2 | THR | A | 183 | 23.603 | −23.870 | −6.239 | 1.00 | 0.00 | H |
| ATOM | 2979 | 2HG2 | THR | A | 183 | 22.720 | −24.082 | −7.726 | 1.00 | 0.00 | H |
| ATOM | 2980 | 3HG2 | THR | A | 183 | 24.436 | −24.447 | −7.678 | 1.00 | 0.00 | H |
| ATOM | 2981 | N | ARG | A | 184 | 22.284 | −23.251 | −10.132 | 1.00 | 0.00 | N |
| ATOM | 2982 | CA | ARG | A | 184 | 20.984 | −23.344 | −10.777 | 1.00 | 0.00 | C |
| ATOM | 2983 | C | ARG | A | 184 | 19.945 | −22.693 | −9.867 | 1.00 | 0.00 | C |
| ATOM | 2984 | O | ARG | A | 184 | 19.047 | −21.995 | −10.338 | 1.00 | 0.00 | O |
| ATOM | 2985 | CB | ARG | A | 184 | 20.620 | −24.808 | −11.038 | 1.00 | 0.00 | C |
| ATOM | 2986 | CG | ARG | A | 184 | 21.564 | −25.503 | −12.033 | 1.00 | 0.00 | C |
| ATOM | 2987 | CD | ARG | A | 184 | 21.143 | −26.948 | −12.324 | 1.00 | 0.00 | C |
| ATOM | 2988 | NE | ARG | A | 184 | 22.029 | −27.572 | −13.317 | 1.00 | 0.00 | N |
| ATOM | 2989 | CZ | ARG | A | 184 | 21.856 | −28.769 | −13.890 | 1.00 | 0.00 | C |
| ATOM | 2990 | NH1 | ARG | A | 184 | 20.805 | −29.527 | −13.582 | 1.00 | 0.00 | N |
| ATOM | 2991 | NH2 | ARG | A | 184 | 22.746 | −29.203 | −14.778 | 1.00 | 0.00 | N |
| ATOM | 2992 | H | ARG | A | 184 | 22.773 | −24.093 | −9.869 | 1.00 | 0.00 | H |
| ATOM | 2993 | HA | ARG | A | 184 | 21.022 | −22.792 | −11.719 | 1.00 | 0.00 | H |
| ATOM | 2994 | 1HB | ARG | A | 184 | 20.602 | −25.364 | −10.098 | 1.00 | 0.00 | H |
| ATOM | 2995 | 2HB | ARG | A | 184 | 19.603 | −24.854 | −11.431 | 1.00 | 0.00 | H |
| ATOM | 2996 | 1HG | ARG | A | 184 | 21.586 | −24.934 | −12.964 | 1.00 | 0.00 | H |
| ATOM | 2997 | 2HG | ARG | A | 184 | 22.583 | −25.496 | −11.643 | 1.00 | 0.00 | H |
| ATOM | 2998 | 1HD | ARG | A | 184 | 21.159 | −27.542 | −11.409 | 1.00 | 0.00 | H |
| ATOM | 2999 | 2HD | ARG | A | 184 | 20.121 | −26.967 | −12.705 | 1.00 | 0.00 | H |
| ATOM | 3000 | HE | ARG | A | 184 | 22.849 | −27.026 | −13.555 | 1.00 | 0.00 | H |
| ATOM | 3001 | 1HH1 | ARG | A | 184 | 20.650 | −30.433 | −14.001 | 1.00 | 0.00 | H |
| ATOM | 3002 | 2HH1 | ARG | A | 184 | 20.118 | −29.224 | −12.910 | 1.00 | 0.00 | H |
| ATOM | 3003 | 1HH2 | ARG | A | 184 | 22.656 | −30.098 | −15.235 | 1.00 | 0.00 | H |
| ATOM | 3004 | 2HH2 | ARG | A | 184 | 23.554 | −28.656 | −15.038 | 1.00 | 0.00 | H |
| ATOM | 3005 | N | GLU | A | 185 | 20.077 | −22.919 | −8.562 | 1.00 | 0.00 | N |
| ATOM | 3006 | CA | GLU | A | 185 | 19.156 | −22.337 | −7.586 | 1.00 | 0.00 | C |
| ATOM | 3007 | C | GLU | A | 185 | 19.314 | −20.815 | −7.566 | 1.00 | 0.00 | C |
| ATOM | 3008 | O | GLU | A | 185 | 18.330 | −20.082 | −7.491 | 1.00 | 0.00 | O |
| ATOM | 3009 | CB | GLU | A | 185 | 19.426 | −22.898 | −6.182 | 1.00 | 0.00 | C |
| ATOM | 3010 | CG | GLU | A | 185 | 18.851 | −24.313 | −5.925 | 1.00 | 0.00 | C |
| ATOM | 3011 | CD | GLU | A | 185 | 19.518 | −25.457 | −6.685 | 1.00 | 0.00 | C |
| ATOM | 3012 | OE1 | GLU | A | 185 | 20.668 | −25.286 | −7.135 | 1.00 | 0.00 | O |
| ATOM | 3013 | OE2 | GLU | A | 185 | 18.867 | −26.510 | −6.820 | 1.00 | 0.00 | O |
| ATOM | 3014 | H | GLU | A | 185 | 20.823 | −23.518 | −8.248 | 1.00 | 0.00 | H |
| ATOM | 3015 | HA | GLU | A | 185 | 18.135 | −22.571 | −7.893 | 1.00 | 0.00 | H |
| ATOM | 3016 | 1HB | GLU | A | 185 | 20.495 | −22.885 | −5.964 | 1.00 | 0.00 | H |
| ATOM | 3017 | 2HB | GLU | A | 185 | 18.973 | −22.237 | −5.441 | 1.00 | 0.00 | H |
| ATOM | 3018 | 1HG | GLU | A | 185 | 18.939 | −24.545 | −4.864 | 1.00 | 0.00 | H |
| ATOM | 3019 | 2HG | GLU | A | 185 | 17.784 | −24.323 | −6.149 | 1.00 | 0.00 | H |
| ATOM | 3020 | N | GLN | A | 186 | 20.557 | −20.349 | −7.634 | 1.00 | 0.00 | N |
| ATOM | 3021 | CA | GLN | A | 186 | 20.839 | −18.920 | −7.635 | 1.00 | 0.00 | C |
| ATOM | 3022 | C | GLN | A | 186 | 20.196 | −18.255 | −8.843 | 1.00 | 0.00 | C |
| ATOM | 3023 | O | GLN | A | 186 | 19.614 | −17.180 | −8.732 | 1.00 | 0.00 | O |
| ATOM | 3024 | CB | GLN | A | 186 | 22.344 | −18.689 | −7.650 | 1.00 | 0.00 | C |
| ATOM | 3025 | CG | GLN | A | 186 | 22.970 | −18.815 | −6.245 | 1.00 | 0.00 | C |
| ATOM | 3026 | CD | GLN | A | 186 | 24.492 | −18.694 | −6.253 | 1.00 | 0.00 | C |
| ATOM | 3027 | OE1 | GLN | A | 186 | 25.156 | −19.281 | −7.102 | 1.00 | 0.00 | O |
| ATOM | 3028 | NE2 | GLN | A | 186 | 25.070 | −17.955 | −5.318 | 1.00 | 0.00 | N |
| ATOM | 3029 | H | GLN | A | 186 | 21.309 | −20.996 | −7.785 | 1.00 | 0.00 | H |
| ATOM | 3030 | HA | GLN | A | 186 | 20.428 | −18.450 | −6.745 | 1.00 | 0.00 | H |
| ATOM | 3031 | 1HB | GLN | A | 186 | 22.827 | −19.353 | −8.361 | 1.00 | 0.00 | H |
| ATOM | 3032 | 2HB | GLN | A | 186 | 22.559 | −17.681 | −8.010 | 1.00 | 0.00 | H |
| ATOM | 3033 | 1HG | GLN | A | 186 | 22.556 | −18.046 | −5.595 | 1.00 | 0.00 | H |
| ATOM | 3034 | 2HG | GLN | A | 186 | 22.711 | −19.774 | −5.796 | 1.00 | 0.00 | H |
| ATOM | 3035 | 1HE2 | GLN | A | 186 | 26.072 | −17.856 | −5.306 | 1.00 | 0.00 | H |
| ATOM | 3036 | 2HE2 | GLN | A | 186 | 24.536 | −17.434 | −4.631 | 1.00 | 0.00 | H |
| ATOM | 3037 | N | ILE | A | 187 | 20.304 | −18.897 | −9.999 | 1.00 | 0.00 | N |
| ATOM | 3038 | CA | ILE | A | 187 | 19.706 | −18.368 | −11.215 | 1.00 | 0.00 | C |
| ATOM | 3039 | C | ILE | A | 187 | 18.194 | −18.326 | −11.027 | 1.00 | 0.00 | C |
| ATOM | 3040 | O | ILE | A | 187 | 17.523 | −17.390 | −11.456 | 1.00 | 0.00 | O |
| ATOM | 3041 | CB | ILE | A | 187 | 20.040 | −19.259 | −12.425 | 1.00 | 0.00 | C |
| ATOM | 3042 | CG1 | ILE | A | 187 | 21.541 | −19.154 | −12.784 | 1.00 | 0.00 | C |
| ATOM | 3043 | CG2 | ILE | A | 187 | 19.211 | −18.944 | −13.699 | 1.00 | 0.00 | C |
| ATOM | 3044 | CD1 | ILE | A | 187 | 22.061 | −20.284 | −13.688 | 1.00 | 0.00 | C |
| ATOM | 3045 | H | ILE | A | 187 | 20.866 | −19.741 | −10.060 | 1.00 | 0.00 | H |
| ATOM | 3046 | HA | ILE | A | 187 | 20.058 | −17.348 | −11.389 | 1.00 | 0.00 | H |
| ATOM | 3047 | HB | ILE | A | 187 | 19.832 | −20.294 | −12.153 | 1.00 | 0.00 | H |
| ATOM | 3048 | 1HG1 | ILE | A | 187 | 21.749 | −18.188 | −13.247 | 1.00 | 0.00 | H |
| ATOM | 3049 | 2HG1 | ILE | A | 187 | 22.138 | −19.170 | −11.879 | 1.00 | 0.00 | H |
| ATOM | 3050 | 1HG2 | ILE | A | 187 | 19.518 | −19.552 | −14.549 | 1.00 | 0.00 | H |
| ATOM | 3051 | 2HG2 | ILE | A | 187 | 18.147 | −19.145 | −13.570 | 1.00 | 0.00 | H |
| ATOM | 3052 | 3HG2 | ILE | A | 187 | 19.316 | −17.899 | −13.992 | 1.00 | 0.00 | H |
| ATOM | 3053 | 1HD1 | ILE | A | 187 | 23.141 | −20.208 | −13.816 | 1.00 | 0.00 | H |
| ATOM | 3054 | 2HD1 | ILE | A | 187 | 21.863 | −21.261 | −13.251 | 1.00 | 0.00 | H |
| ATOM | 3055 | 3HD1 | ILE | A | 187 | 21.619 | −20.259 | −14.683 | 1.00 | 0.00 | H |
| ATOM | 3056 | N | ARG | A | 188 | 17.673 | −19.356 | −10.372 | 1.00 | 0.00 | N |
| ATOM | 3057 | CA | ARG | A | 188 | 16.248 | −19.476 | −10.098 | 1.00 | 0.00 | C |
| ATOM | 3058 | C | ARG | A | 188 | 15.818 | −18.369 | −9.133 | 1.00 | 0.00 | C |
| ATOM | 3059 | O | ARG | A | 188 | 14.718 | −17.825 | −9.246 | 1.00 | 0.00 | O |
| ATOM | 3060 | CB | ARG | A | 188 | 15.966 | −20.858 | −9.495 | 1.00 | 0.00 | C |
| ATOM | 3061 | CG | ARG | A | 188 | 14.486 | −21.265 | −9.439 | 1.00 | 0.00 | C |
| ATOM | 3062 | CD | ARG | A | 188 | 14.276 | −22.597 | −8.706 | 1.00 | 0.00 | C |
| ATOM | 3063 | NE | ARG | A | 188 | 12.858 | −22.981 | −8.699 | 1.00 | 0.00 | N |
| ATOM | 3064 | CZ | ARG | A | 188 | 12.350 | −24.124 | −8.223 | 1.00 | 0.00 | C |
| ATOM | 3065 | NH1 | ARG | A | 188 | 13.141 | −25.055 | −7.691 | 1.00 | 0.00 | N |
| ATOM | 3066 | NH2 | ARG | A | 188 | 11.038 | −24.331 | −8.284 | 1.00 | 0.00 | N |
| ATOM | 3067 | H | ARG | A | 188 | 18.289 | −20.107 | −10.081 | 1.00 | 0.00 | H |
| ATOM | 3068 | HA | ARG | A | 188 | 15.696 | −19.363 | −11.032 | 1.00 | 0.00 | H |
| ATOM | 3069 | 1HB | ARG | A | 188 | 16.497 | −21.618 | −10.068 | 1.00 | 0.00 | H |
| ATOM | 3070 | 2HB | ARG | A | 188 | 16.350 | −20.921 | −8.482 | 1.00 | 0.00 | H |
| ATOM | 3071 | 1HG | ARG | A | 188 | 13.900 | −20.489 | −8.944 | 1.00 | 0.00 | H |
| ATOM | 3072 | 2HG | ARG | A | 188 | 14.107 | −21.359 | −10.456 | 1.00 | 0.00 | H |
| ATOM | 3073 | 1HD | ARG | A | 188 | 14.854 | −23.384 | −9.191 | 1.00 | 0.00 | H |
| ATOM | 3074 | 2HD | ARG | A | 188 | 14.628 | −22.524 | −7.676 | 1.00 | 0.00 | H |
| ATOM | 3075 | HE | ARG | A | 188 | 12.227 | −22.286 | −9.080 | 1.00 | 0.00 | H |
| ATOM | 3076 | 1HH1 | ARG | A | 188 | 12.781 | −25.924 | −7.324 | 1.00 | 0.00 | H |
| ATOM | 3077 | 2HH1 | ARG | A | 188 | 14.139 | −24.924 | −7.629 | 1.00 | 0.00 | H |
| ATOM | 3078 | 1HH2 | ARG | A | 188 | 10.610 | −25.176 | −7.936 | 1.00 | 0.00 | H |
| ATOM | 3079 | 2HH2 | ARG | A | 188 | 10.406 | −23.650 | −8.679 | 1.00 | 0.00 | H |
| ATOM | 3080 | N | GLU | A | 189 | 16.697 | −18.043 | −8.189 | 1.00 | 0.00 | N |
| ATOM | 3081 | CA | GLU | A | 189 | 16.443 | −16.996 | −7.199 | 1.00 | 0.00 | C |
| ATOM | 3082 | C | GLU | A | 189 | 16.323 | −15.623 | −7.864 | 1.00 | 0.00 | C |
| ATOM | 3083 | O | GLU | A | 189 | 15.480 | −14.808 | −7.491 | 1.00 | 0.00 | O |
| ATOM | 3084 | CB | GLU | A | 189 | 17.581 | −16.945 | −6.176 | 1.00 | 0.00 | C |
| ATOM | 3085 | CG | GLU | A | 189 | 17.473 | −18.042 | −5.086 | 1.00 | 0.00 | C |
| ATOM | 3086 | CD | GLU | A | 189 | 16.267 | −17.912 | −4.157 | 1.00 | 0.00 | C |
| ATOM | 3087 | OE1 | GLU | A | 189 | 15.703 | −16.804 | −4.068 | 1.00 | 0.00 | O |
| ATOM | 3088 | OE2 | GLU | A | 189 | 15.900 | −18.935 | −3.548 | 1.00 | 0.00 | O |
| ATOM | 3089 | H | GLU | A | 189 | 17.498 | −18.655 | −8.044 | 1.00 | 0.00 | H |
| ATOM | 3090 | HA | GLU | A | 189 | 15.505 | −17.216 | −6.684 | 1.00 | 0.00 | H |
| ATOM | 3091 | 1HB | GLU | A | 189 | 18.561 | −16.983 | −6.632 | 1.00 | 0.00 | H |
| ATOM | 3092 | 2HB | GLU | A | 189 | 17.560 | −15.985 | −5.660 | 1.00 | 0.00 | H |
| ATOM | 3093 | 1HG | GLU | A | 189 | 17.421 | −19.030 | −5.532 | 1.00 | 0.00 | H |
| ATOM | 3094 | 2HG | GLU | A | 189 | 18.368 | −18.037 | −4.467 | 1.00 | 0.00 | H |
| ATOM | 3095 | N | MET | A | 190 | 17.180 | −15.375 | −8.846 | 1.00 | 0.00 | N |
| ATOM | 3096 | CA | MET | A | 190 | 17.181 | −14.108 | −9.560 | 1.00 | 0.00 | C |
| ATOM | 3097 | C | MET | A | 190 | 15.986 | −13.978 | −10.487 | 1.00 | 0.00 | C |
| ATOM | 3098 | O | MET | A | 190 | 15.235 | −13.008 | −10.418 | 1.00 | 0.00 | O |
| ATOM | 3099 | CB | MET | A | 190 | 18.468 | −13.974 | −10.364 | 1.00 | 0.00 | C |
| ATOM | 3100 | CG | MET | A | 190 | 19.663 | −13.724 | −9.422 | 1.00 | 0.00 | C |
| ATOM | 3101 | SD | MET | A | 190 | 21.280 | −13.518 | −10.203 | 1.00 | 0.00 | S |
| ATOM | 3102 | CE | MET | A | 190 | 21.671 | −15.183 | −10.781 | 1.00 | 0.00 | C |
| ATOM | 3103 | H | MET | A | 190 | 17.856 | −16.094 | −9.082 | 1.00 | 0.00 | H |
| ATOM | 3104 | HA | MET | A | 190 | 17.137 | −13.292 | −8.835 | 1.00 | 0.00 | H |
| ATOM | 3105 | 1HB | MET | A | 190 | 18.645 | −14.868 | −10.964 | 1.00 | 0.00 | H |
| ATOM | 3106 | 2HB | MET | A | 190 | 18.400 | −13.148 | −11.068 | 1.00 | 0.00 | H |
| ATOM | 3107 | 1HG | MET | A | 190 | 19.481 | −12.810 | −8.853 | 1.00 | 0.00 | H |
| ATOM | 3108 | 2HG | MET | A | 190 | 19.750 | −14.520 | −8.682 | 1.00 | 0.00 | H |
| ATOM | 3109 | 1HE | MET | A | 190 | 22.698 | −15.223 | −11.145 | 1.00 | 0.00 | H |
| ATOM | 3110 | 2HE | MET | A | 190 | 21.567 | −15.908 | −9.976 | 1.00 | 0.00 | H |
| ATOM | 3111 | 3HE | MET | A | 190 | 21.012 | −15.443 | −11.607 | 1.00 | 0.00 | H |
| ATOM | 3112 | N | ASN | A | 191 | 15.824 | −14.960 | −11.363 | 1.00 | 0.00 | N |
| ATOM | 3113 | CA | ASN | A | 191 | 14.725 | −14.967 | −12.317 | 1.00 | 0.00 | C |
| ATOM | 3114 | C | ASN | A | 191 | 14.590 | −16.365 | −12.888 | 1.00 | 0.00 | C |
| ATOM | 3115 | O | ASN | A | 191 | 15.363 | −16.768 | −13.761 | 1.00 | 0.00 | O |
| ATOM | 3116 | CB | ASN | A | 191 | 14.992 | −13.980 | −13.456 | 1.00 | 0.00 | C |
| ATOM | 3117 | CG | ASN | A | 191 | 13.774 | −13.827 | −14.390 | 1.00 | 0.00 | C |
| ATOM | 3118 | OD1 | ASN | A | 191 | 12.651 | −14.184 | −14.043 | 1.00 | 0.00 | O |
| ATOM | 3119 | ND2 | ASN | A | 191 | 13.954 | −13.368 | −15.621 | 1.00 | 0.00 | N |
| ATOM | 3120 | H | ASN | A | 191 | 16.456 | −15.759 | −11.356 | 1.00 | 0.00 | H |
| ATOM | 3121 | HA | ASN | A | 191 | 13.831 | −14.636 | −11.791 | 1.00 | 0.00 | H |
| ATOM | 3122 | 1HB | ASN | A | 191 | 15.253 | −13.004 | −13.071 | 1.00 | 0.00 | H |
| ATOM | 3123 | 2HB | ASN | A | 191 | 15.856 | −14.292 | −14.044 | 1.00 | 0.00 | H |
| ATOM | 3124 | 1HD2 | ASN | A | 191 | 13.145 | −13.238 | −16.207 | 1.00 | 0.00 | H |
| ATOM | 3125 | 2HD2 | ASN | A | 191 | 14.848 | −13.001 | −15.953 | 1.00 | 0.00 | H |
| ATOM | 3126 | N | PRO | A | 192 | 13.603 | −17.128 | −12.398 | 1.00 | 0.00 | N |
| ATOM | 3127 | CA | PRO | A | 192 | 13.382 | −18.495 | −12.880 | 1.00 | 0.00 | C |
| ATOM | 3128 | C | PRO | A | 192 | 12.685 | −18.493 | −14.236 | 1.00 | 0.00 | C |
| ATOM | 3129 | O | PRO | A | 192 | 11.578 | −19.013 | −14.372 | 1.00 | 0.00 | O |
| ATOM | 3130 | CB | PRO | A | 192 | 12.516 | −19.116 | −11.791 | 1.00 | 0.00 | C |
| ATOM | 3131 | CG | PRO | A | 192 | 11.660 | −17.967 | −11.276 | 1.00 | 0.00 | C |
| ATOM | 3132 | CD | PRO | A | 192 | 12.577 | −16.749 | −11.414 | 1.00 | 0.00 | C |
| ATOM | 3133 | HA | PRO | A | 192 | 14.319 | −19.047 | −12.971 | 1.00 | 0.00 | H |
| ATOM | 3134 | 1HB | PRO | A | 192 | 11.935 | −19.986 | −12.101 | 1.00 | 0.00 | H |
| ATOM | 3135 | 2HB | PRO | A | 192 | 13.160 | −19.435 | −10.978 | 1.00 | 0.00 | H |
| ATOM | 3136 | 1HG | PRO | A | 192 | 11.315 | −18.118 | −10.253 | 1.00 | 0.00 | H |
| ATOM | 3137 | 2HG | PRO | A | 192 | 10.781 | −17.845 | −11.911 | 1.00 | 0.00 | H |
| ATOM | 3138 | 1HD | PRO | A | 192 | 13.033 | −16.485 | −10.461 | 1.00 | 0.00 | H |
| ATOM | 3139 | 2HD | PRO | A | 192 | 11.998 | −15.888 | −11.753 | 1.00 | 0.00 | H |
| ATOM | 3140 | N | ASN | A | 193 | 13.340 | −17.908 | −15.237 | 1.00 | 0.00 | N |
| ATOM | 3141 | CA | ASN | A | 193 | 12.760 | −17.828 | −16.570 | 1.00 | 0.00 | C |
| ATOM | 3142 | C | ASN | A | 193 | 13.780 | −17.879 | −17.701 | 1.00 | 0.00 | C |
| ATOM | 3143 | O | ASN | A | 193 | 13.772 | −18.814 | −18.501 | 1.00 | 0.00 | O |
| ATOM | 3144 | CB | ASN | A | 193 | 11.929 | −16.546 | −16.701 | 1.00 | 0.00 | C |
| ATOM | 3145 | CG | ASN | A | 193 | 10.576 | −16.521 | −15.990 | 1.00 | 0.00 | C |
| ATOM | 3146 | OD1 | ASN | A | 193 | 9.639 | −17.195 | −16.408 | 1.00 | 0.00 | O |
| ATOM | 3147 | ND2 | ASN | A | 193 | 10.447 | −15.735 | −14.926 | 1.00 | 0.00 | N |
| ATOM | 3148 | H | ASN | A | 193 | 14.269 | −17.530 | −15.066 | 1.00 | 0.00 | H |
| ATOM | 3149 | HA | ASN | A | 193 | 12.099 | −18.681 | −16.743 | 1.00 | 0.00 | H |
| ATOM | 3150 | 1HB | ASN | A | 193 | 12.516 | −15.683 | −16.389 | 1.00 | 0.00 | H |
| ATOM | 3151 | 2HB | ASN | A | 193 | 11.700 | −16.369 | −17.754 | 1.00 | 0.00 | H |
| ATOM | 3152 | 1HD2 | ASN | A | 193 | 9.567 | −15.698 | −14.437 | 1.00 | 0.00 | H |
| ATOM | 3153 | 2HD2 | ASN | A | 193 | 11.229 | −15.167 | −14.592 | 1.00 | 0.00 | H |
| ATOM | 3154 | N | TYR | A | 194 | 14.654 | −16.874 | −17.766 | 1.00 | 0.00 | N |
| ATOM | 3155 | CA | TYR | A | 194 | 15.656 | −16.786 | −18.831 | 1.00 | 0.00 | C |
| ATOM | 3156 | C | TYR | A | 194 | 16.249 | −18.109 | −19.283 | 1.00 | 0.00 | C |
| ATOM | 3157 | O | TYR | A | 194 | 16.817 | −18.865 | −18.490 | 1.00 | 0.00 | O |
| ATOM | 3158 | CB | TYR | A | 194 | 16.768 | −15.819 | −18.439 | 1.00 | 0.00 | C |
| ATOM | 3159 | CG | TYR | A | 194 | 17.758 | −15.365 | −19.496 | 1.00 | 0.00 | C |
| ATOM | 3160 | CD1 | TYR | A | 194 | 17.333 | −14.557 | −20.571 | 1.00 | 0.00 | C |
| ATOM | 3161 | CD2 | TYR | A | 194 | 19.126 | −15.679 | −19.357 | 1.00 | 0.00 | C |
| ATOM | 3162 | CE1 | TYR | A | 194 | 18.273 | −14.060 | −21.495 | 1.00 | 0.00 | C |
| ATOM | 3163 | CE2 | TYR | A | 194 | 20.068 | −15.156 | −20.261 | 1.00 | 0.00 | C |
| ATOM | 3164 | CZ | TYR | A | 194 | 19.641 | −14.353 | −21.337 | 1.00 | 0.00 | C |
| ATOM | 3165 | OH | TYR | A | 194 | 20.546 | −13.861 | −22.230 | 1.00 | 0.00 | O |
| ATOM | 3166 | H | TYR | A | 194 | 14.621 | −16.132 | −17.079 | 1.00 | 0.00 | H |
| ATOM | 3167 | HA | TYR | A | 194 | 15.127 | −16.390 | −19.701 | 1.00 | 0.00 | H |
| ATOM | 3168 | 1HB | TYR | A | 194 | 16.315 | −14.906 | −18.050 | 1.00 | 0.00 | H |
| ATOM | 3169 | 2HB | TYR | A | 194 | 17.321 | −16.236 | −17.595 | 1.00 | 0.00 | H |
| ATOM | 3170 | HD1 | TYR | A | 194 | 16.290 | −14.299 | −20.683 | 1.00 | 0.00 | H |
| ATOM | 3171 | HD2 | TYR | A | 194 | 19.468 | −16.302 | −18.542 | 1.00 | 0.00 | H |
| ATOM | 3172 | HE1 | TYR | A | 194 | 17.944 | −13.441 | −22.317 | 1.00 | 0.00 | H |
| ATOM | 3173 | HE2 | TYR | A | 194 | 21.113 | −15.398 | −20.129 | 1.00 | 0.00 | H |
| ATOM | 3174 | HH | TYR | A | 194 | 21.437 | −14.123 | −22.041 | 1.00 | 0.00 | H |
| ATOM | 3175 | N | THR | A | 195 | 16.114 | −18.356 | −20.584 | 1.00 | 0.00 | N |
| ATOM | 3176 | CA | THR | A | 195 | 16.568 | −19.581 | −21.227 | 1.00 | 0.00 | C |
| ATOM | 3177 | C | THR | A | 195 | 18.072 | −19.847 | −21.241 | 1.00 | 0.00 | C |
| ATOM | 3178 | O | THR | A | 195 | 18.524 | −20.861 | −20.707 | 1.00 | 0.00 | O |
| ATOM | 3179 | CB | THR | A | 195 | 16.051 | −19.638 | −22.674 | 1.00 | 0.00 | C |
| ATOM | 3180 | OG1 | THR | A | 195 | 16.311 | −18.417 | −23.357 | 1.00 | 0.00 | O |
| ATOM | 3181 | CG2 | THR | A | 195 | 14.535 | −19.880 | −22.738 | 1.00 | 0.00 | C |
| ATOM | 3182 | H | THR | A | 195 | 15.562 | −17.759 | −21.188 | 1.00 | 0.00 | H |
| ATOM | 3183 | HA | THR | A | 195 | 16.111 | −20.391 | −20.653 | 1.00 | 0.00 | H |
| ATOM | 3184 | HB | THR | A | 195 | 16.543 | −20.449 | −23.217 | 1.00 | 0.00 | H |
| ATOM | 3185 | HG1 | THR | A | 195 | 16.059 | −18.528 | −24.267 | 1.00 | 0.00 | H |
| ATOM | 3186 | 1HG2 | THR | A | 195 | 14.186 | −19.946 | −23.769 | 1.00 | 0.00 | H |
| ATOM | 3187 | 2HG2 | THR | A | 195 | 14.270 | −20.817 | −22.247 | 1.00 | 0.00 | H |
| ATOM | 3188 | 3HG2 | THR | A | 195 | 13.976 | −19.084 | −22.247 | 1.00 | 0.00 | H |
| ATOM | 3189 | N | GLU | A | 196 | 18.844 | −18.957 | −21.860 | 1.00 | 0.00 | N |
| ATOM | 3190 | CA | GLU | A | 196 | 20.292 | −19.144 | −21.936 | 1.00 | 0.00 | C |
| ATOM | 3191 | C | GLU | A | 196 | 20.877 | −19.537 | −20.592 | 1.00 | 0.00 | C |
| ATOM | 3192 | O | GLU | A | 196 | 20.788 | −18.789 | −19.616 | 1.00 | 0.00 | O |
| ATOM | 3193 | CB | GLU | A | 196 | 20.987 | −17.871 | −22.429 | 1.00 | 0.00 | C |
| ATOM | 3194 | CG | GLU | A | 196 | 21.023 | −17.734 | −23.967 | 1.00 | 0.00 | C |
| ATOM | 3195 | CD | GLU | A | 196 | 21.960 | −18.721 | −24.658 | 1.00 | 0.00 | C |
| ATOM | 3196 | OE1 | GLU | A | 196 | 22.611 | −19.546 | −23.979 | 1.00 | 0.00 | O |
| ATOM | 3197 | OE2 | GLU | A | 196 | 22.054 | −18.693 | −25.901 | 1.00 | 0.00 | O |
| ATOM | 3198 | H | GLU | A | 196 | 18.433 | −18.113 | −22.227 | 1.00 | 0.00 | H |
| ATOM | 3199 | HA | GLU | A | 196 | 20.456 | −19.941 | −22.664 | 1.00 | 0.00 | H |
| ATOM | 3200 | 1HB | GLU | A | 196 | 20.490 | −17.002 | −22.011 | 1.00 | 0.00 | H |
| ATOM | 3201 | 2HB | GLU | A | 196 | 22.016 | −17.822 | −22.064 | 1.00 | 0.00 | H |
| ATOM | 3202 | 1HG | GLU | A | 196 | 20.021 | −17.850 | −24.381 | 1.00 | 0.00 | H |
| ATOM | 3203 | 2HG | GLU | A | 196 | 21.354 | −16.730 | −24.231 | 1.00 | 0.00 | H |
| ATOM | 3204 | N | PHE | A | 197 | 21.468 | −20.723 | −20.549 | 1.00 | 0.00 | N |
| ATOM | 3205 | CA | PHE | A | 197 | 22.087 | −21.230 | −19.335 | 1.00 | 0.00 | C |
| ATOM | 3206 | C | PHE | A | 197 | 23.533 | −21.564 | −19.678 | 1.00 | 0.00 | C |
| ATOM | 3207 | O | PHE | A | 197 | 24.273 | −22.116 | −18.861 | 1.00 | 0.00 | O |
| ATOM | 3208 | CB | PHE | A | 197 | 21.346 | −22.479 | −18.842 | 1.00 | 0.00 | C |
| ATOM | 3209 | CG | PHE | A | 197 | 21.347 | −23.690 | −19.774 | 1.00 | 0.00 | C |
| ATOM | 3210 | CD1 | PHE | A | 197 | 22.332 | −24.693 | −19.635 | 1.00 | 0.00 | C |
| ATOM | 3211 | CD2 | PHE | A | 197 | 20.370 | −23.817 | −20.784 | 1.00 | 0.00 | C |
| ATOM | 3212 | CE1 | PHE | A | 197 | 22.351 | −25.798 | −20.509 | 1.00 | 0.00 | C |
| ATOM | 3213 | CE2 | PHE | A | 197 | 20.387 | −24.922 | −21.657 | 1.00 | 0.00 | C |
| ATOM | 3214 | CZ | PHE | A | 197 | 21.380 | −25.911 | −21.522 | 1.00 | 0.00 | C |
| ATOM | 3215 | H | PHE | A | 197 | 21.406 | −21.336 | −21.347 | 1.00 | 0.00 | H |
| ATOM | 3216 | HA | PHE | A | 197 | 22.192 | −20.490 | −18.540 | 1.00 | 0.00 | H |
| ATOM | 3217 | 1HB | PHE | A | 197 | 21.766 | −22.786 | −17.883 | 1.00 | 0.00 | H |
| ATOM | 3218 | 2HB | PHE | A | 197 | 20.311 | −22.213 | −18.625 | 1.00 | 0.00 | H |
| ATOM | 3219 | HD1 | PHE | A | 197 | 23.087 | −24.612 | −18.865 | 1.00 | 0.00 | H |
| ATOM | 3220 | HD2 | PHE | A | 197 | 19.601 | −23.067 | −20.896 | 1.00 | 0.00 | H |
| ATOM | 3221 | HE1 | PHE | A | 197 | 23.112 | −26.557 | −20.402 | 1.00 | 0.00 | H |
| ATOM | 3222 | HE2 | PHE | A | 197 | 19.638 | −25.010 | −22.431 | 1.00 | 0.00 | H |
| ATOM | 3223 | HZ | PHE | A | 197 | 21.394 | −26.759 | −22.193 | 1.00 | 0.00 | H |
| ATOM | 3224 | N | LYS | A | 198 | 23.921 | −21.213 | −20.902 | 1.00 | 0.00 | N |
| ATOM | 3225 | CA | LYS | A | 198 | 25.271 | −21.453 | −21.395 | 1.00 | 0.00 | C |
| ATOM | 3226 | C | LYS | A | 198 | 26.194 | −20.322 | −20.962 | 1.00 | 0.00 | C |
| ATOM | 3227 | O | LYS | A | 198 | 26.215 | −19.255 | −21.574 | 1.00 | 0.00 | O |
| ATOM | 3228 | CB | LYS | A | 198 | 25.257 | −21.565 | −22.926 | 1.00 | 0.00 | C |
| ATOM | 3229 | CG | LYS | A | 198 | 24.413 | −22.751 | −23.442 | 1.00 | 0.00 | C |
| ATOM | 3230 | CD | LYS | A | 198 | 24.563 | −23.027 | −24.951 | 1.00 | 0.00 | C |
| ATOM | 3231 | CE | LYS | A | 198 | 23.620 | −22.222 | −25.866 | 1.00 | 0.00 | C |
| ATOM | 3232 | NZ | LYS | A | 198 | 23.920 | −20.787 | −25.863 | 1.00 | 0.00 | N |
| ATOM | 3233 | H | LYS | A | 198 | 23.290 | −20.654 | −21.454 | 1.00 | 0.00 | H |
| ATOM | 3234 | HA | LYS | A | 198 | 25.662 | −22.394 | −21.000 | 1.00 | 0.00 | H |
| ATOM | 3235 | 1HB | LYS | A | 198 | 24.897 | −20.632 | −23.357 | 1.00 | 0.00 | H |
| ATOM | 3236 | 2HB | LYS | A | 198 | 26.284 | −21.681 | −23.277 | 1.00 | 0.00 | H |
| ATOM | 3237 | 1HG | LYS | A | 198 | 24.729 | −23.646 | −22.904 | 1.00 | 0.00 | H |
| ATOM | 3238 | 2HG | LYS | A | 198 | 23.361 | −22.614 | −23.188 | 1.00 | 0.00 | H |
| ATOM | 3239 | 1HD | LYS | A | 198 | 25.601 | −22.905 | −25.264 | 1.00 | 0.00 | H |
| ATOM | 3240 | 2HD | LYS | A | 198 | 24.337 | −24.081 | −25.116 | 1.00 | 0.00 | H |
| ATOM | 3241 | 1HE | LYS | A | 198 | 23.715 | −22.583 | −26.890 | 1.00 | 0.00 | H |
| ATOM | 3242 | 2HE | LYS | A | 198 | 22.579 | −22.364 | −25.576 | 1.00 | 0.00 | H |
| ATOM | 3243 | 1HZ | LYS | A | 198 | 23.393 | −20.298 | −26.572 | 1.00 | 0.00 | H |
| ATOM | 3244 | 2HZ | LYS | A | 198 | 23.542 | −20.355 | −25.004 | 1.00 | 0.00 | H |
| ATOM | 3245 | 3HZ | LYS | A | 198 | 24.896 | −20.583 | −25.951 | 1.00 | 0.00 | H |
| ATOM | 3246 | N | PHE | A | 199 | 26.948 | −20.557 | −19.894 | 1.00 | 0.00 | N |
| ATOM | 3247 | CA | PHE | A | 199 | 27.875 | −19.556 | −19.391 | 1.00 | 0.00 | C |
| ATOM | 3248 | C | PHE | A | 199 | 29.100 | −20.203 | −18.774 | 1.00 | 0.00 | C |
| ATOM | 3249 | O | PHE | A | 199 | 28.994 | −20.983 | −17.827 | 1.00 | 0.00 | O |
| ATOM | 3250 | CB | PHE | A | 199 | 27.190 | −18.653 | −18.364 | 1.00 | 0.00 | C |
| ATOM | 3251 | CG | PHE | A | 199 | 26.191 | −17.614 | −18.850 | 1.00 | 0.00 | C |
| ATOM | 3252 | CD1 | PHE | A | 199 | 26.640 | −16.349 | −19.286 | 1.00 | 0.00 | C |
| ATOM | 3253 | CD2 | PHE | A | 199 | 24.806 | −17.886 | −18.813 | 1.00 | 0.00 | C |
| ATOM | 3254 | CE1 | PHE | A | 199 | 25.713 | −15.363 | −19.677 | 1.00 | 0.00 | C |
| ATOM | 3255 | CE2 | PHE | A | 199 | 23.879 | −16.903 | −19.210 | 1.00 | 0.00 | C |
| ATOM | 3256 | CZ | PHE | A | 199 | 24.332 | −15.639 | −19.638 | 1.00 | 0.00 | C |
| ATOM | 3257 | H | PHE | A | 199 | 26.874 | −21.433 | −19.395 | 1.00 | 0.00 | H |
| ATOM | 3258 | HA | PHE | A | 199 | 28.096 | −18.944 | −20.246 | 1.00 | 0.00 | H |
| ATOM | 3259 | 1HB | PHE | A | 199 | 26.708 | −19.269 | −17.603 | 1.00 | 0.00 | H |
| ATOM | 3260 | 2HB | PHE | A | 199 | 27.955 | −18.099 | −17.818 | 1.00 | 0.00 | H |
| ATOM | 3261 | HD1 | PHE | A | 199 | 27.697 | −16.122 | −19.308 | 1.00 | 0.00 | H |
| ATOM | 3262 | HD2 | PHE | A | 199 | 24.450 | −18.848 | −18.473 | 1.00 | 0.00 | H |
| ATOM | 3263 | HE1 | PHE | A | 199 | 26.061 | −14.392 | −19.999 | 1.00 | 0.00 | H |
| ATOM | 3264 | HE2 | PHE | A | 199 | 22.820 | −17.118 | −19.177 | 1.00 | 0.00 | H |
| ATOM | 3265 | HZ | PHE | A | 199 | 23.622 | −14.880 | −19.933 | 1.00 | 0.00 | H |
| ATOM | 3266 | N | PRO | A | 200 | 30.284 | −19.885 | −19.316 | 1.00 | 0.00 | N |
| ATOM | 3267 | CA | PRO | A | 200 | 31.572 | −20.410 | −18.853 | 1.00 | 0.00 | C |
| ATOM | 3268 | C | PRO | A | 200 | 31.635 | −20.619 | −17.345 | 1.00 | 0.00 | C |
| ATOM | 3269 | O | PRO | A | 200 | 31.454 | −19.679 | −16.570 | 1.00 | 0.00 | O |
| ATOM | 3270 | CB | PRO | A | 200 | 32.587 | −19.366 | −19.330 | 1.00 | 0.00 | C |
| ATOM | 3271 | CG | PRO | A | 200 | 32.021 | −18.898 | −20.654 | 1.00 | 0.00 | C |
| ATOM | 3272 | CD | PRO | A | 200 | 30.516 | −18.918 | −20.403 | 1.00 | 0.00 | C |
| ATOM | 3273 | HA | PRO | A | 200 | 31.742 | −21.356 | −19.371 | 1.00 | 0.00 | H |
| ATOM | 3274 | 1HB | PRO | A | 200 | 32.642 | −18.524 | −18.637 | 1.00 | 0.00 | H |
| ATOM | 3275 | 2HB | PRO | A | 200 | 33.592 | −19.778 | −19.427 | 1.00 | 0.00 | H |
| ATOM | 3276 | 1HG | PRO | A | 200 | 32.279 | −19.611 | −21.438 | 1.00 | 0.00 | H |
| ATOM | 3277 | 2HG | PRO | A | 200 | 32.390 | −17.917 | −20.956 | 1.00 | 0.00 | H |
| ATOM | 3278 | 1HD | PRO | A | 200 | 29.989 | −19.191 | −21.319 | 1.00 | 0.00 | H |
| ATOM | 3279 | 2HD | PRO | A | 200 | 30.179 | −17.928 | −20.088 | 1.00 | 0.00 | H |
| ATOM | 3280 | N | GLN | A | 201 | 31.889 | −21.859 | −16.938 | 1.00 | 0.00 | N |
| ATOM | 3281 | CA | GLN | A | 201 | 31.986 | −22.188 | −15.525 | 1.00 | 0.00 | C |
| ATOM | 3282 | C | GLN | A | 201 | 33.292 | −21.645 | −14.969 | 1.00 | 0.00 | C |
| ATOM | 3283 | O | GLN | A | 201 | 34.343 | −22.266 | −15.119 | 1.00 | 0.00 | O |
| ATOM | 3284 | CB | GLN | A | 201 | 31.934 | −23.705 | −15.315 | 1.00 | 0.00 | C |
| ATOM | 3285 | CG | GLN | A | 201 | 30.697 | −24.394 | −15.950 | 1.00 | 0.00 | C |
| ATOM | 3286 | CD | GLN | A | 201 | 29.386 | −24.022 | −15.261 | 1.00 | 0.00 | C |
| ATOM | 3287 | OE1 | GLN | A | 201 | 29.043 | −24.597 | −14.233 | 1.00 | 0.00 | O |
| ATOM | 3288 | NE2 | GLN | A | 201 | 28.660 | −23.045 | −15.788 | 1.00 | 0.00 | N |
| ATOM | 3289 | H | GLN | A | 201 | 32.007 | −22.593 | −17.617 | 1.00 | 0.00 | H |
| ATOM | 3290 | HA | GLN | A | 201 | 31.058 | −21.945 | −15.081 | 1.00 | 0.00 | H |
| ATOM | 3291 | 1HB | GLN | A | 201 | 32.822 | −24.177 | −15.738 | 1.00 | 0.00 | H |
| ATOM | 3292 | 2HB | GLN | A | 201 | 31.962 | −23.929 | −14.247 | 1.00 | 0.00 | H |
| ATOM | 3293 | 1HG | GLN | A | 201 | 30.629 | −24.190 | −17.019 | 1.00 | 0.00 | H |
| ATOM | 3294 | 2HG | GLN | A | 201 | 30.814 | −25.475 | −15.867 | 1.00 | 0.00 | H |
| ATOM | 3295 | 1HE2 | GLN | A | 201 | 27.872 | −22.692 | −15.265 | 1.00 | 0.00 | H |
| ATOM | 3296 | 2HE2 | GLN | A | 201 | 28.970 | −22.507 | −16.593 | 1.00 | 0.00 | H |
| ATOM | 3297 | N | ILE | A | 202 | 33.228 | −20.476 | −14.341 | 1.00 | 0.00 | N |
| ATOM | 3298 | CA | ILE | A | 202 | 34.414 | −19.870 | −13.758 | 1.00 | 0.00 | C |
| ATOM | 3299 | C | ILE | A | 202 | 34.327 | −19.987 | −12.248 | 1.00 | 0.00 | C |
| ATOM | 3300 | O | ILE | A | 202 | 33.264 | −19.788 | −11.662 | 1.00 | 0.00 | O |
| ATOM | 3301 | CB | ILE | A | 202 | 34.540 | −18.379 | −14.148 | 1.00 | 0.00 | C |
| ATOM | 3302 | CG1 | ILE | A | 202 | 34.397 | −18.163 | −15.678 | 1.00 | 0.00 | C |
| ATOM | 3303 | CG2 | ILE | A | 202 | 35.882 | −17.763 | −13.682 | 1.00 | 0.00 | C |
| ATOM | 3304 | CD1 | ILE | A | 202 | 34.660 | −16.736 | −16.190 | 1.00 | 0.00 | C |
| ATOM | 3305 | H | ILE | A | 202 | 32.313 | −20.054 | −14.206 | 1.00 | 0.00 | H |
| ATOM | 3306 | HA | ILE | A | 202 | 35.329 | −20.384 | −14.059 | 1.00 | 0.00 | H |
| ATOM | 3307 | HB | ILE | A | 202 | 33.739 | −17.820 | −13.659 | 1.00 | 0.00 | H |
| ATOM | 3308 | 1HG1 | ILE | A | 202 | 35.041 | −18.860 | −16.215 | 1.00 | 0.00 | H |
| ATOM | 3309 | 2HG1 | ILE | A | 202 | 33.382 | −18.408 | −15.968 | 1.00 | 0.00 | H |
| ATOM | 3310 | 1HG2 | ILE | A | 202 | 35.934 | −16.695 | −13.882 | 1.00 | 0.00 | H |
| ATOM | 3311 | 2HG2 | ILE | A | 202 | 36.035 | −17.854 | −12.612 | 1.00 | 0.00 | H |
| ATOM | 3312 | 3HG2 | ILE | A | 202 | 36.730 | −18.236 | −14.177 | 1.00 | 0.00 | H |
| ATOM | 3313 | 1HD1 | ILE | A | 202 | 34.347 | −16.638 | −17.230 | 1.00 | 0.00 | H |
| ATOM | 3314 | 2HD1 | ILE | A | 202 | 34.113 | −15.992 | −15.612 | 1.00 | 0.00 | H |
| ATOM | 3315 | 3HD1 | ILE | A | 202 | 35.719 | −16.480 | −16.151 | 1.00 | 0.00 | H |
| ATOM | 3316 | N | LYS | A | 203 | 35.446 | −20.326 | −11.624 | 1.00 | 0.00 | N |
| ATOM | 3317 | CA | LYS | A | 203 | 35.491 | −20.455 | −10.179 | 1.00 | 0.00 | C |
| ATOM | 3318 | C | LYS | A | 203 | 35.466 | −19.053 | −9.587 | 1.00 | 0.00 | C |
| ATOM | 3319 | O | LYS | A | 203 | 35.975 | −18.106 | −10.190 | 1.00 | 0.00 | O |
| ATOM | 3320 | CB | LYS | A | 203 | 36.766 | −21.191 | −9.749 | 1.00 | 0.00 | C |
| ATOM | 3321 | CG | LYS | A | 203 | 36.854 | −21.530 | −8.247 | 1.00 | 0.00 | C |
| ATOM | 3322 | CD | LYS | A | 203 | 38.108 | −22.346 | −7.899 | 1.00 | 0.00 | C |
| ATOM | 3323 | CE | LYS | A | 203 | 38.214 | −22.639 | −6.394 | 1.00 | 0.00 | C |
| ATOM | 3324 | NZ | LYS | A | 203 | 39.420 | −23.416 | −6.079 | 1.00 | 0.00 | N |
| ATOM | 3325 | H | LYS | A | 203 | 36.296 | −20.439 | −12.143 | 1.00 | 0.00 | H |
| ATOM | 3326 | HA | LYS | A | 203 | 34.622 | −21.020 | −9.833 | 1.00 | 0.00 | H |
| ATOM | 3327 | 1HB | LYS | A | 203 | 36.832 | −22.122 | −10.313 | 1.00 | 0.00 | H |
| ATOM | 3328 | 2HB | LYS | A | 203 | 37.641 | −20.605 | −10.037 | 1.00 | 0.00 | H |
| ATOM | 3329 | 1HG | LYS | A | 203 | 36.866 | −20.612 | −7.658 | 1.00 | 0.00 | H |
| ATOM | 3330 | 2HG | LYS | A | 203 | 35.964 | −22.083 | −7.944 | 1.00 | 0.00 | H |
| ATOM | 3331 | 1HD | LYS | A | 203 | 38.096 | −23.283 | −8.457 | 1.00 | 0.00 | H |
| ATOM | 3332 | 2HD | LYS | A | 203 | 38.993 | −21.800 | −8.227 | 1.00 | 0.00 | H |
| ATOM | 3333 | 1HE | LYS | A | 203 | 38.242 | −21.708 | −5.827 | 1.00 | 0.00 | H |
| ATOM | 3334 | 2HE | LYS | A | 203 | 37.341 | −23.196 | −6.053 | 1.00 | 0.00 | H |
| ATOM | 3335 | 1HZ | LYS | A | 203 | 39.463 | −23.592 | −5.091 | 1.00 | 0.00 | H |
| ATOM | 3336 | 2HZ | LYS | A | 203 | 39.402 | −24.295 | −6.561 | 1.00 | 0.00 | H |
| ATOM | 3337 | 3HZ | LYS | A | 203 | 40.241 | −22.908 | −6.351 | 1.00 | 0.00 | H |
| ATOM | 3338 | N | ALA | A | 204 | 34.859 | −18.918 | −8.414 | 1.00 | 0.00 | N |
| ATOM | 3339 | CA | ALA | A | 204 | 34.783 | −17.625 | −7.757 | 1.00 | 0.00 | C |
| ATOM | 3340 | C | ALA | A | 204 | 36.187 | −17.057 | −7.625 | 1.00 | 0.00 | C |
| ATOM | 3341 | O | ALA | A | 204 | 37.121 | −17.768 | −7.255 | 1.00 | 0.00 | O |
| ATOM | 3342 | CB | ALA | A | 204 | 34.146 | −17.771 | −6.384 | 1.00 | 0.00 | C |
| ATOM | 3343 | H | ALA | A | 204 | 34.484 | −19.728 | −7.948 | 1.00 | 0.00 | H |
| ATOM | 3344 | HA | ALA | A | 204 | 34.178 | −16.955 | −8.369 | 1.00 | 0.00 | H |
| ATOM | 3345 | 1HB | ALA | A | 204 | 34.082 | −16.804 | −5.882 | 1.00 | 0.00 | H |
| ATOM | 3346 | 2HB | ALA | A | 204 | 33.130 | −18.159 | −6.464 | 1.00 | 0.00 | H |
| ATOM | 3347 | 3HB | ALA | A | 204 | 34.716 | −18.443 | −5.741 | 1.00 | 0.00 | H |
| ATOM | 3348 | N | HIS | A | 205 | 36.335 | −15.778 | −7.946 | 1.00 | 0.00 | N |
| ATOM | 3349 | CA | HIS | A | 205 | 37.627 | −15.117 | −7.843 | 1.00 | 0.00 | C |
| ATOM | 3350 | C | HIS | A | 205 | 37.785 | −14.566 | −6.423 | 1.00 | 0.00 | C |
| ATOM | 3351 | O | HIS | A | 205 | 37.006 | −13.714 | −5.989 | 1.00 | 0.00 | O |
| ATOM | 3352 | CB | HIS | A | 205 | 37.711 | −13.982 | −8.863 | 1.00 | 0.00 | C |
| ATOM | 3353 | CG | HIS | A | 205 | 39.045 | −13.287 | −8.992 | 1.00 | 0.00 | C |
| ATOM | 3354 | ND1 | HIS | A | 205 | 39.485 | −12.315 | −8.111 | 1.00 | 0.00 | N |
| ATOM | 3355 | CD2 | HIS | A | 205 | 40.044 | −13.409 | −9.932 | 1.00 | 0.00 | C |
| ATOM | 3356 | CE1 | HIS | A | 205 | 40.635 | −11.841 | −8.585 | 1.00 | 0.00 | C |
| ATOM | 3357 | NE2 | HIS | A | 205 | 41.061 | −12.488 | −9.665 | 1.00 | 0.00 | N |
| ATOM | 3358 | H | HIS | A | 205 | 35.516 | −15.198 | −8.120 | 1.00 | 0.00 | H |
| ATOM | 3359 | HA | HIS | A | 205 | 38.363 | −15.850 | −8.153 | 1.00 | 0.00 | H |
| ATOM | 3360 | 1HB | HIS | A | 205 | 37.535 | −14.423 | −9.834 | 1.00 | 0.00 | H |
| ATOM | 3361 | 2HB | HIS | A | 205 | 36.971 | −13.231 | −8.635 | 1.00 | 0.00 | H |
| ATOM | 3362 | HD1 | HIS | A | 205 | 39.003 | −11.977 | −7.283 | 1.00 | 0.00 | H |
| ATOM | 3363 | HD2 | HIS | A | 205 | 40.088 | −14.063 | −10.793 | 1.00 | 0.00 | H |
| ATOM | 3364 | HE1 | HIS | A | 205 | 41.174 | −11.024 | −8.133 | 1.00 | 0.00 | H |
| ATOM | 3365 | N | PRO | A | 206 | 38.795 | −15.051 | −5.680 | 1.00 | 0.00 | N |
| ATOM | 3366 | CA | PRO | A | 206 | 39.032 | −14.594 | −4.305 | 1.00 | 0.00 | C |
| ATOM | 3367 | C | PRO | A | 206 | 38.888 | −13.080 | −4.172 | 1.00 | 0.00 | C |
| ATOM | 3368 | O | PRO | A | 206 | 39.703 | −12.317 | −4.695 | 1.00 | 0.00 | O |
| ATOM | 3369 | CB | PRO | A | 206 | 40.457 | −15.073 | −3.997 | 1.00 | 0.00 | C |
| ATOM | 3370 | CG | PRO | A | 206 | 40.650 | −16.310 | −4.852 | 1.00 | 0.00 | C |
| ATOM | 3371 | CD | PRO | A | 206 | 39.801 | −16.037 | −6.090 | 1.00 | 0.00 | C |
| ATOM | 3372 | HA | PRO | A | 206 | 38.321 | −15.096 | −3.646 | 1.00 | 0.00 | H |
| ATOM | 3373 | 1HB | PRO | A | 206 | 41.201 | −14.328 | −4.287 | 1.00 | 0.00 | H |
| ATOM | 3374 | 2HB | PRO | A | 206 | 40.604 | −15.272 | −2.935 | 1.00 | 0.00 | H |
| ATOM | 3375 | 1HG | PRO | A | 206 | 40.258 | −17.181 | −4.324 | 1.00 | 0.00 | H |
| ATOM | 3376 | 2HG | PRO | A | 206 | 41.697 | −16.508 | −5.088 | 1.00 | 0.00 | H |
| ATOM | 3377 | 1HD | PRO | A | 206 | 39.364 | −16.965 | −6.457 | 1.00 | 0.00 | H |
| ATOM | 3378 | 2HD | PRO | A | 206 | 40.416 | −15.609 | −6.884 | 1.00 | 0.00 | H |
| ATOM | 3379 | N | TRP | A | 207 | 37.840 | −12.661 | −3.469 | 1.00 | 0.00 | N |
| ATOM | 3380 | CA | TRP | A | 207 | 37.551 | −11.249 | −3.264 | 1.00 | 0.00 | C |
| ATOM | 3381 | C | TRP | A | 207 | 38.782 | −10.447 | −2.843 | 1.00 | 0.00 | C |
| ATOM | 3382 | O | TRP | A | 207 | 38.872 | −9.249 | −3.117 | 1.00 | 0.00 | O |
| ATOM | 3383 | CB | TRP | A | 207 | 36.445 | −11.091 | −2.217 | 1.00 | 0.00 | C |
| ATOM | 3384 | CG | TRP | A | 207 | 35.077 | −10.955 | −2.805 | 1.00 | 0.00 | C |
| ATOM | 3385 | CD1 | TRP | A | 207 | 34.055 | −11.827 | −2.671 | 1.00 | 0.00 | C |
| ATOM | 3386 | CD2 | TRP | A | 207 | 34.573 | −9.870 | −3.637 | 1.00 | 0.00 | C |
| ATOM | 3387 | NE1 | TRP | A | 207 | 32.947 | −11.338 | −3.334 | 1.00 | 0.00 | N |
| ATOM | 3388 | CE3 | TRP | A | 207 | 35.138 | −8.684 | −4.159 | 1.00 | 0.00 | C |
| ATOM | 3389 | CE2 | TRP | A | 207 | 33.209 | −10.137 | −3.959 | 1.00 | 0.00 | C |
| ATOM | 3390 | CZ3 | TRP | A | 207 | 34.385 | −7.807 | −4.961 | 1.00 | 0.00 | C |
| ATOM | 3391 | CZ2 | TRP | A | 207 | 32.444 | −9.267 | −4.754 | 1.00 | 0.00 | C |
| ATOM | 3392 | CH2 | TRP | A | 207 | 33.044 | −8.104 | −5.268 | 1.00 | 0.00 | C |
| ATOM | 3393 | H | TRP | A | 207 | 37.160 | −13.336 | −3.156 | 1.00 | 0.00 | H |
| ATOM | 3394 | HA | TRP | A | 207 | 37.216 | −10.833 | −4.215 | 1.00 | 0.00 | H |
| ATOM | 3395 | 1HB | TRP | A | 207 | 36.466 | −11.899 | −1.485 | 1.00 | 0.00 | H |
| ATOM | 3396 | 2HB | TRP | A | 207 | 36.583 | −10.176 | −1.651 | 1.00 | 0.00 | H |
| ATOM | 3397 | HD1 | TRP | A | 207 | 34.104 | −12.749 | −2.110 | 1.00 | 0.00 | H |
| ATOM | 3398 | HE1 | TRP | A | 207 | 32.056 | −11.821 | −3.328 | 1.00 | 0.00 | H |
| ATOM | 3399 | HE3 | TRP | A | 207 | 36.172 | −8.455 | −3.947 | 1.00 | 0.00 | H |
| ATOM | 3400 | HZ3 | TRP | A | 207 | 34.842 | −6.907 | −5.347 | 1.00 | 0.00 | H |
| ATOM | 3401 | HZ2 | TRP | A | 207 | 31.414 | −9.491 | −4.980 | 1.00 | 0.00 | H |
| ATOM | 3402 | HH2 | TRP | A | 207 | 32.481 | −7.443 | −5.908 | 1.00 | 0.00 | H |
| ATOM | 3403 | N | THR | A | 208 | 39.731 | −11.108 | −2.185 | 1.00 | 0.00 | N |
| ATOM | 3404 | CA | THR | A | 208 | 40.946 | −10.435 | −1.737 | 1.00 | 0.00 | C |
| ATOM | 3405 | C | THR | A | 208 | 41.849 | −10.043 | −2.906 | 1.00 | 0.00 | C |
| ATOM | 3406 | O | THR | A | 208 | 42.491 | −8.990 | −2.871 | 1.00 | 0.00 | O |
| ATOM | 3407 | CB | THR | A | 208 | 41.748 | −11.316 | −0.758 | 1.00 | 0.00 | C |
| ATOM | 3408 | OG1 | THR | A | 208 | 41.886 | −12.636 | −1.267 | 1.00 | 0.00 | O |
| ATOM | 3409 | CG2 | THR | A | 208 | 41.062 | −11.419 | 0.612 | 1.00 | 0.00 | C |
| ATOM | 3410 | H | THR | A | 208 | 39.584 | −12.070 | −1.927 | 1.00 | 0.00 | H |
| ATOM | 3411 | HA | THR | A | 208 | 40.667 | −9.521 | −1.218 | 1.00 | 0.00 | H |
| ATOM | 3412 | HB | THR | A | 208 | 42.745 | −10.893 | −0.610 | 1.00 | 0.00 | H |
| ATOM | 3413 | HG1 | THR | A | 208 | 42.465 | −13.118 | −0.688 | 1.00 | 0.00 | H |
| ATOM | 3414 | 1HG2 | THR | A | 208 | 41.648 | −12.022 | 1.306 | 1.00 | 0.00 | H |
| ATOM | 3415 | 2HG2 | THR | A | 208 | 40.940 | −10.433 | 1.062 | 1.00 | 0.00 | H |
| ATOM | 3416 | 3HG2 | THR | A | 208 | 40.072 | −11.868 | 0.534 | 1.00 | 0.00 | H |
| ATOM | 3417 | N | LYS | A | 209 | 41.894 | −10.885 | −3.936 | 1.00 | 0.00 | N |
| ATOM | 3418 | CA | LYS | A | 209 | 42.721 | −10.604 | −5.108 | 1.00 | 0.00 | C |
| ATOM | 3419 | C | LYS | A | 209 | 42.057 | −9.573 | −6.022 | 1.00 | 0.00 | C |
| ATOM | 3420 | O | LYS | A | 209 | 42.677 | −9.070 | −6.958 | 1.00 | 0.00 | O |
| ATOM | 3421 | CB | LYS | A | 209 | 42.992 | −11.895 | −5.896 | 1.00 | 0.00 | C |
| ATOM | 3422 | CG | LYS | A | 209 | 43.934 | −11.856 | −7.118 | 1.00 | 0.00 | C |
| ATOM | 3423 | CD | LYS | A | 209 | 44.026 | −13.238 | −7.793 | 1.00 | 0.00 | C |
| ATOM | 3424 | CE | LYS | A | 209 | 44.674 | −13.220 | −9.187 | 1.00 | 0.00 | C |
| ATOM | 3425 | NZ | LYS | A | 209 | 43.871 | −12.453 | −10.153 | 1.00 | 0.00 | N |
| ATOM | 3426 | H | LYS | A | 209 | 41.191 | −11.618 | −4.008 | 1.00 | 0.00 | H |
| ATOM | 3427 | HA | LYS | A | 209 | 43.680 | −10.191 | −4.788 | 1.00 | 0.00 | H |
| ATOM | 3428 | 1HB | LYS | A | 209 | 43.402 | −12.624 | −5.195 | 1.00 | 0.00 | H |
| ATOM | 3429 | 2HB | LYS | A | 209 | 42.033 | −12.316 | −6.201 | 1.00 | 0.00 | H |
| ATOM | 3430 | 1HG | LYS | A | 209 | 43.571 | −11.140 | −7.850 | 1.00 | 0.00 | H |
| ATOM | 3431 | 2HG | LYS | A | 209 | 44.926 | −11.514 | −6.820 | 1.00 | 0.00 | H |
| ATOM | 3432 | 1HD | LYS | A | 209 | 44.590 | −13.912 | −7.146 | 1.00 | 0.00 | H |
| ATOM | 3433 | 2HD | LYS | A | 209 | 43.031 | −13.679 | −7.876 | 1.00 | 0.00 | H |
| ATOM | 3434 | 1HE | LYS | A | 209 | 45.682 | −12.808 | −9.145 | 1.00 | 0.00 | H |
| ATOM | 3435 | 2HE | LYS | A | 209 | 44.763 | −14.242 | −9.556 | 1.00 | 0.00 | H |
| ATOM | 3436 | 1HZ | LYS | A | 209 | 44.189 | −12.625 | −11.090 | 1.00 | 0.00 | H |
| ATOM | 3437 | 2HZ | LYS | A | 209 | 42.888 | −12.702 | −10.080 | 1.00 | 0.00 | H |
| ATOM | 3438 | 3HZ | LYS | A | 209 | 43.948 | −11.471 | −9.957 | 1.00 | 0.00 | H |
| ATOM | 3439 | N | VAL | A | 210 | 40.797 | −9.255 | −5.741 | 1.00 | 0.00 | N |
| ATOM | 3440 | CA | VAL | A | 210 | 40.054 | −8.285 | −6.538 | 1.00 | 0.00 | C |
| ATOM | 3441 | C | VAL | A | 210 | 40.530 | −6.856 | −6.286 | 1.00 | 0.00 | C |
| ATOM | 3442 | O | VAL | A | 210 | 40.646 | −6.061 | −7.217 | 1.00 | 0.00 | O |
| ATOM | 3443 | CB | VAL | A | 210 | 38.541 | −8.358 | −6.235 | 1.00 | 0.00 | C |
| ATOM | 3444 | CG1 | VAL | A | 210 | 37.685 | −7.314 | −6.990 | 1.00 | 0.00 | C |
| ATOM | 3445 | CG2 | VAL | A | 210 | 37.978 | −9.749 | −6.573 | 1.00 | 0.00 | C |
| ATOM | 3446 | H | VAL | A | 210 | 40.334 | −9.702 | −4.963 | 1.00 | 0.00 | H |
| ATOM | 3447 | HA | VAL | A | 210 | 40.211 | −8.501 | −7.598 | 1.00 | 0.00 | H |
| ATOM | 3448 | HB | VAL | A | 210 | 38.377 | −8.160 | −5.179 | 1.00 | 0.00 | H |
| ATOM | 3449 | 1HG1 | VAL | A | 210 | 36.620 | −7.502 | −6.854 | 1.00 | 0.00 | H |
| ATOM | 3450 | 2HG1 | VAL | A | 210 | 37.865 | −6.299 | −6.633 | 1.00 | 0.00 | H |
| ATOM | 3451 | 3HG1 | VAL | A | 210 | 37.885 | −7.332 | −8.062 | 1.00 | 0.00 | H |
| ATOM | 3452 | 1HG2 | VAL | A | 210 | 36.932 | −9.825 | −6.277 | 1.00 | 0.00 | H |
| ATOM | 3453 | 2HG2 | VAL | A | 210 | 38.028 | −9.944 | −7.645 | 1.00 | 0.00 | H |
| ATOM | 3454 | 3HG2 | VAL | A | 210 | 38.515 | −10.542 | −6.062 | 1.00 | 0.00 | H |
| ATOM | 3455 | N | PHE | A | 211 | 40.812 | −6.537 | −5.026 | 1.00 | 0.00 | N |
| ATOM | 3456 | CA | PHE | A | 211 | 41.255 | −5.194 | −4.665 | 1.00 | 0.00 | C |
| ATOM | 3457 | C | PHE | A | 211 | 42.758 | −5.098 | −4.395 | 1.00 | 0.00 | C |
| ATOM | 3458 | O | PHE | A | 211 | 43.434 | −6.116 | −4.241 | 1.00 | 0.00 | O |
| ATOM | 3459 | CB | PHE | A | 211 | 40.490 | −4.719 | −3.426 | 1.00 | 0.00 | C |
| ATOM | 3460 | CG | PHE | A | 211 | 38.976 | −4.758 | −3.504 | 1.00 | 0.00 | C |
| ATOM | 3461 | CD1 | PHE | A | 211 | 38.276 | −3.695 | −4.107 | 1.00 | 0.00 | C |
| ATOM | 3462 | CD2 | PHE | A | 211 | 38.260 | −5.835 | −2.940 | 1.00 | 0.00 | C |
| ATOM | 3463 | CE1 | PHE | A | 211 | 36.871 | −3.716 | −4.161 | 1.00 | 0.00 | C |
| ATOM | 3464 | CE2 | PHE | A | 211 | 36.852 | −5.840 | −2.970 | 1.00 | 0.00 | C |
| ATOM | 3465 | CZ | PHE | A | 211 | 36.159 | −4.785 | −3.590 | 1.00 | 0.00 | C |
| ATOM | 3466 | H | PHE | A | 211 | 40.791 | −7.254 | −4.316 | 1.00 | 0.00 | H |
| ATOM | 3467 | HA | PHE | A | 211 | 41.028 | −4.481 | −5.460 | 1.00 | 0.00 | H |
| ATOM | 3468 | 1HB | PHE | A | 211 | 40.808 | −5.287 | −2.549 | 1.00 | 0.00 | H |
| ATOM | 3469 | 2HB | PHE | A | 211 | 40.755 | −3.684 | −3.221 | 1.00 | 0.00 | H |
| ATOM | 3470 | HD1 | PHE | A | 211 | 38.814 | −2.859 | −4.529 | 1.00 | 0.00 | H |
| ATOM | 3471 | HD2 | PHE | A | 211 | 38.784 | −6.657 | −2.473 | 1.00 | 0.00 | H |
| ATOM | 3472 | HE1 | PHE | A | 211 | 36.339 | −2.908 | −4.636 | 1.00 | 0.00 | H |
| ATOM | 3473 | HE2 | PHE | A | 211 | 36.303 | −6.653 | −2.522 | 1.00 | 0.00 | H |
| ATOM | 3474 | HZ | PHE | A | 211 | 35.080 | −4.792 | −3.629 | 1.00 | 0.00 | H |
| ATOM | 3475 | N | LYS | A | 212 | 43.269 | −3.869 | −4.342 | 1.00 | 0.00 | N |
| ATOM | 3476 | CA | LYS | A | 212 | 44.684 | −3.630 | −4.064 | 1.00 | 0.00 | C |
| ATOM | 3477 | C | LYS | A | 212 | 45.019 | −4.225 | −2.690 | 1.00 | 0.00 | C |
| ATOM | 3478 | O | LYS | A | 212 | 44.139 | −4.391 | −1.842 | 1.00 | 0.00 | O |
| ATOM | 3479 | CB | LYS | A | 212 | 44.982 | −2.128 | −4.082 | 1.00 | 0.00 | C |
| ATOM | 3480 | CG | LYS | A | 212 | 44.421 | −1.348 | −5.291 | 1.00 | 0.00 | C |
| ATOM | 3481 | CD | LYS | A | 212 | 44.516 | 0.179 | −5.103 | 1.00 | 0.00 | C |
| ATOM | 3482 | CE | LYS | A | 212 | 43.797 | 0.977 | −6.204 | 1.00 | 0.00 | C |
| ATOM | 3483 | NZ | LYS | A | 212 | 42.339 | 0.781 | −6.154 | 1.00 | 0.00 | N |
| ATOM | 3484 | H | LYS | A | 212 | 42.658 | −3.074 | −4.439 | 1.00 | 0.00 | H |
| ATOM | 3485 | HA | LYS | A | 212 | 45.276 | −4.131 | −4.833 | 1.00 | 0.00 | H |
| ATOM | 3486 | 1HB | LYS | A | 212 | 44.569 | −1.686 | −3.173 | 1.00 | 0.00 | H |
| ATOM | 3487 | 2HB | LYS | A | 212 | 46.060 | −1.971 | −4.018 | 1.00 | 0.00 | H |
| ATOM | 3488 | 1HG | LYS | A | 212 | 44.945 | −1.650 | −6.199 | 1.00 | 0.00 | H |
| ATOM | 3489 | 2HG | LYS | A | 212 | 43.376 | −1.607 | −5.448 | 1.00 | 0.00 | H |
| ATOM | 3490 | 1HD | LYS | A | 212 | 44.114 | 0.466 | −4.130 | 1.00 | 0.00 | H |
| ATOM | 3491 | 2HD | LYS | A | 212 | 45.569 | 0.467 | −5.085 | 1.00 | 0.00 | H |
| ATOM | 3492 | 1HE | LYS | A | 212 | 44.012 | 2.041 | −6.102 | 1.00 | 0.00 | H |
| ATOM | 3493 | 2HE | LYS | A | 212 | 44.164 | 0.678 | −7.187 | 1.00 | 0.00 | H |
| ATOM | 3494 | 1HZ | LYS | A | 212 | 41.892 | 1.224 | −6.953 | 1.00 | 0.00 | H |
| ATOM | 3495 | 2HZ | LYS | A | 212 | 42.134 | −0.202 | −6.208 | 1.00 | 0.00 | H |
| ATOM | 3496 | 3HZ | LYS | A | 212 | 41.917 | 1.138 | −5.305 | 1.00 | 0.00 | H |
| ATOM | 3497 | N | SER | A | 213 | 46.300 | −4.548 | −2.449 | 1.00 | 0.00 | N |
| ATOM | 3498 | CA | SER | A | 213 | 46.726 | −5.132 | −1.170 | 1.00 | 0.00 | C |
| ATOM | 3499 | C | SER | A | 213 | 46.391 | −4.332 | 0.091 | 1.00 | 0.00 | C |
| ATOM | 3500 | O | SER | A | 213 | 46.026 | −4.905 | 1.116 | 1.00 | 0.00 | O |
| ATOM | 3501 | CB | SER | A | 213 | 48.236 | −5.327 | −1.343 | 1.00 | 0.00 | C |
| ATOM | 3502 | OG | SER | A | 213 | 48.897 | −4.127 | −1.732 | 1.00 | 0.00 | O |
| ATOM | 3503 | H | SER | A | 213 | 47.028 | −4.338 | −3.116 | 1.00 | 0.00 | H |
| ATOM | 3504 | HA | SER | A | 213 | 46.255 | −6.112 | −1.067 | 1.00 | 0.00 | H |
| ATOM | 3505 | 1HB | SER | A | 213 | 48.677 | −5.694 | −0.414 | 1.00 | 0.00 | H |
| ATOM | 3506 | 2HB | SER | A | 213 | 48.430 | −6.095 | −2.094 | 1.00 | 0.00 | H |
| ATOM | 3507 | HG | SER | A | 213 | 49.824 | −4.324 | −1.824 | 1.00 | 0.00 | H |
| ATOM | 3508 | N | ARG | A | 214 | 46.515 | −3.014 | 0.017 | 1.00 | 0.00 | N |
| ATOM | 3509 | CA | ARG | A | 214 | 46.237 | −2.158 | 1.168 | 1.00 | 0.00 | C |
| ATOM | 3510 | C | ARG | A | 214 | 44.773 | −2.164 | 1.631 | 1.00 | 0.00 | C |
| ATOM | 3511 | O | ARG | A | 214 | 44.480 | −1.813 | 2.776 | 1.00 | 0.00 | O |
| ATOM | 3512 | CB | ARG | A | 214 | 46.639 | −0.713 | 0.855 | 1.00 | 0.00 | C |
| ATOM | 3513 | CG | ARG | A | 214 | 48.134 | −0.529 | 0.540 | 1.00 | 0.00 | C |
| ATOM | 3514 | CD | ARG | A | 214 | 48.493 | 0.942 | 0.290 | 1.00 | 0.00 | C |
| ATOM | 3515 | NE | ARG | A | 214 | 49.925 | 1.109 | 0.007 | 1.00 | 0.00 | N |
| ATOM | 3516 | CZ | ARG | A | 214 | 50.535 | 2.251 | −0.335 | 1.00 | 0.00 | C |
| ATOM | 3517 | NH1 | ARG | A | 214 | 49.846 | 3.385 | −0.451 | 1.00 | 0.00 | N |
| ATOM | 3518 | NH2 | ARG | A | 214 | 51.846 | 2.250 | −0.560 | 1.00 | 0.00 | N |
| ATOM | 3519 | H | ARG | A | 214 | 46.889 | −2.623 | −0.835 | 1.00 | 0.00 | H |
| ATOM | 3520 | HA | ARG | A | 214 | 46.837 | −2.507 | 2.011 | 1.00 | 0.00 | H |
| ATOM | 3521 | 1HB | ARG | A | 214 | 46.044 | −0.339 | 0.020 | 1.00 | 0.00 | H |
| ATOM | 3522 | 2HB | ARG | A | 214 | 46.388 | −0.078 | 1.707 | 1.00 | 0.00 | H |
| ATOM | 3523 | 1HG | ARG | A | 214 | 48.727 | −0.914 | 1.371 | 1.00 | 0.00 | H |
| ATOM | 3524 | 2HG | ARG | A | 214 | 48.408 | −1.121 | −0.334 | 1.00 | 0.00 | H |
| ATOM | 3525 | 1HD | ARG | A | 214 | 47.920 | 1.327 | −0.555 | 1.00 | 0.00 | H |
| ATOM | 3526 | 2HD | ARG | A | 214 | 48.235 | 1.549 | 1.158 | 1.00 | 0.00 | H |
| ATOM | 3527 | HE | ARG | A | 214 | 50.478 | 0.264 | 0.100 | 1.00 | 0.00 | H |
| ATOM | 3528 | 1HH1 | ARG | A | 214 | 50.284 | 4.259 | −0.707 | 1.00 | 0.00 | H |
| ATOM | 3529 | 2HH1 | ARG | A | 214 | 48.853 | 3.417 | −0.286 | 1.00 | 0.00 | H |
| ATOM | 3530 | 1HH2 | ARG | A | 214 | 52.348 | 3.087 | −0.819 | 1.00 | 0.00 | H |
| ATOM | 3531 | 2HH2 | ARG | A | 214 | 52.403 | 1.412 | −0.482 | 1.00 | 0.00 | H |
| ATOM | 3532 | N | THR | A | 215 | 43.862 | −2.554 | 0.741 | 1.00 | 0.00 | N |
| ATOM | 3533 | CA | THR | A | 215 | 42.430 | −2.575 | 1.041 | 1.00 | 0.00 | C |
| ATOM | 3534 | C | THR | A | 215 | 42.036 | −3.250 | 2.350 | 1.00 | 0.00 | C |
| ATOM | 3535 | O | THR | A | 215 | 42.322 | −4.424 | 2.563 | 1.00 | 0.00 | O |
| ATOM | 3536 | CB | THR | A | 215 | 41.649 | −3.247 | −0.098 | 1.00 | 0.00 | C |
| ATOM | 3537 | OG1 | THR | A | 215 | 42.070 | −2.697 | −1.335 | 1.00 | 0.00 | O |
| ATOM | 3538 | CG2 | THR | A | 215 | 40.129 | −3.074 | −0.006 | 1.00 | 0.00 | C |
| ATOM | 3539 | H | THR | A | 215 | 44.171 | −2.923 | −0.146 | 1.00 | 0.00 | H |
| ATOM | 3540 | HA | THR | A | 215 | 42.152 | −1.528 | 0.990 | 1.00 | 0.00 | H |
| ATOM | 3541 | HB | THR | A | 215 | 41.876 | −4.315 | −0.130 | 1.00 | 0.00 | H |
| ATOM | 3542 | HG1 | THR | A | 215 | 42.816 | −3.228 | −1.616 | 1.00 | 0.00 | H |
| ATOM | 3543 | 1HG2 | THR | A | 215 | 39.618 | −3.414 | −0.907 | 1.00 | 0.00 | H |
| ATOM | 3544 | 2HG2 | THR | A | 215 | 39.708 | −3.626 | 0.833 | 1.00 | 0.00 | H |
| ATOM | 3545 | 3HG2 | THR | A | 215 | 39.892 | −2.030 | 0.153 | 1.00 | 0.00 | H |
| ATOM | 3546 | N | PRO | A | 216 | 41.353 | −2.509 | 3.239 | 1.00 | 0.00 | N |
| ATOM | 3547 | CA | PRO | A | 216 | 40.914 | −3.047 | 4.535 | 1.00 | 0.00 | C |
| ATOM | 3548 | C | PRO | A | 216 | 40.061 | −4.302 | 4.375 | 1.00 | 0.00 | C |
| ATOM | 3549 | O | PRO | A | 216 | 39.128 | −4.328 | 3.575 | 1.00 | 0.00 | O |
| ATOM | 3550 | CB | PRO | A | 216 | 40.119 | −1.898 | 5.158 | 1.00 | 0.00 | C |
| ATOM | 3551 | CG | PRO | A | 216 | 40.590 | −0.619 | 4.504 | 1.00 | 0.00 | C |
| ATOM | 3552 | CD | PRO | A | 216 | 41.107 | −1.062 | 3.145 | 1.00 | 0.00 | C |
| ATOM | 3553 | HA | PRO | A | 216 | 41.834 | −3.204 | 5.095 | 1.00 | 0.00 | H |
| ATOM | 3554 | 1HB | PRO | A | 216 | 39.051 | −2.013 | 4.965 | 1.00 | 0.00 | H |
| ATOM | 3555 | 2HB | PRO | A | 216 | 40.232 | −1.871 | 6.243 | 1.00 | 0.00 | H |
| ATOM | 3556 | 1HG | PRO | A | 216 | 41.410 | −0.197 | 5.087 | 1.00 | 0.00 | H |
| ATOM | 3557 | 2HG | PRO | A | 216 | 39.808 | 0.139 | 4.437 | 1.00 | 0.00 | H |
| ATOM | 3558 | 1HD | PRO | A | 216 | 42.010 | −0.500 | 2.900 | 1.00 | 0.00 | H |
| ATOM | 3559 | 2HD | PRO | A | 216 | 40.356 | −0.871 | 2.377 | 1.00 | 0.00 | H |
| ATOM | 3560 | N | PRO | A | 217 | 40.376 | −5.367 | 5.134 | 1.00 | 0.00 | N |
| ATOM | 3561 | CA | PRO | A | 217 | 39.613 | −6.614 | 5.050 | 1.00 | 0.00 | C |
| ATOM | 3562 | C | PRO | A | 217 | 38.127 | −6.421 | 5.324 | 1.00 | 0.00 | C |
| ATOM | 3563 | O | PRO | A | 217 | 37.289 | −7.117 | 4.753 | 1.00 | 0.00 | O |
| ATOM | 3564 | CB | PRO | A | 217 | 40.271 | −7.522 | 6.093 | 1.00 | 0.00 | C |
| ATOM | 3565 | CG | PRO | A | 217 | 41.091 | −6.646 | 7.021 | 1.00 | 0.00 | C |
| ATOM | 3566 | CD | PRO | A | 217 | 41.355 | −5.376 | 6.226 | 1.00 | 0.00 | C |
| ATOM | 3567 | HA | PRO | A | 217 | 39.725 | −7.043 | 4.052 | 1.00 | 0.00 | H |
| ATOM | 3568 | 1HB | PRO | A | 217 | 39.557 | −8.139 | 6.642 | 1.00 | 0.00 | H |
| ATOM | 3569 | 2HB | PRO | A | 217 | 40.940 | −8.212 | 5.577 | 1.00 | 0.00 | H |
| ATOM | 3570 | 1HG | PRO | A | 217 | 42.006 | −7.128 | 7.368 | 1.00 | 0.00 | H |
| ATOM | 3571 | 2HG | PRO | A | 217 | 40.496 | −6.398 | 7.902 | 1.00 | 0.00 | H |
| ATOM | 3572 | 1HD | PRO | A | 217 | 42.358 | −5.400 | 5.798 | 1.00 | 0.00 | H |
| ATOM | 3573 | 2HD | PRO | A | 217 | 41.282 | −4.499 | 6.871 | 1.00 | 0.00 | H |
| ATOM | 3574 | N | GLU | A | 218 | 37.796 | −5.484 | 6.204 | 1.00 | 0.00 | N |
| ATOM | 3575 | CA | GLU | A | 218 | 36.394 | −5.231 | 6.519 | 1.00 | 0.00 | C |
| ATOM | 3576 | C | GLU | A | 218 | 35.637 | −4.708 | 5.295 | 1.00 | 0.00 | C |
| ATOM | 3577 | O | GLU | A | 218 | 34.476 | −5.056 | 5.085 | 1.00 | 0.00 | O |
| ATOM | 3578 | CB | GLU | A | 218 | 36.278 | −4.232 | 7.672 | 1.00 | 0.00 | C |
| ATOM | 3579 | CG | GLU | A | 218 | 37.095 | −4.550 | 8.949 | 1.00 | 0.00 | C |
| ATOM | 3580 | CD | GLU | A | 218 | 38.534 | −4.035 | 8.947 | 1.00 | 0.00 | C |
| ATOM | 3581 | OE1 | GLU | A | 218 | 39.061 | −3.756 | 7.849 | 1.00 | 0.00 | O |
| ATOM | 3582 | OE2 | GLU | A | 218 | 39.114 | −3.949 | 10.045 | 1.00 | 0.00 | O |
| ATOM | 3583 | H | GLU | A | 218 | 38.512 | −4.986 | 6.707 | 1.00 | 0.00 | H |
| ATOM | 3584 | HA | GLU | A | 218 | 35.947 | −6.208 | 6.656 | 1.00 | 0.00 | H |
| ATOM | 3585 | 1HB | GLU | A | 218 | 36.537 | −3.229 | 7.328 | 1.00 | 0.00 | H |
| ATOM | 3586 | 2HB | GLU | A | 218 | 35.226 | −4.178 | 7.956 | 1.00 | 0.00 | H |
| ATOM | 3587 | 1HG | GLU | A | 218 | 36.598 | −4.088 | 9.802 | 1.00 | 0.00 | H |
| ATOM | 3588 | 2HG | GLU | A | 218 | 37.099 | −5.623 | 9.143 | 1.00 | 0.00 | H |
| ATOM | 3589 | N | ALA | A | 219 | 36.297 | −3.874 | 4.495 | 1.00 | 0.00 | N |
| ATOM | 3590 | CA | ALA | A | 219 | 35.674 | −3.333 | 3.290 | 1.00 | 0.00 | C |
| ATOM | 3591 | C | ALA | A | 219 | 35.390 | −4.467 | 2.304 | 1.00 | 0.00 | C |
| ATOM | 3592 | O | ALA | A | 219 | 34.339 | −4.502 | 1.668 | 1.00 | 0.00 | O |
| ATOM | 3593 | CB | ALA | A | 219 | 36.581 | −2.297 | 2.643 | 1.00 | 0.00 | C |
| ATOM | 3594 | H | ALA | A | 219 | 37.281 | −3.720 | 4.649 | 1.00 | 0.00 | H |
| ATOM | 3595 | HA | ALA | A | 219 | 34.730 | −2.856 | 3.565 | 1.00 | 0.00 | H |
| ATOM | 3596 | 1HB | ALA | A | 219 | 36.019 | −1.695 | 1.931 | 1.00 | 0.00 | H |
| ATOM | 3597 | 2HB | ALA | A | 219 | 37.000 | −1.614 | 3.378 | 1.00 | 0.00 | H |
| ATOM | 3598 | 3HB | ALA | A | 219 | 37.419 | −2.750 | 2.111 | 1.00 | 0.00 | H |
| ATOM | 3599 | N | ILE | A | 220 | 36.338 | −5.389 | 2.176 | 1.00 | 0.00 | N |
| ATOM | 3600 | CA | ILE | A | 220 | 36.180 | −6.521 | 1.274 | 1.00 | 0.00 | C |
| ATOM | 3601 | C | ILE | A | 220 | 35.042 | −7.418 | 1.769 | 1.00 | 0.00 | C |
| ATOM | 3602 | O | ILE | A | 220 | 34.182 | −7.847 | 0.988 | 1.00 | 0.00 | O |
| ATOM | 3603 | CB | ILE | A | 220 | 37.499 | −7.333 | 1.182 | 1.00 | 0.00 | C |
| ATOM | 3604 | CG1 | ILE | A | 220 | 38.694 | −6.417 | 0.811 | 1.00 | 0.00 | C |
| ATOM | 3605 | CG2 | ILE | A | 220 | 37.414 | −8.507 | 0.189 | 1.00 | 0.00 | C |
| ATOM | 3606 | CD1 | ILE | A | 220 | 40.071 | −7.093 | 0.836 | 1.00 | 0.00 | C |
| ATOM | 3607 | H | ILE | A | 220 | 37.176 | −5.309 | 2.735 | 1.00 | 0.00 | H |
| ATOM | 3608 | HA | ILE | A | 220 | 35.915 | −6.149 | 0.281 | 1.00 | 0.00 | H |
| ATOM | 3609 | HB | ILE | A | 220 | 37.708 | −7.756 | 2.166 | 1.00 | 0.00 | H |
| ATOM | 3610 | 1HG1 | ILE | A | 220 | 38.527 | −5.958 | −0.163 | 1.00 | 0.00 | H |
| ATOM | 3611 | 2HG1 | ILE | A | 220 | 38.751 | −5.590 | 1.510 | 1.00 | 0.00 | H |
| ATOM | 3612 | 1HG2 | ILE | A | 220 | 38.330 | −9.096 | 0.174 | 1.00 | 0.00 | H |
| ATOM | 3613 | 2HG2 | ILE | A | 220 | 36.613 | −9.200 | 0.444 | 1.00 | 0.00 | H |
| ATOM | 3614 | 3HG2 | ILE | A | 220 | 37.237 | −8.144 | −0.823 | 1.00 | 0.00 | H |
| ATOM | 3615 | 1HD1 | ILE | A | 220 | 40.866 | −6.350 | 0.777 | 1.00 | 0.00 | H |
| ATOM | 3616 | 2HD1 | ILE | A | 220 | 40.223 | −7.662 | 1.753 | 1.00 | 0.00 | H |
| ATOM | 3617 | 3HD1 | ILE | A | 220 | 40.197 | −7.763 | −0.010 | 1.00 | 0.00 | H |
| ATOM | 3618 | N | ALA | A | 221 | 35.029 | −7.687 | 3.072 | 1.00 | 0.00 | N |
| ATOM | 3619 | CA | ALA | A | 221 | 33.993 | −8.529 | 3.671 | 1.00 | 0.00 | C |
| ATOM | 3620 | C | ALA | A | 221 | 32.595 | −7.949 | 3.440 | 1.00 | 0.00 | C |
| ATOM | 3621 | O | ALA | A | 221 | 31.674 | −8.675 | 3.070 | 1.00 | 0.00 | O |
| ATOM | 3622 | CB | ALA | A | 221 | 34.253 | −8.688 | 5.167 | 1.00 | 0.00 | C |
| ATOM | 3623 | H | ALA | A | 221 | 35.768 | −7.322 | 3.668 | 1.00 | 0.00 | H |
| ATOM | 3624 | HA | ALA | A | 221 | 34.033 | −9.513 | 3.199 | 1.00 | 0.00 | H |
| ATOM | 3625 | 1HB | ALA | A | 221 | 33.512 | −9.344 | 5.625 | 1.00 | 0.00 | H |
| ATOM | 3626 | 2HB | ALA | A | 221 | 35.235 | −9.123 | 5.353 | 1.00 | 0.00 | H |
| ATOM | 3627 | 3HB | ALA | A | 221 | 34.211 | −7.733 | 5.695 | 1.00 | 0.00 | H |
| ATOM | 3628 | N | LEU | A | 222 | 32.438 | −6.647 | 3.671 | 1.00 | 0.00 | N |
| ATOM | 3629 | CA | LEU | A | 222 | 31.149 | −5.988 | 3.463 | 1.00 | 0.00 | C |
| ATOM | 3630 | C | LEU | A | 222 | 30.750 | −6.156 | 1.994 | 1.00 | 0.00 | C |
| ATOM | 3631 | O | LEU | A | 222 | 29.617 | −6.510 | 1.668 | 1.00 | 0.00 | O |
| ATOM | 3632 | CB | LEU | A | 222 | 31.252 | −4.498 | 3.805 | 1.00 | 0.00 | C |
| ATOM | 3633 | CG | LEU | A | 222 | 30.010 | −3.617 | 3.515 | 1.00 | 0.00 | C |
| ATOM | 3634 | CD1 | LEU | A | 222 | 28.719 | −4.151 | 4.154 | 1.00 | 0.00 | C |
| ATOM | 3635 | CD2 | LEU | A | 222 | 30.254 | −2.166 | 3.950 | 1.00 | 0.00 | C |
| ATOM | 3636 | H | LEU | A | 222 | 33.207 | −6.115 | 4.074 | 1.00 | 0.00 | H |
| ATOM | 3637 | HA | LEU | A | 222 | 30.397 | −6.468 | 4.091 | 1.00 | 0.00 | H |
| ATOM | 3638 | 1HB | LEU | A | 222 | 31.516 | −4.401 | 4.855 | 1.00 | 0.00 | H |
| ATOM | 3639 | 2HB | LEU | A | 222 | 32.100 | −4.070 | 3.268 | 1.00 | 0.00 | H |
| ATOM | 3640 | HG | LEU | A | 222 | 29.859 | −3.594 | 2.436 | 1.00 | 0.00 | H |
| ATOM | 3641 | 1HD1 | LEU | A | 222 | 27.872 | −3.499 | 3.936 | 1.00 | 0.00 | H |
| ATOM | 3642 | 2HD1 | LEU | A | 222 | 28.467 | −5.137 | 3.768 | 1.00 | 0.00 | H |
| ATOM | 3643 | 3HD1 | LEU | A | 222 | 28.808 | −4.227 | 5.236 | 1.00 | 0.00 | H |
| ATOM | 3644 | 1HD2 | LEU | A | 222 | 29.421 | −1.522 | 3.666 | 1.00 | 0.00 | H |
| ATOM | 3645 | 2HD2 | LEU | A | 222 | 30.377 | −2.093 | 5.030 | 1.00 | 0.00 | H |
| ATOM | 3646 | 3HD2 | LEU | A | 222 | 31.152 | −1.760 | 3.480 | 1.00 | 0.00 | H |
| ATOM | 3647 | N | CYS | A | 223 | 31.706 | −5.912 | 1.109 | 1.00 | 0.00 | N |
| ATOM | 3648 | CA | CYS | A | 223 | 31.478 | −6.039 | −0.317 | 1.00 | 0.00 | C |
| ATOM | 3649 | C | CYS | A | 223 | 30.900 | −7.394 | −0.718 | 1.00 | 0.00 | C |
| ATOM | 3650 | O | CYS | A | 223 | 29.919 | −7.471 | −1.459 | 1.00 | 0.00 | O |
| ATOM | 3651 | CB | CYS | A | 223 | 32.791 | −5.819 | −1.048 | 1.00 | 0.00 | C |
| ATOM | 3652 | SG | CYS | A | 223 | 32.472 | −5.262 | −2.746 | 1.00 | 0.00 | S |
| ATOM | 3653 | H | CYS | A | 223 | 32.620 | −5.621 | 1.439 | 1.00 | 0.00 | H |
| ATOM | 3654 | HA | CYS | A | 223 | 30.760 | −5.264 | −0.592 | 1.00 | 0.00 | H |
| ATOM | 3655 | 1HB | CYS | A | 223 | 33.375 | −5.030 | −0.578 | 1.00 | 0.00 | H |
| ATOM | 3656 | 2HB | CYS | A | 223 | 33.422 | −6.707 | −1.086 | 1.00 | 0.00 | H |
| ATOM | 3657 | HG | CYS | A | 223 | 31.821 | −6.364 | −3.138 | 1.00 | 0.00 | H |
| ATOM | 3658 | N | SER | A | 224 | 31.517 | −8.464 | −0.230 | 1.00 | 0.00 | N |
| ATOM | 3659 | CA | SER | A | 224 | 31.072 | −9.813 | −0.552 | 1.00 | 0.00 | C |
| ATOM | 3660 | C | SER | A | 224 | 29.677 | −10.103 | −0.036 | 1.00 | 0.00 | C |
| ATOM | 3661 | O | SER | A | 224 | 29.008 | −11.021 | −0.520 | 1.00 | 0.00 | O |
| ATOM | 3662 | CB | SER | A | 224 | 32.040 | −10.832 | 0.030 | 1.00 | 0.00 | C |
| ATOM | 3663 | OG | SER | A | 224 | 31.805 | −12.182 | −0.332 | 1.00 | 0.00 | O |
| ATOM | 3664 | H | SER | A | 224 | 32.294 | −8.361 | 0.417 | 1.00 | 0.00 | H |
| ATOM | 3665 | HA | SER | A | 224 | 31.063 | −9.920 | −1.639 | 1.00 | 0.00 | H |
| ATOM | 3666 | 1HB | SER | A | 224 | 33.051 | −10.581 | −0.295 | 1.00 | 0.00 | H |
| ATOM | 3667 | 2HB | SER | A | 224 | 32.051 | −10.764 | 1.120 | 1.00 | 0.00 | H |
| ATOM | 3668 | HG | SER | A | 224 | 32.544 | −12.701 | −0.041 | 1.00 | 0.00 | H |
| ATOM | 3669 | N | SER | A | 225 | 29.236 | −9.319 | 0.943 | 1.00 | 0.00 | N |
| ATOM | 3670 | CA | SER | A | 225 | 27.910 | −9.494 | 1.532 | 1.00 | 0.00 | C |
| ATOM | 3671 | C | SER | A | 225 | 26.834 | −8.655 | 0.848 | 1.00 | 0.00 | C |
| ATOM | 3672 | O | SER | A | 225 | 25.648 | −8.811 | 1.126 | 1.00 | 0.00 | O |
| ATOM | 3673 | CB | SER | A | 225 | 27.953 | −9.164 | 3.029 | 1.00 | 0.00 | C |
| ATOM | 3674 | OG | SER | A | 225 | 27.128 | −10.026 | 3.794 | 1.00 | 0.00 | O |
| ATOM | 3675 | H | SER | A | 225 | 29.866 | −8.633 | 1.340 | 1.00 | 0.00 | H |
| ATOM | 3676 | HA | SER | A | 225 | 27.608 | −10.541 | 1.439 | 1.00 | 0.00 | H |
| ATOM | 3677 | 1HB | SER | A | 225 | 28.967 | −9.281 | 3.414 | 1.00 | 0.00 | H |
| ATOM | 3678 | 2HB | SER | A | 225 | 27.694 | −8.120 | 3.206 | 1.00 | 0.00 | H |
| ATOM | 3679 | HG | SER | A | 225 | 27.075 | −9.687 | 4.677 | 1.00 | 0.00 | H |
| ATOM | 3680 | N | LEU | A | 226 | 27.249 | −7.759 | −0.039 | 1.00 | 0.00 | N |
| ATOM | 3681 | CA | LEU | A | 226 | 26.301 | −6.926 | −0.772 | 1.00 | 0.00 | C |
| ATOM | 3682 | C | LEU | A | 226 | 26.140 | −7.479 | −2.182 | 1.00 | 0.00 | C |
| ATOM | 3683 | O | LEU | A | 226 | 25.026 | −7.594 | −2.700 | 1.00 | 0.00 | O |
| ATOM | 3684 | CB | LEU | A | 226 | 26.797 | −5.477 | −0.851 | 1.00 | 0.00 | C |
| ATOM | 3685 | CG | LEU | A | 226 | 26.940 | −4.749 | 0.506 | 1.00 | 0.00 | C |
| ATOM | 3686 | CD1 | LEU | A | 226 | 27.816 | −3.496 | 0.359 | 1.00 | 0.00 | C |
| ATOM | 3687 | CD2 | LEU | A | 226 | 25.588 | −4.365 | 1.117 | 1.00 | 0.00 | C |
| ATOM | 3688 | H | LEU | A | 226 | 28.243 | −7.670 | −0.214 | 1.00 | 0.00 | H |
| ATOM | 3689 | HA | LEU | A | 226 | 25.309 | −6.923 | −0.319 | 1.00 | 0.00 | H |
| ATOM | 3690 | 1HB | LEU | A | 226 | 27.767 | −5.470 | −1.351 | 1.00 | 0.00 | H |
| ATOM | 3691 | 2HB | LEU | A | 226 | 26.134 | −4.896 | −1.497 | 1.00 | 0.00 | H |
| ATOM | 3692 | HG | LEU | A | 226 | 27.436 | −5.410 | 1.214 | 1.00 | 0.00 | H |
| ATOM | 3693 | 1HD1 | LEU | A | 226 | 27.911 | −2.968 | 1.308 | 1.00 | 0.00 | H |
| ATOM | 3694 | 2HD1 | LEU | A | 226 | 28.821 | −3.750 | 0.022 | 1.00 | 0.00 | H |
| ATOM | 3695 | 3HD1 | LEU | A | 226 | 27.384 | −2.800 | −0.358 | 1.00 | 0.00 | H |
| ATOM | 3696 | 1HD2 | LEU | A | 226 | 25.714 | −3.913 | 2.101 | 1.00 | 0.00 | H |
| ATOM | 3697 | 2HD2 | LEU | A | 226 | 25.076 | −3.639 | 0.486 | 1.00 | 0.00 | H |
| ATOM | 3698 | 3HD2 | LEU | A | 226 | 24.938 | −5.231 | 1.239 | 1.00 | 0.00 | H |
| ATOM | 3699 | N | LEU | A | 227 | 27.265 | −7.818 | −2.800 | 1.00 | 0.00 | N |
| ATOM | 3700 | CA | LEU | A | 227 | 27.255 | −8.347 | −4.156 | 1.00 | 0.00 | C |
| ATOM | 3701 | C | LEU | A | 227 | 27.075 | −9.868 | −4.161 | 1.00 | 0.00 | C |
| ATOM | 3702 | O | LEU | A | 227 | 28.013 | −10.621 | −4.421 | 1.00 | 0.00 | O |
| ATOM | 3703 | CB | LEU | A | 227 | 28.545 | −7.935 | −4.861 | 1.00 | 0.00 | C |
| ATOM | 3704 | CG | LEU | A | 227 | 28.693 | −6.395 | −5.003 | 1.00 | 0.00 | C |
| ATOM | 3705 | CD1 | LEU | A | 227 | 30.064 | −6.013 | −5.569 | 1.00 | 0.00 | C |
| ATOM | 3706 | CD2 | LEU | A | 227 | 27.568 | −5.769 | −5.844 | 1.00 | 0.00 | C |
| ATOM | 3707 | H | LEU | A | 227 | 28.157 | −7.738 | −2.321 | 1.00 | 0.00 | H |
| ATOM | 3708 | HA | LEU | A | 227 | 26.416 | −7.946 | −4.724 | 1.00 | 0.00 | H |
| ATOM | 3709 | 1HB | LEU | A | 227 | 29.400 | −8.332 | −4.311 | 1.00 | 0.00 | H |
| ATOM | 3710 | 2HB | LEU | A | 227 | 28.585 | −8.392 | −5.852 | 1.00 | 0.00 | H |
| ATOM | 3711 | HG | LEU | A | 227 | 28.647 | −5.944 | −4.011 | 1.00 | 0.00 | H |
| ATOM | 3712 | 1HD1 | LEU | A | 227 | 30.170 | −4.932 | −5.669 | 1.00 | 0.00 | H |
| ATOM | 3713 | 2HD1 | LEU | A | 227 | 30.851 | −6.349 | −4.899 | 1.00 | 0.00 | H |
| ATOM | 3714 | 3HD1 | LEU | A | 227 | 30.239 | −6.457 | −6.547 | 1.00 | 0.00 | H |
| ATOM | 3715 | 1HD2 | LEU | A | 227 | 27.781 | −4.732 | −6.101 | 1.00 | 0.00 | H |
| ATOM | 3716 | 2HD2 | LEU | A | 227 | 27.418 | −6.312 | −6.776 | 1.00 | 0.00 | H |
| ATOM | 3717 | 3HD2 | LEU | A | 227 | 26.617 | −5.770 | −5.311 | 1.00 | 0.00 | H |
| ATOM | 3718 | N | GLU | A | 228 | 25.854 | −10.300 | −3.854 | 1.00 | 0.00 | N |
| ATOM | 3719 | CA | GLU | A | 228 | 25.494 | −11.718 | −3.812 | 1.00 | 0.00 | C |
| ATOM | 3720 | C | GLU | A | 228 | 24.463 | −11.995 | −4.897 | 1.00 | 0.00 | C |
| ATOM | 3721 | O | GLU | A | 228 | 23.582 | −11.172 | −5.128 | 1.00 | 0.00 | O |
| ATOM | 3722 | CB | GLU | A | 228 | 24.879 | −12.083 | −2.452 | 1.00 | 0.00 | C |
| ATOM | 3723 | CG | GLU | A | 228 | 25.318 | −13.466 | −1.901 | 1.00 | 0.00 | C |
| ATOM | 3724 | CD | GLU | A | 228 | 24.974 | −14.648 | −2.808 | 1.00 | 0.00 | C |
| ATOM | 3725 | OE1 | GLU | A | 228 | 23.868 | −15.204 | −2.659 | 1.00 | 0.00 | O |
| ATOM | 3726 | OE2 | GLU | A | 228 | 25.776 | −14.928 | −3.723 | 1.00 | 0.00 | O |
| ATOM | 3727 | H | GLU | A | 228 | 25.158 | −9.606 | −3.621 | 1.00 | 0.00 | H |
| ATOM | 3728 | HA | GLU | A | 228 | 26.362 | −12.362 | −3.976 | 1.00 | 0.00 | H |
| ATOM | 3729 | 1HB | GLU | A | 228 | 25.177 | −11.342 | −1.709 | 1.00 | 0.00 | H |
| ATOM | 3730 | 2HB | GLU | A | 228 | 23.787 | −12.041 | −2.474 | 1.00 | 0.00 | H |
| ATOM | 3731 | 1HG | GLU | A | 228 | 26.396 | −13.469 | −1.737 | 1.00 | 0.00 | H |
| ATOM | 3732 | 2HG | GLU | A | 228 | 24.866 | −13.635 | −0.925 | 1.00 | 0.00 | H |
| ATOM | 3733 | N | TYR | A | 229 | 24.566 | −13.153 | −5.550 | 1.00 | 0.00 | N |
| ATOM | 3734 | CA | TYR | A | 229 | 23.618 | −13.534 | −6.598 | 1.00 | 0.00 | C |
| ATOM | 3735 | C | TYR | A | 229 | 22.181 | −13.621 | −6.055 | 1.00 | 0.00 | C |
| ATOM | 3736 | O | TYR | A | 229 | 21.261 | −13.013 | −6.595 | 1.00 | 0.00 | O |
| ATOM | 3737 | CB | TYR | A | 229 | 23.997 | −14.896 | −7.206 | 1.00 | 0.00 | C |
| ATOM | 3738 | CG | TYR | A | 229 | 25.094 | −14.850 | −8.243 | 1.00 | 0.00 | C |
| ATOM | 3739 | CD1 | TYR | A | 229 | 24.938 | −14.066 | −9.406 | 1.00 | 0.00 | C |
| ATOM | 3740 | CD2 | TYR | A | 229 | 26.253 | −15.628 | −8.068 | 1.00 | 0.00 | C |
| ATOM | 3741 | CE1 | TYR | A | 229 | 25.930 | −14.075 | −10.401 | 1.00 | 0.00 | C |
| ATOM | 3742 | CE2 | TYR | A | 229 | 27.241 | −15.640 | −9.063 | 1.00 | 0.00 | C |
| ATOM | 3743 | CZ | TYR | A | 229 | 27.076 | −14.870 | −10.232 | 1.00 | 0.00 | C |
| ATOM | 3744 | OH | TYR | A | 229 | 28.017 | −14.905 | −11.210 | 1.00 | 0.00 | O |
| ATOM | 3745 | H | TYR | A | 229 | 25.330 | −13.776 | −5.333 | 1.00 | 0.00 | H |
| ATOM | 3746 | HA | TYR | A | 229 | 23.640 | −12.738 | −7.340 | 1.00 | 0.00 | H |
| ATOM | 3747 | 1HB | TYR | A | 229 | 24.253 | −15.597 | −6.412 | 1.00 | 0.00 | H |
| ATOM | 3748 | 2HB | TYR | A | 229 | 23.131 | −15.327 | −7.712 | 1.00 | 0.00 | H |
| ATOM | 3749 | HD1 | TYR | A | 229 | 24.053 | −13.464 | −9.552 | 1.00 | 0.00 | H |
| ATOM | 3750 | HD2 | TYR | A | 229 | 26.386 | −16.228 | −7.179 | 1.00 | 0.00 | H |
| ATOM | 3751 | HE1 | TYR | A | 229 | 25.807 | −13.483 | −11.296 | 1.00 | 0.00 | H |
| ATOM | 3752 | HE2 | TYR | A | 229 | 28.120 | −16.248 | −8.908 | 1.00 | 0.00 | H |
| ATOM | 3753 | HH | TYR | A | 229 | 28.675 | −15.577 | −11.015 | 1.00 | 0.00 | H |
| ATOM | 3754 | N | THR | A | 230 | 21.982 | −14.393 | −4.992 | 1.00 | 0.00 | N |
| ATOM | 3755 | CA | THR | A | 230 | 20.645 | −14.537 | −4.421 | 1.00 | 0.00 | C |
| ATOM | 3756 | C | THR | A | 230 | 20.240 | −13.248 | −3.729 | 1.00 | 0.00 | C |
| ATOM | 3757 | O | THR | A | 230 | 20.828 | −12.867 | −2.717 | 1.00 | 0.00 | O |
| ATOM | 3758 | CB | THR | A | 230 | 20.584 | −15.690 | −3.396 | 1.00 | 0.00 | C |
| ATOM | 3759 | OG1 | THR | A | 230 | 20.824 | −16.901 | −4.095 | 1.00 | 0.00 | O |
| ATOM | 3760 | CG2 | THR | A | 230 | 19.249 | −15.866 | −2.660 | 1.00 | 0.00 | C |
| ATOM | 3761 | H | THR | A | 230 | 22.734 | −14.914 | −4.570 | 1.00 | 0.00 | H |
| ATOM | 3762 | HA | THR | A | 230 | 19.989 | −14.843 | −5.233 | 1.00 | 0.00 | H |
| ATOM | 3763 | HB | THR | A | 230 | 21.382 | −15.565 | −2.668 | 1.00 | 0.00 | H |
| ATOM | 3764 | HG1 | THR | A | 230 | 21.063 | −17.558 | −3.456 | 1.00 | 0.00 | H |
| ATOM | 3765 | 1HG2 | THR | A | 230 | 19.226 | −16.789 | −2.080 | 1.00 | 0.00 | H |
| ATOM | 3766 | 2HG2 | THR | A | 230 | 19.097 | −15.056 | −1.949 | 1.00 | 0.00 | H |
| ATOM | 3767 | 3HG2 | THR | A | 230 | 18.399 | −15.874 | −3.341 | 1.00 | 0.00 | H |
| ATOM | 3768 | N | PRO | A | 231 | 19.223 | −12.562 | −4.262 | 1.00 | 0.00 | N |
| ATOM | 3769 | CA | PRO | A | 231 | 18.735 | −11.296 | −3.696 | 1.00 | 0.00 | C |
| ATOM | 3770 | C | PRO | A | 231 | 18.515 | −11.291 | −2.180 | 1.00 | 0.00 | C |
| ATOM | 3771 | O | PRO | A | 231 | 18.943 | −10.366 | −1.484 | 1.00 | 0.00 | O |
| ATOM | 3772 | CB | PRO | A | 231 | 17.442 | −11.031 | −4.468 | 1.00 | 0.00 | C |
| ATOM | 3773 | CG | PRO | A | 231 | 17.522 | −11.792 | −5.765 | 1.00 | 0.00 | C |
| ATOM | 3774 | CD | PRO | A | 231 | 18.404 | −12.971 | −5.420 | 1.00 | 0.00 | C |
| ATOM | 3775 | HA | PRO | A | 231 | 19.467 | −10.527 | −3.945 | 1.00 | 0.00 | H |
| ATOM | 3776 | 1HB | PRO | A | 231 | 16.572 | −11.384 | −3.912 | 1.00 | 0.00 | H |
| ATOM | 3777 | 2HB | PRO | A | 231 | 17.291 | −9.964 | −4.642 | 1.00 | 0.00 | H |
| ATOM | 3778 | 1HG | PRO | A | 231 | 18.020 | −11.175 | −6.515 | 1.00 | 0.00 | H |
| ATOM | 3779 | 2HG | PRO | A | 231 | 16.549 | −12.083 | −6.164 | 1.00 | 0.00 | H |
| ATOM | 3780 | 1HD | PRO | A | 231 | 18.943 | −13.286 | −6.309 | 1.00 | 0.00 | H |
| ATOM | 3781 | 2HD | PRO | A | 231 | 17.775 | −13.805 | −5.109 | 1.00 | 0.00 | H |
| ATOM | 3782 | N | SER | A | 232 | 17.852 | −12.327 | −1.672 | 1.00 | 0.00 | N |
| ATOM | 3783 | CA | SER | A | 232 | 17.564 | −12.431 | −0.249 | 1.00 | 0.00 | C |
| ATOM | 3784 | C | SER | A | 232 | 18.807 | −12.659 | 0.596 | 1.00 | 0.00 | C |
| ATOM | 3785 | O | SER | A | 232 | 18.768 | −12.489 | 1.811 | 1.00 | 0.00 | O |
| ATOM | 3786 | CB | SER | A | 232 | 16.575 | −13.580 | 0.030 | 1.00 | 0.00 | C |
| ATOM | 3787 | OG | SER | A | 232 | 16.991 | −14.792 | −0.569 | 1.00 | 0.00 | O |
| ATOM | 3788 | H | SER | A | 232 | 17.535 | −13.091 | −2.250 | 1.00 | 0.00 | H |
| ATOM | 3789 | HA | SER | A | 232 | 17.111 | −11.498 | 0.092 | 1.00 | 0.00 | H |
| ATOM | 3790 | 1HB | SER | A | 232 | 16.433 | −13.731 | 1.102 | 1.00 | 0.00 | H |
| ATOM | 3791 | 2HB | SER | A | 232 | 15.595 | −13.319 | −0.372 | 1.00 | 0.00 | H |
| ATOM | 3792 | HG | SER | A | 232 | 16.298 | −15.434 | −0.461 | 1.00 | 0.00 | H |
| ATOM | 3793 | N | SER | A | 233 | 19.911 | −13.041 | −0.034 | 1.00 | 0.00 | N |
| ATOM | 3794 | CA | SER | A | 233 | 21.141 | −13.287 | 0.708 | 1.00 | 0.00 | C |
| ATOM | 3795 | C | SER | A | 233 | 21.913 | −12.007 | 1.013 | 1.00 | 0.00 | C |
| ATOM | 3796 | O | SER | A | 233 | 22.782 | −11.994 | 1.888 | 1.00 | 0.00 | O |
| ATOM | 3797 | CB | SER | A | 233 | 22.026 | −14.245 | −0.051 | 1.00 | 0.00 | C |
| ATOM | 3798 | OG | SER | A | 233 | 21.443 | −15.535 | −0.112 | 1.00 | 0.00 | O |
| ATOM | 3799 | H | SER | A | 233 | 19.925 | −13.159 | −1.039 | 1.00 | 0.00 | H |
| ATOM | 3800 | HA | SER | A | 233 | 20.914 | −13.749 | 1.672 | 1.00 | 0.00 | H |
| ATOM | 3801 | 1HB | SER | A | 233 | 22.265 | −13.884 | −1.049 | 1.00 | 0.00 | H |
| ATOM | 3802 | 2HB | SER | A | 233 | 22.986 | −14.354 | 0.458 | 1.00 | 0.00 | H |
| ATOM | 3803 | HG | SER | A | 233 | 22.023 | −16.063 | −0.651 | 1.00 | 0.00 | H |
| ATOM | 3804 | N | ARG | A | 234 | 21.599 | −10.930 | 0.300 | 1.00 | 0.00 | N |
| ATOM | 3805 | CA | ARG | A | 234 | 22.291 | −9.660 | 0.514 | 1.00 | 0.00 | C |
| ATOM | 3806 | C | ARG | A | 234 | 21.896 | −9.003 | 1.832 | 1.00 | 0.00 | C |
| ATOM | 3807 | O | ARG | A | 234 | 20.766 | −9.144 | 2.295 | 1.00 | 0.00 | O |
| ATOM | 3808 | CB | ARG | A | 234 | 21.994 | −8.695 | −0.640 | 1.00 | 0.00 | C |
| ATOM | 3809 | CG | ARG | A | 234 | 22.263 | −9.306 | −2.042 | 1.00 | 0.00 | C |
| ATOM | 3810 | CD | ARG | A | 234 | 21.879 | −8.410 | −3.238 | 1.00 | 0.00 | C |
| ATOM | 3811 | NE | ARG | A | 234 | 21.794 | −9.200 | −4.484 | 1.00 | 0.00 | N |
| ATOM | 3812 | CZ | ARG | A | 234 | 20.923 | −9.061 | −5.495 | 1.00 | 0.00 | C |
| ATOM | 3813 | NH1 | ARG | A | 234 | 20.009 | −8.092 | −5.507 | 1.00 | 0.00 | N |
| ATOM | 3814 | NH2 | ARG | A | 234 | 20.967 | −9.922 | −6.510 | 1.00 | 0.00 | N |
| ATOM | 3815 | H | ARG | A | 234 | 20.790 | −10.957 | −0.307 | 1.00 | 0.00 | H |
| ATOM | 3816 | HA | ARG | A | 234 | 23.365 | −9.857 | 0.525 | 1.00 | 0.00 | H |
| ATOM | 3817 | 1HB | ARG | A | 234 | 20.953 | −8.376 | −0.582 | 1.00 | 0.00 | H |
| ATOM | 3818 | 2HB | ARG | A | 234 | 22.591 | −7.789 | −0.525 | 1.00 | 0.00 | H |
| ATOM | 3819 | 1HG | ARG | A | 234 | 23.320 | −9.563 | −2.113 | 1.00 | 0.00 | H |
| ATOM | 3820 | 2HG | ARG | A | 234 | 21.722 | −10.246 | −2.148 | 1.00 | 0.00 | H |
| ATOM | 3821 | 1HD | ARG | A | 234 | 20.909 | −7.959 | −3.050 | 1.00 | 0.00 | H |
| ATOM | 3822 | 2HD | ARG | A | 234 | 22.577 | −7.584 | −3.366 | 1.00 | 0.00 | H |
| ATOM | 3823 | HE | ARG | A | 234 | 22.512 | −9.926 | −4.601 | 1.00 | 0.00 | H |
| ATOM | 3824 | 1HH1 | ARG | A | 234 | 19.274 | −8.121 | −6.269 | 1.00 | 0.00 | H |
| ATOM | 3825 | 2HH1 | ARG | A | 234 | 19.933 | −7.373 | −4.799 | 1.00 | 0.00 | H |
| ATOM | 3826 | 1HH2 | ARG | A | 234 | 20.359 | −9.788 | −7.361 | 1.00 | 0.00 | H |
| ATOM | 3827 | 2HH2 | ARG | A | 234 | 21.525 | −10.768 | −6.510 | 1.00 | 0.00 | H |
| ATOM | 3828 | N | LEU | A | 235 | 22.841 | −8.288 | 2.434 | 1.00 | 0.00 | N |
| ATOM | 3829 | CA | LEU | A | 235 | 22.582 | −7.580 | 3.683 | 1.00 | 0.00 | C |
| ATOM | 3830 | C | LEU | A | 235 | 21.479 | −6.561 | 3.443 | 1.00 | 0.00 | C |
| ATOM | 3831 | O | LEU | A | 235 | 21.297 | −6.099 | 2.319 | 1.00 | 0.00 | O |
| ATOM | 3832 | CB | LEU | A | 235 | 23.829 | −6.817 | 4.127 | 1.00 | 0.00 | C |
| ATOM | 3833 | CG | LEU | A | 235 | 25.002 | −7.661 | 4.646 | 1.00 | 0.00 | C |
| ATOM | 3834 | CD1 | LEU | A | 235 | 26.163 | −6.735 | 5.044 | 1.00 | 0.00 | C |
| ATOM | 3835 | CD2 | LEU | A | 235 | 24.621 | −8.571 | 5.826 | 1.00 | 0.00 | C |
| ATOM | 3836 | H | LEU | A | 235 | 23.773 | −8.263 | 2.037 | 1.00 | 0.00 | H |
| ATOM | 3837 | HA | LEU | A | 235 | 22.249 | −8.287 | 4.444 | 1.00 | 0.00 | H |
| ATOM | 3838 | 1HB | LEU | A | 235 | 24.171 | −6.183 | 3.307 | 1.00 | 0.00 | H |
| ATOM | 3839 | 2HB | LEU | A | 235 | 23.551 | −6.123 | 4.923 | 1.00 | 0.00 | H |
| ATOM | 3840 | HG | LEU | A | 235 | 25.323 | −8.289 | 3.818 | 1.00 | 0.00 | H |
| ATOM | 3841 | 1HD1 | LEU | A | 235 | 27.041 | −7.298 | 5.362 | 1.00 | 0.00 | H |
| ATOM | 3842 | 2HD1 | LEU | A | 235 | 26.457 | −6.107 | 4.204 | 1.00 | 0.00 | H |
| ATOM | 3843 | 3HD1 | LEU | A | 235 | 25.881 | −6.076 | 5.866 | 1.00 | 0.00 | H |
| ATOM | 3844 | 1HD2 | LEU | A | 235 | 25.494 | −9.081 | 6.232 | 1.00 | 0.00 | H |
| ATOM | 3845 | 2HD2 | LEU | A | 235 | 24.162 | −8.004 | 6.636 | 1.00 | 0.00 | H |
| ATOM | 3846 | 3HD2 | LEU | A | 235 | 23.916 | −9.346 | 5.525 | 1.00 | 0.00 | H |
| ATOM | 3847 | N | SER | A | 236 | 20.725 | −6.219 | 4.480 | 1.00 | 0.00 | N |
| ATOM | 3848 | CA | SER | A | 236 | 19.713 | −5.184 | 4.329 | 1.00 | 0.00 | C |
| ATOM | 3849 | C | SER | A | 236 | 20.512 | −3.902 | 4.589 | 1.00 | 0.00 | C |
| ATOM | 3850 | O | SER | A | 236 | 21.611 | −3.957 | 5.145 | 1.00 | 0.00 | O |
| ATOM | 3851 | CB | SER | A | 236 | 18.633 | −5.256 | 5.410 | 1.00 | 0.00 | C |
| ATOM | 3852 | OG | SER | A | 236 | 17.732 | −4.173 | 5.580 | 1.00 | 0.00 | O |
| ATOM | 3853 | H | SER | A | 236 | 20.867 | −6.702 | 5.370 | 1.00 | 0.00 | H |
| ATOM | 3854 | HA | SER | A | 236 | 19.256 | −5.211 | 3.339 | 1.00 | 0.00 | H |
| ATOM | 3855 | 1HB | SER | A | 236 | 18.036 | −6.150 | 5.225 | 1.00 | 0.00 | H |
| ATOM | 3856 | 2HB | SER | A | 236 | 19.105 | −5.427 | 6.379 | 1.00 | 0.00 | H |
| ATOM | 3857 | HG | SER | A | 236 | 17.006 | −4.472 | 6.112 | 1.00 | 0.00 | H |
| ATOM | 3858 | N | PRO | A | 237 | 19.995 | −2.742 | 4.165 | 1.00 | 0.00 | N |
| ATOM | 3859 | CA | PRO | A | 237 | 20.730 | −1.496 | 4.409 | 1.00 | 0.00 | C |
| ATOM | 3860 | C | PRO | A | 237 | 21.062 | −1.262 | 5.885 | 1.00 | 0.00 | C |
| ATOM | 3861 | O | PRO | A | 237 | 22.162 | −0.821 | 6.209 | 1.00 | 0.00 | O |
| ATOM | 3862 | CB | PRO | A | 237 | 19.806 | −0.420 | 3.845 | 1.00 | 0.00 | C |
| ATOM | 3863 | CG | PRO | A | 237 | 18.986 | −1.091 | 2.761 | 1.00 | 0.00 | C |
| ATOM | 3864 | CD | PRO | A | 237 | 18.892 | −2.546 | 3.212 | 1.00 | 0.00 | C |
| ATOM | 3865 | HA | PRO | A | 237 | 21.658 | −1.525 | 3.834 | 1.00 | 0.00 | H |
| ATOM | 3866 | 1HB | PRO | A | 237 | 19.132 | −0.051 | 4.617 | 1.00 | 0.00 | H |
| ATOM | 3867 | 2HB | PRO | A | 237 | 20.365 | 0.438 | 3.473 | 1.00 | 0.00 | H |
| ATOM | 3868 | 1HG | PRO | A | 237 | 19.522 | −1.032 | 1.814 | 1.00 | 0.00 | H |
| ATOM | 3869 | 2HG | PRO | A | 237 | 18.009 | −0.626 | 2.617 | 1.00 | 0.00 | H |
| ATOM | 3870 | 1HD | PRO | A | 237 | 18.956 | −3.218 | 2.357 | 1.00 | 0.00 | H |
| ATOM | 3871 | 2HD | PRO | A | 237 | 17.924 | −2.700 | 3.682 | 1.00 | 0.00 | H |
| ATOM | 3872 | N | LEU | A | 238 | 20.118 | −1.557 | 6.778 | 1.00 | 0.00 | N |
| ATOM | 3873 | CA | LEU | A | 238 | 20.365 | −1.362 | 8.208 | 1.00 | 0.00 | C |
| ATOM | 3874 | C | LEU | A | 238 | 21.502 | −2.275 | 8.690 | 1.00 | 0.00 | C |
| ATOM | 3875 | O | LEU | A | 238 | 22.347 | −1.852 | 9.477 | 1.00 | 0.00 | O |
| ATOM | 3876 | CB | LEU | A | 238 | 19.085 | −1.615 | 9.025 | 1.00 | 0.00 | C |
| ATOM | 3877 | CG | LEU | A | 238 | 19.000 | −0.906 | 10.392 | 1.00 | 0.00 | C |
| ATOM | 3878 | CD1 | LEU | A | 238 | 19.073 | 0.629 | 10.269 | 1.00 | 0.00 | C |
| ATOM | 3879 | CD2 | LEU | A | 238 | 17.702 | −1.307 | 11.110 | 1.00 | 0.00 | C |
| ATOM | 3880 | H | LEU | A | 238 | 19.216 | −1.880 | 6.465 | 1.00 | 0.00 | H |
| ATOM | 3881 | HA | LEU | A | 238 | 20.695 | −0.330 | 8.320 | 1.00 | 0.00 | H |
| ATOM | 3882 | 1HB | LEU | A | 238 | 18.227 | −1.282 | 8.438 | 1.00 | 0.00 | H |
| ATOM | 3883 | 2HB | LEU | A | 238 | 18.939 | −2.689 | 9.158 | 1.00 | 0.00 | H |
| ATOM | 3884 | HG | LEU | A | 238 | 19.837 | −1.246 | 11.002 | 1.00 | 0.00 | H |
| ATOM | 3885 | 1HD1 | LEU | A | 238 | 18.901 | 1.115 | 11.226 | 1.00 | 0.00 | H |
| ATOM | 3886 | 2HD1 | LEU | A | 238 | 20.049 | 0.974 | 9.929 | 1.00 | 0.00 | H |
| ATOM | 3887 | 3HD1 | LEU | A | 238 | 18.320 | 1.012 | 9.579 | 1.00 | 0.00 | H |
| ATOM | 3888 | 1HD2 | LEU | A | 238 | 17.694 | −0.958 | 12.143 | 1.00 | 0.00 | H |
| ATOM | 3889 | 2HD2 | LEU | A | 238 | 16.827 | −0.895 | 10.605 | 1.00 | 0.00 | H |
| ATOM | 3890 | 3HD2 | LEU | A | 238 | 17.574 | −2.386 | 11.137 | 1.00 | 0.00 | H |
| ATOM | 3891 | N | GLU | A | 239 | 21.533 | −3.516 | 8.209 | 1.00 | 0.00 | N |
| ATOM | 3892 | CA | GLU | A | 239 | 22.593 | −4.443 | 8.588 | 1.00 | 0.00 | C |
| ATOM | 3893 | C | GLU | A | 239 | 23.937 | −3.927 | 8.080 | 1.00 | 0.00 | C |
| ATOM | 3894 | O | GLU | A | 239 | 24.960 | −4.051 | 8.765 | 1.00 | 0.00 | O |
| ATOM | 3895 | CB | GLU | A | 239 | 22.327 | −5.837 | 8.012 | 1.00 | 0.00 | C |
| ATOM | 3896 | CG | GLU | A | 239 | 21.071 | −6.500 | 8.611 | 1.00 | 0.00 | C |
| ATOM | 3897 | CD | GLU | A | 239 | 20.733 | −7.800 | 7.902 | 1.00 | 0.00 | C |
| ATOM | 3898 | OE1 | GLU | A | 239 | 20.777 | −7.787 | 6.656 | 1.00 | 0.00 | O |
| ATOM | 3899 | OE2 | GLU | A | 239 | 20.426 | −8.784 | 8.600 | 1.00 | 0.00 | O |
| ATOM | 3900 | H | GLU | A | 239 | 20.883 | −3.783 | 7.488 | 1.00 | 0.00 | H |
| ATOM | 3901 | HA | GLU | A | 239 | 22.649 | −4.508 | 9.677 | 1.00 | 0.00 | H |
| ATOM | 3902 | 1HB | GLU | A | 239 | 22.250 | −5.776 | 6.927 | 1.00 | 0.00 | H |
| ATOM | 3903 | 2HB | GLU | A | 239 | 23.184 | −6.483 | 8.212 | 1.00 | 0.00 | H |
| ATOM | 3904 | 1HG | GLU | A | 239 | 21.218 | −6.695 | 9.674 | 1.00 | 0.00 | H |
| ATOM | 3905 | 2HG | GLU | A | 239 | 20.204 | −5.847 | 8.529 | 1.00 | 0.00 | H |
| ATOM | 3906 | N | ALA | A | 240 | 23.936 | −3.350 | 6.877 | 1.00 | 0.00 | N |
| ATOM | 3907 | CA | ALA | A | 240 | 25.161 | −2.801 | 6.303 | 1.00 | 0.00 | C |
| ATOM | 3908 | C | ALA | A | 240 | 25.680 | −1.699 | 7.228 | 1.00 | 0.00 | C |
| ATOM | 3909 | O | ALA | A | 240 | 26.861 | −1.665 | 7.550 | 1.00 | 0.00 | O |
| ATOM | 3910 | CB | ALA | A | 240 | 24.891 | −2.235 | 4.890 | 1.00 | 0.00 | C |
| ATOM | 3911 | H | ALA | A | 240 | 23.075 | −3.311 | 6.342 | 1.00 | 0.00 | H |
| ATOM | 3912 | HA | ALA | A | 240 | 25.913 | −3.589 | 6.236 | 1.00 | 0.00 | H |
| ATOM | 3913 | 1HB | ALA | A | 240 | 25.821 | −1.910 | 4.424 | 1.00 | 0.00 | H |
| ATOM | 3914 | 2HB | ALA | A | 240 | 24.448 | −2.989 | 4.239 | 1.00 | 0.00 | H |
| ATOM | 3915 | 3HB | ALA | A | 240 | 24.224 | −1.375 | 4.903 | 1.00 | 0.00 | H |
| ATOM | 3916 | N | CYS | A | 241 | 24.789 | −0.814 | 7.670 | 1.00 | 0.00 | N |
| ATOM | 3917 | CA | CYS | A | 241 | 25.170 | 0.280 | 8.568 | 1.00 | 0.00 | C |
| ATOM | 3918 | C | CYS | A | 241 | 25.867 | −0.251 | 9.831 | 1.00 | 0.00 | C |
| ATOM | 3919 | O | CYS | A | 241 | 26.801 | 0.368 | 10.346 | 1.00 | 0.00 | O |
| ATOM | 3920 | CB | CYS | A | 241 | 23.927 | 1.088 | 8.982 | 1.00 | 0.00 | C |
| ATOM | 3921 | SG | CYS | A | 241 | 23.239 | 2.059 | 7.620 | 1.00 | 0.00 | S |
| ATOM | 3922 | H | CYS | A | 241 | 23.835 | −0.862 | 7.324 | 1.00 | 0.00 | H |
| ATOM | 3923 | HA | CYS | A | 241 | 25.875 | 0.934 | 8.052 | 1.00 | 0.00 | H |
| ATOM | 3924 | 1HB | CYS | A | 241 | 23.148 | 0.461 | 9.406 | 1.00 | 0.00 | H |
| ATOM | 3925 | 2HB | CYS | A | 241 | 24.204 | 1.803 | 9.759 | 1.00 | 0.00 | H |
| ATOM | 3926 | HG | CYS | A | 241 | 22.292 | 2.665 | 8.342 | 1.00 | 0.00 | H |
| ATOM | 3927 | N | ALA | A | 242 | 25.409 | −1.406 | 10.314 | 1.00 | 0.00 | N |
| ATOM | 3928 | CA | ALA | A | 242 | 25.958 | −2.019 | 11.524 | 1.00 | 0.00 | C |
| ATOM | 3929 | C | ALA | A | 242 | 27.241 | −2.826 | 11.307 | 1.00 | 0.00 | C |
| ATOM | 3930 | O | ALA | A | 242 | 27.843 | −3.315 | 12.271 | 1.00 | 0.00 | O |
| ATOM | 3931 | CB | ALA | A | 242 | 24.889 | −2.899 | 12.176 | 1.00 | 0.00 | C |
| ATOM | 3932 | H | ALA | A | 242 | 24.663 | −1.882 | 9.819 | 1.00 | 0.00 | H |
| ATOM | 3933 | HA | ALA | A | 242 | 26.206 | −1.235 | 12.243 | 1.00 | 0.00 | H |
| ATOM | 3934 | 1HB | ALA | A | 242 | 25.245 | −3.330 | 13.113 | 1.00 | 0.00 | H |
| ATOM | 3935 | 2HB | ALA | A | 242 | 23.991 | −2.324 | 12.405 | 1.00 | 0.00 | H |
| ATOM | 3936 | 3HB | ALA | A | 242 | 24.597 | −3.723 | 11.523 | 1.00 | 0.00 | H |
| ATOM | 3937 | N | HIS | A | 243 | 27.666 | −2.954 | 10.051 | 1.00 | 0.00 | N |
| ATOM | 3938 | CA | HIS | A | 243 | 28.874 | −3.707 | 9.708 | 1.00 | 0.00 | C |
| ATOM | 3939 | C | HIS | A | 243 | 30.151 | −3.130 | 10.322 | 1.00 | 0.00 | C |
| ATOM | 3940 | O | HIS | A | 243 | 30.278 | −1.917 | 10.505 | 1.00 | 0.00 | O |
| ATOM | 3941 | CB | HIS | A | 243 | 29.029 | −3.773 | 8.184 | 1.00 | 0.00 | C |
| ATOM | 3942 | CG | HIS | A | 243 | 29.795 | −4.988 | 7.708 | 1.00 | 0.00 | C |
| ATOM | 3943 | ND1 | HIS | A | 243 | 29.213 | −6.243 | 7.621 | 1.00 | 0.00 | N |
| ATOM | 3944 | CD2 | HIS | A | 243 | 31.104 | −5.169 | 7.321 | 1.00 | 0.00 | C |
| ATOM | 3945 | CE1 | HIS | A | 243 | 30.143 | −7.091 | 7.179 | 1.00 | 0.00 | C |
| ATOM | 3946 | NE2 | HIS | A | 243 | 31.315 | −6.505 | 6.976 | 1.00 | 0.00 | N |
| ATOM | 3947 | H | HIS | A | 243 | 27.144 | −2.508 | 9.303 | 1.00 | 0.00 | H |
| ATOM | 3948 | HA | HIS | A | 243 | 28.731 | −4.723 | 10.083 | 1.00 | 0.00 | H |
| ATOM | 3949 | 1HB | HIS | A | 243 | 28.050 | −3.846 | 7.713 | 1.00 | 0.00 | H |
| ATOM | 3950 | 2HB | HIS | A | 243 | 29.488 | −2.864 | 7.791 | 1.00 | 0.00 | H |
| ATOM | 3951 | HD1 | HIS | A | 243 | 28.253 | −6.486 | 7.829 | 1.00 | 0.00 | H |
| ATOM | 3952 | HD2 | HIS | A | 243 | 31.911 | −4.455 | 7.248 | 1.00 | 0.00 | H |
| ATOM | 3953 | HE1 | HIS | A | 243 | 29.964 | −8.143 | 7.004 | 1.00 | 0.00 | H |
| ATOM | 3954 | N | SER | A | 244 | 31.103 | −4.013 | 10.614 | 1.00 | 0.00 | N |
| ATOM | 3955 | CA | SER | A | 244 | 32.388 | −3.634 | 11.206 | 1.00 | 0.00 | C |
| ATOM | 3956 | C | SER | A | 244 | 33.172 | −2.601 | 10.411 | 1.00 | 0.00 | C |
| ATOM | 3957 | O | SER | A | 244 | 33.958 | −1.842 | 10.978 | 1.00 | 0.00 | O |
| ATOM | 3958 | CB | SER | A | 244 | 33.258 | −4.875 | 11.394 | 1.00 | 0.00 | C |
| ATOM | 3959 | OG | SER | A | 244 | 33.387 | −5.609 | 10.182 | 1.00 | 0.00 | O |
| ATOM | 3960 | H | SER | A | 244 | 30.941 | −5.001 | 10.475 | 1.00 | 0.00 | H |
| ATOM | 3961 | HA | SER | A | 244 | 32.170 | −3.208 | 12.182 | 1.00 | 0.00 | H |
| ATOM | 3962 | 1HB | SER | A | 244 | 34.247 | −4.602 | 11.769 | 1.00 | 0.00 | H |
| ATOM | 3963 | 2HB | SER | A | 244 | 32.816 | −5.522 | 12.152 | 1.00 | 0.00 | H |
| ATOM | 3964 | HG | SER | A | 244 | 33.888 | −6.395 | 10.361 | 1.00 | 0.00 | H |
| ATOM | 3965 | N | PHE | A | 245 | 32.971 | −2.575 | 9.099 | 1.00 | 0.00 | N |
| ATOM | 3966 | CA | PHE | A | 245 | 33.674 | −1.615 | 8.257 | 1.00 | 0.00 | C |
| ATOM | 3967 | C | PHE | A | 245 | 33.382 | −0.192 | 8.726 | 1.00 | 0.00 | C |
| ATOM | 3968 | O | PHE | A | 245 | 34.203 | 0.700 | 8.567 | 1.00 | 0.00 | O |
| ATOM | 3969 | CB | PHE | A | 245 | 33.246 | −1.783 | 6.788 | 1.00 | 0.00 | C |
| ATOM | 3970 | CG | PHE | A | 245 | 33.754 | −0.711 | 5.837 | 1.00 | 0.00 | C |
| ATOM | 3971 | CD1 | PHE | A | 245 | 35.131 | −0.410 | 5.752 | 1.00 | 0.00 | C |
| ATOM | 3972 | CD2 | PHE | A | 245 | 32.823 | 0.089 | 5.147 | 1.00 | 0.00 | C |
| ATOM | 3973 | CE1 | PHE | A | 245 | 35.550 | 0.760 | 5.090 | 1.00 | 0.00 | C |
| ATOM | 3974 | CE2 | PHE | A | 245 | 33.239 | 1.253 | 4.483 | 1.00 | 0.00 | C |
| ATOM | 3975 | CZ | PHE | A | 245 | 34.596 | 1.612 | 4.499 | 1.00 | 0.00 | C |
| ATOM | 3976 | H | PHE | A | 245 | 32.358 | −3.269 | 8.704 | 1.00 | 0.00 | H |
| ATOM | 3977 | HA | PHE | A | 245 | 34.748 | −1.776 | 8.355 | 1.00 | 0.00 | H |
| ATOM | 3978 | 1HB | PHE | A | 245 | 33.587 | −2.748 | 6.413 | 1.00 | 0.00 | H |
| ATOM | 3979 | 2HB | PHE | A | 245 | 32.157 | −1.809 | 6.731 | 1.00 | 0.00 | H |
| ATOM | 3980 | HD1 | PHE | A | 245 | 35.860 | −1.017 | 6.267 | 1.00 | 0.00 | H |
| ATOM | 3981 | HD2 | PHE | A | 245 | 31.772 | −0.140 | 5.187 | 1.00 | 0.00 | H |
| ATOM | 3982 | HE1 | PHE | A | 245 | 36.595 | 1.031 | 5.081 | 1.00 | 0.00 | H |
| ATOM | 3983 | HE2 | PHE | A | 245 | 32.510 | 1.897 | 4.012 | 1.00 | 0.00 | H |
| ATOM | 3984 | HZ | PHE | A | 245 | 34.888 | 2.552 | 4.060 | 1.00 | 0.00 | H |
| ATOM | 3985 | N | PHE | A | 246 | 32.217 | 0.020 | 9.324 | 1.00 | 0.00 | N |
| ATOM | 3986 | CA | PHE | A | 246 | 31.851 | 1.360 | 9.781 | 1.00 | 0.00 | C |
| ATOM | 3987 | C | PHE | A | 246 | 32.162 | 1.636 | 11.248 | 1.00 | 0.00 | C |
| ATOM | 3988 | O | PHE | A | 246 | 31.736 | 2.660 | 11.787 | 1.00 | 0.00 | O |
| ATOM | 3989 | CB | PHE | A | 246 | 30.362 | 1.612 | 9.509 | 1.00 | 0.00 | C |
| ATOM | 3990 | CG | PHE | A | 246 | 29.447 | 2.532 | 10.325 | 1.00 | 0.00 | C |
| ATOM | 3991 | CD1 | PHE | A | 246 | 29.229 | 3.866 | 9.933 | 1.00 | 0.00 | C |
| ATOM | 3992 | CD2 | PHE | A | 246 | 28.787 | 2.039 | 11.472 | 1.00 | 0.00 | C |
| ATOM | 3993 | CE1 | PHE | A | 246 | 28.399 | 4.705 | 10.709 | 1.00 | 0.00 | C |
| ATOM | 3994 | CE2 | PHE | A | 246 | 27.891 | 2.853 | 12.191 | 1.00 | 0.00 | C |
| ATOM | 3995 | CZ | PHE | A | 246 | 27.714 | 4.197 | 11.825 | 1.00 | 0.00 | C |
| ATOM | 3996 | H | PHE | A | 246 | 31.576 | −0.746 | 9.494 | 1.00 | 0.00 | H |
| ATOM | 3997 | HA | PHE | A | 246 | 32.397 | 2.127 | 9.229 | 1.00 | 0.00 | H |
| ATOM | 3998 | 1HB | PHE | A | 246 | 30.281 | 1.954 | 8.482 | 1.00 | 0.00 | H |
| ATOM | 3999 | 2HB | PHE | A | 246 | 29.844 | 0.651 | 9.508 | 1.00 | 0.00 | H |
| ATOM | 4000 | HD1 | PHE | A | 246 | 29.723 | 4.243 | 9.051 | 1.00 | 0.00 | H |
| ATOM | 4001 | HD2 | PHE | A | 246 | 28.924 | 1.008 | 11.767 | 1.00 | 0.00 | H |
| ATOM | 4002 | HE1 | PHE | A | 246 | 28.274 | 5.745 | 10.459 | 1.00 | 0.00 | H |
| ATOM | 4003 | HE2 | PHE | A | 246 | 27.331 | 2.446 | 13.016 | 1.00 | 0.00 | H |
| ATOM | 4004 | HZ | PHE | A | 246 | 27.047 | 4.833 | 12.390 | 1.00 | 0.00 | H |
| ATOM | 4005 | N | ASP | A | 247 | 32.916 | 0.743 | 11.888 | 1.00 | 0.00 | N |
| ATOM | 4006 | CA | ASP | A | 247 | 33.257 | 0.905 | 13.302 | 1.00 | 0.00 | C |
| ATOM | 4007 | C | ASP | A | 247 | 34.010 | 2.195 | 13.625 | 1.00 | 0.00 | C |
| ATOM | 4008 | O | ASP | A | 247 | 33.798 | 2.787 | 14.686 | 1.00 | 0.00 | O |
| ATOM | 4009 | CB | ASP | A | 247 | 34.064 | −0.299 | 13.794 | 1.00 | 0.00 | C |
| ATOM | 4010 | CG | ASP | A | 247 | 33.244 | −1.486 | 14.288 | 1.00 | 0.00 | C |
| ATOM | 4011 | OD1 | ASP | A | 247 | 32.174 | −1.250 | 14.890 | 1.00 | 0.00 | O |
| ATOM | 4012 | OD2 | ASP | A | 247 | 33.725 | −2.629 | 14.147 | 1.00 | 0.00 | O |
| ATOM | 4013 | H | ASP | A | 247 | 33.237 | −0.090 | 11.402 | 1.00 | 0.00 | H |
| ATOM | 4014 | HA | ASP | A | 247 | 32.326 | 0.990 | 13.866 | 1.00 | 0.00 | H |
| ATOM | 4015 | 1HB | ASP | A | 247 | 34.785 | −0.625 | 13.045 | 1.00 | 0.00 | H |
| ATOM | 4016 | 2HB | ASP | A | 247 | 34.666 | −0.014 | 14.658 | 1.00 | 0.00 | H |
| ATOM | 4017 | N | GLU | A | 248 | 34.884 | 2.639 | 12.729 | 1.00 | 0.00 | N |
| ATOM | 4018 | CA | GLU | A | 248 | 35.626 | 3.870 | 12.971 | 1.00 | 0.00 | C |
| ATOM | 4019 | C | GLU | A | 248 | 34.689 | 5.066 | 13.116 | 1.00 | 0.00 | C |
| ATOM | 4020 | O | GLU | A | 248 | 34.970 | 5.993 | 13.876 | 1.00 | 0.00 | O |
| ATOM | 4021 | CB | GLU | A | 248 | 36.615 | 4.150 | 11.836 | 1.00 | 0.00 | C |
| ATOM | 4022 | CG | GLU | A | 248 | 37.355 | 5.526 | 11.877 | 1.00 | 0.00 | C |
| ATOM | 4023 | CD | GLU | A | 248 | 38.497 | 5.697 | 10.871 | 1.00 | 0.00 | C |
| ATOM | 4024 | OE1 | GLU | A | 248 | 38.891 | 4.699 | 10.239 | 1.00 | 0.00 | O |
| ATOM | 4025 | OE2 | GLU | A | 248 | 38.968 | 6.849 | 10.720 | 1.00 | 0.00 | O |
| ATOM | 4026 | H | GLU | A | 248 | 35.024 | 2.121 | 11.874 | 1.00 | 0.00 | H |
| ATOM | 4027 | HA | GLU | A | 248 | 36.184 | 3.768 | 13.905 | 1.00 | 0.00 | H |
| ATOM | 4028 | 1HB | GLU | A | 248 | 37.361 | 3.354 | 11.834 | 1.00 | 0.00 | H |
| ATOM | 4029 | 2HB | GLU | A | 248 | 36.105 | 4.070 | 10.875 | 1.00 | 0.00 | H |
| ATOM | 4030 | 1HG | GLU | A | 248 | 36.657 | 6.343 | 11.696 | 1.00 | 0.00 | H |
| ATOM | 4031 | 2HG | GLU | A | 248 | 37.774 | 5.689 | 12.870 | 1.00 | 0.00 | H |
| ATOM | 4032 | N | LEU | A | 249 | 33.579 | 5.054 | 12.382 | 1.00 | 0.00 | N |
| ATOM | 4033 | CA | LEU | A | 249 | 32.620 | 6.153 | 12.453 | 1.00 | 0.00 | C |
| ATOM | 4034 | C | LEU | A | 249 | 31.978 | 6.233 | 13.840 | 1.00 | 0.00 | C |
| ATOM | 4035 | O | LEU | A | 249 | 31.524 | 7.297 | 14.257 | 1.00 | 0.00 | O |
| ATOM | 4036 | CB | LEU | A | 249 | 31.529 | 5.985 | 11.384 | 1.00 | 0.00 | C |
| ATOM | 4037 | CG | LEU | A | 249 | 31.979 | 6.114 | 9.914 | 1.00 | 0.00 | C |
| ATOM | 4038 | CD1 | LEU | A | 249 | 30.942 | 6.849 | 9.041 | 1.00 | 0.00 | C |
| ATOM | 4039 | CD2 | LEU | A | 249 | 33.313 | 6.836 | 9.762 | 1.00 | 0.00 | C |
| ATOM | 4040 | H | LEU | A | 249 | 33.348 | 4.241 | 11.827 | 1.00 | 0.00 | H |
| ATOM | 4041 | HA | LEU | A | 249 | 33.141 | 7.100 | 12.299 | 1.00 | 0.00 | H |
| ATOM | 4042 | 1HB | LEU | A | 249 | 31.006 | 5.044 | 11.539 | 1.00 | 0.00 | H |
| ATOM | 4043 | 2HB | LEU | A | 249 | 30.774 | 6.751 | 11.570 | 1.00 | 0.00 | H |
| ATOM | 4044 | HG | LEU | A | 249 | 32.109 | 5.104 | 9.521 | 1.00 | 0.00 | H |
| ATOM | 4045 | 1HD1 | LEU | A | 249 | 31.233 | 6.826 | 7.990 | 1.00 | 0.00 | H |
| ATOM | 4046 | 2HD1 | LEU | A | 249 | 29.950 | 6.416 | 9.110 | 1.00 | 0.00 | H |
| ATOM | 4047 | 3HD1 | LEU | A | 249 | 30.857 | 7.896 | 9.334 | 1.00 | 0.00 | H |
| ATOM | 4048 | 1HD2 | LEU | A | 249 | 33.557 | 6.854 | 8.717 | 1.00 | 0.00 | H |
| ATOM | 4049 | 2HD2 | LEU | A | 249 | 33.276 | 7.869 | 10.109 | 1.00 | 0.00 | H |
| ATOM | 4050 | 3HD2 | LEU | A | 249 | 34.143 | 6.328 | 10.248 | 1.00 | 0.00 | H |
| ATOM | 4051 | N | ARG | A | 250 | 31.953 | 5.110 | 14.550 | 1.00 | 0.00 | N |
| ATOM | 4052 | CA | ARG | A | 250 | 31.363 | 5.065 | 15.886 | 1.00 | 0.00 | C |
| ATOM | 4053 | C | ARG | A | 250 | 32.377 | 5.360 | 17.004 | 1.00 | 0.00 | C |
| ATOM | 4054 | O | ARG | A | 250 | 32.045 | 5.285 | 18.195 | 1.00 | 0.00 | O |
| ATOM | 4055 | CB | ARG | A | 250 | 30.699 | 3.700 | 16.111 | 1.00 | 0.00 | C |
| ATOM | 4056 | CG | ARG | A | 250 | 29.616 | 3.267 | 15.108 | 1.00 | 0.00 | C |
| ATOM | 4057 | CD | ARG | A | 250 | 28.861 | 2.016 | 15.595 | 1.00 | 0.00 | C |
| ATOM | 4058 | NE | ARG | A | 250 | 29.751 | 0.861 | 15.821 | 1.00 | 0.00 | N |
| ATOM | 4059 | CZ | ARG | A | 250 | 29.744 | 0.023 | 16.867 | 1.00 | 0.00 | C |
| ATOM | 4060 | NH1 | ARG | A | 250 | 28.841 | 0.134 | 17.840 | 1.00 | 0.00 | N |
| ATOM | 4061 | NH2 | ARG | A | 250 | 30.653 | −0.940 | 16.936 | 1.00 | 0.00 | N |
| ATOM | 4062 | H | ARG | A | 250 | 32.406 | 4.280 | 14.186 | 1.00 | 0.00 | H |
| ATOM | 4063 | HA | ARG | A | 250 | 30.587 | 5.823 | 15.997 | 1.00 | 0.00 | H |
| ATOM | 4064 | 1HB | ARG | A | 250 | 31.487 | 2.946 | 16.071 | 1.00 | 0.00 | H |
| ATOM | 4065 | 2HB | ARG | A | 250 | 30.346 | 3.617 | 17.135 | 1.00 | 0.00 | H |
| ATOM | 4066 | 1HG | ARG | A | 250 | 28.901 | 4.079 | 14.970 | 1.00 | 0.00 | H |
| ATOM | 4067 | 2HG | ARG | A | 250 | 30.060 | 3.086 | 14.127 | 1.00 | 0.00 | H |
| ATOM | 4068 | 1HD | ARG | A | 250 | 28.352 | 2.280 | 16.520 | 1.00 | 0.00 | H |
| ATOM | 4069 | 2HD | ARG | A | 250 | 28.083 | 1.718 | 14.896 | 1.00 | 0.00 | H |
| ATOM | 4070 | HE | ARG | A | 250 | 30.407 | 0.650 | 15.075 | 1.00 | 0.00 | H |
| ATOM | 4071 | 1HH1 | ARG | A | 250 | 28.858 | −0.452 | 18.659 | 1.00 | 0.00 | H |
| ATOM | 4072 | 2HH1 | ARG | A | 250 | 27.985 | 0.663 | 17.660 | 1.00 | 0.00 | H |
| ATOM | 4073 | 1HH2 | ARG | A | 250 | 30.713 | −1.599 | 17.694 | 1.00 | 0.00 | H |
| ATOM | 4074 | 2HH2 | ARG | A | 250 | 31.311 | −1.105 | 16.128 | 1.00 | 0.00 | H |
| ATOM | 4075 | N | CYS | A | 251 | 33.605 | 5.696 | 16.618 | 1.00 | 0.00 | N |
| ATOM | 4076 | CA | CYS | A | 251 | 34.661 | 6.021 | 17.577 | 1.00 | 0.00 | C |
| ATOM | 4077 | C | CYS | A | 251 | 34.425 | 7.448 | 18.056 | 1.00 | 0.00 | C |
| ATOM | 4078 | O | CYS | A | 251 | 34.305 | 8.365 | 17.249 | 1.00 | 0.00 | O |
| ATOM | 4079 | CB | CYS | A | 251 | 36.033 | 5.924 | 16.897 | 1.00 | 0.00 | C |
| ATOM | 4080 | SG | CYS | A | 251 | 37.379 | 6.025 | 18.118 | 1.00 | 0.00 | S |
| ATOM | 4081 | H | CYS | A | 251 | 33.797 | 5.808 | 15.629 | 1.00 | 0.00 | H |
| ATOM | 4082 | HA | CYS | A | 251 | 34.626 | 5.323 | 18.416 | 1.00 | 0.00 | H |
| ATOM | 4083 | 1HB | CYS | A | 251 | 36.127 | 4.981 | 16.358 | 1.00 | 0.00 | H |
| ATOM | 4084 | 2HB | CYS | A | 251 | 36.173 | 6.717 | 16.178 | 1.00 | 0.00 | H |
| ATOM | 4085 | HG | CYS | A | 251 | 38.373 | 5.922 | 17.228 | 1.00 | 0.00 | H |
| ATOM | 4086 | N | LEU | A | 252 | 34.377 | 7.660 | 19.382 | 1.00 | 0.00 | N |
| ATOM | 4087 | CA | LEU | A | 252 | 34.150 | 8.996 | 19.943 | 1.00 | 0.00 | C |
| ATOM | 4088 | C | LEU | A | 252 | 35.168 | 10.063 | 19.542 | 1.00 | 0.00 | C |
| ATOM | 4089 | O | LEU | A | 252 | 34.868 | 11.257 | 19.586 | 1.00 | 0.00 | O |
| ATOM | 4090 | CB | LEU | A | 252 | 34.147 | 8.756 | 21.452 | 1.00 | 0.00 | C |
| ATOM | 4091 | CG | LEU | A | 252 | 35.447 | 8.220 | 22.123 | 1.00 | 0.00 | C |
| ATOM | 4092 | CD1 | LEU | A | 252 | 35.614 | 8.815 | 23.529 | 1.00 | 0.00 | C |
| ATOM | 4093 | CD2 | LEU | A | 252 | 35.498 | 6.682 | 22.200 | 1.00 | 0.00 | C |
| ATOM | 4094 | H | LEU | A | 252 | 34.477 | 6.884 | 20.018 | 1.00 | 0.00 | H |
| ATOM | 4095 | HA | LEU | A | 252 | 33.164 | 9.339 | 19.627 | 1.00 | 0.00 | H |
| ATOM | 4096 | 1HB | LEU | A | 252 | 33.891 | 9.713 | 21.911 | 1.00 | 0.00 | H |
| ATOM | 4097 | 2HB | LEU | A | 252 | 33.310 | 8.110 | 21.709 | 1.00 | 0.00 | H |
| ATOM | 4098 | HG | LEU | A | 252 | 36.320 | 8.546 | 21.559 | 1.00 | 0.00 | H |
| ATOM | 4099 | 1HD1 | LEU | A | 252 | 36.530 | 8.464 | 24.004 | 1.00 | 0.00 | H |
| ATOM | 4100 | 2HD1 | LEU | A | 252 | 35.666 | 9.904 | 23.495 | 1.00 | 0.00 | H |
| ATOM | 4101 | 3HD1 | LEU | A | 252 | 34.781 | 8.544 | 24.177 | 1.00 | 0.00 | H |
| ATOM | 4102 | 1HD2 | LEU | A | 252 | 36.385 | 6.350 | 22.741 | 1.00 | 0.00 | H |
| ATOM | 4103 | 2HD2 | LEU | A | 252 | 34.630 | 6.279 | 22.723 | 1.00 | 0.00 | H |
| ATOM | 4104 | 3HD2 | LEU | A | 252 | 35.546 | 6.214 | 21.218 | 1.00 | 0.00 | H |
| ATOM | 4105 | N | GLY | A | 253 | 36.367 | 9.647 | 19.146 | 1.00 | 0.00 | N |
| ATOM | 4106 | CA | GLY | A | 253 | 37.397 | 10.612 | 18.760 | 1.00 | 0.00 | C |
| ATOM | 4107 | C | GLY | A | 253 | 37.564 | 10.830 | 17.262 | 1.00 | 0.00 | C |
| ATOM | 4108 | O | GLY | A | 253 | 38.476 | 11.540 | 16.843 | 1.00 | 0.00 | O |
| ATOM | 4109 | H | GLY | A | 253 | 36.562 | 8.657 | 19.104 | 1.00 | 0.00 | H |
| ATOM | 4110 | 1HA | GLY | A | 253 | 37.271 | 11.583 | 19.242 | 1.00 | 0.00 | H |
| ATOM | 4111 | 2HA | GLY | A | 253 | 38.347 | 10.230 | 19.135 | 1.00 | 0.00 | H |
| ATOM | 4112 | N | THR | A | 254 | 36.696 | 10.224 | 16.457 | 1.00 | 0.00 | N |
| ATOM | 4113 | CA | THR | A | 254 | 36.788 | 10.376 | 15.011 | 1.00 | 0.00 | C |
| ATOM | 4114 | C | THR | A | 254 | 36.574 | 11.832 | 14.601 | 1.00 | 0.00 | C |
| ATOM | 4115 | O | THR | A | 254 | 35.750 | 12.544 | 15.176 | 1.00 | 0.00 | O |
| ATOM | 4116 | CB | THR | A | 254 | 35.758 | 9.468 | 14.277 | 1.00 | 0.00 | C |
| ATOM | 4117 | OG1 | THR | A | 254 | 36.354 | 8.194 | 14.348 | 1.00 | 0.00 | O |
| ATOM | 4118 | CG2 | THR | A | 254 | 35.578 | 9.698 | 12.762 | 1.00 | 0.00 | C |
| ATOM | 4119 | H | THR | A | 254 | 35.907 | 9.719 | 16.845 | 1.00 | 0.00 | H |
| ATOM | 4120 | HA | THR | A | 254 | 37.791 | 10.062 | 14.712 | 1.00 | 0.00 | H |
| ATOM | 4121 | HB | THR | A | 254 | 34.792 | 9.471 | 14.785 | 1.00 | 0.00 | H |
| ATOM | 4122 | HG1 | THR | A | 254 | 35.722 | 7.515 | 14.103 | 1.00 | 0.00 | H |
| ATOM | 4123 | 1HG2 | THR | A | 254 | 34.922 | 8.945 | 12.325 | 1.00 | 0.00 | H |
| ATOM | 4124 | 2HG2 | THR | A | 254 | 35.136 | 10.670 | 12.544 | 1.00 | 0.00 | H |
| ATOM | 4125 | 3HG2 | THR | A | 254 | 36.531 | 9.646 | 12.234 | 1.00 | 0.00 | H |
| ATOM | 4126 | N | GLN | A | 255 | 37.338 | 12.264 | 13.606 | 1.00 | 0.00 | N |
| ATOM | 4127 | CA | GLN | A | 255 | 37.261 | 13.624 | 13.094 | 1.00 | 0.00 | C |
| ATOM | 4128 | C | GLN | A | 255 | 37.462 | 13.610 | 11.586 | 1.00 | 0.00 | C |
| ATOM | 4129 | O | GLN | A | 255 | 38.040 | 12.671 | 11.038 | 1.00 | 0.00 | O |
| ATOM | 4130 | CB | GLN | A | 255 | 38.354 | 14.484 | 13.729 | 1.00 | 0.00 | C |
| ATOM | 4131 | CG | GLN | A | 255 | 38.145 | 14.833 | 15.218 | 1.00 | 0.00 | C |
| ATOM | 4132 | CD | GLN | A | 255 | 36.954 | 15.760 | 15.466 | 1.00 | 0.00 | C |
| ATOM | 4133 | OE1 | GLN | A | 255 | 37.042 | 16.967 | 15.276 | 1.00 | 0.00 | O |
| ATOM | 4134 | NE2 | GLN | A | 255 | 35.833 | 15.215 | 15.913 | 1.00 | 0.00 | N |
| ATOM | 4135 | H | GLN | A | 255 | 37.891 | 11.604 | 13.074 | 1.00 | 0.00 | H |
| ATOM | 4136 | HA | GLN | A | 255 | 36.281 | 14.068 | 13.277 | 1.00 | 0.00 | H |
| ATOM | 4137 | 1HB | GLN | A | 255 | 39.321 | 13.993 | 13.612 | 1.00 | 0.00 | H |
| ATOM | 4138 | 2HB | GLN | A | 255 | 38.443 | 15.427 | 13.183 | 1.00 | 0.00 | H |
| ATOM | 4139 | 1HG | GLN | A | 255 | 38.060 | 13.935 | 15.828 | 1.00 | 0.00 | H |
| ATOM | 4140 | 2HG | GLN | A | 255 | 39.034 | 15.347 | 15.584 | 1.00 | 0.00 | H |
| ATOM | 4141 | 1HE2 | GLN | A | 255 | 35.044 | 15.796 | 16.140 | 1.00 | 0.00 | H |
| ATOM | 4142 | 2HE2 | GLN | A | 255 | 35.752 | 14.197 | 15.935 | 1.00 | 0.00 | H |
| ATOM | 4143 | N | LEU | A | 256 | 36.990 | 14.649 | 10.913 | 1.00 | 0.00 | N |
| ATOM | 4144 | CA | LEU | A | 256 | 37.167 | 14.733 | 9.471 | 1.00 | 0.00 | C |
| ATOM | 4145 | C | LEU | A | 256 | 38.603 | 15.182 | 9.181 | 1.00 | 0.00 | C |
| ATOM | 4146 | O | LEU | A | 256 | 39.232 | 15.860 | 10.001 | 1.00 | 0.00 | O |
| ATOM | 4147 | CB | LEU | A | 256 | 36.195 | 15.751 | 8.865 | 1.00 | 0.00 | C |
| ATOM | 4148 | CG | LEU | A | 256 | 34.699 | 15.401 | 9.034 | 1.00 | 0.00 | C |
| ATOM | 4149 | CD1 | LEU | A | 256 | 33.817 | 16.341 | 8.199 | 1.00 | 0.00 | C |
| ATOM | 4150 | CD2 | LEU | A | 256 | 34.373 | 13.942 | 8.684 | 1.00 | 0.00 | C |
| ATOM | 4151 | H | LEU | A | 256 | 36.574 | 15.438 | 11.405 | 1.00 | 0.00 | H |
| ATOM | 4152 | HA | LEU | A | 256 | 36.972 | 13.751 | 9.047 | 1.00 | 0.00 | H |
| ATOM | 4153 | 1HB | LEU | A | 256 | 36.374 | 16.729 | 9.315 | 1.00 | 0.00 | H |
| ATOM | 4154 | 2HB | LEU | A | 256 | 36.397 | 15.895 | 7.805 | 1.00 | 0.00 | H |
| ATOM | 4155 | HG | LEU | A | 256 | 34.437 | 15.550 | 10.081 | 1.00 | 0.00 | H |
| ATOM | 4156 | 1HD1 | LEU | A | 256 | 32.759 | 16.140 | 8.370 | 1.00 | 0.00 | H |
| ATOM | 4157 | 2HD1 | LEU | A | 256 | 33.980 | 17.384 | 8.450 | 1.00 | 0.00 | H |
| ATOM | 4158 | 3HD1 | LEU | A | 256 | 34.009 | 16.231 | 7.133 | 1.00 | 0.00 | H |
| ATOM | 4159 | 1HD2 | LEU | A | 256 | 33.298 | 13.780 | 8.625 | 1.00 | 0.00 | H |
| ATOM | 4160 | 2HD2 | LEU | A | 256 | 34.799 | 13.670 | 7.723 | 1.00 | 0.00 | H |
| ATOM | 4161 | 3HD2 | LEU | A | 256 | 34.756 | 13.248 | 9.433 | 1.00 | 0.00 | H |
| ATOM | 4162 | N | PRO | A | 257 | 39.150 | 14.779 | 8.026 | 1.00 | 0.00 | N |
| ATOM | 4163 | CA | PRO | A | 257 | 40.517 | 15.192 | 7.691 | 1.00 | 0.00 | C |
| ATOM | 4164 | C | PRO | A | 257 | 40.696 | 16.714 | 7.713 | 1.00 | 0.00 | C |
| ATOM | 4165 | O | PRO | A | 257 | 41.807 | 17.203 | 7.924 | 1.00 | 0.00 | O |
| ATOM | 4166 | CB | PRO | A | 257 | 40.739 | 14.596 | 6.301 | 1.00 | 0.00 | C |
| ATOM | 4167 | CG | PRO | A | 257 | 39.404 | 14.152 | 5.740 | 1.00 | 0.00 | C |
| ATOM | 4168 | CD | PRO | A | 257 | 38.477 | 14.054 | 6.941 | 1.00 | 0.00 | C |
| ATOM | 4169 | HA | PRO | A | 257 | 41.204 | 14.737 | 8.408 | 1.00 | 0.00 | H |
| ATOM | 4170 | 1HB | PRO | A | 257 | 41.253 | 15.277 | 5.620 | 1.00 | 0.00 | H |
| ATOM | 4171 | 2HB | PRO | A | 257 | 41.385 | 13.723 | 6.399 | 1.00 | 0.00 | H |
| ATOM | 4172 | 1HG | PRO | A | 257 | 39.486 | 13.218 | 5.187 | 1.00 | 0.00 | H |
| ATOM | 4173 | 2HG | PRO | A | 257 | 39.028 | 14.908 | 5.049 | 1.00 | 0.00 | H |
| ATOM | 4174 | 1HD | PRO | A | 257 | 38.353 | 13.010 | 7.230 | 1.00 | 0.00 | H |
| ATOM | 4175 | 2HD | PRO | A | 257 | 37.493 | 14.441 | 6.684 | 1.00 | 0.00 | H |
| ATOM | 4176 | N | ASN | A | 258 | 39.617 | 17.466 | 7.504 | 1.00 | 0.00 | N |
| ATOM | 4177 | CA | ASN | A | 258 | 39.734 | 18.920 | 7.524 | 1.00 | 0.00 | C |
| ATOM | 4178 | C | ASN | A | 258 | 39.613 | 19.491 | 8.934 | 1.00 | 0.00 | C |
| ATOM | 4179 | O | ASN | A | 258 | 39.513 | 20.701 | 9.117 | 1.00 | 0.00 | O |
| ATOM | 4180 | CB | ASN | A | 258 | 38.709 | 19.573 | 6.578 | 1.00 | 0.00 | C |
| ATOM | 4181 | CG | ASN | A | 258 | 37.253 | 19.123 | 6.763 | 1.00 | 0.00 | C |
| ATOM | 4182 | OD1 | ASN | A | 258 | 36.800 | 18.893 | 7.879 | 1.00 | 0.00 | O |
| ATOM | 4183 | ND2 | ASN | A | 258 | 36.516 | 18.923 | 5.678 | 1.00 | 0.00 | N |
| ATOM | 4184 | H | ASN | A | 258 | 38.753 | 17.030 | 7.222 | 1.00 | 0.00 | H |
| ATOM | 4185 | HA | ASN | A | 258 | 40.712 | 19.228 | 7.149 | 1.00 | 0.00 | H |
| ATOM | 4186 | 1HB | ASN | A | 258 | 38.741 | 20.660 | 6.663 | 1.00 | 0.00 | H |
| ATOM | 4187 | 2HB | ASN | A | 258 | 39.008 | 19.358 | 5.552 | 1.00 | 0.00 | H |
| ATOM | 4188 | 1HD2 | ASN | A | 258 | 35.560 | 18.622 | 5.776 | 1.00 | 0.00 | H |
| ATOM | 4189 | 2HD2 | ASN | A | 258 | 36.881 | 19.101 | 4.754 | 1.00 | 0.00 | H |
| ATOM | 4190 | N | ASN | A | 259 | 39.630 | 18.611 | 9.928 | 1.00 | 0.00 | N |
| ATOM | 4191 | CA | ASN | A | 259 | 39.552 | 19.061 | 11.305 | 1.00 | 0.00 | C |
| ATOM | 4192 | C | ASN | A | 259 | 38.159 | 19.208 | 11.881 | 1.00 | 0.00 | C |
| ATOM | 4193 | O | ASN | A | 259 | 38.000 | 19.318 | 13.093 | 1.00 | 0.00 | O |
| ATOM | 4194 | CB | ASN | A | 259 | 40.564 | 20.175 | 11.714 | 1.00 | 0.00 | C |
| ATOM | 4195 | CG | ASN | A | 259 | 41.045 | 20.072 | 13.164 | 1.00 | 0.00 | C |
| ATOM | 4196 | OD1 | ASN | A | 259 | 41.714 | 19.111 | 13.533 | 1.00 | 0.00 | O |
| ATOM | 4197 | ND2 | ASN | A | 259 | 40.722 | 21.050 | 14.001 | 1.00 | 0.00 | N |
| ATOM | 4198 | H | ASN | A | 259 | 39.739 | 17.613 | 9.760 | 1.00 | 0.00 | H |
| ATOM | 4199 | HA | ASN | A | 259 | 39.907 | 18.161 | 11.811 | 1.00 | 0.00 | H |
| ATOM | 4200 | 1HB | ASN | A | 259 | 41.457 | 20.112 | 11.092 | 1.00 | 0.00 | H |
| ATOM | 4201 | 2HB | ASN | A | 259 | 40.137 | 21.163 | 11.539 | 1.00 | 0.00 | H |
| ATOM | 4202 | 1HD2 | ASN | A | 259 | 41.016 | 21.005 | 14.965 | 1.00 | 0.00 | H |
| ATOM | 4203 | 2HD2 | ASN | A | 259 | 40.152 | 21.824 | 13.694 | 1.00 | 0.00 | H |
| ATOM | 4204 | N | ARG | A | 260 | 37.144 | 19.211 | 11.028 | 1.00 | 0.00 | N |
| ATOM | 4205 | CA | ARG | A | 260 | 35.764 | 19.349 | 11.494 | 1.00 | 0.00 | C |
| ATOM | 4206 | C | ARG | A | 260 | 35.248 | 18.065 | 12.136 | 1.00 | 0.00 | C |
| ATOM | 4207 | O | ARG | A | 260 | 35.837 | 16.997 | 11.982 | 1.00 | 0.00 | O |
| ATOM | 4208 | CB | ARG | A | 260 | 34.847 | 19.695 | 10.328 | 1.00 | 0.00 | C |
| ATOM | 4209 | CG | ARG | A | 260 | 35.166 | 21.042 | 9.655 | 1.00 | 0.00 | C |
| ATOM | 4210 | CD | ARG | A | 260 | 34.142 | 21.409 | 8.566 | 1.00 | 0.00 | C |
| ATOM | 4211 | NE | ARG | A | 260 | 33.047 | 22.214 | 9.130 | 1.00 | 0.00 | N |
| ATOM | 4212 | CZ | ARG | A | 260 | 32.131 | 22.913 | 8.449 | 1.00 | 0.00 | C |
| ATOM | 4213 | NH1 | ARG | A | 260 | 32.071 | 22.869 | 7.119 | 1.00 | 0.00 | N |
| ATOM | 4214 | NH2 | ARG | A | 260 | 31.265 | 23.669 | 9.117 | 1.00 | 0.00 | N |
| ATOM | 4215 | H | ARG | A | 260 | 37.335 | 19.242 | 10.040 | 1.00 | 0.00 | H |
| ATOM | 4216 | HA | ARG | A | 260 | 35.722 | 20.152 | 12.233 | 1.00 | 0.00 | H |
| ATOM | 4217 | 1HB | ARG | A | 260 | 34.925 | 18.910 | 9.582 | 1.00 | 0.00 | H |
| ATOM | 4218 | 2HB | ARG | A | 260 | 33.798 | 19.695 | 10.619 | 1.00 | 0.00 | H |
| ATOM | 4219 | 1HG | ARG | A | 260 | 35.210 | 21.828 | 10.410 | 1.00 | 0.00 | H |
| ATOM | 4220 | 2HG | ARG | A | 260 | 36.162 | 21.015 | 9.213 | 1.00 | 0.00 | H |
| ATOM | 4221 | 1HD | ARG | A | 260 | 34.637 | 22.011 | 7.803 | 1.00 | 0.00 | H |
| ATOM | 4222 | 2HD | ARG | A | 260 | 33.753 | 20.524 | 8.060 | 1.00 | 0.00 | H |
| ATOM | 4223 | HE | ARG | A | 260 | 33.019 | 22.232 | 10.142 | 1.00 | 0.00 | H |
| ATOM | 4224 | 1HH1 | ARG | A | 260 | 31.415 | 23.430 | 6.599 | 1.00 | 0.00 | H |
| ATOM | 4225 | 2HH1 | ARG | A | 260 | 32.593 | 22.180 | 6.572 | 1.00 | 0.00 | H |
| ATOM | 4226 | 1HH2 | ARG | A | 260 | 30.555 | 24.215 | 8.654 | 1.00 | 0.00 | H |
| ATOM | 4227 | 2HH2 | ARG | A | 260 | 31.281 | 23.736 | 10.124 | 1.00 | 0.00 | H |
| ATOM | 4228 | N | PRO | A | 261 | 34.146 | 18.172 | 12.869 | 1.00 | 0.00 | N |
| ATOM | 4229 | CA | PRO | A | 261 | 33.557 | 16.985 | 13.473 | 1.00 | 0.00 | C |
| ATOM | 4230 | C | PRO | A | 261 | 32.792 | 16.285 | 12.358 | 1.00 | 0.00 | C |
| ATOM | 4231 | O | PRO | A | 261 | 32.459 | 16.895 | 11.338 | 1.00 | 0.00 | O |
| ATOM | 4232 | CB | PRO | A | 261 | 32.571 | 17.356 | 14.578 | 1.00 | 0.00 | C |
| ATOM | 4233 | CG | PRO | A | 261 | 32.245 | 18.811 | 14.217 | 1.00 | 0.00 | C |
| ATOM | 4234 | CD | PRO | A | 261 | 33.437 | 19.350 | 13.388 | 1.00 | 0.00 | C |
| ATOM | 4235 | HA | PRO | A | 261 | 34.320 | 16.311 | 13.866 | 1.00 | 0.00 | H |
| ATOM | 4236 | 1HB | PRO | A | 261 | 31.683 | 16.723 | 14.640 | 1.00 | 0.00 | H |
| ATOM | 4237 | 2HB | PRO | A | 261 | 33.075 | 17.319 | 15.544 | 1.00 | 0.00 | H |
| ATOM | 4238 | 1HG | PRO | A | 261 | 32.034 | 19.424 | 15.094 | 1.00 | 0.00 | H |
| ATOM | 4239 | 2HG | PRO | A | 261 | 31.349 | 18.823 | 13.594 | 1.00 | 0.00 | H |
| ATOM | 4240 | 1HD | PRO | A | 261 | 34.116 | 19.909 | 14.033 | 1.00 | 0.00 | H |
| ATOM | 4241 | 2HD | PRO | A | 261 | 33.085 | 20.026 | 12.611 | 1.00 | 0.00 | H |
| ATOM | 4242 | N | LEU | A | 262 | 32.519 | 15.005 | 12.548 | 1.00 | 0.00 | N |
| ATOM | 4243 | CA | LEU | A | 262 | 31.771 | 14.259 | 11.548 | 1.00 | 0.00 | C |
| ATOM | 4244 | C | LEU | A | 262 | 30.335 | 14.806 | 11.532 | 1.00 | 0.00 | C |
| ATOM | 4245 | O | LEU | A | 262 | 29.880 | 15.405 | 12.508 | 1.00 | 0.00 | O |
| ATOM | 4246 | CB | LEU | A | 262 | 31.738 | 12.769 | 11.908 | 1.00 | 0.00 | C |
| ATOM | 4247 | CG | LEU | A | 262 | 32.053 | 12.344 | 13.374 | 1.00 | 0.00 | C |
| ATOM | 4248 | CD1 | LEU | A | 262 | 30.983 | 12.806 | 14.379 | 1.00 | 0.00 | C |
| ATOM | 4249 | CD2 | LEU | A | 262 | 32.250 | 10.824 | 13.497 | 1.00 | 0.00 | C |
| ATOM | 4250 | H | LEU | A | 262 | 32.772 | 14.550 | 13.410 | 1.00 | 0.00 | H |
| ATOM | 4251 | HA | LEU | A | 262 | 32.231 | 14.394 | 10.568 | 1.00 | 0.00 | H |
| ATOM | 4252 | 1HB | LEU | A | 262 | 30.791 | 12.319 | 11.606 | 1.00 | 0.00 | H |
| ATOM | 4253 | 2HB | LEU | A | 262 | 32.477 | 12.287 | 11.266 | 1.00 | 0.00 | H |
| ATOM | 4254 | HG | LEU | A | 262 | 33.012 | 12.774 | 13.666 | 1.00 | 0.00 | H |
| ATOM | 4255 | 1HD1 | LEU | A | 262 | 31.205 | 12.444 | 15.383 | 1.00 | 0.00 | H |
| ATOM | 4256 | 2HD1 | LEU | A | 262 | 30.917 | 13.892 | 14.441 | 1.00 | 0.00 | H |
| ATOM | 4257 | 3HD1 | LEU | A | 262 | 29.995 | 12.435 | 14.104 | 1.00 | 0.00 | H |
| ATOM | 4258 | 1HD2 | LEU | A | 262 | 32.722 | 10.570 | 14.446 | 1.00 | 0.00 | H |
| ATOM | 4259 | 2HD2 | LEU | A | 262 | 31.311 | 10.279 | 13.465 | 1.00 | 0.00 | H |
| ATOM | 4260 | 3HD2 | LEU | A | 262 | 32.875 | 10.429 | 12.698 | 1.00 | 0.00 | H |
| ATOM | 4261 | N | PRO | A | 263 | 29.613 | 14.626 | 10.413 | 1.00 | 0.00 | N |
| ATOM | 4262 | CA | PRO | A | 263 | 28.232 | 15.119 | 10.344 | 1.00 | 0.00 | C |
| ATOM | 4263 | C | PRO | A | 263 | 27.324 | 14.214 | 11.176 | 1.00 | 0.00 | C |
| ATOM | 4264 | O | PRO | A | 263 | 27.803 | 13.320 | 11.873 | 1.00 | 0.00 | O |
| ATOM | 4265 | CB | PRO | A | 263 | 27.898 | 15.040 | 8.855 | 1.00 | 0.00 | C |
| ATOM | 4266 | CG | PRO | A | 263 | 28.870 | 14.038 | 8.244 | 1.00 | 0.00 | C |
| ATOM | 4267 | CD | PRO | A | 263 | 30.084 | 14.048 | 9.155 | 1.00 | 0.00 | C |
| ATOM | 4268 | HA | PRO | A | 263 | 28.160 | 16.147 | 10.703 | 1.00 | 0.00 | H |
| ATOM | 4269 | 1HB | PRO | A | 263 | 26.887 | 14.733 | 8.606 | 1.00 | 0.00 | H |
| ATOM | 4270 | 2HB | PRO | A | 263 | 28.074 | 16.021 | 8.417 | 1.00 | 0.00 | H |
| ATOM | 4271 | 1HG | PRO | A | 263 | 29.119 | 14.264 | 7.206 | 1.00 | 0.00 | H |
| ATOM | 4272 | 2HG | PRO | A | 263 | 28.439 | 13.042 | 8.267 | 1.00 | 0.00 | H |
| ATOM | 4273 | 1HD | PRO | A | 263 | 30.856 | 14.688 | 8.727 | 1.00 | 0.00 | H |
| ATOM | 4274 | 2HD | PRO | A | 263 | 30.497 | 13.045 | 9.274 | 1.00 | 0.00 | H |
| ATOM | 4275 | N | PRO | A | 264 | 26.019 | 14.445 | 11.104 | 1.00 | 0.00 | N |
| ATOM | 4276 | CA | PRO | A | 264 | 25.058 | 13.623 | 11.846 | 1.00 | 0.00 | C |
| ATOM | 4277 | C | PRO | A | 264 | 25.143 | 12.176 | 11.348 | 1.00 | 0.00 | C |
| ATOM | 4278 | O | PRO | A | 264 | 25.121 | 11.928 | 10.138 | 1.00 | 0.00 | O |
| ATOM | 4279 | CB | PRO | A | 264 | 23.644 | 14.170 | 11.646 | 1.00 | 0.00 | C |
| ATOM | 4280 | CG | PRO | A | 264 | 23.894 | 15.602 | 11.184 | 1.00 | 0.00 | C |
| ATOM | 4281 | CD | PRO | A | 264 | 25.323 | 15.644 | 10.608 | 1.00 | 0.00 | C |
| ATOM | 4282 | HA | PRO | A | 264 | 25.317 | 13.674 | 12.906 | 1.00 | 0.00 | H |
| ATOM | 4283 | 1HB | PRO | A | 264 | 23.106 | 13.628 | 10.865 | 1.00 | 0.00 | H |
| ATOM | 4284 | 2HB | PRO | A | 264 | 23.041 | 14.117 | 12.553 | 1.00 | 0.00 | H |
| ATOM | 4285 | 1HG | PRO | A | 264 | 23.841 | 16.265 | 12.049 | 1.00 | 0.00 | H |
| ATOM | 4286 | 2HG | PRO | A | 264 | 23.145 | 15.947 | 10.470 | 1.00 | 0.00 | H |
| ATOM | 4287 | 1HD | PRO | A | 264 | 25.833 | 16.565 | 10.893 | 1.00 | 0.00 | H |
| ATOM | 4288 | 2HD | PRO | A | 264 | 25.242 | 15.627 | 9.523 | 1.00 | 0.00 | H |
| ATOM | 4289 | N | LEU | A | 265 | 25.235 | 11.225 | 12.274 | 1.00 | 0.00 | N |
| ATOM | 4290 | CA | LEU | A | 265 | 25.341 | 9.815 | 11.904 | 1.00 | 0.00 | C |
| ATOM | 4291 | C | LEU | A | 265 | 24.496 | 8.886 | 12.756 | 1.00 | 0.00 | C |
| ATOM | 4292 | O | LEU | A | 265 | 24.266 | 7.730 | 12.387 | 1.00 | 0.00 | O |
| ATOM | 4293 | CB | LEU | A | 265 | 26.789 | 9.338 | 12.042 | 1.00 | 0.00 | C |
| ATOM | 4294 | CG | LEU | A | 265 | 27.863 | 9.938 | 11.113 | 1.00 | 0.00 | C |
| ATOM | 4295 | CD1 | LEU | A | 265 | 29.244 | 9.808 | 11.761 | 1.00 | 0.00 | C |
| ATOM | 4296 | CD2 | LEU | A | 265 | 27.867 | 9.268 | 9.743 | 1.00 | 0.00 | C |
| ATOM | 4297 | H | LEU | A | 265 | 25.388 | 11.491 | 13.232 | 1.00 | 0.00 | H |
| ATOM | 4298 | HA | LEU | A | 265 | 25.021 | 9.635 | 10.876 | 1.00 | 0.00 | H |
| ATOM | 4299 | 1HB | LEU | A | 265 | 27.090 | 9.529 | 13.074 | 1.00 | 0.00 | H |
| ATOM | 4300 | 2HB | LEU | A | 265 | 26.836 | 8.250 | 11.946 | 1.00 | 0.00 | H |
| ATOM | 4301 | HG | LEU | A | 265 | 27.681 | 10.999 | 10.964 | 1.00 | 0.00 | H |
| ATOM | 4302 | 1HD1 | LEU | A | 265 | 30.032 | 10.179 | 11.106 | 1.00 | 0.00 | H |
| ATOM | 4303 | 2HD1 | LEU | A | 265 | 29.273 | 10.393 | 12.679 | 1.00 | 0.00 | H |
| ATOM | 4304 | 3HD1 | LEU | A | 265 | 29.479 | 8.773 | 12.012 | 1.00 | 0.00 | H |
| ATOM | 4305 | 1HD2 | LEU | A | 265 | 28.761 | 9.533 | 9.180 | 1.00 | 0.00 | H |
| ATOM | 4306 | 2HD2 | LEU | A | 265 | 27.852 | 8.183 | 9.833 | 1.00 | 0.00 | H |
| ATOM | 4307 | 3HD2 | LEU | A | 265 | 26.997 | 9.593 | 9.179 | 1.00 | 0.00 | H |
| ATOM | 4308 | N | PHE | A | 266 | 24.044 | 9.380 | 13.901 | 1.00 | 0.00 | N |
| ATOM | 4309 | CA | PHE | A | 266 | 23.307 | 8.523 | 14.824 | 1.00 | 0.00 | C |
| ATOM | 4310 | C | PHE | A | 266 | 21.887 | 8.933 | 15.196 | 1.00 | 0.00 | C |
| ATOM | 4311 | O | PHE | A | 266 | 21.284 | 8.311 | 16.079 | 1.00 | 0.00 | O |
| ATOM | 4312 | CB | PHE | A | 266 | 24.140 | 8.351 | 16.101 | 1.00 | 0.00 | C |
| ATOM | 4313 | CG | PHE | A | 266 | 24.469 | 9.777 | 16.588 | 1.00 | 0.00 | C |
| ATOM | 4314 | CD1 | PHE | A | 266 | 25.548 | 10.500 | 16.023 | 1.00 | 0.00 | C |
| ATOM | 4315 | CD2 | PHE | A | 266 | 23.551 | 10.465 | 17.407 | 1.00 | 0.00 | C |
| ATOM | 4316 | CE1 | PHE | A | 266 | 25.655 | 11.894 | 16.205 | 1.00 | 0.00 | C |
| ATOM | 4317 | CE2 | PHE | A | 266 | 23.681 | 11.850 | 17.629 | 1.00 | 0.00 | C |
| ATOM | 4318 | CZ | PHE | A | 266 | 24.725 | 12.568 | 17.017 | 1.00 | 0.00 | C |
| ATOM | 4319 | H | PHE | A | 266 | 24.322 | 10.300 | 14.193 | 1.00 | 0.00 | H |
| ATOM | 4320 | HA | PHE | A | 266 | 23.175 | 7.516 | 14.427 | 1.00 | 0.00 | H |
| ATOM | 4321 | 1HB | PHE | A | 266 | 23.624 | 7.781 | 16.875 | 1.00 | 0.00 | H |
| ATOM | 4322 | 2HB | PHE | A | 266 | 25.062 | 7.810 | 15.885 | 1.00 | 0.00 | H |
| ATOM | 4323 | HD1 | PHE | A | 266 | 26.265 | 9.995 | 15.390 | 1.00 | 0.00 | H |
| ATOM | 4324 | HD2 | PHE | A | 266 | 22.699 | 9.930 | 17.798 | 1.00 | 0.00 | H |
| ATOM | 4325 | HE1 | PHE | A | 266 | 26.465 | 12.440 | 15.742 | 1.00 | 0.00 | H |
| ATOM | 4326 | HE2 | PHE | A | 266 | 22.956 | 12.371 | 18.238 | 1.00 | 0.00 | H |
| ATOM | 4327 | HZ | PHE | A | 266 | 24.812 | 13.634 | 17.170 | 1.00 | 0.00 | H |
| ATOM | 4328 | N | ASN | A | 267 | 21.342 | 9.950 | 14.530 | 1.00 | 0.00 | N |
| ATOM | 4329 | CA | ASN | A | 267 | 19.990 | 10.395 | 14.840 | 1.00 | 0.00 | C |
| ATOM | 4330 | C | ASN | A | 267 | 18.922 | 9.514 | 14.194 | 1.00 | 0.00 | C |
| ATOM | 4331 | O | ASN | A | 267 | 18.068 | 9.988 | 13.451 | 1.00 | 0.00 | O |
| ATOM | 4332 | CB | ASN | A | 267 | 19.799 | 11.869 | 14.444 | 1.00 | 0.00 | C |
| ATOM | 4333 | CG | ASN | A | 267 | 20.097 | 12.197 | 12.965 | 1.00 | 0.00 | C |
| ATOM | 4334 | OD1 | ASN | A | 267 | 21.175 | 11.901 | 12.451 | 1.00 | 0.00 | O |
| ATOM | 4335 | ND2 | ASN | A | 267 | 19.134 | 12.744 | 12.235 | 1.00 | 0.00 | N |
| ATOM | 4336 | H | ASN | A | 267 | 21.937 | 10.501 | 13.927 | 1.00 | 0.00 | H |
| ATOM | 4337 | HA | ASN | A | 267 | 19.793 | 10.346 | 15.909 | 1.00 | 0.00 | H |
| ATOM | 4338 | 1HB | ASN | A | 267 | 18.789 | 12.197 | 14.697 | 1.00 | 0.00 | H |
| ATOM | 4339 | 2HB | ASN | A | 267 | 20.463 | 12.486 | 15.049 | 1.00 | 0.00 | H |
| ATOM | 4340 | 1HD2 | ASN | A | 267 | 19.293 | 12.919 | 11.254 | 1.00 | 0.00 | H |
| ATOM | 4341 | 2HD2 | ASN | A | 267 | 18.228 | 12.930 | 12.638 | 1.00 | 0.00 | H |
| ATOM | 4342 | N | PHE | A | 268 | 18.980 | 8.220 | 14.490 | 1.00 | 0.00 | N |
| ATOM | 4343 | CA | PHE | A | 268 | 18.018 | 7.267 | 13.951 | 1.00 | 0.00 | C |
| ATOM | 4344 | C | PHE | A | 268 | 16.640 | 7.461 | 14.576 | 1.00 | 0.00 | C |
| ATOM | 4345 | O | PHE | A | 268 | 16.521 | 7.843 | 15.744 | 1.00 | 0.00 | O |
| ATOM | 4346 | CB | PHE | A | 268 | 18.464 | 5.836 | 14.240 | 1.00 | 0.00 | C |
| ATOM | 4347 | CG | PHE | A | 268 | 19.664 | 5.354 | 13.443 | 1.00 | 0.00 | C |
| ATOM | 4348 | CD1 | PHE | A | 268 | 19.468 | 4.708 | 12.205 | 1.00 | 0.00 | C |
| ATOM | 4349 | CD2 | PHE | A | 268 | 20.975 | 5.542 | 13.928 | 1.00 | 0.00 | C |
| ATOM | 4350 | CE1 | PHE | A | 268 | 20.571 | 4.279 | 11.444 | 1.00 | 0.00 | C |
| ATOM | 4351 | CE2 | PHE | A | 268 | 22.080 | 5.128 | 13.159 | 1.00 | 0.00 | C |
| ATOM | 4352 | CZ | PHE | A | 268 | 21.879 | 4.498 | 11.916 | 1.00 | 0.00 | C |
| ATOM | 4353 | H | PHE | A | 268 | 19.693 | 7.900 | 15.136 | 1.00 | 0.00 | H |
| ATOM | 4354 | HA | PHE | A | 268 | 17.943 | 7.405 | 12.871 | 1.00 | 0.00 | H |
| ATOM | 4355 | 1HB | PHE | A | 268 | 18.670 | 5.710 | 15.304 | 1.00 | 0.00 | H |
| ATOM | 4356 | 2HB | PHE | A | 268 | 17.644 | 5.154 | 14.018 | 1.00 | 0.00 | H |
| ATOM | 4357 | HD1 | PHE | A | 268 | 18.471 | 4.552 | 11.821 | 1.00 | 0.00 | H |
| ATOM | 4358 | HD2 | PHE | A | 268 | 21.140 | 6.015 | 14.883 | 1.00 | 0.00 | H |
| ATOM | 4359 | HE1 | PHE | A | 268 | 20.408 | 3.803 | 10.488 | 1.00 | 0.00 | H |
| ATOM | 4360 | HE2 | PHE | A | 268 | 23.087 | 5.290 | 13.519 | 1.00 | 0.00 | H |
| ATOM | 4361 | HZ | PHE | A | 268 | 22.729 | 4.189 | 11.324 | 1.00 | 0.00 | H |
| ATOM | 4362 | N | SER | A | 269 | 15.604 | 7.182 | 13.789 | 1.00 | 0.00 | N |
| ATOM | 4363 | CA | SER | A | 269 | 14.224 | 7.271 | 14.256 | 1.00 | 0.00 | C |
| ATOM | 4364 | C | SER | A | 269 | 13.676 | 5.845 | 14.301 | 1.00 | 0.00 | C |
| ATOM | 4365 | O | SER | A | 269 | 14.277 | 4.910 | 13.758 | 1.00 | 0.00 | O |
| ATOM | 4366 | CB | SER | A | 269 | 13.334 | 8.090 | 13.294 | 1.00 | 0.00 | C |
| ATOM | 4367 | OG | SER | A | 269 | 12.008 | 8.292 | 13.760 | 1.00 | 0.00 | O |
| ATOM | 4368 | H | SER | A | 269 | 15.778 | 6.856 | 12.847 | 1.00 | 0.00 | H |
| ATOM | 4369 | HA | SER | A | 269 | 14.153 | 7.706 | 15.255 | 1.00 | 0.00 | H |
| ATOM | 4370 | 1HB | SER | A | 269 | 13.785 | 9.072 | 13.147 | 1.00 | 0.00 | H |
| ATOM | 4371 | 2HB | SER | A | 269 | 13.298 | 7.624 | 12.307 | 1.00 | 0.00 | H |
| ATOM | 4372 | HG | SER | A | 269 | 11.584 | 8.915 | 13.179 | 1.00 | 0.00 | H |
| ATOM | 4373 | N | ALA | A | 270 | 12.538 | 5.675 | 14.963 | 1.00 | 0.00 | N |
| ATOM | 4374 | CA | ALA | A | 270 | 11.909 | 4.369 | 15.055 | 1.00 | 0.00 | C |
| ATOM | 4375 | C | ALA | A | 270 | 11.649 | 3.817 | 13.654 | 1.00 | 0.00 | C |
| ATOM | 4376 | O | ALA | A | 270 | 11.919 | 2.646 | 13.376 | 1.00 | 0.00 | O |
| ATOM | 4377 | CB | ALA | A | 270 | 10.580 | 4.471 | 15.811 | 1.00 | 0.00 | C |
| ATOM | 4378 | H | ALA | A | 270 | 12.052 | 6.497 | 15.295 | 1.00 | 0.00 | H |
| ATOM | 4379 | HA | ALA | A | 270 | 12.578 | 3.686 | 15.583 | 1.00 | 0.00 | H |
| ATOM | 4380 | 1HB | ALA | A | 270 | 10.093 | 3.497 | 15.877 | 1.00 | 0.00 | H |
| ATOM | 4381 | 2HB | ALA | A | 270 | 10.737 | 4.824 | 16.830 | 1.00 | 0.00 | H |
| ATOM | 4382 | 3HB | ALA | A | 270 | 9.884 | 5.157 | 15.325 | 1.00 | 0.00 | H |
| ATOM | 4383 | N | GLY | A | 271 | 11.129 | 4.671 | 12.779 | 1.00 | 0.00 | N |
| ATOM | 4384 | CA | GLY | A | 271 | 10.826 | 4.290 | 11.399 | 1.00 | 0.00 | C |
| ATOM | 4385 | C | GLY | A | 271 | 12.067 | 3.731 | 10.697 | 1.00 | 0.00 | C |
| ATOM | 4386 | O | GLY | A | 271 | 12.018 | 2.679 | 10.062 | 1.00 | 0.00 | O |
| ATOM | 4387 | H | GLY | A | 271 | 10.956 | 5.619 | 13.081 | 1.00 | 0.00 | H |
| ATOM | 4388 | 1HA | GLY | A | 271 | 10.030 | 3.544 | 11.386 | 1.00 | 0.00 | H |
| ATOM | 4389 | 2HA | GLY | A | 271 | 10.464 | 5.159 | 10.849 | 1.00 | 0.00 | H |
| ATOM | 4390 | N | GLU | A | 272 | 13.184 | 4.438 | 10.825 | 1.00 | 0.00 | N |
| ATOM | 4391 | CA | GLU | A | 272 | 14.441 | 4.021 | 10.190 | 1.00 | 0.00 | C |
| ATOM | 4392 | C | GLU | A | 272 | 14.979 | 2.681 | 10.708 | 1.00 | 0.00 | C |
| ATOM | 4393 | O | GLU | A | 272 | 15.601 | 1.920 | 9.961 | 1.00 | 0.00 | O |
| ATOM | 4394 | CB | GLU | A | 272 | 15.500 | 5.116 | 10.393 | 1.00 | 0.00 | C |
| ATOM | 4395 | CG | GLU | A | 272 | 16.691 | 5.087 | 9.433 | 1.00 | 0.00 | C |
| ATOM | 4396 | CD | GLU | A | 272 | 17.605 | 6.281 | 9.652 | 1.00 | 0.00 | C |
| ATOM | 4397 | OE1 | GLU | A | 272 | 17.271 | 7.208 | 10.425 | 1.00 | 0.00 | O |
| ATOM | 4398 | OE2 | GLU | A | 272 | 18.704 | 6.265 | 9.083 | 1.00 | 0.00 | O |
| ATOM | 4399 | H | GLU | A | 272 | 13.190 | 5.242 | 11.432 | 1.00 | 0.00 | H |
| ATOM | 4400 | HA | GLU | A | 272 | 14.251 | 3.912 | 9.120 | 1.00 | 0.00 | H |
| ATOM | 4401 | 1HB | GLU | A | 272 | 15.008 | 6.083 | 10.272 | 1.00 | 0.00 | H |
| ATOM | 4402 | 2HB | GLU | A | 272 | 15.864 | 5.099 | 11.422 | 1.00 | 0.00 | H |
| ATOM | 4403 | 1HG | GLU | A | 272 | 17.274 | 4.175 | 9.560 | 1.00 | 0.00 | H |
| ATOM | 4404 | 2HG | GLU | A | 272 | 16.353 | 5.106 | 8.397 | 1.00 | 0.00 | H |
| ATOM | 4405 | N | LEU | A | 273 | 14.740 | 2.391 | 11.987 | 1.00 | 0.00 | N |
| ATOM | 4406 | CA | LEU | A | 273 | 15.221 | 1.150 | 12.594 | 1.00 | 0.00 | C |
| ATOM | 4407 | C | LEU | A | 273 | 14.205 | 0.020 | 12.537 | 1.00 | 0.00 | C |
| ATOM | 4408 | O | LEU | A | 273 | 14.508 | −1.105 | 12.928 | 1.00 | 0.00 | O |
| ATOM | 4409 | CB | LEU | A | 273 | 15.575 | 1.387 | 14.069 | 1.00 | 0.00 | C |
| ATOM | 4410 | CG | LEU | A | 273 | 16.731 | 2.368 | 14.351 | 1.00 | 0.00 | C |
| ATOM | 4411 | CD1 | LEU | A | 273 | 16.569 | 3.016 | 15.736 | 1.00 | 0.00 | C |
| ATOM | 4412 | CD2 | LEU | A | 273 | 18.096 | 1.673 | 14.259 | 1.00 | 0.00 | C |
| ATOM | 4413 | H | LEU | A | 273 | 14.265 | 3.073 | 12.567 | 1.00 | 0.00 | H |
| ATOM | 4414 | HA | LEU | A | 273 | 16.122 | 0.801 | 12.091 | 1.00 | 0.00 | H |
| ATOM | 4415 | 1HB | LEU | A | 273 | 14.674 | 1.751 | 14.567 | 1.00 | 0.00 | H |
| ATOM | 4416 | 2HB | LEU | A | 273 | 15.809 | 0.443 | 14.561 | 1.00 | 0.00 | H |
| ATOM | 4417 | HG | LEU | A | 273 | 16.701 | 3.163 | 13.606 | 1.00 | 0.00 | H |
| ATOM | 4418 | 1HD1 | LEU | A | 273 | 17.406 | 3.674 | 15.970 | 1.00 | 0.00 | H |
| ATOM | 4419 | 2HD1 | LEU | A | 273 | 15.660 | 3.615 | 15.786 | 1.00 | 0.00 | H |
| ATOM | 4420 | 3HD1 | LEU | A | 273 | 16.514 | 2.263 | 16.523 | 1.00 | 0.00 | H |
| ATOM | 4421 | 1HD2 | LEU | A | 273 | 18.912 | 2.389 | 14.365 | 1.00 | 0.00 | H |
| ATOM | 4422 | 2HD2 | LEU | A | 273 | 18.214 | 0.914 | 15.034 | 1.00 | 0.00 | H |
| ATOM | 4423 | 3HD2 | LEU | A | 273 | 18.220 | 1.178 | 13.300 | 1.00 | 0.00 | H |
| ATOM | 4424 | N | SER | A | 274 | 13.009 | 0.314 | 12.043 | 1.00 | 0.00 | N |
| ATOM | 4425 | CA | SER | A | 274 | 11.927 | −0.670 | 12.018 | 1.00 | 0.00 | C |
| ATOM | 4426 | C | SER | A | 274 | 12.212 | −2.053 | 11.440 | 1.00 | 0.00 | C |
| ATOM | 4427 | O | SER | A | 274 | 11.613 | −3.036 | 11.880 | 1.00 | 0.00 | O |
| ATOM | 4428 | CB | SER | A | 274 | 10.680 | −0.066 | 11.354 | 1.00 | 0.00 | C |
| ATOM | 4429 | OG | SER | A | 274 | 9.489 | −0.793 | 11.618 | 1.00 | 0.00 | O |
| ATOM | 4430 | H | SER | A | 274 | 12.811 | 1.274 | 11.791 | 1.00 | 0.00 | H |
| ATOM | 4431 | HA | SER | A | 274 | 11.670 | −0.840 | 13.066 | 1.00 | 0.00 | H |
| ATOM | 4432 | 1HB | SER | A | 274 | 10.521 | 0.950 | 11.718 | 1.00 | 0.00 | H |
| ATOM | 4433 | 2HB | SER | A | 274 | 10.825 | 0.018 | 10.275 | 1.00 | 0.00 | H |
| ATOM | 4434 | HG | SER | A | 274 | 9.617 | −1.693 | 11.333 | 1.00 | 0.00 | H |
| ATOM | 4435 | N | ILE | A | 275 | 13.127 | −2.159 | 10.485 | 1.00 | 0.00 | N |
| ATOM | 4436 | CA | ILE | A | 275 | 13.412 | −3.469 | 9.906 | 1.00 | 0.00 | C |
| ATOM | 4437 | C | ILE | A | 275 | 14.167 | −4.391 | 10.865 | 1.00 | 0.00 | C |
| ATOM | 4438 | O | ILE | A | 275 | 14.248 | −5.599 | 10.630 | 1.00 | 0.00 | O |
| ATOM | 4439 | CB | ILE | A | 275 | 14.212 | −3.318 | 8.604 | 1.00 | 0.00 | C |
| ATOM | 4440 | CG1 | ILE | A | 275 | 13.808 | −2.079 | 7.750 | 1.00 | 0.00 | C |
| ATOM | 4441 | CG2 | ILE | A | 275 | 14.142 | −4.572 | 7.702 | 1.00 | 0.00 | C |
| ATOM | 4442 | CD1 | ILE | A | 275 | 14.651 | −0.819 | 8.020 | 1.00 | 0.00 | C |
| ATOM | 4443 | H | ILE | A | 275 | 13.596 | −1.334 | 10.142 | 1.00 | 0.00 | H |
| ATOM | 4444 | HA | ILE | A | 275 | 12.468 | −3.959 | 9.657 | 1.00 | 0.00 | H |
| ATOM | 4445 | HB | ILE | A | 275 | 15.266 | −3.190 | 8.860 | 1.00 | 0.00 | H |
| ATOM | 4446 | 1HG1 | ILE | A | 275 | 13.928 | −2.302 | 6.689 | 1.00 | 0.00 | H |
| ATOM | 4447 | 2HG1 | ILE | A | 275 | 12.745 | −1.858 | 7.863 | 1.00 | 0.00 | H |
| ATOM | 4448 | 1HG2 | ILE | A | 275 | 14.707 | −4.431 | 6.780 | 1.00 | 0.00 | H |
| ATOM | 4449 | 2HG2 | ILE | A | 275 | 14.560 | −5.458 | 8.178 | 1.00 | 0.00 | H |
| ATOM | 4450 | 3HG2 | ILE | A | 275 | 13.113 | −4.801 | 7.421 | 1.00 | 0.00 | H |
| ATOM | 4451 | 1HD1 | ILE | A | 275 | 14.351 | −0.005 | 7.359 | 1.00 | 0.00 | H |
| ATOM | 4452 | 2HD1 | ILE | A | 275 | 14.552 | −0.450 | 9.040 | 1.00 | 0.00 | H |
| ATOM | 4453 | 3HD1 | ILE | A | 275 | 15.710 | −1.003 | 7.839 | 1.00 | 0.00 | H |
| ATOM | 4454 | N | GLN | A | 276 | 14.714 | −3.831 | 11.943 | 1.00 | 0.00 | N |
| ATOM | 4455 | CA | GLN | A | 276 | 15.476 | −4.620 | 12.915 | 1.00 | 0.00 | C |
| ATOM | 4456 | C | GLN | A | 276 | 15.891 | −3.715 | 14.078 | 1.00 | 0.00 | C |
| ATOM | 4457 | O | GLN | A | 276 | 17.063 | −3.365 | 14.225 | 1.00 | 0.00 | O |
| ATOM | 4458 | CB | GLN | A | 276 | 16.718 | −5.207 | 12.235 | 1.00 | 0.00 | C |
| ATOM | 4459 | CG | GLN | A | 276 | 17.455 | −6.263 | 13.097 | 1.00 | 0.00 | C |
| ATOM | 4460 | CD | GLN | A | 276 | 16.621 | −7.516 | 13.368 | 1.00 | 0.00 | C |
| ATOM | 4461 | OE1 | GLN | A | 276 | 16.068 | −8.112 | 12.450 | 1.00 | 0.00 | O |
| ATOM | 4462 | NE2 | GLN | A | 276 | 16.500 | −7.920 | 14.625 | 1.00 | 0.00 | N |
| ATOM | 4463 | H | GLN | A | 276 | 14.595 | −2.835 | 12.118 | 1.00 | 0.00 | H |
| ATOM | 4464 | HA | GLN | A | 276 | 14.835 | −5.421 | 13.286 | 1.00 | 0.00 | H |
| ATOM | 4465 | 1HB | GLN | A | 276 | 16.509 | −5.695 | 11.287 | 1.00 | 0.00 | H |
| ATOM | 4466 | 2HB | GLN | A | 276 | 17.417 | −4.418 | 11.972 | 1.00 | 0.00 | H |
| ATOM | 4467 | 1HG | GLN | A | 276 | 18.364 | −6.577 | 12.582 | 1.00 | 0.00 | H |
| ATOM | 4468 | 2HG | GLN | A | 276 | 17.775 | −5.837 | 14.046 | 1.00 | 0.00 | H |
| ATOM | 4469 | 1HE2 | GLN | A | 276 | 15.957 | −8.745 | 14.831 | 1.00 | 0.00 | H |
| ATOM | 4470 | 2HE2 | GLN | A | 276 | 16.955 | −7.423 | 15.395 | 1.00 | 0.00 | H |
| ATOM | 4471 | N | PRO | A | 277 | 14.922 | −3.323 | 14.920 | 1.00 | 0.00 | N |
| ATOM | 4472 | CA | PRO | A | 277 | 15.181 | −2.449 | 16.071 | 1.00 | 0.00 | C |
| ATOM | 4473 | C | PRO | A | 277 | 16.396 | −2.798 | 16.931 | 1.00 | 0.00 | C |
| ATOM | 4474 | O | PRO | A | 277 | 17.144 | −1.914 | 17.337 | 1.00 | 0.00 | O |
| ATOM | 4475 | CB | PRO | A | 277 | 13.872 | −2.501 | 16.854 | 1.00 | 0.00 | C |
| ATOM | 4476 | CG | PRO | A | 277 | 12.783 | −2.843 | 15.864 | 1.00 | 0.00 | C |
| ATOM | 4477 | CD | PRO | A | 277 | 13.502 | −3.685 | 14.825 | 1.00 | 0.00 | C |
| ATOM | 4478 | HA | PRO | A | 277 | 15.323 | −1.443 | 15.678 | 1.00 | 0.00 | H |
| ATOM | 4479 | 1HB | PRO | A | 277 | 13.911 | −3.283 | 17.614 | 1.00 | 0.00 | H |
| ATOM | 4480 | 2HB | PRO | A | 277 | 13.673 | −1.564 | 17.375 | 1.00 | 0.00 | H |
| ATOM | 4481 | 1HG | PRO | A | 277 | 12.417 | −1.927 | 15.397 | 1.00 | 0.00 | H |
| ATOM | 4482 | 2HG | PRO | A | 277 | 11.931 | −3.353 | 16.315 | 1.00 | 0.00 | H |
| ATOM | 4483 | 1HD | PRO | A | 277 | 13.072 | −3.504 | 13.846 | 1.00 | 0.00 | H |
| ATOM | 4484 | 2HD | PRO | A | 277 | 13.386 | −4.745 | 15.053 | 1.00 | 0.00 | H |
| ATOM | 4485 | N | SER | A | 278 | 16.608 | −4.087 | 17.233 | 1.00 | 0.00 | N |
| ATOM | 4486 | CA | SER | A | 278 | 17.761 | −4.468 | 18.057 | 1.00 | 0.00 | C |
| ATOM | 4487 | C | SER | A | 278 | 19.128 | −3.959 | 17.603 | 1.00 | 0.00 | C |
| ATOM | 4488 | O | SER | A | 278 | 20.081 | −3.948 | 18.387 | 1.00 | 0.00 | O |
| ATOM | 4489 | CB | SER | A | 278 | 17.703 | −5.995 | 18.073 | 1.00 | 0.00 | C |
| ATOM | 4490 | OG | SER | A | 278 | 17.798 | −6.560 | 16.767 | 1.00 | 0.00 | O |
| ATOM | 4491 | H | SER | A | 278 | 16.031 | −4.822 | 16.853 | 1.00 | 0.00 | H |
| ATOM | 4492 | HA | SER | A | 278 | 17.597 | −4.098 | 19.071 | 1.00 | 0.00 | H |
| ATOM | 4493 | 1HB | SER | A | 278 | 18.511 | −6.394 | 18.690 | 1.00 | 0.00 | H |
| ATOM | 4494 | 2HB | SER | A | 278 | 16.775 | −6.332 | 18.539 | 1.00 | 0.00 | H |
| ATOM | 4495 | HG | SER | A | 278 | 18.684 | −6.406 | 16.458 | 1.00 | 0.00 | H |
| ATOM | 4496 | N | LEU | A | 279 | 19.241 | −3.547 | 16.345 | 1.00 | 0.00 | N |
| ATOM | 4497 | CA | LEU | A | 279 | 20.520 | −3.048 | 15.844 | 1.00 | 0.00 | C |
| ATOM | 4498 | C | LEU | A | 279 | 20.895 | −1.692 | 16.451 | 1.00 | 0.00 | C |
| ATOM | 4499 | O | LEU | A | 279 | 22.047 | −1.261 | 16.362 | 1.00 | 0.00 | O |
| ATOM | 4500 | CB | LEU | A | 279 | 20.485 | −2.946 | 14.316 | 1.00 | 0.00 | C |
| ATOM | 4501 | CG | LEU | A | 279 | 20.581 | −4.285 | 13.553 | 1.00 | 0.00 | C |
| ATOM | 4502 | CD1 | LEU | A | 279 | 20.419 | −4.053 | 12.043 | 1.00 | 0.00 | C |
| ATOM | 4503 | CD2 | LEU | A | 279 | 21.896 | −5.040 | 13.811 | 1.00 | 0.00 | C |
| ATOM | 4504 | H | LEU | A | 279 | 18.433 | −3.537 | 15.729 | 1.00 | 0.00 | H |
| ATOM | 4505 | HA | LEU | A | 279 | 21.322 | −3.714 | 16.160 | 1.00 | 0.00 | H |
| ATOM | 4506 | 1HB | LEU | A | 279 | 19.586 | −2.408 | 14.010 | 1.00 | 0.00 | H |
| ATOM | 4507 | 2HB | LEU | A | 279 | 21.316 | −2.324 | 13.977 | 1.00 | 0.00 | H |
| ATOM | 4508 | HG | LEU | A | 279 | 19.765 | −4.918 | 13.887 | 1.00 | 0.00 | H |
| ATOM | 4509 | 1HD1 | LEU | A | 279 | 20.398 | −4.995 | 11.494 | 1.00 | 0.00 | H |
| ATOM | 4510 | 2HD1 | LEU | A | 279 | 19.493 | −3.528 | 11.824 | 1.00 | 0.00 | H |
| ATOM | 4511 | 3HD1 | LEU | A | 279 | 21.237 | −3.453 | 11.642 | 1.00 | 0.00 | H |
| ATOM | 4512 | 1HD2 | LEU | A | 279 | 21.952 | −5.944 | 13.204 | 1.00 | 0.00 | H |
| ATOM | 4513 | 2HD2 | LEU | A | 279 | 22.760 | −4.424 | 13.560 | 1.00 | 0.00 | H |
| ATOM | 4514 | 3HD2 | LEU | A | 279 | 21.996 | −5.355 | 14.849 | 1.00 | 0.00 | H |
| ATOM | 4515 | N | ASN | A | 280 | 19.923 | −1.026 | 17.072 | 1.00 | 0.00 | N |
| ATOM | 4516 | CA | ASN | A | 280 | 20.161 | 0.275 | 17.697 | 1.00 | 0.00 | C |
| ATOM | 4517 | C | ASN | A | 280 | 21.312 | 0.189 | 18.690 | 1.00 | 0.00 | C |
| ATOM | 4518 | O | ASN | A | 280 | 22.036 | 1.155 | 18.906 | 1.00 | 0.00 | O |
| ATOM | 4519 | CB | ASN | A | 280 | 18.904 | 0.751 | 18.405 | 1.00 | 0.00 | C |
| ATOM | 4520 | CG | ASN | A | 280 | 18.522 | 0.135 | 19.764 | 1.00 | 0.00 | C |
| ATOM | 4521 | OD1 | ASN | A | 280 | 18.943 | 0.620 | 20.809 | 1.00 | 0.00 | O |
| ATOM | 4522 | ND2 | ASN | A | 280 | 17.710 | −0.911 | 19.780 | 1.00 | 0.00 | N |
| ATOM | 4523 | H | ASN | A | 280 | 18.989 | −1.426 | 17.083 | 1.00 | 0.00 | H |
| ATOM | 4524 | HA | ASN | A | 280 | 20.415 | 0.990 | 16.916 | 1.00 | 0.00 | H |
| ATOM | 4525 | 1HB | ASN | A | 280 | 19.016 | 1.822 | 18.584 | 1.00 | 0.00 | H |
| ATOM | 4526 | 2HB | ASN | A | 280 | 18.051 | 0.676 | 17.730 | 1.00 | 0.00 | H |
| ATOM | 4527 | 1HD2 | ASN | A | 280 | 17.426 | −1.317 | 20.658 | 1.00 | 0.00 | H |
| ATOM | 4528 | 2HD2 | ASN | A | 280 | 17.370 | −1.296 | 18.896 | 1.00 | 0.00 | H |
| ATOM | 4529 | N | ALA | A | 281 | 21.471 | −0.982 | 19.293 | 1.00 | 0.00 | N |
| ATOM | 4530 | CA | ALA | A | 281 | 22.522 | −1.230 | 20.268 | 1.00 | 0.00 | C |
| ATOM | 4531 | C | ALA | A | 281 | 23.903 | −1.049 | 19.659 | 1.00 | 0.00 | C |
| ATOM | 4532 | O | ALA | A | 281 | 24.865 | −0.699 | 20.354 | 1.00 | 0.00 | O |
| ATOM | 4533 | CB | ALA | A | 281 | 22.391 | −2.651 | 20.815 | 1.00 | 0.00 | C |
| ATOM | 4534 | H | ALA | A | 281 | 20.843 | −1.738 | 19.055 | 1.00 | 0.00 | H |
| ATOM | 4535 | HA | ALA | A | 281 | 22.413 | −0.517 | 21.088 | 1.00 | 0.00 | H |
| ATOM | 4536 | 1HB | ALA | A | 281 | 23.160 | −2.858 | 21.560 | 1.00 | 0.00 | H |
| ATOM | 4537 | 2HB | ALA | A | 281 | 21.424 | −2.797 | 21.296 | 1.00 | 0.00 | H |
| ATOM | 4538 | 3HB | ALA | A | 281 | 22.485 | −3.398 | 20.025 | 1.00 | 0.00 | H |
| ATOM | 4539 | N | ILE | A | 282 | 24.003 | −1.304 | 18.359 | 1.00 | 0.00 | N |
| ATOM | 4540 | CA | ILE | A | 282 | 25.269 | −1.153 | 17.651 | 1.00 | 0.00 | C |
| ATOM | 4541 | C | ILE | A | 282 | 25.325 | 0.209 | 16.984 | 1.00 | 0.00 | C |
| ATOM | 4542 | O | ILE | A | 282 | 26.340 | 0.905 | 17.051 | 1.00 | 0.00 | O |
| ATOM | 4543 | CB | ILE | A | 282 | 25.432 | −2.208 | 16.543 | 1.00 | 0.00 | C |
| ATOM | 4544 | CG1 | ILE | A | 282 | 25.215 | −3.643 | 17.094 | 1.00 | 0.00 | C |
| ATOM | 4545 | CG2 | ILE | A | 282 | 26.801 | −2.123 | 15.817 | 1.00 | 0.00 | C |
| ATOM | 4546 | CD1 | ILE | A | 282 | 25.188 | −4.743 | 16.022 | 1.00 | 0.00 | C |
| ATOM | 4547 | H | ILE | A | 282 | 23.166 | −1.508 | 17.824 | 1.00 | 0.00 | H |
| ATOM | 4548 | HA | ILE | A | 282 | 26.124 | −1.231 | 18.327 | 1.00 | 0.00 | H |
| ATOM | 4549 | HB | ILE | A | 282 | 24.658 | −2.045 | 15.789 | 1.00 | 0.00 | H |
| ATOM | 4550 | 1HG1 | ILE | A | 282 | 25.980 | −3.875 | 17.837 | 1.00 | 0.00 | H |
| ATOM | 4551 | 2HG1 | ILE | A | 282 | 24.263 | −3.704 | 17.621 | 1.00 | 0.00 | H |
| ATOM | 4552 | 1HG2 | ILE | A | 282 | 26.881 | −2.847 | 15.008 | 1.00 | 0.00 | H |
| ATOM | 4553 | 2HG2 | ILE | A | 282 | 26.976 | −1.153 | 15.351 | 1.00 | 0.00 | H |
| ATOM | 4554 | 3HG2 | ILE | A | 282 | 27.627 | −2.308 | 16.504 | 1.00 | 0.00 | H |
| ATOM | 4555 | 1HD1 | ILE | A | 282 | 24.893 | −5.697 | 16.459 | 1.00 | 0.00 | H |
| ATOM | 4556 | 2HD1 | ILE | A | 282 | 24.474 | −4.507 | 15.235 | 1.00 | 0.00 | H |
| ATOM | 4557 | 3HD1 | ILE | A | 282 | 26.164 | −4.892 | 15.561 | 1.00 | 0.00 | H |
| ATOM | 4558 | N | LEU | A | 283 | 24.213 | 0.578 | 16.349 | 1.00 | 0.00 | N |
| ATOM | 4559 | CA | LEU | A | 283 | 24.102 | 1.824 | 15.601 | 1.00 | 0.00 | C |
| ATOM | 4560 | C | LEU | A | 283 | 24.227 | 3.126 | 16.376 | 1.00 | 0.00 | C |
| ATOM | 4561 | O | LEU | A | 283 | 24.730 | 4.114 | 15.836 | 1.00 | 0.00 | O |
| ATOM | 4562 | CB | LEU | A | 283 | 22.800 | 1.816 | 14.799 | 1.00 | 0.00 | C |
| ATOM | 4563 | CG | LEU | A | 283 | 22.712 | 0.708 | 13.725 | 1.00 | 0.00 | C |
| ATOM | 4564 | CD1 | LEU | A | 283 | 21.276 | 0.572 | 13.208 | 1.00 | 0.00 | C |
| ATOM | 4565 | CD2 | LEU | A | 283 | 23.673 | 0.950 | 12.553 | 1.00 | 0.00 | C |
| ATOM | 4566 | H | LEU | A | 283 | 23.420 | −0.058 | 16.347 | 1.00 | 0.00 | H |
| ATOM | 4567 | HA | LEU | A | 283 | 24.947 | 1.853 | 14.914 | 1.00 | 0.00 | H |
| ATOM | 4568 | 1HB | LEU | A | 283 | 21.965 | 1.723 | 15.491 | 1.00 | 0.00 | H |
| ATOM | 4569 | 2HB | LEU | A | 283 | 22.660 | 2.780 | 14.312 | 1.00 | 0.00 | H |
| ATOM | 4570 | HG | LEU | A | 283 | 22.978 | −0.251 | 14.167 | 1.00 | 0.00 | H |
| ATOM | 4571 | 1HD1 | LEU | A | 283 | 21.210 | −0.209 | 12.453 | 1.00 | 0.00 | H |
| ATOM | 4572 | 2HD1 | LEU | A | 283 | 20.591 | 0.306 | 14.013 | 1.00 | 0.00 | H |
| ATOM | 4573 | 3HD1 | LEU | A | 283 | 20.923 | 1.502 | 12.759 | 1.00 | 0.00 | H |
| ATOM | 4574 | 1HD2 | LEU | A | 283 | 23.546 | 0.181 | 11.791 | 1.00 | 0.00 | H |
| ATOM | 4575 | 2HD2 | LEU | A | 283 | 23.492 | 1.917 | 12.084 | 1.00 | 0.00 | H |
| ATOM | 4576 | 3HD2 | LEU | A | 283 | 24.718 | 0.919 | 12.862 | 1.00 | 0.00 | H |
| ATOM | 4577 | N | ILE | A | 284 | 23.757 | 3.151 | 17.619 | 1.00 | 0.00 | N |
| ATOM | 4578 | CA | ILE | A | 284 | 23.873 | 4.361 | 18.422 | 1.00 | 0.00 | C |
| ATOM | 4579 | C | ILE | A | 284 | 24.882 | 4.060 | 19.521 | 1.00 | 0.00 | C |
| ATOM | 4580 | O | ILE | A | 284 | 24.547 | 3.431 | 20.524 | 1.00 | 0.00 | O |
| ATOM | 4581 | CB | ILE | A | 284 | 22.516 | 4.773 | 19.047 | 1.00 | 0.00 | C |
| ATOM | 4582 | CG1 | ILE | A | 284 | 21.424 | 4.904 | 17.950 | 1.00 | 0.00 | C |
| ATOM | 4583 | CG2 | ILE | A | 284 | 22.623 | 6.092 | 19.854 | 1.00 | 0.00 | C |
| ATOM | 4584 | CD1 | ILE | A | 284 | 20.014 | 5.205 | 18.476 | 1.00 | 0.00 | C |
| ATOM | 4585 | H | ILE | A | 284 | 23.348 | 2.323 | 18.045 | 1.00 | 0.00 | H |
| ATOM | 4586 | HA | ILE | A | 284 | 24.154 | 5.226 | 17.828 | 1.00 | 0.00 | H |
| ATOM | 4587 | HB | ILE | A | 284 | 22.189 | 3.986 | 19.730 | 1.00 | 0.00 | H |
| ATOM | 4588 | 1HG1 | ILE | A | 284 | 21.712 | 5.674 | 17.232 | 1.00 | 0.00 | H |
| ATOM | 4589 | 2HG1 | ILE | A | 284 | 21.355 | 3.977 | 17.380 | 1.00 | 0.00 | H |
| ATOM | 4590 | 1HG2 | ILE | A | 284 | 21.687 | 6.334 | 20.355 | 1.00 | 0.00 | H |
| ATOM | 4591 | 2HG2 | ILE | A | 284 | 23.377 | 6.042 | 20.640 | 1.00 | 0.00 | H |
| ATOM | 4592 | 3HG2 | ILE | A | 284 | 22.878 | 6.935 | 19.209 | 1.00 | 0.00 | H |
| ATOM | 4593 | 1HD1 | ILE | A | 284 | 19.284 | 5.156 | 17.668 | 1.00 | 0.00 | H |
| ATOM | 4594 | 2HD1 | ILE | A | 284 | 19.713 | 4.488 | 19.240 | 1.00 | 0.00 | H |
| ATOM | 4595 | 3HD1 | ILE | A | 284 | 19.954 | 6.206 | 18.900 | 1.00 | 0.00 | H |
| ATOM | 4596 | N | PRO | A | 285 | 26.144 | 4.479 | 19.327 | 1.00 | 0.00 | N |
| ATOM | 4597 | CA | PRO | A | 285 | 27.205 | 4.245 | 20.311 | 1.00 | 0.00 | C |
| ATOM | 4598 | C | PRO | A | 285 | 26.956 | 4.940 | 21.652 | 1.00 | 0.00 | C |
| ATOM | 4599 | O | PRO | A | 285 | 26.177 | 5.886 | 21.744 | 1.00 | 0.00 | O |
| ATOM | 4600 | CB | PRO | A | 285 | 28.469 | 4.748 | 19.609 | 1.00 | 0.00 | C |
| ATOM | 4601 | CG | PRO | A | 285 | 28.140 | 5.138 | 18.191 | 1.00 | 0.00 | C |
| ATOM | 4602 | CD | PRO | A | 285 | 26.645 | 5.095 | 18.099 | 1.00 | 0.00 | C |
| ATOM | 4603 | HA | PRO | A | 285 | 27.257 | 3.165 | 20.438 | 1.00 | 0.00 | H |
| ATOM | 4604 | 1HB | PRO | A | 285 | 28.976 | 5.535 | 20.163 | 1.00 | 0.00 | H |
| ATOM | 4605 | 2HB | PRO | A | 285 | 29.204 | 3.942 | 19.586 | 1.00 | 0.00 | H |
| ATOM | 4606 | 1HG | PRO | A | 285 | 28.492 | 4.361 | 17.528 | 1.00 | 0.00 | H |
| ATOM | 4607 | 2HG | PRO | A | 285 | 28.592 | 6.058 | 17.833 | 1.00 | 0.00 | H |
| ATOM | 4608 | 1HD | PRO | A | 285 | 26.444 | 4.476 | 17.243 | 1.00 | 0.00 | H |
| ATOM | 4609 | 2HD | PRO | A | 285 | 26.226 | 6.090 | 17.944 | 1.00 | 0.00 | H |
| ATOM | 4610 | N | PRO | A | 286 | 27.615 | 4.459 | 22.716 | 1.00 | 0.00 | N |
| ATOM | 4611 | CA | PRO | A | 286 | 27.474 | 5.022 | 24.067 | 1.00 | 0.00 | C |
| ATOM | 4612 | C | PRO | A | 286 | 27.540 | 6.544 | 24.183 | 1.00 | 0.00 | C |
| ATOM | 4613 | O | PRO | A | 286 | 26.744 | 7.152 | 24.895 | 1.00 | 0.00 | O |
| ATOM | 4614 | CB | PRO | A | 286 | 28.589 | 4.335 | 24.854 | 1.00 | 0.00 | C |
| ATOM | 4615 | CG | PRO | A | 286 | 28.845 | 3.011 | 24.167 | 1.00 | 0.00 | C |
| ATOM | 4616 | CD | PRO | A | 286 | 28.493 | 3.281 | 22.711 | 1.00 | 0.00 | C |
| ATOM | 4617 | HA | PRO | A | 286 | 26.505 | 4.694 | 24.449 | 1.00 | 0.00 | H |
| ATOM | 4618 | 1HB | PRO | A | 286 | 29.505 | 4.929 | 24.824 | 1.00 | 0.00 | H |
| ATOM | 4619 | 2HB | PRO | A | 286 | 28.328 | 4.209 | 25.906 | 1.00 | 0.00 | H |
| ATOM | 4620 | 1HG | PRO | A | 286 | 28.171 | 2.255 | 24.574 | 1.00 | 0.00 | H |
| ATOM | 4621 | 2HG | PRO | A | 286 | 29.867 | 2.651 | 24.299 | 1.00 | 0.00 | H |
| ATOM | 4622 | 1HD | PRO | A | 286 | 28.016 | 2.397 | 22.287 | 1.00 | 0.00 | H |
| ATOM | 4623 | 2HD | PRO | A | 286 | 29.393 | 3.498 | 22.135 | 1.00 | 0.00 | H |
| ATOM | 4624 | N | HIS | A | 287 | 28.479 | 7.161 | 23.475 | 1.00 | 0.00 | N |
| ATOM | 4625 | CA | HIS | A | 287 | 28.658 | 8.609 | 23.534 | 1.00 | 0.00 | C |
| ATOM | 4626 | C | HIS | A | 287 | 27.669 | 9.428 | 22.724 | 1.00 | 0.00 | C |
| ATOM | 4627 | O | HIS | A | 287 | 27.476 | 10.615 | 22.989 | 1.00 | 0.00 | O |
| ATOM | 4628 | CB | HIS | A | 287 | 30.068 | 8.965 | 23.069 | 1.00 | 0.00 | C |
| ATOM | 4629 | CG | HIS | A | 287 | 31.131 | 8.257 | 23.877 | 1.00 | 0.00 | C |
| ATOM | 4630 | ND1 | HIS | A | 287 | 31.550 | 8.693 | 25.123 | 1.00 | 0.00 | N |
| ATOM | 4631 | CD2 | HIS | A | 287 | 31.803 | 7.073 | 23.660 | 1.00 | 0.00 | C |
| ATOM | 4632 | CE1 | HIS | A | 287 | 32.422 | 7.799 | 25.593 | 1.00 | 0.00 | C |
| ATOM | 4633 | NE2 | HIS | A | 287 | 32.623 | 6.789 | 24.754 | 1.00 | 0.00 | N |
| ATOM | 4634 | H | HIS | A | 287 | 29.079 | 6.631 | 22.866 | 1.00 | 0.00 | H |
| ATOM | 4635 | HA | HIS | A | 287 | 28.569 | 8.929 | 24.574 | 1.00 | 0.00 | H |
| ATOM | 4636 | 1HB | HIS | A | 287 | 30.201 | 8.693 | 22.021 | 1.00 | 0.00 | H |
| ATOM | 4637 | 2HB | HIS | A | 287 | 30.241 | 10.041 | 23.130 | 1.00 | 0.00 | H |
| ATOM | 4638 | HD1 | HIS | A | 287 | 31.257 | 9.531 | 25.613 | 1.00 | 0.00 | H |
| ATOM | 4639 | HD2 | HIS | A | 287 | 31.743 | 6.397 | 22.818 | 1.00 | 0.00 | H |
| ATOM | 4640 | HE1 | HIS | A | 287 | 32.912 | 7.888 | 26.553 | 1.00 | 0.00 | H |
| ATOM | 4641 | N | LEU | A | 288 | 27.038 | 8.802 | 21.741 | 1.00 | 0.00 | N |
| ATOM | 4642 | CA | LEU | A | 288 | 26.115 | 9.503 | 20.856 | 1.00 | 0.00 | C |
| ATOM | 4643 | C | LEU | A | 288 | 24.764 | 9.927 | 21.423 | 1.00 | 0.00 | C |
| ATOM | 4644 | O | LEU | A | 288 | 24.137 | 9.204 | 22.198 | 1.00 | 0.00 | O |
| ATOM | 4645 | CB | LEU | A | 288 | 25.899 | 8.670 | 19.604 | 1.00 | 0.00 | C |
| ATOM | 4646 | CG | LEU | A | 288 | 27.129 | 8.909 | 18.661 | 1.00 | 0.00 | C |
| ATOM | 4647 | CD1 | LEU | A | 288 | 27.447 | 10.396 | 18.401 | 1.00 | 0.00 | C |
| ATOM | 4648 | CD2 | LEU | A | 288 | 28.434 | 8.286 | 19.163 | 1.00 | 0.00 | C |
| ATOM | 4649 | H | LEU | A | 288 | 27.104 | 7.795 | 21.671 | 1.00 | 0.00 | H |
| ATOM | 4650 | HA | LEU | A | 288 | 26.622 | 10.465 | 20.776 | 1.00 | 0.00 | H |
| ATOM | 4651 | 1HB | LEU | A | 288 | 25.761 | 7.614 | 19.836 | 1.00 | 0.00 | H |
| ATOM | 4652 | 2HB | LEU | A | 288 | 24.991 | 8.974 | 19.089 | 1.00 | 0.00 | H |
| ATOM | 4653 | HG | LEU | A | 288 | 26.880 | 8.439 | 17.709 | 1.00 | 0.00 | H |
| ATOM | 4654 | 1HD1 | LEU | A | 288 | 27.936 | 10.533 | 17.436 | 1.00 | 0.00 | H |
| ATOM | 4655 | 2HD1 | LEU | A | 288 | 26.546 | 11.002 | 18.435 | 1.00 | 0.00 | H |
| ATOM | 4656 | 3HD1 | LEU | A | 288 | 28.116 | 10.825 | 19.147 | 1.00 | 0.00 | H |
| ATOM | 4657 | 1HD2 | LEU | A | 288 | 29.142 | 8.120 | 18.351 | 1.00 | 0.00 | H |
| ATOM | 4658 | 2HD2 | LEU | A | 288 | 28.944 | 8.906 | 19.899 | 1.00 | 0.00 | H |
| ATOM | 4659 | 3HD2 | LEU | A | 288 | 28.209 | 7.356 | 19.659 | 1.00 | 0.00 | H |
| ATOM | 4660 | N | ARG | A | 289 | 24.332 | 11.117 | 21.015 | 1.00 | 0.00 | N |
| ATOM | 4661 | CA | ARG | A | 289 | 23.044 | 11.678 | 21.401 | 1.00 | 0.00 | C |
| ATOM | 4662 | C | ARG | A | 289 | 22.766 | 11.755 | 22.897 | 1.00 | 0.00 | C |
| ATOM | 4663 | O | ARG | A | 289 | 21.607 | 11.735 | 23.316 | 1.00 | 0.00 | O |
| ATOM | 4664 | CB | ARG | A | 289 | 21.925 | 10.884 | 20.723 | 1.00 | 0.00 | C |
| ATOM | 4665 | CG | ARG | A | 289 | 20.614 | 11.652 | 20.430 | 1.00 | 0.00 | C |
| ATOM | 4666 | CD | ARG | A | 289 | 19.744 | 10.988 | 19.344 | 1.00 | 0.00 | C |
| ATOM | 4667 | NE | ARG | A | 289 | 19.136 | 9.727 | 19.798 | 1.00 | 0.00 | N |
| ATOM | 4668 | CZ | ARG | A | 289 | 18.292 | 8.949 | 19.102 | 1.00 | 0.00 | C |
| ATOM | 4669 | NH1 | ARG | A | 289 | 17.888 | 9.295 | 17.878 | 1.00 | 0.00 | N |
| ATOM | 4670 | NH2 | ARG | A | 289 | 17.848 | 7.819 | 19.645 | 1.00 | 0.00 | N |
| ATOM | 4671 | H | ARG | A | 289 | 24.913 | 11.638 | 20.375 | 1.00 | 0.00 | H |
| ATOM | 4672 | HA | ARG | A | 289 | 23.019 | 12.703 | 21.030 | 1.00 | 0.00 | H |
| ATOM | 4673 | 1HB | ARG | A | 289 | 22.295 | 10.514 | 19.776 | 1.00 | 0.00 | H |
| ATOM | 4674 | 2HB | ARG | A | 289 | 21.695 | 9.978 | 21.287 | 1.00 | 0.00 | H |
| ATOM | 4675 | 1HG | ARG | A | 289 | 20.030 | 11.786 | 21.341 | 1.00 | 0.00 | H |
| ATOM | 4676 | 2HG | ARG | A | 289 | 20.859 | 12.658 | 20.087 | 1.00 | 0.00 | H |
| ATOM | 4677 | 1HD | ARG | A | 289 | 18.940 | 11.670 | 19.064 | 1.00 | 0.00 | H |
| ATOM | 4678 | 2HD | ARG | A | 289 | 20.329 | 10.803 | 18.441 | 1.00 | 0.00 | H |
| ATOM | 4679 | HE | ARG | A | 289 | 19.426 | 9.436 | 20.722 | 1.00 | 0.00 | H |
| ATOM | 4680 | 1HH1 | ARG | A | 289 | 17.309 | 8.696 | 17.285 | 1.00 | 0.00 | H |
| ATOM | 4681 | 2HH1 | ARG | A | 289 | 18.160 | 10.179 | 17.488 | 1.00 | 0.00 | H |
| ATOM | 4682 | 1HH2 | ARG | A | 289 | 17.216 | 7.206 | 19.153 | 1.00 | 0.00 | H |
| ATOM | 4683 | 2HH2 | ARG | A | 289 | 18.132 | 7.521 | 20.565 | 1.00 | 0.00 | H |
| ATOM | 4684 | N | SER | A | 290 | 23.818 | 11.836 | 23.707 | 1.00 | 0.00 | N |
| ATOM | 4685 | CA | SER | A | 290 | 23.625 | 11.923 | 25.150 | 1.00 | 0.00 | C |
| ATOM | 4686 | C | SER | A | 290 | 23.079 | 13.312 | 25.468 | 1.00 | 0.00 | C |
| ATOM | 4687 | O | SER | A | 290 | 23.754 | 14.324 | 25.244 | 1.00 | 0.00 | O |
| ATOM | 4688 | CB | SER | A | 290 | 24.958 | 11.711 | 25.918 | 1.00 | 0.00 | C |
| ATOM | 4689 | OG | SER | A | 290 | 24.769 | 11.638 | 27.321 | 1.00 | 0.00 | O |
| ATOM | 4690 | H | SER | A | 290 | 24.751 | 11.831 | 23.324 | 1.00 | 0.00 | H |
| ATOM | 4691 | HA | SER | A | 290 | 22.934 | 11.141 | 25.471 | 1.00 | 0.00 | H |
| ATOM | 4692 | 1HB | SER | A | 290 | 25.420 | 10.777 | 25.592 | 1.00 | 0.00 | H |
| ATOM | 4693 | 2HB | SER | A | 290 | 25.675 | 12.503 | 25.690 | 1.00 | 0.00 | H |
| ATOM | 4694 | HG | SER | A | 290 | 25.615 | 11.466 | 27.721 | 1.00 | 0.00 | H |
| ATOM | 4695 | N | PRO | A | 291 | 21.848 | 13.355 | 25.971 | 1.00 | 0.00 | N |
| ATOM | 4696 | CA | PRO | A | 291 | 21.215 | 14.621 | 26.320 | 1.00 | 0.00 | C |
| ATOM | 4697 | C | PRO | A | 291 | 20.277 | 14.491 | 27.528 | 1.00 | 0.00 | C |
| ATOM | 4698 | O | PRO | A | 291 | 19.319 | 13.714 | 27.516 | 1.00 | 0.00 | O |
| ATOM | 4699 | CB | PRO | A | 291 | 20.459 | 15.181 | 25.103 | 1.00 | 0.00 | C |
| ATOM | 4700 | CG | PRO | A | 291 | 19.868 | 13.783 | 24.666 | 1.00 | 0.00 | C |
| ATOM | 4701 | CD | PRO | A | 291 | 20.661 | 12.652 | 25.465 | 1.00 | 0.00 | C |
| ATOM | 4702 | HA | PRO | A | 291 | 21.975 | 15.355 | 26.597 | 1.00 | 0.00 | H |
| ATOM | 4703 | 1HB | PRO | A | 291 | 19.698 | 15.924 | 25.349 | 1.00 | 0.00 | H |
| ATOM | 4704 | 2HB | PRO | A | 291 | 21.144 | 15.584 | 24.357 | 1.00 | 0.00 | H |
| ATOM | 4705 | 1HG | PRO | A | 291 | 19.977 | 13.661 | 23.588 | 1.00 | 0.00 | H |
| ATOM | 4706 | 2HG | PRO | A | 291 | 18.801 | 13.716 | 24.879 | 1.00 | 0.00 | H |
| ATOM | 4707 | 1HD | PRO | A | 291 | 20.690 | 11.613 | 25.178 | 1.00 | 0.00 | H |
| ATOM | 4708 | 2HD | PRO | A | 291 | 20.106 | 12.195 | 26.266 | 1.00 | 0.00 | H |
| ATOM | 4709 | N | ALA | A | 292 | 20.580 | 15.251 | 28.576 | 1.00 | 0.00 | N |
| ATOM | 4710 | CA | ALA | A | 292 | 19.777 | 15.249 | 29.796 | 1.00 | 0.00 | C |
| ATOM | 4711 | C | ALA | A | 292 | 18.630 | 16.253 | 29.672 | 1.00 | 0.00 | C |
| ATOM | 4712 | O | ALA | A | 292 | 18.573 | 17.031 | 28.719 | 1.00 | 0.00 | O |
| ATOM | 4713 | CB | ALA | A | 292 | 20.655 | 15.589 | 30.998 | 1.00 | 0.00 | C |
| ATOM | 4714 | H | ALA | A | 292 | 21.339 | 15.912 | 28.505 | 1.00 | 0.00 | H |
| ATOM | 4715 | HA | ALA | A | 292 | 19.347 | 14.259 | 29.964 | 1.00 | 0.00 | H |
| ATOM | 4716 | 1HB | ALA | A | 292 | 20.092 | 15.551 | 31.931 | 1.00 | 0.00 | H |
| ATOM | 4717 | 2HB | ALA | A | 292 | 21.478 | 14.881 | 31.092 | 1.00 | 0.00 | H |
| ATOM | 4718 | 3HB | ALA | A | 292 | 21.084 | 16.588 | 30.912 | 1.00 | 0.00 | H |
| ATOM | 4719 | N | GLY | A | 293 | 17.722 | 16.232 | 30.642 | 1.00 | 0.00 | N |
| ATOM | 4720 | CA | GLY | A | 293 | 16.577 | 17.135 | 30.637 | 1.00 | 0.00 | C |
| ATOM | 4721 | C | GLY | A | 293 | 16.790 | 18.334 | 31.560 | 1.00 | 0.00 | C |
| ATOM | 4722 | O | GLY | A | 293 | 15.827 | 18.970 | 31.993 | 1.00 | 0.00 | O |
| ATOM | 4723 | H | GLY | A | 293 | 17.825 | 15.586 | 31.411 | 1.00 | 0.00 | H |
| ATOM | 4724 | 1HA | GLY | A | 293 | 16.317 | 17.493 | 29.639 | 1.00 | 0.00 | H |
| ATOM | 4725 | 2HA | GLY | A | 293 | 15.709 | 16.581 | 30.994 | 1.00 | 0.00 | H |
| ATOM | 4726 | N | THR | A | 294 | 18.055 | 18.635 | 31.856 | 1.00 | 0.00 | N |
| ATOM | 4727 | CA | THR | A | 294 | 18.411 | 19.765 | 32.712 | 1.00 | 0.00 | C |
| ATOM | 4728 | C | THR | A | 294 | 17.773 | 19.691 | 34.103 | 1.00 | 0.00 | C |
| ATOM | 4729 | O | THR | A | 294 | 16.557 | 19.960 | 34.217 | 1.00 | 0.00 | O |
| ATOM | 4730 | CB | THR | A | 294 | 18.029 | 21.085 | 32.024 | 1.00 | 0.00 | C |
| ATOM | 4731 | OG1 | THR | A | 294 | 18.639 | 21.124 | 30.740 | 1.00 | 0.00 | O |
| ATOM | 4732 | CG2 | THR | A | 294 | 18.531 | 22.344 | 32.754 | 1.00 | 0.00 | C |
| ATOM | 4733 | H | THR | A | 294 | 18.792 | 18.104 | 31.423 | 1.00 | 0.00 | H |
| ATOM | 4734 | HA | THR | A | 294 | 19.495 | 19.737 | 32.832 | 1.00 | 0.00 | H |
| ATOM | 4735 | HB | THR | A | 294 | 16.950 | 21.179 | 31.892 | 1.00 | 0.00 | H |
| ATOM | 4736 | HG1 | THR | A | 294 | 18.268 | 21.858 | 30.262 | 1.00 | 0.00 | H |
| ATOM | 4737 | 1HG2 | THR | A | 294 | 18.295 | 23.248 | 32.192 | 1.00 | 0.00 | H |
| ATOM | 4738 | 2HG2 | THR | A | 294 | 18.067 | 22.454 | 33.734 | 1.00 | 0.00 | H |
| ATOM | 4739 | 3HG2 | THR | A | 294 | 19.612 | 22.317 | 32.901 | 1.00 | 0.00 | H |
| ATOM | 4740 | N | NMA | A | 295 | 18.543 | 19.329 | 35.133 | 1.00 | 0.00 | N |
| ATOM | 4741 | CA | NMA | A | 295 | 18.068 | 19.211 | 36.504 | 1.00 | 0.00 | C |
| ATOM | 4742 | H | NMA | A | 295 | 19.515 | 19.117 | 34.949 | 1.00 | 0.00 | H |
| ATOM | 4743 | 1HA | NMA | A | 295 | 18.882 | 18.899 | 37.159 | 1.00 | 0.00 | H |
| ATOM | 4744 | 2HA | NMA | A | 295 | 17.685 | 20.165 | 36.866 | 1.00 | 0.00 | H |
| ATOM | 4745 | 3HA | NMA | A | 295 | 17.270 | 18.470 | 36.576 | 1.00 | 0.00 | H |

ENDMDL

TABLE 2
Conservative substitutions

| | | |
|---|---|---|
| ALIPHATIC | Non - polar | G A P |
| | | I L V |
| | Polar - uncharged | C S T M |
| | | N Q |
| | Polar - charged | D E |
| | | K R |
| AROMATIC | | H F W Y |

TABLE 3
Scoring Function of GSK Alpha Ligand bound to the GSK-3 Alpha Model

| Glide Score | Glide Energy | Interacting Residue |
|---|---|---|
| −8.97 | 40.977 | GLU 39 |

TABLE 4
Scoring Function of GSK Beta Ligand bound to GSK-3 Alpha Model

| Glide Score | Glide Energy | Interacting Residues |
|---|---|---|
| −6.78 | −23.47 | PHE 107, VAL 41, CYS 105 |

### REFERENCES

1. Haq S, Choukroun G, Kang Z B, Ranu H, Matsui T, Rosenzweig A, Molkentin J D, Alessandrini A, Woodgett J, Hajjar R, Michael A, Force T, Glycogen synthase kinase-3beta is a negative regulator of cardiomyocyte hypertrophy, (2000) J. Cell. Biol., 151, 117-30
2. Altschul S F, Gish W, Miller W, Myers E W, Lipman D J. Basic local alignment search tool, (1999) J Mol Biol, 215(3), 403-101.
3. Meng, E. C., Shoichet, B. K. and Kuntz, I. D., Automated docking with grid-based energy evaluation, (1992) J. Comp. Chem., 13, 505-524
4. Thornberry, N. A., Chapman, K. T. & Nicholson, D. W., Determination of caspase specificities using a peptide combinatorial library, (2000) Methods Enzymol., 322, 100-110
5. Simon R J, Kania R S, Zuckermann R N, Huebner V D, Jewell D A, Banville S, Ng S, Wang L, Rosenberg S, Marlowe C K 1992, Peptoids: a modular approach to drug discovery. 89(20) 9367-9371
6. Horwell D C. 1995: The ‘peptoid’ approach to the design of non-peptide, small molecule agonists and antagonists of neuropeptides. Trends Biotechnol. April; 13(4):132-134

I/We claim:

1. A three-dimensional structure of Glycogen Synthase Kinase 3-Alpha (GSK3-α) or a variant thereof for screening a modulator that modulates the activity of GSK3-α, wherein said structure is defined by at least a portion of structure co-ordinates structure co-ordinates as shown in Table 1.

2. The three-dimensional structure of claim 1, wherein the GSK3-α comprises the amino acid sequence set forth in SEQ ID NO: 1.

3. The three-dimensional structure of claim 1, wherein said structure co-ordinate comprises a ligand binding domain defined by amino acid residue selected from a group consisting of VAL 49, ASP 106, GLU 39, GLN 91, ASN 92, LYS 89 and combinations thereof.

4. A computer assisted system for producing a three-dimensional structure of GSK3-α or a variant thereof of claim 1, wherein the system comprises:
(a) a computer readable data storage medium comprising a data storage material encoded with computer readable data, wherein said data comprises at least a portion of the structure co-ordinates of Table 1
(b) a working memory for storing instructions for processing said computer readable data;
(c) a central processing unit coupled to said computer readable data storage medium and said working memory for processing said computer readable data into said three-dimensional structure; and
(d) a display coupled to said central processing unit for displaying said three-dimensional structure.

5. A method of generating three-dimensional structure of GSK3-α or a variant thereof wherein said method comprises employing the computer assisted system of claim 4.

6. The method of generating a three-dimensional structure of GSK3-α or a variant thereof of claim 5, wherein the GSK3-α comprises the amino acid sequence set forth in SEQ ID NO: 1.

7. The method of generating a three-dimensional structure of GSK3-α or a variant thereof of claim 5, wherein said structure co-ordinate comprises a ligand binding domain defined by amino acid residue selected from a group consisting of VAL 49, ASP 106, GLU 39, GLN 91, ASN 92, LYS 89 and combinations thereof.

8. A method of screening a modulator for modulating the activity of GSK3-α or a variant thereof, wherein said method comprises using at least a portion of the structure co-ordinates of Table 1.

9. The method of claim 8, wherein said method comprises:
a. employing said structure co-ordinates to identify a modulator of GSK3-α or variant;
b. contacting said modulator with GSK3-α or variant, and
c. determining the modulating activity of said modulator.

10. The method of screening a modulator of claim 8, wherein the GSK3-α comprises the amino acid sequence set forth in SEQ ID NO: 1.

11. The method of screening a modulator of claim 8, wherein said structure co-ordinate comprises a ligand binding domain defined by amino acid residue selected from a group consisting of VAL 49, ASP 106, GLU 39, GLN 91, ASN 92, LYS 89 and combinations thereof.

12. The method of screening a modulator of claim 8, wherein the modulator inhibits GSK3-α activity.

13. The method of screening a modulator of claim 8, wherein the modulator is associated with preventing and/or treating GSK3-α related diseases selected from a group consisting of HIV/AIDS, angiogenesis, breast cancer, colon cancer, type II diabetes, glioblastoma, pancreatic cancer, xaelanoma, prostrate cancer, and schizophrenia.

14. Use of the three-dimensional structure of claim 1 for screening a modulator that modulates the activity of GSK3-α.

15. Use of the three-dimensional structure of claim 1 for designing, selecting and synthesizing a modulator that modulates the activity of GSK3-α.

16. Use of a computer assisted system of claim 4 for producing a three-dimensional structure of GSK3-α or a variant thereof for screening a modulator that modulates the activity of GSK3-α.

17. Use of a computer assisted system of claim 4 for producing a three-dimensional structure of GSK3-α or a variant thereof for designing, selecting and synthesizing a modulator that modulates the activity of GSK3-α.

18. A compound identified by the method of claim 8, wherein said compound modulates the activity of GSK3-α or a variant thereof.
